(19) United States
(12) Patent Application Publication
(10) Pub. No.: US 2023/0235407 A1
(43) Pub. Date: Jul. 27, 2023

(54) SYSTEMS AND METHODS FOR DETECTING TUMOR DNA IN MAMMALIAN BLOOD

(71) Applicant: Gene Solutions Joint Stock Company, Ho Chi Minh City (VN)

(72) Inventors: Hoai Nghia NGUYEN, Ho Chi Minh City (VN); Hoa Giang, Ho Chi Minh City (VN); Minh Duy Phan, Indooroopilly (AU); Le Son Tran, Ho Chi Minh City (VN)

(21) Appl. No.: 17/930,705

(22) Filed: Sep. 8, 2022

Related U.S. Application Data
(60) Provisional application No. 63/373,012, filed on Aug. 19, 2022.

(30) Foreign Application Priority Data
Jan. 25, 2022 (VN) 1-2022-00556 SC

(51) Int. Cl. C12Q 1/6886; C12N 15/10

(52) U.S. Cl. CPC C12Q 1/6886; C12N 15/1065; C12Q 2600/154; C12Q 2523/125

(57) ABSTRACT

Provided are systems and methods for detecting the presence of cancer DNA in blood and for identifying the cancer origin in a test subject. Also provided are systems and methods for monitoring likelihood of cancer recurrence in a subject previously treated for cancer, systems and methods for assessing the efficacy of a cancer treatment in a subject suffering from cancer, and systems and methods for treating cancer in a subject in need thereof. The disclosed systems and methods comprise various elements such as (a) bisulfite treating cell free DNA (cfDNA) from a liquid biopsy sample of the test subject; (b) using the bisulfite treated cfDNA to prepare a first sequencing library for (i) a plurality of specific target genomic regions and (ii) a second sequencing library for a genome from a flow through of the first sequencing library; (c) sequencing the prepared first and second sequencing libraries, thereby producing a corresponding first and second plurality of sequencing results; and (d) analyzing the corresponding first and second plurality of sequencing results; and (e) receiving output from a machine learning model.

### CROSS REFERENCE TO RELATED PATENT APPLICATION

The present disclosure claims the benefit of Vietnam Patent Application No.: 1-2022-00556 SC, filed Jan. 25, 2022, entitled “BIOPSY PROCEDURE FOR DETECTING TUMOR DNA IN MAMMALIAN BLOOD,” and of U.S. Provisional Patent Application No. 63/373,012, filed Aug. 19, 2022, entitled “SYSTEMS AND METHODS FOR DETECTING TUMOR DNA IN MAMMALIAN BLOOD,” which are incorporated herein by reference in their entirety.

### INCORPORATION BY REFERENCE OF TABLES SUBMITTED AS TEXT FILES VIA EFS-WEB

The instant application contains Tables 24 and 25, which have each been submitted as a computer readable text file in ASCII format via EFS-Web and are hereby incorporated in their entirety by reference herein. The text files, which were created on Aug. 15, 2022, are named Table_24_Genomic_Regions_132753-5001 (referred to in the present disclosure as “Table 24”), and Table_25_DNA_probes_132753-5001 (referred to in the present disclosure as “Table 25”) and are respectively 123 kilobytes, and 384 kilobytes in size.

LENGTHY TABLES
The patent application contains a lengthy table section. A copy of the table is available in electronic form from the USPTO web site (https://seqdata.uspto.gov/?pageRequest=docDetail&DocID=US20230235407A1). An electronic copy of the table will also be available from the USPTO upon request and payment of the fee set forth in 37 CFR 1.19(b)(3).

### SEQUENCE LISTING

The instant application contains a Sequence Listing that has been submitted electronically in XML file format and is hereby incorporated by reference in its entirety. The Sequence Listing for this application is labeled “132753-5001-US-Sequence Listing XML”, which was created on Sep. 8, 2022, and is 3,474 kilobytes in size.

### TECHNICAL FIELD

The present disclosure relates to the field of detecting cancer by screening for methylation patterns and size of cell-free DNA (cfDNA), also known as SPOT-MAS (Screening for Presence of Tumor by Methylation and Size of cfDNA) in biological samples.

### BACKGROUND

In 2020, there was 19.2 million new cancer cases worldwide and 9.9 million cancer deaths in 2020. Among the most common types of cancer are liver cancer, lung cancer, breast cancer, stomach cancer, and colorectal cancer.

Patients with cancer found at an early stage have an increased chance of successful treatment. For post-treatment cancer patients, the early detection of cancer recurrence will also help promptly introduce new treatment regimens and increase survival time for patients.

Conventional cancer screening tests, such as endoscopic ultrasound, positron emission tomography and computed tomography (PET/CT), and biochemical tests based on marker proteins have many limitations in terms of sensitivity, specificity, invasiveness, and patient accessibility.

Recently, non-invasive testing (also known as liquid biopsy) has been proven to have potential applications in cancer diagnosis based on specific genetic variation (mutation carrier, variation in the number of genes, methylation, and size variation) of cell-free DNA (cfDNA) molecule of tumor in blood. However, many publications show that the sensitivity and specificity of cancer detection of these methods is limited by the quantity and individualization of these genetic variations. Most of the published tests used only one variable characteristic of the cfDNA molecule, so the sensitivity and specificity of detection is low and inconsistent in different types of cancer.

There are various known methods of early cancer screening based on the liquid biopsy technology such as CancerSEEK, PanSeer, Delfi and GRAIL which are detailed below herein.

CancerSEEK Method

The CancerSEEK method, developed by the Ludwig Cancer Research at Johns Hopkins University (Cohen J D, et al., Science. 2018 Feb. 23; 359(6378):926-930), can detect 8 different types of cancer (including ovarian cancer, liver cancer, stomach cancer, pancreatic cancer, esophageal cancer, colon cancer, lung cancer and breast cancer). The CancerSEEK test method relied on detecting mutations of 16 specific cancer genes and combined with 8 biochemical markers to give conclusions on cancer risk.

16 cancer-related genes were selected based on the somatic mutation dataset in cancer (Catalogue of Somatic Mutations in Cancer—COSMIC). These genes include: TP53, GNAS, PPP2R1A, HRAS, KRAS, AKT1, PTEN, FGFR2, CDKN2A, BRAF, EGFR, APC, FBXW7, PIK3CA, CTNNB1 and NRAS. The presence of the mutation-carrying cfDNA molecule in the blood and combined with information from biochemical markers (CEA, CA-125, CA19-9, PRL, HGF, OPN, MPO and TIMP-1) was used to assess cancer risk.

The CancerSEEK test was performed sequentially in the following main steps:

Step 1: Collect Samples, Extract Genetic Material, Prepare Library and do Sequencing.

Collect 10 ml of blood from patients with ovarian, liver, bronchial, pancreatic, stomach, colorectal, lung or breast cancers that are considered at stage I to III before surgery. The blood sample was then processed to obtain plasma. cfDNA was extracted from plasma using the commercial QIAsymphony DSP Circulating DNA Kit (937556).

DNA from samples of leukemic cells and tissue embedded in paraffin from cancer patients was extracted using the commercial QIAsymphony DSP DNA Midi Kit (937255).

Sequencing library was prepared by amplification of DNA obtained from plasma using 61 primer pairs designed to amplify the regions of interest in 16 genes of 66 to 80 base pairs in length. This library containing DNA regions (16 genes) of interest that have been purified and passed through the second amplification step to include indexing and compatible sequences for Illumina sequencing technology. Library samples were sequenced using an Illumina MiSeq or HiSeq4000 system.

Step 2: Detect Gene Mutations from cfDNA.

Gene mutations must meet one of the following two conditions: (i) being recognized in the COSMIC oncogenic somatic mutation database, or (ii) being predicted to cause inactivation of tumor suppressor genes (including nonsense mutations, addition or deletion of out-of-region fragments, classic splice site mutations). Synonymous mutations except for terminal exon and intron mutations excluding splice area were removed. The highlight of this procedure is the use of readings with unique molecular identifier (UMI) to identify each DNA fragment so that mutations with low variant allele frequency (VAF) can be detected.

Step 3: Evaluate Cancer Marker Protein in Plasma.

The concentration of biochemical markers in plasma samples (CEA, CA-125, CA19-9, PRL, HGF, OPN, MPO and TIMP-1) were measured using the Bioplex 200 platform system (Biorad, Hercules Calif.). The method was based on immunological principles using Luminex magnetic beads (Millipore, Bilerica NY) to help quantify the concentration indirectly through the calibration curve built (with Bioplex Manager 6.0 software) from standard samples and control samples available.

Step 4: Combine Gene and Protein Mutation Analysis to Detect Tumor DNA.

The VAF values of mutations detected in the DNA sample of cancer tissue and white blood cells will be used to build a probabilistic model that predicts the likelihood of mutations coming from tumor DNA. The model for the probability value of a mutation coming from the tumor is called Omega. This Omega value will be combined with the concentration of 8 biochemical markers in plasma to evaluate the probability of a diagnostic blood sample (diagnostic value of CancerSEEK) coming from 1 of 8 types of cancer surveyed. The average sensitivity of the CancerSEEK test for 8 published cancer types ranged from 33% to 98% and the specificity was 99%. In which, the detection sensitivity is less than 70% for 6/8 types of cancer surveyed, the sensitivity of the procedure to detect breast cancer is the lowest, reaching only 33%.

The CancerSEEK test for cancer detection was based on the detection of cfDNA carrying oncogenic mutations. Therefore, in the case of cancer at a very early stage, the amount of cfDNA carrying mutations existing in the blood is too small to be detected. For detection, it is necessary to increase the sequencing capacity many times over, but this significantly increases the cost of implementation. In addition, the majority of detected gene mutations can be benign mutations from white blood cells, mutations caused by cancer cells account for a small part and have individual characteristics. In order to eliminate benign mutations from white blood cells, sequencing is required twice, one for cfDNA and one for DNA from white blood cells. Combined sequencing with biochemical markers requires patients to have two tests simultaneously (with different natures in methodology) to have a basis for concluding cancer condition.

PanSeer Method

The PanSeer method relied on methylation variations of the cfDNA molecule for predictive cancer detection (Chen X, et al., Nat Commun. 2020 Jul. 21; 11(1):3475). The PanSeer test was implemented in the Taizhou Longitudinal (TZL) study, where collecting blood samples started from 2007 to 2016 in Taixing, Gaogang and Hailin counties. A total of 123,115 individuals aged 30-75 participated in the study, with an average condition monitoring of 8.1 years, focusing on researching 5 types of cancer, including stomach, esophagus, colorectal, lung and liver cancer.

DNA regions in the genome with different methylation states among cancer groups and normal people were selected through biological database banks such as: whole genome bisulfite sequencing (WGBS) data, methylation data from a variety of cancer tissues based on RRBS (Reduced Representation Bisulfite Sequencing) data of the research team and data from other scientific publications. From the above resources, a total of 595 DNA regions were selected to investigate the methylation states between cancer patients and healthy people.

The PanSeer test was performed sequentially in the following main steps:

Step 1: Collect Samples and Extract Genetic Material.

10 ml of blood from study subjects was collected and processed for plasma collection. cfDNA was extracted from plasma using the commercial QIAamp Circulating Nucleic Acid Kit (Qiagen, 55114).

DNA from cancer tissue samples and normal human tissue samples were used from the Biochain biobank, DNA sample from the tissue was fragmented into DNA pieces with the size of about 150 nucleotides to simulate the size of cfDNA molecules using the Covaris system (which used physical force to fragment DNA).

Step 2: Bisulfite Processing, Library Preparation and Sequencing.

The cfDNA samples and DNA of tissue samples were treated with bisulfite using the Methylcode Bisulfite Conversion Kit (provided by ThermoFisher, MECOV50). After bisulfite processing, cfDNA molecules will be assigned sequences carrying a unique molecular identifier (UMI). The DNA sequence region of interest (595 regions of the genome containing 11,787 CpG points) was amplified using PCR (Polymerase Chain Reaction) with a specific primer set. The library containing the DNA sequence regions of interest was purified and passed through the second amplification step to include indexing and compatible sequences for Illumina sequencing technology. Library samples were sequenced on the Illumina NextSeq 500 system, paired-end sequencing mode with 300 cycles.

Step 3: Evaluate the Methylation Fraction and Select the DNA Sequence Region of Interest.

The average methylation fraction (AMF) for each sequence region was calculated as the total number of C nucleotides at all CpG sites in the sequence region of interest divided by the total number of C nucleotides and T nucleotides at all CpG sites in this sequence region of interest. This fraction was calculated using the following formula:

$$\frac{{Σ}_{i}^{M} {N}_{C , i}}{{Σ}_{i}^{M} ( {N}_{C , i} + {N}_{T , i} )}$$

where
i: The i^{th} CpG site in the region of interest;
M: Total number of CpG in the sequence region of interest;
N_{T,i}: Number of T nucleotides observed at the i^{th} CpG site; and
N_{C,i}: Number of C nucleotides observed at the i^{th} CpG site.

AMF fractions in each sequence region of interest were compared between cancerous and healthy tissue samples. The dataset of 160 cancer tissue samples and 40 healthy tissue samples from Biochain was used to select DNA regions with different AMF values between these 2 groups of samples. The difference of AMF was tested using t-test (with Benjamini-Hochberg correction). Statistical test results showed that a total of 477 DNA regions (containing 10,613 CpG points) had clearly different AMF between the two groups of samples.

Step 4: Build an Algorithm Model to Predict Cancer Detection.

To distinguish incoming plasma samples of cancer patients from the ones of healthy individuals, the PanSeer test used a logistic regression (LR) classification model that was built on the training dataset of average methylation fraction (AMF) of 477 regions of samples known as cancerous or non-cancerous samples, accompanied by a cross validation model to avoid overfitting during algorithm training. This classification model was then evaluated on the model evaluation dataset.

The limitation of the PanSeer method is that it can only distinguish between cancerous or healthy samples, in case of positive samples (classified as cancerous), the patient needs to have other blood tests and tumor monitoring with imaging tests to determine the tissue of origin.

DELFI Method

The analytical DELFI test evaluated the length of cfDNA molecules obtained from blood, to predict whether the analyzed blood sample contains the cfDNA molecule of cancer cells (Cristiano S, et al., Nature. 2019 June; 570(7761):385-389; Mathios D, et al., Nat Commun. 2021 Aug. 20; 12(1):5060). Because size-specific variations of DNA occur across the entire chromosome of cancer cells, this procedure can overcome sensitivity limitations compared with mutational markers that occur at individual sites. The DELFI procedure was implemented on 215 healthy volunteers and 208 patients in 7 cancer groups including breast cancer, colorectal cancer, lung cancer, ovarian cancer, prostate cancer, stomach cancer and gallbladder cancer.

The DELFI procedure was performed sequentially in the following main steps:

Step 1: Collect Samples and Extract Genetic Material.

10 ml of blood from study subjects was collected and processed for plasma collection and monocyte subclass. cfDNA was extracted from plasma using the commercial QIAamp Circulating Nucleic Acid Kit (Qiagen, 55114). The quality of cfDNA was assessed using the Bioanalyzer 2100 electrophoresis system (Agilent Technologies).

Step 2: Create Sequencing Library.

The cfDNA sample was carried out to prepare the sequencing library using commercially available kits (NEBNext DNA library Prep kit) suitable for the Illumina sequencing technology. The cfDNA library was sequenced on Hiseq 2000/2500 system (Illumina), set to paired-end sequencing mode with 100 cycles. The DELFI test used genome-wide sequencing and DNA region-sequencing technology to evaluate abnormalities in the length of cfDNA molecules.

Step 3: Evaluate Variation in Length of cfDNA.

Sequencing data includes reads of paired-end sequences of cfDNA molecule. Typically, a cfDNA fragment will range from 50 bp to 200 bp in length. For cost savings, only sequencing about 50 bp in length was performed at each end of the cfDNA fragment. The sequencing results are put through a processing procedure to locate 2 ends of the cfDNA fragment on the original genome, thereby determining the length of that cfDNA fragment. The length of this cfDNA fragment will be used to distinguish between cancer and healthy samples. In addition, the sequencing results also give indication of mutations appearing on cfDNA and DNA from leukocytes, aiding to perform the following steps in building the predictive model.

Step 4: Build a Predictive Model to Detect Cancer Samples in Two Groups of People.

The predictive model was built based on the anomalous attributes in the length of the tumor-derived cfDNA molecule. These attributes used to train the algorithm include:

The length difference between cfDNA fragments carrying mutations from the tumor and those without mutations was evaluated using Welch's two-sample t-test on 100 mutation-carrying fragments.
The length difference of cfDNA between cancer patients and healthy subjects showed that, on average, samples from healthy subjects had longer cfDNA fragments than cancer samples (Wilcoxon rank sum test).
The length difference of cfDNA among samples at different cancer stages and after cancer treatment.

The “Gradient tree boosting model” machine learning algorithm model was applied on 208 patients (54 breast cancer patients, 27 colorectal cancer patients, 12 lung cancer patients, 28 uterine cancer patients, 34 pancreatic cancer patients, 27 stomach cancer patients and 26 bile duct cancer patients) and 215 healthy subjects. To build a machine learning model, the algorithm divided the data into ten parts, and the algorithm used 9 parts in turn to find the differences between two groups of samples in the above 504 regions, selected those regions as characteristics to identify groups of sick and healthy people, and then rechecked the rest of samples. Since there are ten parts, the algorithm performed this calculation 10 times and found the best characteristics to help predict the two groups of samples. The DELFI model achieved a sensitivity of 80% and a specificity of 95%. This model also identified the location of cancer and achieved an accuracy of 61%. When combined with mutations detected on cell-free DNA, the model achieved a sensitivity of 91% and a specificity of 98%.

The DELFI procedure achieved a high specificity-sensitivity in patients with stage III (91%) and stage IV (82%) cancer but a lower sensitivity in patients with stage I (73%) and stage II (78%) cancer with a specificity of 95%. In addition, the procedure achieved different sensitivities, depending on the type of cancer, the highest is 100% in lung cancer, and the lowest is 70% in breast cancer and 71% in pancreatic cancer. The effectiveness of the DELFI model has not been proven through clinical trials with large samples.

GALLERI® Method

GALLERI (Grail) is a test to screen for >50 types of early-stage cancers based on specific methylation variation of tumor DNA released into the bloodstream (Liu M C, et al., Ann Oncol. 2020 June; 31(6):745-759; Liu L, et al., Ann Oncol. 2018 Jun. 1; 29(6):1445-1453). These variations are often related to mechanisms that control the expression of many oncogenes and occur at an early stage in tumor formation and development. Using data of potential methylation markers from the whole genome sequencing and the human genome data system associated with all common cancers (The Cancer Genome Atlas—TCGA), the research team designed a hybrid capture detector that covers more than 100,000 target sequence regions and over 1,000,000 CpG.

The GALLERI procedure comprises the following main steps:

Step 1: Collect Samples and Extract Genetic Material.

cfDNA was obtained from 10 ml of blood in cancer patients and healthy subjects in the same way as the above procedures.

Step 2: Create Sequencing Library.

The sequencing library was prepared by performing bisulfite transformation of cfDNA fragments extracted from plasma. The cfDNA was then tagged with the reads needed for sequencing by the Illumina system and identifiers before being hybrid captured by the probes designed for 100,000 targets mentioned above. The entire cfDNA library was 150 bp sequenced from 2 ends of an Illumina's NovaSeq system. Target sequence fragments were aligned with the standard genome to determine the methylation status of known CpGs. Then, based on data on methylation levels at target regions in healthy people and cancer patients, the team built models to assess the probability of this sequence from cancer patients.

Step 3: Build a Model to Distinguish Cancer Samples and Tumor Tissue Origins.

The data was randomly divided into 2 sets including training set and control set so that the proportion of cancer samples and control samples was equivalent. In order to find the origin of sequence fragments, a model was built to detect methylation markers in each target sequence region, comparing them with the markers specific to each cancer type. Finally, a set of 2 machine learning models based on logistic regression algorithms are applied for 2 purposes: i) to distinguish the cancer group and the control group; ii) to determine the origin of tumor DNA. The effectiveness of this model combination has been verified in clinical trials. Specifically, a recent study applying this method of the author group with the participation of about 4,000 volunteers (including 2800 cancer patients and 1200 healthy people) achieved an average sensitivity of 51.5% at a specificity of 99.5%. For some common cancers, sensitivity was improved at 67.6%.

The GALLERI test is a non-invasive method to detect cancer at early stages (I-IIIA). Moreover, this method can also distinguish tumor origin with high accuracy. However, due to the requirements of the analytical method, rather large sequencing capacity (30,000×) increases testing costs and reduces patient accessibility. Considering the current situation, when the cost of next-generation sequencing is still high for developing countries, reducing requirements for the depth of the sequencing method will contribute to making this research direction easier to access and soon achieve practical results.

Despite the recent development of non-invasive testing for early detection of cancer, there remains a need in the art for systems and methods to overcome the limitations of existing testing procedures. The present disclosure addresses this need.

### SUMMARY OF THE INVENTION

Disclosed herein are systems and methods for detecting tumor DNA in mammalian blood cells by screening for methylation patterns and size of cell-free DNA (cfDNA).

In one aspect, the present disclosure provides methods for detecting the presence of a cancer and for identifying the cancer origin in a test subject.

The disclosed methods comprise the steps of: (a) bisulfite treating cell free DNA (cfDNA) from a liquid biopsy sample of the test subject; (b) using the bisulfite treated cfDNA to prepare (i) a first sequencing library for a plurality of specific target genomic regions and (ii) a second sequencing library for a genome from a flow through of the first sequencing library; (c) sequencing the prepared first and second sequencing libraries, thereby producing a corresponding first and second plurality of sequencing results; (d) analyzing the corresponding first and second plurality of sequencing results by measuring:
i. a plurality of site specific methylation densities, using the first plurality of sequencing results, for the plurality of specific target genomic regions of the test subject relative to a plurality of site specific methylation densities determined using a plurality of sequencing results for the plurality of specific target genomic regions in a plurality of liquid biopsies obtained from a cohort of healthy subjects;
ii. a methylation density for the genome, using the second plurality of sequencing results, of the test subject relative a methylation density for the genome determined from a plurality of genome wide sequencing results for the plurality of liquid biopsies obtained from the cohort of healthy subjects;
iii. a respective copy number of cfDNA in a plurality of first bins across the genome, using the second plurality of sequencing results, of the test subject relative to a respective copy number of cfDNA in the plurality of first bins across the genome determined using a plurality of genome wide sequencing results of the plurality of liquid biopsies obtained from the cohort of healthy subjects, and
iv. a fragment size pattern distribution of cfDNA across the genome, using the second plurality of sequence results, of the test subject relative to a fragment size distribution of cfDNA determined using a plurality of genome sequencing results for a plurality of liquid biopsies obtained from a cohort of a healthy subject; and

(e) responsive to inputting into a combination model each of the analyzed sequencing results from (d)(i)-(d)(iv), receiving as output from the model:
i. a categorical indication of a presence or absence of the cancer in the test subject, and in the case where the model determines presence of the cancer in the test subject, an origin of the cancer.

In some embodiments, the plurality of specific target genomic regions comprises at least 200, at least 250, at least 300, at least 350, at least 400, at least 450, at least 500 or more cancer specific regions. In some embodiments, the plurality of specific target genomic regions comprises between 400 and 500 cancer specific gene regions. In some embodiments, wherein the plurality of specific target genomic regions consists of between 17,500 and 18,500 CpG sites. In some embodiments, the plurality of specific target genomic regions comprises at least five nucleic acid sequences selected from SEQ ID NOs: 1-450. In some embodiments, the plurality of specific target genomic regions comprises at least 50 nucleic acid sequences selected from SEQ ID NOs: 1-450. In some embodiments, the plurality of specific target genomic regions comprises at least 200 nucleic acid sequences selected from SEQ ID NOs: 1-450. In some embodiments, the plurality of specific target genomic regions comprises at least 300 nucleic acid sequences selected from SEQ ID NOs: 1-450. In some embodiments, each respective target genomic region in the plurality of specific target genomic regions encompasses a sequence selected from SEQ ID NOs: 1-450.

In some embodiments, at least 20 respective cancer specific genomic regions in the plurality of cancer specific genomic regions encompass an oncogene and/or a tumor suppressor gene listed in Table 23. In some embodiments, the plurality of cancer specific genomic regions, their respective chromosomal locations and their sequences (SEQ ID Nos: 1-450) are listed in Table 24.

In some embodiments, the plurality of specific target genomics regions is captured by a set of DNA probes. In some embodiments, the set of DNA probes comprises DNA fragments with a size ranging between 40 base-pair (bp) and 50 bp, between 51 bp and 60 bp, between 61 bp and 70 bp, between 71 bp and 80 bp, between 81 bp and 90 bp, between 91 bp and 100 bp, between 101 bp and 110 bp, between 111 bp and 120 bp, between 121 bp and 130 bp, between 131 bp and 140 bp, between 141 bp and 150 bp, between 151 bp and 160 bp, between 161 bp and 170 bp, between 171 bp and 180 bp, between 181 bp and 190 bp, between 191 bp and 200 bp or more. In some embodiments, the set DNA probes comprises DNA fragments with a size ranging between 111 bp and 120 pb or between 121 bp and 130 bp. In some embodiments, the set of DNA probes consists of between 400 DNA probes and 500 DNA probes, between 501 DNA probes and 1000 DNA probes, between 1001 DNA probes and 1500 DNA probes, between 1501 DNA probes and 2000 DNA probes, between 2001 DNA probes and 2100 DNA probes, between 2101 DNA probes and 2150 DNA probes, between 2151 DNA probes and 2200 DNA probes, between 2201 DNA probes and 2250 DNA probes, between 2251 DNA probes and 2300 DNA probes, between 2301 DNA probes and 2350 DNA probes, between 2351 DNA probes and 2400 DNA probes, between 2401 DNA probes and 2450 DNA probes, between 2451 DNA probes and 2500 DNA probes, between 2501 DNA probes and 3000 DNA probes, between 3001 DNA probes and 3500 DNA probes, or between 3501 DNA probes and 4000 DNA probes, or more. In some embodiments, the set DNA probes consists of between 2201 DNA probes and 2250 DNA probes or between 2251 DNA probes and 2300 DNA probes. In some embodiments, the set of DNA probes comprises at least 10 nucleic acid sequences selected from SEQ ID NOs: 451-2700. In some embodiments, the set of DNA probes comprises at least 100 nucleic acid sequences selected from SEQ ID NOs: 451-2700. In some embodiments, the set of DNA probes comprises at least 200 nucleic acid sequences selected from SEQ ID NOs: 451-2700. In some embodiments, the set of DNA probes, their respective chromosomal locations, their sequences (SEQ ID NOs: 451-2700) and size (120 pb) are listed in Table 25.

In some embodiments, the first sequencing library is prepared for paired-end sequencing.

In some embodiments, the plurality of specific target genomic regions have a different methylation percentage between the test subject and the cohort of healthy subjects. In some embodiments, the plurality of specific target genomic regions have a methylation percentage higher in the test subject as compared to the cohort of healthy subjects.

In some embodiments, the methylation in the test subject is about two-fold higher than the methylation in the cohort of healthy subjects.

In some embodiments, the second sequencing library comprises universal adapter sequences. In some embodiments, the genomic sequencing comprises rolling circle sequencing or MGI-DNBseq sequencing.

In some embodiments, the analysis of the sequencing results from (d)(ii)-(d)(iv) is performed by measuring non-duplicating fragments in the genome. In some embodiments, the genome comprises 22 chromosomes.

In some embodiments, the methylation density for the genome in (d)(ii) is determined for each respective second bin region is between 2500 second bin regions and 3000 second bin regions. In some embodiments, each respective second bin region consists of between 800,000 nucleotides and 1,200,000 nucleotides. In some embodiments, the measuring of the methylation density identifies second bin regions in the between 2500 second bin regions and 3000 second bin regions that are differentially methylated between the test subject suffering and the cohort of healthy subjects. In some embodiments, the methylation density in each respective second bin region is evaluated based on a Z score value.

In some embodiments, the plurality of first bins is between 2500 first bin regions and 3000 first bins. In some embodiments, each first bin consists of between 800,000 nucleotides and 1,200,000 nucleotides.

In some embodiments, the measuring of respective copy number of cfDNA identifies a subset of first bins in the plurality of first bins with variation in the number of copies of DNA per bin between the test subject and the cohort of healthy subjects. In some embodiments, the variation in the number of copies of DNA between the test subject and the cohort of healthy subjects in each first bin is evaluated based on a Z score value. In some embodiments, the Z score identifies regions of instability in the genome.

In some embodiments, the measuring of the fragment size pattern distribution of cfDNA across the genome comprises determining a fragment size pattern distribution in each third bin in a plurality of third binds, wherein the plurality of third bins consists of between 500 third bins and 600 third bins. In some embodiments, each respective third bin consists of between 4.5 million nucleotides (4.5 megabases) and 5.5 million nucleotides (5.5 megabases).

In some embodiments, the measuring of the fragment size pattern distribution of cfDNA identifies a subset of third bins with a variation in the fragment size pattern distribution of cfDNA per bin between the test subject and the cohort of healthy subjects. In some embodiments, the variation in the fragment size pattern distribution of the cfDNA in each third bin in the plurality of third bins is evaluated based on cfDNA fragment length ratio (RF) value. In some embodiments, the RF value identifies presence of cancer, wherein cfDNA fragment length released from tumor cells from the test subject is shorter than cfDNA fragment length released by cells of the cohort of healthy subjects. In some embodiments, the cohort of healthy subjects consists of between 5 and 50 healthy subjects, between 5 and 100 healthy subjects, between 5 and 1000 healthy subjects, between 5 and 5000 healthy subjects, between 50 and 500 healthy subjects, between 50 and 1000 healthy subjects, between 50 and 5000 healthy subjects, between 100 and 500 healthy subjects, between 100 and 1000 healthy subjects, between 100 and 5000 healthy subjects, between 500 and 1000 healthy subjects, or between 500 and 5000 healthy subjects, or more.

In some embodiments, the liquid biopsy sample comprises a body fluid, blood, or plasma. In some embodiments, the origin of the cancer comprises colorectal cancer (CRC), liver cancer, lung cancer, breast cancer, or gastric cancer. In some embodiments, the subject is a human.

In some embodiments, the model is a composite model comprising four attribute models and a combination model, wherein each respective attribute model in the four attribute models produces an initial categorical classification upon input of a different one of the analyzed sequencing results from (d)(i)-(d)(iv), and wherein the combination model combines the respective categorical indication of the presence or absence of cancer in the test subject of each attribute model in the four attribute models by a weighted combination of the four attribute models. In some embodiments, the combination model is a logistic regression combined linear model of the four attribute models, in which each of the four attribute models is independently assigned a different probability weight. In some embodiments, the model comprises at least 100 parameters. In some embodiments, the model comprises a logistic regression, a deep neural network, a fully connected neural network, a convolutional neural network, a graph based neural network, or a support vector machine. In some embodiments, the deep neural network specifies a tissue for cancer origin.

In one aspect, the present disclosure provides methods for monitoring likelihood of cancer recurrence in a subject previously treated for cancer. The disclosed methods comprise the steps (a)-(e) as described above herein, wherein the detection of a cancer is indicative of cancer recurrence and need of resuming treatment to the subject.

In another aspect, the present disclosure provides methods for assessing the efficacy of a cancer treatment in a subject suffering from cancer. The disclosed methods comprise the steps (a)-(e) as described above herein, wherein the detection of a cancer is indicative of efficacy of treatment and need of continuing, modifying or discontinuing treatment of the subject.

In a further aspect, the present disclosure provides methods treating cancer in a subject in need thereof. The disclosed methods comprise the steps (a)-(e) as described above herein, wherein the detection of a cancer and the identification of the cancer origin are indicative of the need to treat the subject and the type of treatment that is the most efficacious given the cancer origin.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIGS. 1A, 1i, and 1C collectively illustrate a computer system for detecting tumor DNA in mammalian blood, in accordance with an embodiment of the present disclosure.

FIGS. 2A, 2B, and 2C, collectively provide a flow chart illustrating exemplary methods for detecting tumor DNA in mammalian blood, in which dashed boxes indicate optional features, in accordance with some embodiments of the present disclosure.

FIG. 3 shows a schematic diagram of the protocol for detecting tumor DNA in peripheral blood using the SPOT-MAS test procedure according to an embodiment of the present disclosure.

FIG. 4 illustrates 353 sequence regions out of 450 target sequence regions to be surveyed with statistically significant differences in methyl density (p-value≤0.05) between a liver cancer group and a healthy group specified when performing the SPOT-MAS test procedure according to an embodiment of the present disclosure.

FIG. 5 is a heatmap illustrating the clustering of target sequence regions between liver cancer patients and healthy subjects obtained after performing the SPOT-MAS test procedure according to an embodiment of the present disclosure.

FIG. 6 illustrates the results of analysis of mean values of methylation density on all survey bins belonging to 22 chromosomes of patients with colorectal cancer (CRC) and a group of healthy people who underwent the SPOT-MAS test procedure according to an embodiment of the present disclosure.

FIG. 7 shows a graph illustrating the hypomethylation change (decreased methyl ratio) on all the ‘bin’ regions of 22 chromosomes of the CRC group compared with the healthy group who underwent the SPOT-MAS test procedure according to an embodiment of the present disclosure.

FIG. 8 shows a graph illustrating the percentage of bins that are determined to be hypomethylated between the group of colorectal cancer patients and the group of healthy people who underwent the SPOT-MAS test procedure according to an embodiment of the present disclosure.

FIG. 9 is a chart illustrating the variation of DNA copy number on all 22 chromosomes of the group of colorectal cancer patients and the group of healthy people who underwent the SPOT-MAS test procedure according to an embodiment of the present disclosure.

FIG. 10 is a chart comparing the percentage (%) of CNA bins in the total number of surveyed bins between the CRC group and the healthy group who underwent the SPOT-MAS test procedure according to an embodiment of the present disclosure.

FIG. 11 is a histogram showing the size distribution of cfDNA fragments in colorectal cancer samples and healthy subjects who underwent the SPOT-MAS test procedure according to an embodiment of the present disclosure.

FIG. 12 is a chart showing comparison of the ratio of small size (<=150) cfDNA fragments to large size (>150 bp) ones between CRC patients and healthy people who underwent the SPOT-MAS test procedure according to an embodiment of the present disclosure.

FIG. 13 is a chart illustrating the results of evaluating the effectiveness of blood sample classification of four groups of patients with liver cancer, lung cancer, colorectal cancer, and breast cancer with blood samples of healthy people who underwent the SPOT-MAS test procedure according to an embodiment of the present disclosure.

FIG. 14 is a diagram showing the test results of blood samples from patients with liver cancer, lung cancer, colorectal cancer, and breast cancer using the SPOT-MAS test procedure according to an embodiment of the present disclosure.

FIG. 15 is a diagram depicting a Deep Neural Network (DNN) model for determining the tissue of origin for cancer. The model is built from epigenetic signatures including GC methylation, fragment length and motif end.

FIG. 16 is a table depicting the tissue of origin for cancer classification performance of DNN model. The model provided probability scores of 5 cancer types (breast cancer, gastric cancer, colorectal cancer, liver cancer and lung cancer) and probability scores of unknown cancer.

### DETAILED DESCRIPTION OF THE PRESENT DISCLOSURE

The present disclosure relates to the medical field, specifically relating to a liquid biopsy procedure based on screening for the presence of tumor(s) by methylation and size of cell-free DNA (cfDNA), also known as SPOT-MAS (Screening for Presence of Tumor by Methylation and Size of cfDNA) test procedure to detect tumor DNA in blood for application in screening and early detection of cancer and monitor the likelihood of post-treatment recurrence in mammals.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for screening liquid biopsy samples for detecting cancer based on the methylation and size of cfDNA, also known as SPOT-MAS (Screening for Presence Of Tumor by Methylation and Size of cfDNA) test procedure.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

As used herein, the term “about” or “approximately” mean within an acceptable error range for the particular value as determined by one of ordinary skill in the art, which depends in part on how the value is measured or determined, e.g., the limitations of the measurement system. For example, in some embodiments, the term “about” refers to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. In some embodiments “about” mean within 1 or more than 1 standard deviation, per the practice in the art. In some embodiments, “about” means a range of 20%, +10%, +5%, or +1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. In some embodiments, the term “about” refers to ±10%. In some embodiments, the term “about” refers to +5%.

As used herein, the terms “control,” “control sample,” “reference,” “reference sample,” “normal,” and “normal sample” describe a sample from a non-diseased tissue. In some embodiments, such a sample is from a subject that does not have a particular condition (e.g., cancer). In other embodiments, such a sample is an internal control from a subject, e.g., who may or may not have the particular disease (e.g., cancer), but is from a healthy tissue of the subject. For example, where a liquid or solid tumor sample is obtained from a subject with cancer, an internal control sample may be obtained from a healthy tissue of the subject, e.g., a white blood cell sample from a subject without a blood cancer or a solid germline tissue sample from the subject. Accordingly, a reference sample can be obtained from the subject or from a database, e.g., from a second subject who does not have the particular disease (e.g., cancer).

As used herein the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

A “disease” is a state of health of an animal where the animal cannot maintain homeostasis, and where if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the terms “biological sample,” “patient sample,” and “sample” are interchangeably used and refer to any sample taken from a subject, which can reflect a biological state associated with the subject. In some embodiments such samples contain cell-free nucleic acids such as cell-free DNA. In some embodiments, such samples include nucleic acids other than or in addition to cell-free nucleic acids. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject. A biological sample can include any tissue or material derived from a living or dead subject. A biological sample can be a cell-free sample. A biological sample can comprise a nucleic acid (e.g., DNA or RNA) or a fragment thereof. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample). In some embodiments, a biological sample is derived from one tissue type (e.g., from a single organ such as breast, lung, prostate, colorectal, renal, uterine, pancreatic, esophageal, lymph, ovarian, cervical, epidermal, thyroid, bladder, or gastric). In some embodiments, a biological sample is derived from a two or more tissue types (e.g., a combination of tissue from two or more organs). In some embodiments, a biological sample is derived from one or more cell types (e.g., cells originating from a single organ or from a predetermined set of organs).

As used herein, the term “tissue” corresponds to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “liquid biopsy” refers to a technique performed on non-solid biological tissue by detecting cells and cell-free DNA that have entered body fluids, primarily blood. Liquid biopsy refers to real-time monitoring of dynamic changes of the disease by detecting free tumor cells, cfDNA, exosomes, etc. This technique has great application value as a tool for early diagnosis of diseases, monitoring of progression in real time, observation and evaluation of treatment effect, prognosis assessment and metastasis risk analysis with the added benefit of being non-invasive and flexible for repeated tumor sampling.

As used herein, the term “liquid biopsy sample” refers to a liquid sample obtained from a subject that includes cell-free DNA. Examples of liquid biopsy samples include, but are not limited to, blood, whole blood, plasma, serum, urine, cerebrospinal fluid, fecal material, saliva, sweat, tears, pleural fluid, pericardial fluid, or peritoneal fluid of the subject. In some embodiments, a liquid biopsy sample is a cell-free sample, e.g., a cell free blood sample. In some embodiments, a liquid biopsy sample is obtained from a subject with cancer. In some embodiments, a liquid biopsy sample is collected from a subject with an unknown cancer status, e.g., for use in determining a cancer status of the subject. Likewise, in some embodiments, a liquid biopsy is collected from a subject with a non-cancerous disorder, e.g., a cardiovascular disease. In some embodiments, a liquid biopsy is collected from a subject with an unknown status for a non-cancerous disorder, e.g., for use in determining a non-cancerous disorder status of the subject.

As used herein, the term “cell-free DNA” and “cfDNA” interchangeably refer to DNA fragments that circulate in a subject's body (e.g., bloodstream) and originate from one or more healthy cells and/or from one or more cancer cells. These DNA molecules are found outside cells, in bodily fluids such as blood, whole blood, plasma, serum, urine, cerebrospinal fluid, fecal material, saliva, sweat, sweat, tears, pleural fluid, pericardial fluid, or peritoneal fluid of a subject, and are believed to be fragments of genomic DNA expelled from healthy and/or cancerous cells, e.g., upon apoptosis and lysis of the cellular envelope. In some embodiments cell-free DNA (cfDNA) refers to degraded DNA fragments ranging from 50 bp to 200 bp in size that can be derived from both normal and diseased cells. cfDNA can be used to describe various forms of DNA that circulate freely in body fluids including, but not limited to, blood, sputum, urine, cerebrospinal fluid, or ascites from dead and necrosis cells. These different forms of DNA include circulating tumor DNA (ctDNA), circulating cell-free mitochondrial DNA (ccf mtDNA) and cell-free fetal DNA (cffDNA). Variations in concentrations, integrity, genetics, and epigenetics in cfDNA can suggest pathological conditions of the body, such as inflammatory diseases, autoimmune diseases, stress or even malignancies. High levels of cfDNA are commonly observed in many types of cancer, especially in advanced cancers. Clinical detection of cfDNA is a major application of liquid biopsy and is used for early diagnosis of clinical tumors, real-time monitoring of progression, observation and assessment of treatment efficacy, and prognosis assessment and metastatic risk analysis of cancer.

As used herein, the term “fragment” is used interchangeably with “nucleic acid fragment” (e.g., a DNA fragment), and refers to a portion of a polynucleotide or polypeptide sequence that comprises at least three consecutive nucleotides. In the context of sequencing of cell-free nucleic acid molecules found in a biological sample, the terms “fragment” and “nucleic acid fragment” interchangeably refer to a cell-free nucleic acid molecule that is found in the biological sample or a representation thereof. In such a context, sequencing data (e.g., sequence reads from whole genome sequencing, targeted sequencing, etc.) are used to derive one or more copies of all or a portion of such a nucleic acid fragment. Such sequence reads, which in fact may be obtained from sequencing of PCR duplicates of the original nucleic acid fragment, therefore “represent” or “support” the nucleic acid fragment. There may be a plurality of sequence reads that each represent or support a particular nucleic acid fragment in the biological sample (e.g., PCR duplicates). In some embodiments, nucleic acid fragments can be considered cell-free nucleic acids. In some embodiments, sequence reads from PCR duplicates can be misleading; for example, when the abundance level of a particular cell-free nucleic acid molecule needs to be determined. In such embodiments, only one copy of a nucleic acid fragment is used to represent the original cell-free nucleic acid molecule (e.g., duplicates are removed through molecular identifiers that are attached to the cell-free nucleic acid molecule during the library preparation process). In some embodiments, methylation sequencing data can be used to further distinguish these nucleic acid fragments. For example, two nucleic acid fragments that share identical or near identical sequences may still correspond to different original cell-free nucleic acid molecules if they each harbor a different methylation pattern.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where. n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments, n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

The term, “polynucleotide” includes cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semisynthetic nucleotide bases. Also, included within the scope of the invention are alterations of a wild type or synthetic gene, including but not limited to deletion, insertion, substitution of one or more nucleotides, or fusion to other polynucleotide sequences.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” or “protein” are used interchangeably, and refer to a compound comprised of amino acid residues covalently linked by peptide bonds. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs and fusion proteins, among others. The polypeptides include natural peptides, recombinant peptides, synthetic peptides or a combination thereof. A peptide that is not cyclic will have a N-terminal and a C-terminal. The N-terminal will have an amino group, which may be free (i.e., as a NH2 group) or appropriately protected (for example, with a BOC or a Fmoc group). The C-terminal will have a carboxylic group, which may be free (i.e., as a COOH group) or appropriately protected (for example, as a benzyl or a methyl ester). A cyclic peptide does not have free N- or C-terminal, since they are covalently bonded through an amide bond to form the cyclic structure. Amino acids may be represented by their full names (for example, leucine), 3-letter abbreviations (for example, Leu) and 1-letter abbreviations (for example, L). The structure of amino acids and their abbreviations may be found in the chemical literature, such as in Stryer, “Biochemistry”, 3rd Ed., W. H. Freeman and Co., New York, 1988. tLeu represents tert-leucine. neo-Trp represents 2-amino-3-(1H-indol-4-y])-propanoic acid. DAB is 2,4-diaminobutyric acid. Orn is ornithine. N-Me-Arg or N-methyl-Arg is 5-guanidino-2-(methylamino) pentanoic acid.

The terms “subject”, “patient”, “individual”, and the like are used interchangeably herein, and refer to any animal, or cells thereof whether in vitro or in situ, amenable to the methods described herein. In certain non-limiting embodiments, the patient, subject or individual is a human. Non-human mammals include, for example, livestock and pets, such as ovine, bovine, porcine, canine, feline and murine mammals. Preferably, the subject is human. The term “subject” does not denote a particular age or sex. In some embodiments, the subject from whom a sample is taken, or is treated by any of the methods or compositions described herein can be of any age and can be an adult, infant or child. In some cases, the subject, e.g., patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old). A particular class of subjects, e.g., patients that can benefit from a method of the present disclosure is subjects, e.g., patients over the age of 40.

Another particular class of subjects, e.g., patients that can benefit from a method of the present disclosure is pediatric patients, who can be at higher risk of chronic heart symptoms. Furthermore, a subject, e.g., patient from whom a sample is taken, or is treated by any of the methods or compositions described herein, can be male or female.

The term “measuring” according to the present invention relates to determining the amount or concentration, preferably semi-quantitatively or quantitatively. Measuring can be done directly.

As used herein the term “amount” refers to the abundance or quantity of a constituent in a mixture.

The term “concentration” refers to the abundance of a constituent divided by the total volume of a mixture. The term concentration can be applied to any kind of chemical mixture, but most frequently it refers to solutes and solvents in solutions.

As used herein, the term “primers” or “probes” refers to DNA strands which can prime the synthesis of DNA. DNA polymerase cannot synthesize DNA de novo without primers: it can only extend an existing DNA strand in a reaction in which the complementary strand is used as a template to direct the order of nucleotides to be assembled. The synthetic oligonucleotide molecules which are used in a polymerase chain reaction (PCR) as primers are referred to as “primers”.

As used herein, the term “methylation status” (also called methylation profile) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide other than cytosine; however, these are rarer occurrences. In this present disclosure, methylation is discussed in reference to CpG sites for the sake of clarity. Anomalous cfDNA methylation can identified as hypermethylation or hypomethylation, both of which may be indicative of cancer status. As is well known in the art, DNA methylation anomalies (compared to healthy controls) can cause different effects, which may contribute to cancer. Various challenges arise in the identification of anomalously methylated cfDNA fragments. First, determining a subject's cfDNA to be anomalously methylated only holds weight in comparison with a group of control subjects, such that if the control group is small in number, the determination loses confidence with the small control group. Additionally, among a group of control subjects' methylation status can vary which can be difficult to account for when determining a subject's cfDNA to be anomalously methylated. On another note, methylation of a cytosine at a CpG site causally influences methylation at a subsequent CpG site. Those of skill in the art will appreciate that the principles described herein are equally applicable for the detection of methylation in a non-CpG context, including non-cytosine methylation.

As used herein, the terms “cut-off” or “threshold” or “reference” are used interchangeably, and refer to a value that is used as a constant and unchanging standard of comparison. In some embodiments, the terms “cutoff” and “threshold” refer to predetermined numbers used in an operation. In one example, a cutoff size refers to a size above which fragments are excluded. In some embodiments, a threshold value is a value above or below which a particular classification applies. Either of these terms can be used in either of these contexts.

As used herein, the term “ratio” refers to any comparison of a first metric X, or a first mathematical transformation thereof X′ (e.g., measurement of a number of units of a genomic sequence in a first one or more biological samples or a first mathematical transformation thereof) to another metric Y or a second mathematical transformation thereof Y′ (e.g., the number of units of a respective genomic sequence in a second one or more biological samples or a second mathematical transformation thereof) expressed as X/Y, Y/X, log N(X/Y), log N(Y/X), X′/Y, Y/X′, log N(X′/Y), or log N(Y/X′), X/Y′, Y′/X, log N(X/Y′), log N(Y′/X), X′/Y′, Y′/X′, log N(X′/Y′), or log N(Y′/X′), where N is any real number greater than 1 and where example mathematical transformations of X and Y include, but are limited to. raising X or Y to a power Z, multiplying X or Y by a constant Q, where Z and Q are any real numbers, and/or taking an M based logarithm of X and/or Y, where M is a real number greater than 1. In one non-limiting example, X is transformed to X′ prior to ratio calculation by raising X by the power of two (X2) and Y is transformed to Y′ prior to ratio calculation by raising Y by the power of 3.2 (Y3.2) and the ratio of X and Y is computed as log 2(X′/Y′).

As used herein, the terms “sequencing,” “sequence determination,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript or a genomic locus. Many sequencing techniques are available and known in the art such as but not limited to, Sanger sequencing, paired-end sequencing, pyrosequencing, and SMRT sequencing and DNB generation (e.g., Rolling circle and MGI-DNBseq G-400 sequencing).

As used herein, the term “DNA amplification” will be typically used to denote the in vitro synthesis of double-stranded DNA molecules using PCR. It is noted that other amplification methods exist and they may be used in the present invention without departing from the gist.

The term “genome”, as used herein, relates to a material or mixture of materials, containing genetic material from an organism. The term “genomic DNA” as used herein refers to deoxyribonucleic acids that are obtained from an organism. The terms “genome” and “genomic DNA” encompass genetic material that may have undergone amplification, purification, or fragmentation.

The term “sequence variation”, as used herein, refers to a difference in nucleic acid sequence between a test sample and a reference sample that may vary over a range of 1 to 10 bases, 10 to 100 bases, 100 to 100 kb, or 100 kb to 10 MB. Sequence variation may include single nucleotide polymorphism and genetic mutations relative to wild-type. In certain embodiments, sequence variation results from one or more parts of a chromosome being rearranged within a single chromosome or between chromosomes relative to a reference. In certain cases, a sequence variation may reflect a difference, e.g. abnormality, in chromosome structure, such as an inversion, a deletion, an insertion or a translocation relative to a reference chromosome, for example.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing, for example, can provide sequence reads that can vary in size from tens to hundreds to thousands of base pairs. Illumina® parallel sequencing, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “read count” refers to the total number of nucleic acid reads generated, which may or may not be equivalent to the number of nucleic acid molecules generated, during a nucleic acid sequencing reaction.

As used herein, the term “read-depth,” “sequencing depth,” or “depth” can refer to a total number of unique nucleic acid fragments encompassing a particular locus or region of the genome of a subject that are sequenced in a particular sequencing reaction. Sequencing depth can be expressed as “Y×”, e.g., 50×, 100×, etc., where “Y” refers to the number of unique nucleic acid fragments encompassing a particular locus that are sequenced in a sequencing reaction. In such a case, Y is necessarily an integer, because it represents the actual sequencing depth for a particular locus. Alternatively, read-depth, sequencing depth, or depth can refer to a measure of central tendency (e.g., a mean or mode) of the number of unique nucleic acid fragments that encompass one of a plurality of loci or regions of the genome of a subject that are sequenced in a particular sequencing reaction. For example, in some embodiments, sequencing depth refers to the average depth of every locus across an arm of a chromosome, a targeted sequencing panel, an exome, or an entire genome. In such case, Y may be expressed as a fraction or a decimal, because it refers to an average coverage across a plurality of loci. When a mean depth is recited, the actual depth for any particular locus may be different than the overall recited depth. Metrics can be determined that provide a range of sequencing depths in which a defined percentage of the total number of loci fall. For instance, a range of sequencing depths within which 90% or 95%, or 99% of the loci fall. As understood by the skilled artisan, different sequencing technologies provide different sequencing depths. For instance, low-pass whole genome sequencing can refer to technologies that provide a sequencing depth of less than 5×, less than 4×, less than 3×, or less than 2×, e.g., from about 0.5× to about 3×.

As used herein, the term “reference genome” refers to any sequenced or otherwise characterized genome, whether partial or complete, of any organism or pathogen that may be used to reference identified sequences from a subject. Typically, a reference genome will be derived from a subject of the same species as the subject whose sequences are being evaluated. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or pathogen, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). For a haploid genome, there can be only one nucleotide at each locus. For a diploid genome, heterozygous loci can be identified; each heterozygous locus can have two alleles, where either allele can allow a match for alignment to the locus.

As disclosed herein, the term “regions of a reference genome,” “genomic region,” or “chromosomal region” refers to any portion of a reference genome, contiguous or non-contiguous. It can also be referred to, for example, as a bin, a partition, a genomic portion, a portion of a reference genome, a portion of a chromosome and the like. In some embodiments, a genomic section is based on a particular length of genomic sequence. In some embodiments, a method can include analysis of multiple mapped nucleic acid fragments to a plurality of genomic regions. Genomic regions can be approximately the same length or the genomic sections can be different lengths. In some embodiments, genomic regions are of about equal length. In some embodiments genomic regions of different lengths are adjusted or weighted. In some embodiments, a genomic region is about 10 kilobases (kb) to about 500 kb, about 20 kb to about 400 kb, about 30 kb to about 300 kb, about 40 kb to about 200 kb, and sometimes about 50 kb to about 100 kb. In some embodiments, a genomic region is about 100 kb to about 200 kb. A genomic region is not limited to contiguous runs of sequence. Thus, genomic regions can be made up of contiguous and/or non-contiguous sequences. A genomic region is not limited to a single chromosome. In some embodiments, a genomic region includes all or part of one chromosome or all or part of two or more chromosomes. In some embodiments, genomic regions may span one, two, or more entire chromosomes. In addition, the genomic regions may span joint or disjointed portions of multiple chromosomes.

As used herein, the term “specificity” or “true negative” or “true negative rate” refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the therapeutic agent being administered.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

Reference will now be made in detail to embodiments, examples of which are illustrated in the accompanying drawings. In the following detailed description, numerous specific details are set forth in order to provide a thorough understanding of the present disclosure, including example systems, methods, techniques, instruction sequences, and computing machine program products that embody illustrative implementations. However, the illustrative discussions below are not intended to be exhaustive or to limit the implementations to the precise forms disclosed. Many modifications and variations are possible in view of the above teachings. The features described herein are not limited by the illustrated ordering of acts or events, as some acts can occur in different orders and/or concurrently with other acts or events.

The implementations provided herein are chosen and described in order to best explain the principles and their practical applications, to thereby enable others skilled in the art to best utilize the various embodiments with various modifications as are suited to the particular use contemplated. In some instances, well-known methods, procedures, components, circuits, and networks have not been described in detail so as not to unnecessarily obscure aspects of the embodiments. In other instances, it will be apparent to one of ordinary skill in the art that the present disclosure may be practiced without one or more of the specific details.

It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that though such a design effort might be complex and time-consuming, it will nevertheless be a routine undertaking of engineering for those of ordering skill in the art having the benefit of the present disclosure.

### DESCRIPTION

To overcome the limitations of existing test methods for early detection of cancer, the systems and method of the present disclosure provide a novel liquid biopsy test procedure based on the screening of cancer cells for presence of tumor by methylation and size of cfDNA, also known as SPOT-MAS (Screening for Presence Of Tumor by Methylation and Size of cfDNA) test procedure. This SPOT-MAS test procedure allows simultaneous detection of four patterns of characteristic variations of tumor DNA including: i) methylation at specific sites of genes related to tumor growth; ii) genome-wide methylation of tumor DNA; iii) genome-wide copy number abnormalities of tumor DNA; and iv) the typical size of the DNA released by the tumor into the bloodstream.

The present disclosure provides simultaneous combination of four patterns of characteristic variations of tumor DNA in the SPOT-MAS liquid biopsy test procedure helps to improve the detection efficiency of early-stage cancers, differentiate benign from malignant tumor, monitor post-treatment recurrence of tumor and locate tumor. Moreover, different types of cancer carry different characteristic variations, therefore the investigation of many attributes helps to pinpoint the exact origin of the cancer. Simultaneous analysis of many different attributes of tumor DNA is the basis for the SPOT-MAS test procedure to increase the sensitivity of cancer detection compared with procedures that rely solely on one type of attribute such as gene mutations or methyl changes in certain regions.

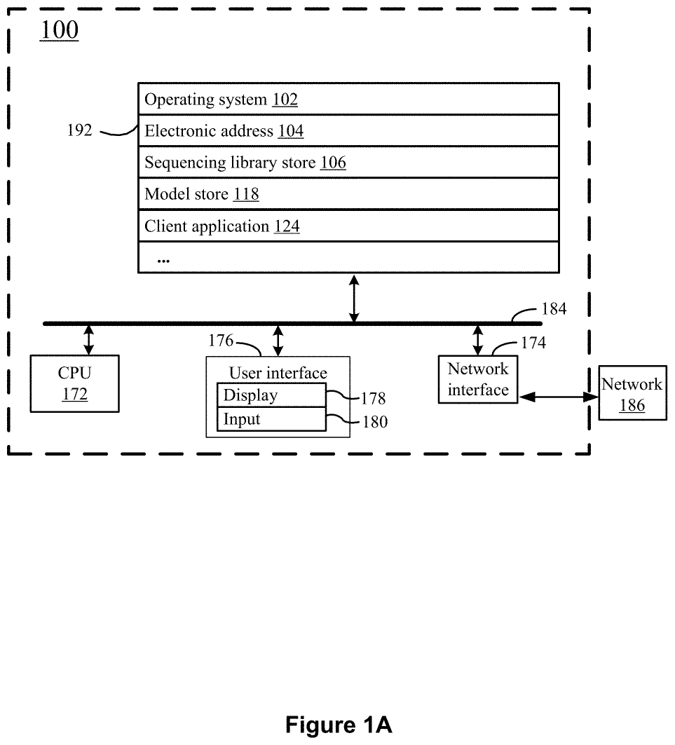

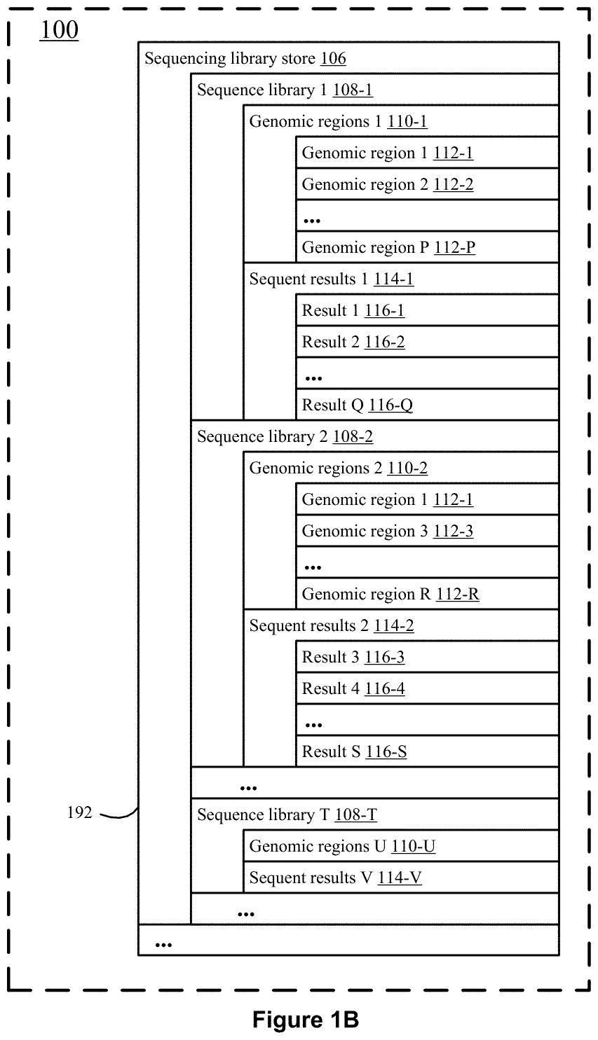

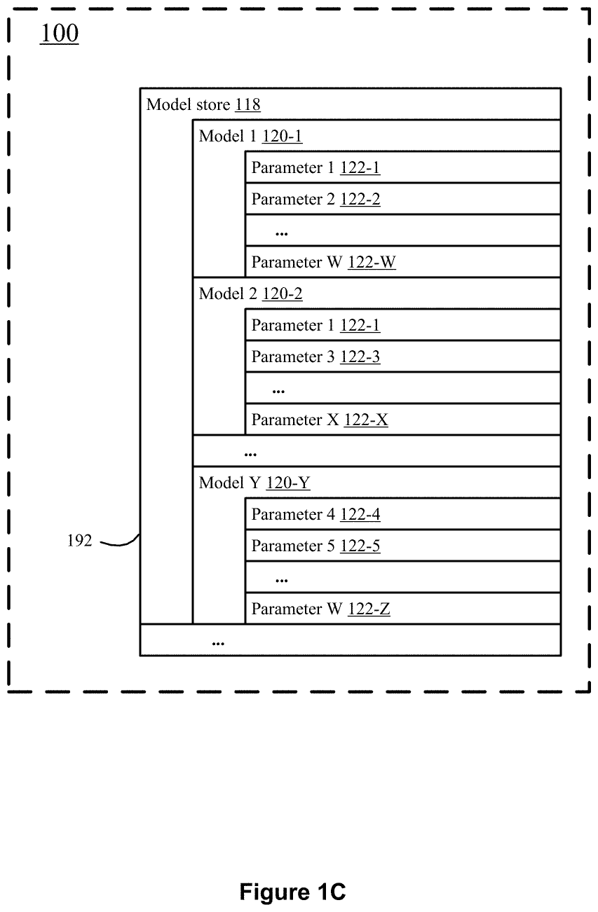

In the present disclosure, unless expressly stated otherwise, descriptions of devices and systems will include implementations of one or more computers. For instance, and for purposes of illustration in FIGS. 1A, 1, and 1C, a computer system 100 is represented as a single device that includes all the functionality of the computer system 100. However, the present disclosure is not limited thereto. For instance, in some embodiments, the functionality of the computer system 100 is spread across any number of networked computers and/or reside on each of several networked computers and/or by hosted on one or more virtual machines and/or containers at a remote location accessible across a communications network (e.g., communications network 186 of FIG. 1A). One of skill in the art will appreciate that a wide array of different computer topologies is possible for the computer system 100, and other devices and systems of the preset disclosure, and that all such topologies are within the scope of the present disclosure. Moreover, rather than relying on a physical communications network 186, the illustrated devices and systems may wirelessly transmit information between each other. As such, the exemplary topology shown in FIGS. 1A, 1B, and 1C merely serves to describe the features of an embodiment of the present disclosure in a manner that will be readily understood to one of skill in the art.

FIGS. 1A, 1i, and 1C collectively depicts a block diagram of a distributed computer system (e.g., computer system 100) according to some embodiments of the present disclosure. The computer system 100 at least facilitates detecting the presence of a cancer and cancer origin in a test subject.

In some embodiments, the communication network 186 optionally includes the Internet, one or more local area networks (LANs), one or more wide area networks (WANs), other types of networks, or a combination of such networks.

Examples of communication networks 186 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs) 172, a network or other communications interface 174, and memory 192.

In some embodiments, the computer system 100 includes a user interface 176. The user interface 176 typically includes a display 178 for presenting media, such as a result by a respective model (e.g., first model 122-1, second model 122-2, . . . , model Y 120-Y of FIG. 1C). In some embodiments, the display 178 is integrated within the computer systems (e.g., housed in the same chassis as the CPU 172 and memory 192). In some embodiments, the computer system 100 includes one or more input device(s) 180, which allow a subject to interact with the computer system 100. In some embodiments, input devices 180 include a keyboard, a mouse, and/or other input mechanisms. Alternatively, or in addition, in some embodiments, the display 178 includes a touch-sensitive surface (e.g., where display 178 is a touch-sensitive display or computer system 100 includes a touch pad).

In some embodiments, the computer system 100 presents media to a user through the display 178. Examples of media presented by the display 178 include one or more images, a video, audio (e.g., waveforms of an audio sample), or a combination thereof. In typical embodiments, the one or more images, the video, the audio, or the combination thereof is presented by the display 178 through a client application 120. In some embodiments, the audio is presented through an external device (e.g., speakers, headphones, input/output (I/O) subsystem, etc.) that receives audio information from the computer system 100 and presents audio data based on this audio information. In some embodiments, the user interface 176 also includes an audio output device, such as speakers or an audio output for connecting with speakers, earphones, or headphones.

Memory 192 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 192 may optionally include one or more storage devices remotely located from the CPU(s) 172. Memory 192, or alternatively the non-volatile memory device(s) within memory 192, includes a non-transitory computer readable storage medium. Access to memory 192 by other components of the computer system 100, such as the CPU(s) 172, is, optionally, controlled by a controller. In some embodiments, memory 192 can include mass storage that is remotely located with respect to the CPU(s) 172. In other words, some data stored in memory 192 may in fact be hosted on devices that are external to the computer system 100, but that can be electronically accessed by the computer system 100 over an Internet, intranet, or other form of network 186 or electronic cable using communication interface 184.

In some embodiments, the memory 192 of the computer system 100 for detecting the presence of a cancer and for identifying the cancer origin in a test subject stores:
an operating system 102 (e.g., ANDROID, iOS, DARWIN, RTXC, LINUX, UNIX, OS X, WINDOWS, or an embedded operating system such as VxWorks) that includes procedures for handling various basic system services;
optionally, an electronic address 104 associated with the computer system 100 that identifies the computer system 100 (e.g., within the communication network 186);
a sequencing library store 106 that retains a record of a plurality of sequencing libraries (e.g., first sequence library 108-1, second sequence library 108-2, . . . , sequence library T 108-T of FIG. 1C), each sequence library prepared for a plurality of specific target genomic regions (e.g., first plurality of genomic regions 110 of FIG. 1i), whereby one or more sequency libraries 108 includes a corresponding plurality of sequencing results produced therefrom that is utilized by one or more models 122 for detecting tumor DNA in mammalian blood; and
a model library 118 that retains a plurality of models (e.g., first model 120-1, second model 120-2, . . . , model Y 122-X of FIG. 1C), each respective model 120 utilized for providing, at least in part, for detecting tumor DNA in mammalian blood based on one or more parameters of a corresponding model 120 (e.g., first parameter 122-1, second parameter 122-2, . . . , parameter W 122-W of first model 120-1 of FIG. 1C); and
a client application 124 for presenting information (e.g., media) using a display 178 of the computer system 100.

As indicated above, an optional electronic address 104 is associated with the computer system 100. The optional electronic address 204 is utilized to at least uniquely identify the computer system 100 from other devices and components of the distributed system 100, such as other devices having access to the communications network 186. For instance, in some embodiments, the electronic address 104 is utilized to receive a request from a remote device to detect tumor DNA in mammalian blood.

Referring to FIG. 1B, the sequence library 106 stores a record of a plurality of sequence libraries 108. In some embodiments, each sequencing library 108 includes data associated with a plurality of specific target genomic regions including reads of paired-end sequences of cfDNA molecule. In some such embodiments, each sequencing library 108 includes a plurality of sequencing results, such as a first plurality of sequencing results that are utilized to locate two ends of a cfDNA fragment on an original genome, thereby determining a length of that cfDNA fragment as a respective result 116.

Referring to FIG. 1C, the computer system includes a model library 118 that stores a plurality of models 120 (e.g., classifiers, regressors, clustering, etc.). In some embodiments, the model library 118 stores two more models 120 (e.g., a first model 120-1 and a second model 120-2), three or more models 120, four or more models 120, ten or more models 120, 50 or more models 120, or 100 or more models 120.

In some embodiments, a model 120 in the plurality of models is implemented as an artificial intelligence engine for the subject question and answering system (QAS). For instance, in some embodiments, the model 120 includes one or more gradient boosting models 120, one or more random forest models 120, one or more neural network (NN) models 120, one or more regression models, one or more Naïve Bayes models 120, one or more machine learning algorithms (MLA) 116, or a combination thereof. In some embodiments, an MLA or a NN is trained from a training data set that includes one or more features identified from a data set. MLAs include supervised algorithms (such as algorithms where the features/classifications in the data set are annotated) using linear regression, logistic regression, decision trees, classification and regression trees, Naïve Bayes, nearest neighbor clustering; unsupervised algorithms (such as algorithms where no features/classification in the data set are annotated a priori), such as means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as minimum cut, harmonic function, manifold regularization, etc.), heuristic approaches, or support vector machines.

In some embodiments, a model 120 is in the form of a hybrid deep learning (DL) model such as a Long Short Term Memory (LSTM) model, or a bidirectional LSTM (BiLSTM) model with an attention layer based on a neural network (NN). In some embodiments a model 120 is a deep learning model in the context of a network topology and word embedding technique customized for QAS. In some embodiments, a model 120 is a conditional random fields model 120, a convolutional neural network (CNN) model 120, an attention based neural network model 120, a deep learning model 120, a long short term memory network model 120, or another form of neural network model 120.

While MLA and neural networks identify distinct approaches to machine learning, the terms may be used interchangeably herein. Thus, a reference to MLA may include a corresponding NN or a reference to NN may include a corresponding MLA unless explicitly stated otherwise. In some embodiments, the training of a respective model 120 includes providing one or more optimized datasets, labeling these features as they occur (e.g., in sequence results), and training the MLA to predict or classify based on new inputs. Artificial NNs are efficient computing models which have shown their strengths in solving hard problems in artificial intelligence. For instance, artificial NNs have also been shown to be universal approximators, that is, they can represent a wide variety of functions when given appropriate parameters.

One of skill in the art will readily appreciate other models 120 that are applicable to the systems and methods of the present disclosure. In some embodiments, the systems and methods of the present disclosure utilize more than one model 120 to provide an evaluation (e.g., arrive at an evaluation given one or more inputs), such as detecting tumor DNA in mammalian blood with an increased accuracy. For instance, in some embodiments, each respective model 120 arrives at a corresponding evaluation when provided a respective data set. Accordingly, in some embodiments, each respective model 120 independently arrives at a result and then the result of each respective model 120 is collectively verified through a comparison or amalgamation of the models 120. From this, a cumulative result is provided by the models 120. However, the present disclosure is not limited thereto.

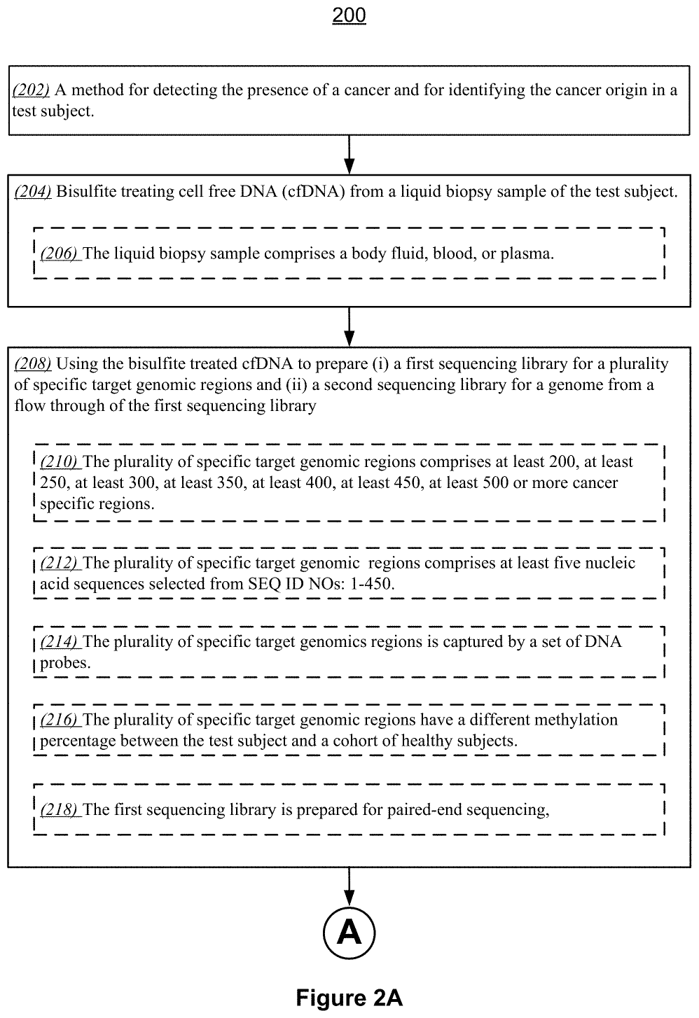

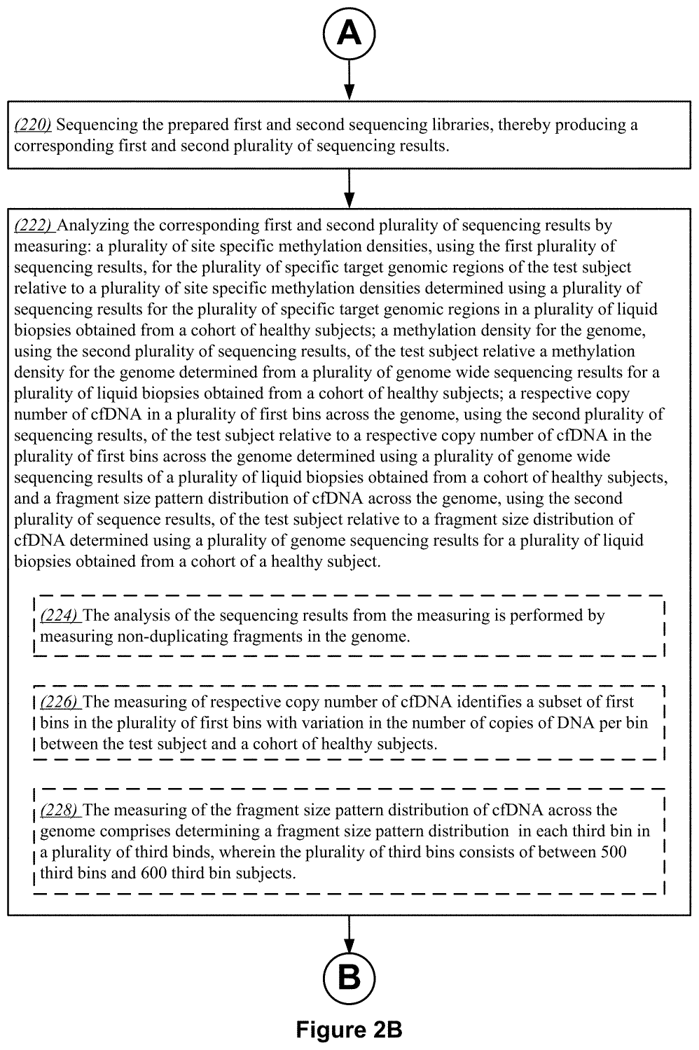

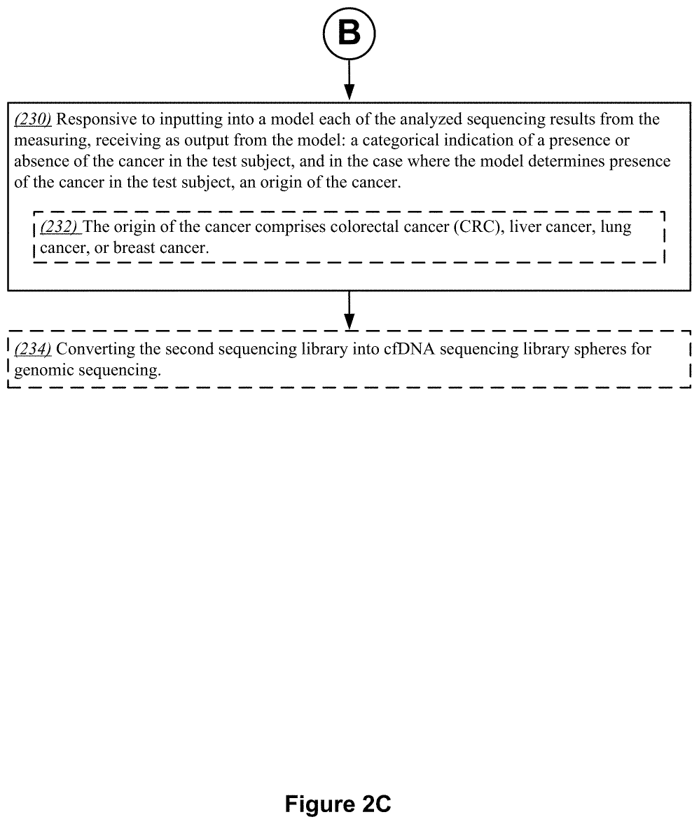

In some embodiments, a respective model 120 is tasked with performing a corresponding activity. As a non-limiting example, in some embodiments, the task performed by the respective model 120 includes, but is not limited to, detecting a presence of a cancer and identifying a cancer origin in a test subject (e.g., block 202 of FIG. 2A, block 230 of FIG. 2C), preparing a first sequence library 108-1 and/or a second sequency library 108-2 (e.g., block 208 of FIG. 2A), sequencing the prepared first and/or second sequencing libraries (e.g., block 220 of FIG. 2B), producing a corresponding first and/or second plurality of sequencing results 114 (e.g., block 220 of FIG. 2B), analyzing the corresponding first and second plurality of sequencing results (e.g., block 222 of FIG. 2B), determining a categorical indication of a presence or absence of the cancer in the test subject (e.g., block 230 of FIG. 1C), converting the second sequencing library into cfDNA sequencing library spheres for genomic sequencing (e.g., block 234 of FIG. 2C) or any combination thereof.

In some embodiments, each respective model 120 of the present disclosure makes use of 10 or more parameters, 100 or more parameters, 1000 or more parameters, 10,000 or more parameters, or 100,000 or more parameters. In some embodiments, each respective model of the present disclosure cannot be mentally performed.

In some embodiments, a client application 124 is a group of instructions that, when executed by the processor 174, generates content for presentation to the user, such as a result provided by one or more models 120. In some embodiments, the client application 124 generates content in response to one or more inputs received from the user through the computer system 100, such as the inputs 180 of the computer system 100.

Each of the above identified modules and applications correspond to a set of executable instructions for performing one or more functions described above and the methods described in the present disclosure (e.g., the computer-implemented methods and other information processing methods described herein; method 200 of FIGS. 2A through 2C; etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 192 optionally stores a subset of the modules and data structures identified above. Furthermore, in some embodiments, the memory 192 stores additional modules and data structures not described above.

It should be appreciated that the computer system 100 of FIGS. 1A, 1, and 1C is only one example of a computer system 100, and that the computer system 100 optionally has more or fewer components than shown, optionally combines two or more components, or optionally has a different configuration or arrangement of the components. The various components shown in FIGS. 1A, 1B, and 1C are implemented in hardware, software, firmware, or a combination thereof, including one or more signal processing and/or application specific integrated circuits.

Now that a general topology of the distributed system 100 has been described in accordance with various embodiments of the present disclosures, details regarding some processes in accordance with FIGS. 2A through 2C will be described.

FIGS. 2A through 2C illustrate a flow chart of methods (e.g., method 200) for detecting a presence of a cancer and identifying a cancer origin in a test subject, in accordance with embodiments of the present disclosure. Specifically, an exemplary method 200 for detecting a presence of a cancer and identifying a cancer origin in a test subject is provided, in accordance with some embodiments of the present disclosure. In the flow charts, the preferred parts of the methods are shown in solid line boxes, whereas optional variants of the methods, or optional equipment used by the methods, are shown in dashed line boxes.

Various modules in the memory 192 of the computer system 100 (e.g., sequence library 106, model library 118, client application 124, or a combination thereof of FIGS. 1A, 1i, and 1C), the memory 192 of the computer system 100, or both perform certain processes of the methods 200 described in FIGS. 2A through 2C, unless expressly stated otherwise. Furthermore, it will be appreciated that the processes in FIGS. 2A through 2C can be encoded in a single module or any combination of modules.

Block 202. Referring to block 202 of FIG. 2A, a method 200 detecting the presence of a cancer and for identifying the cancer origin in a test subject is provided.

In some embodiments, the method 200 is implemented at a computer system (e.g., computer system 100 of FIGS. 1A, 1i, and 1C). The computer system includes one or more processors (e.g., CPU 174 of FIG. 1A) and a memory (e.g., memory 192 of FIGS. 1A, 1B, and 1C) coupled to the one or more processors 174. The memory 192 includes one or more programs (e.g., sequence library 106, model library 118, client application 124, or a combination thereof of FIGS. 1A, 1B, and 1C) configured to be executed by the one or more processors 174. Accordingly, in such embodiments, the one or more programs, when executed by the one or more processors, perform the method 200. As such, portions of the method 200 require a computer (e.g., computer system 100 of FIGS. 1A, 1B, and 1C) to be used because the considerations used by the systems and methods of the present disclosure, on the scale performed by the systems and methods of the present disclosure, cannot be mentally performed. In other words, given an input to a model 120 to collectively consider each respective result, the model 120 output needs to be determined using the computer rather than mentally in such embodiments.

In one aspect, provided herein is a method for detecting the presence of a cancer and for identifying the cancer origin in a test subject. In one aspect, disclosed herein is a method for monitoring likelihood of cancer recurrence in a subject previously treated for cancer. In another aspect, provided herein is a method for assessing the efficacy of a cancer treatment in a subject suffering from cancer. In yet another aspect the present disclosure provides a method for treating cancer in a subject in need thereof.

The various disclosed methods comprise the following: (a) bisulfite treating cell free DNA (cfDNA) from a liquid biopsy sample of the test subject (e.g., block 204 of FIG. 2A); (b) using the bisulfite treated cfDNA to prepare a first sequencing library for (i) a plurality of specific target genomic regions and (ii) a second sequencing library for a genome from a flow through of the first sequencing library (e.g., block 208 of FIG. 2A); (c) sequencing the prepared first and second sequencing libraries, thereby producing a corresponding first and second plurality of sequencing results (e.g., block 220 of FIG. 2B); (d) analyzing the corresponding first and second plurality of sequencing results by measuring:

i. a plurality of site specific methylation densities, using the first plurality of sequencing results, for the plurality of specific target genomic regions of the test subject relative to a plurality of site specific methylation densities determined using a plurality of sequencing results for the plurality of specific target genomic regions in a plurality of liquid biopsies obtained from a cohort of healthy subjects;

ii. a methylation density for the genome, using the second plurality of sequencing results, of the test subject relative a methylation density for the genome determined from a plurality of genome wide sequencing results for a plurality of liquid biopsies obtained from a cohort of healthy subjects;

iii. a respective copy number of cfDNA in a plurality of first bins across the genome, using the second plurality of sequencing results, of the test subject relative to a respective copy number of cfDNA in the plurality of first bins across the genome determined using a plurality of genome wide sequencing results of a plurality of liquid biopsies obtained from a cohort of healthy subjects, and

iv. a fragment size pattern distribution of cfDNA across the genome, using the second plurality of sequence results, of the test subject relative to a fragment size distribution of cfDNA determined using a plurality of genome sequencing results for a plurality of liquid biopsies obtained from a cohort of a healthy subject (e.g., block 222 of FIG. 2B); and

(e) responsive to inputting into a model each of the analyzed sequencing results from (d)(i)-(d)(iv), receiving as output from the model:

i. a categorical indication of a presence or absence of the cancer in the test subject, and

in the case where the model determines presence of the cancer in the test subject, an origin of the cancer (e.g., block 230 of FIG. 2C).

In some embodiments, the plurality of specific target genomic regions comprises at least 2550, at least 75, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, at least 625, at least 650, at least 775, at least 800, at least 825, at least 850, at least 875, at least 900, at least 925, at least 950, at least 975, at least 1000, or more cancer specific regions.

In some embodiments, the plurality of specific target genomic regions comprises at least 200, at least 250, at least 300, at least 350, at least 400, at least 450, at least 500 or more cancer specific regions. In some embodiments, the plurality of specific target genomic regions comprises at least 400, at least 410, at least 420, at least 430, at least 440, at least 450, at least 460, at least 470, at least 480, at least 500 or more cancer specific regions (e.g., block 210 of FIG. 2A). In some embodiments, the plurality of specific target genomic regions comprises at least 440, at least 441, at least 442, at least 443, at least 444, at least 445, at least 446, at least 447, at least 448, at least 449, at least 450, at least 451, at least 452, at least 453, at least 454, at least 455, at least 456, at least 457, at least 458, at least 459, at least 460 or more cancer specific regions. In some embodiments, the plurality of specific target genomic regions comprises 450 cancer specific regions. In some embodiments the 450 cancer specific regions are disclosed in Table 23 as provided elsewhere herein (SEQ ID NOs: 1-450).

In some embodiments, the methylation status comprises a methylation state of each respective CpG site in a corresponding plurality of CpG sites. In some embodiments, the plurality of specific target genomic regions consists of between 10,000 and 11,000 CpG sites, between 11,000 and 12,000 CpG sites, between 12,000 and 13,000 CpG sites, between 14,000 and 15,000 CpG sites, between 15,000 and 16,000 CpG sites, between 16,000 and 17,000 CpG sites, between 17,000 and 18,000 CpG sites, between 18,000 and 19,000 CpG sites, between 19,000 and 20,000 CpG sites, between 20,000 and 21,000 CpG sites, between 21,000 and 22,000 CpG sites, between 22,000 and 23,000 CpG sites, between 23,000 and 24,000 CpG sites, between 24,000 and 25,000 CpG sites, or more. In some embodiments, the plurality of specific target genomic regions consists of between 17,500 and 18,500 CpG sites, between 17,600 and 18,400 CpG sites, between 17,700 and 18,300 CpG sites, between 17,800 and 18,200 CpG sites, or between 17,900 and 18,100 CpG sites. In some embodiments, the plurality of specific target genomic regions consists of 18,000 CpG sites.

In some embodiments, the plurality of specific target genomic regions comprises at least 3, at least 5, at least 10, at least 15, at least 20, at least 25, at least 30, at least 35, at least 40, at least 45, at least 50, least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 441, at least 442, at least 443, at least 444, at least 445, at least 446, at least 447, at least 443, at least 444, at least 445, at least 446, at least 447, at least 448, at least 449 nucleic acid sequences selected from SEQ ID NOs: 1-450 (e.g., block 212 of FIG. 2A).

In some embodiments, the plurality of specific target genomic regions comprises at least 50 nucleic acid sequences selected from SEQ ID NOs: 1-450. In some embodiments, the plurality of specific target genomic regions comprises at least 200 nucleic acid sequences selected from SEQ ID NOs: 1-450. In some embodiments, the plurality of specific target genomic regions comprises at least 300 nucleic acid sequences selected from SEQ ID NOs: 1-450. In some embodiments, each respective target genomic region in the plurality of specific target genomic regions encompasses a sequence selected from SEQ ID NOs: 1-450.

In some embodiments, at least 5, at least 10, at least 20, at least 25, at least 30, at least 35, at least 40, at least 45, at least 50, least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 441, at least 442, at least 443, at least 444, at least 445, at least 446, at least 447, at least 443, at least 444, at least 445, at least 446, at least 447, at least 448, at least 449 respective cancer specific genomic regions in the plurality of cancer specific genomic regions encompass an oncogene and/or a tumor suppressor gene listed in Table 23.

In some embodiments, the plurality of specific target genomics regions is captured by a set of DNA probes (e.g., block 214 of FIG. 2A). In some embodiments, the set of DNA probes comprises DNA fragments with a size ranging between 2 base-pair (bp) and 9 bp, between 10 bp and 19 bp, between 20 bp and 39 bp, between 40 bp and 50 bp, between 51 bp and 60 between 40 bp and 50 bp, between 51 bp and 60 bp, between 61 bp and 70 bp, between 71 bp and 80 bp, between 81 bp and 90 bp, between 91 bp and 100 bp, between 101 bp and 110 bp, between 111 bp and 120 bp, between 121 bp and 130 bp, between 131 bp and 140 bp, between 141 bp and 150 bp, between 151 bp and 160 bp, between 161 bp and 170 bp, between 171 bp and 180 bp, between 181 bp and 190 bp, between 191 bp and 200 bp or more. In some embodiments, the set DNA probes comprises DNA fragments with a size ranging between 111 bp and 120 pb or between 121 bp and 130 bp. In some embodiments, the set DNA probes comprises DNA fragments having a size of 111 bp, 112 bp, 113 bp, 114 bp, 115 bp, 116 bp, 117 bp, 118 bp, 119 bp, 120 bp, 121 bp, 122 bp, 123 bp, 124 bp, 125 bp, 126 bp, 127 bp, 128 bp, 129 bp, 130 bp. In some embodiments, the set DNA probes comprises DNA fragments having a size of 120 bp.

In some embodiments, the set of DNA probes consists of between 50 DNA probes and 99 DNA probes, between 100 DNA probes and 199 DNA probes, between 200 DNA probes and 299 DNA probes, between 300 DNA probes and 399 DNA probes, between 400 DNA probes and 500 DNA probes, between 501 DNA probes and 1000 DNA probes, between 1001 DNA probes and 1500 DNA probes, between 1501 DNA probes and 2000 DNA probes, between 2001 DNA probes and 2100 DNA probes, between 2101 DNA probes and 2150 DNA probes, between 2151 DNA probes and 2200 DNA probes, between 2201 DNA probes and 2250 DNA probes, between 2251 DNA probes and 2300 DNA probes, between 2301 DNA probes and 2350 DNA probes, between 2351 DNA probes and 2400 DNA probes, between 2401 DNA probes and 2450 DNA probes, between 2451 DNA probes and 2500 DNA probes, between 2501 DNA probes and 3000 DNA probes, between 3001 DNA probes and 3500 DNA probes, or between 3501 DNA probes and 4000 DNA probes, or more. In some embodiments, the set DNA probes consists of between 2201 DNA probes and 2250 DNA probes or between 2251 DNA probes and 2300 DNA probes.

In some embodiments, the set DNA probes consists of 2240 DNA probes, 2241 DNA probes, 2242 DNA probes, 2243 DNA probes, 2244 DNA probes, 2245 DNA probes, 2246 DNA probes, 2247 DNA, 2248 DNA probes, 2249 DNA probes, 2250 DNA probes, 2251 DNA probes, 2252 DNA probes, 2253 DNA probes, 2254 DNA probes, 2255 DNA probes, 2256 DNA probes, 2257 DNA probes and 2258 DNA probes, 2259 DNA probes or 2260 DNA probes. In some embodiments, the set DNA probes consists of 2250 DNA probes (Table 25).

In some embodiments, the of DNA probes comprises at least 5, at least 10, at least 50, at least 100, at least 150, at least 200, at least 250, at least 300, at least 350, least 400, at least 450, at least 500, at least 550, at least 600, at least 650, at least 700, at least 750, at least 800, at least 900, at least 1000, least 1100, at least 1150, at least 1200, at least 1250, at least 1300, at least 1350, least 1400, at least 1450, at least 1500, at least 1550, at least 1600, at least 1650, at least 1700, at least 1750, at least 1800, at least 1900, at least 2000, at least 2100, at least 2150, at least 2200, at least 2210, at least 2220, at least 2230, least 2240, at least 2249 nucleic acid sequence selected from SEQ ID NOs: 451-2700.

In some embodiments, the of DNA probes comprises at least 10 nucleic acid sequence selected from SEQ ID NOs: 451-2700. In some embodiments, the set of DNA probes comprises at least 100 nucleic acid sequences selected from SEQ ID NOs: 451-2700. In some embodiments, the set of DNA probes comprises at least 200 nucleic acid sequences selected from SEQ ID NOs: 451-2700. In some embodiments, the set of DNA probes comprises 2250 nucleic acid sequences selected from SEQ ID NOs: 451-2700 (Table 25).

In some embodiments, the first sequencing library is prepared for paired-end sequencing. Details of exemplary sequencing library preparation are provided elsewhere herein. In some embodiments, the sequencing library allows proceeding with genomic sequencing, such as but not limited to Illumina sequencing technology (e.g., ILLUMINA MISEQ® or HISEQ4000® system).

In some embodiments, the genome comprises 22 chromosomes.

In some embodiments, the plurality of specific target genomic regions have a different methylation percentage between the test subject and a cohort of healthy subjects (e.g., block 216 of FIG. 2A).

In some embodiments, the methylation in the test subject is about one fold, about two fold, about three fold, about four fold, or about five fold higher or more than the methylation in the cohort of healthy subjects.

In some embodiments, the second sequencing library comprises universal adapter sequences. Usage of universal adapter and their sequences are well known in the art. In some embodiments, the universal adapters comprise a biotin-bound probes such as but not limited to, biotin-bound P5/P7 probes (Integrated DNA Technologies—IDT, USA). In some embodiments, the second sequencing library is converted into cfDNA sequencing library spheres for genomic sequencing. In some embodiments, the genomic sequencing comprises, but is not limited to, rolling circle sequencing or MGI-DNBseq G-400 sequencing.

In some embodiments, the analysis of the sequencing results from the presently disclosed methods (e.g., (d)(ii)-(d)(iv)) is performed by measuring non-duplicating fragments in the genome (e.g., block 224 of FIG. 2B).

In some embodiments, the methylation density for the genome in (d)(ii) of the disclosed methods is determined for each respective second bin region in between 1500 second bin regions and 2000 second bin regions, in between 200 second bin regions and 2500 second bin regions, in between 2500 second bin regions and 3000 second bin regions, or in between 3000 second bin regions and 3500 second bin regions. In some embodiments, the methylation density for the genome in (d)(ii) of the disclosed methods is determined for each respective second bin region in between 2500 second bin regions and 3000 second bin regions. In some embodiments, the methylation density for the genome in (d)(ii) of the disclosed methods is determined for each respective second bin region of about 2730, about 2731, about 2732, about 2733, about 2734, about 2735, about 2736, about 2737, about 2738, about 2739, or about 2740 second bin regions.

In some embodiments, each respective second bin region consists of between 500,000 nucleotides and 600,000 nucleotides, between 600,000 nucleotides and 700,000 nucleotides, between 700,000 nucleotides and 800,000 nucleotides, between 900,000 nucleotides and 1,000,000 nucleotides, between 1,000,000 nucleotides and 1,100,000 nucleotides, between 1,200,000 nucleotides and 1,300,000 nucleotides, between 1,300,000 nucleotides and 1,400,000 nucleotides, or between 1,400,000 nucleotides and 1,500,000 nucleotides. In some embodiments, each respective second bin region consists of between 600,000 nucleotides and 1,000,000 nucleotides, between 700,000 nucleotides and 1,100,000 nucleotides, between 800,000 nucleotides and 1,300,000 nucleotides, between 900,000 nucleotides and 1,400,000 nucleotides, or between 1,000,000 nucleotides and 1,500,000 nucleotides. In some embodiments, each respective second bin region consists of between 1,000,000 nucleotides (1 megabase).

In some embodiment, the measuring of the methylation density identifies second bin regions in the between 2500 second bin regions and 3000 second bin regions that are differentially methylated between the test subject suffering and a cohort of healthy subjects. In some embodiment, the measuring of the methylation density identifies second bin regions of about 2730, about 2731, about 2732, about 2733, about 2734, about 2735, about 2736, about 2737, about 2738, about 2739, or about 2740 second bin regions that are differentially methylated between the test subject suffering and a cohort of healthy subjects.

In some embodiments, the methylation density in each respective second bin region is evaluated based on a Z score value. In some embodiments, as provided in details elsewhere herein, variation in values of methylation density in each bin is evaluated based on the “Z score” value as computed based the following formula:

$$Zscore = \frac{\begin{matrix}MD in surveyed bin - Mean MD in corresponding \\ bin of the reference group\end{matrix}}{\begin{matrix}Standard deviation MD in corresponding bin \\ in the reference group\end{matrix}}$$

In some embodiments, the plurality of first bins is between 1500 first bin regions and 2000 first bin regions, between 200 first bin regions and 2500 first bin regions, between 2500 first bin regions and 3000 first bin regions, or between 3000 first bin regions and 3500 first bin regions. In some embodiments, the plurality of first bins is between 2500 first bin regions and 3000 first bin regions. In some embodiments, the plurality of first bins is about 2730, about 2731, about 2732, about 2733, about 2734, about 2735, about 2736, about 2737, about 2738, about 2739, or about 2740 first bin regions.

In some embodiments, each first bin consists of between 500,000 nucleotides and 600,000 nucleotides, between 600,000 nucleotides and 700,000 nucleotides, between 700,000 nucleotides and 800,000 nucleotides, between 900,000 nucleotides and 1,000,000 nucleotides, between 1,000,000 nucleotides and 1,100,000 nucleotides, between 1,200,000 nucleotides and 1,300,000 nucleotides, between 1,300,000 nucleotides and 1,400,000 nucleotides, or between 1,400,000 nucleotides and 1,500,000 nucleotides. In some embodiments, each first bin consists of between 600,000 nucleotides and 1,000,000 nucleotides, between 700,000 nucleotides and 1,100,000 nucleotides, between 800,000 nucleotides and 1,300,000 nucleotides, between 900,000 nucleotides and 1,400,000 nucleotides, or between 1,000,000 nucleotides and 1,500,000 nucleotides. In some embodiments, each first bin consists of about 1,000,000 nucleotides (1 megabase).

In some embodiment, the measuring of respective copy number of cfDNA identifies a subset of first bins in the plurality of first bins with variation in the number of copies of DNA per bin between the test subject and a cohort of healthy subjects. In some embodiments, the variation in the number of copies of DNA between the test subject and a cohort of healthy subjects in each first bin is evaluated based on a Z score value.

In some embodiment, as provided in details elsewhere herein, variation of gene copy number in each bin is evaluated based on the “Z score” value as computed in the following formula:

$$Zscore = \frac{\begin{matrix}number of reads in surveyed bin - Average number \\ of reads in corresponding bin of the reference group\end{matrix}}{\begin{matrix}Standard deviation of the number of reads \\ in the corresponding bin in the reference group\end{matrix}}$$

In some embodiments, the measuring of the fragment size pattern distribution of cfDNA across the genome comprises determining a fragment size pattern distribution in each third bin in a plurality of third binds, where the plurality of third bins consists of between 500 third bins and 600 third bins (e.g., block 228 of FIG. 2B).

In some embodiment, the measuring of the fragment size pattern distribution of cfDNA across the genome comprises determining a fragment size pattern distribution in each third bin in a plurality of third binds, where the plurality of third bins consists of between 100 third bins and 200 third bins, between 200 third bins and 300 third bins, between 300 third bins and 400 third bins, between 400 third bins and 500 third bins, between 500 third bins and 600 third bins, between 600 third bins and 700 third bins, between 800 third bins and 900 third bins, or between 900 third bins and 1,000 third bins. In some embodiment, the measuring of the fragment size pattern distribution of cfDNA across the genome comprises determining a fragment size pattern distribution in each third bin in a plurality of third binds, where the plurality of third bins consists of between 500 third bins and 600 third bins. In some embodiment, the measuring of the fragment size pattern distribution of cfDNA across the genome comprises determining a fragment size pattern distribution in each third bin in a plurality of third binds, where the plurality of third bins consists of between 550 third bins and 600 third bins. In some embodiment, the measuring of the fragment size pattern distribution of cfDNA across the genome comprises determining a fragment size pattern distribution in each third bin in a plurality of third binds, where the plurality of third bins consists of about 550, about 570, about 580, about 590, or about 600 third bins. In some embodiment, the measuring of the fragment size pattern distribution of cfDNA across the genome comprises determining a fragment size pattern distribution in each third bin in a plurality of third binds, where the plurality of third bins consists of 580, 581, 582, 583, 584, 585, 586, 587, 588, 589, 590, or 600 third bins.

In some embodiments, each respective third bin consists of between 1 million (1 megabase) nucleotides and 1.5 million nucleotides, between 1.5 million nucleotides and 2 million nucleotides, between 2 million nucleotides and 2.5 million nucleotides, between 2.5 million nucleotides and 3 million nucleotides, between 3.5 million nucleotides and 4 million nucleotides, between 4 million nucleotides and 4.5 million nucleotides, between 5 million nucleotides and 5.5 million nucleotides, between 5.5 million nucleotides and 6 million nucleotides, between 6.5 million nucleotides and 7 million nucleotides, between 7 million nucleotides and 7.5 million nucleotides, or between 7.5 million nucleotides and 8 million nucleotides. In some embodiments, each respective third bin consists of between 4.5 million nucleotides (4.5 megabases) and 5.5 million nucleotides (5.5 megabases). In some embodiments, each respective third bin consists of 5 million nucleotides (5 megabases).

In some embodiments, the measuring of the fragment size pattern distribution of cfDNA identifies a subset of third bins with a variation in the fragment size pattern distribution of cfDNA per bin between the test subject and a cohort of healthy subjects (e.g., block 226 of FIG. 2B). In some embodiments, the variation in the fragment size pattern distribution of the cfDNA in each third bin in the plurality of third bins is evaluated based on cfDNA fragment length ratio (RF) value. In some embodiments, the RF value identifies presence of cancer, where cfDNA fragment length released from tumor cells from the test subject is shorter than cfDNA fragment length released by cells of a cohort of healthy subjects.

In some embodiments, the plurality of specific target genomic regions have a methylation percentage higher in the test subject as compared to a cohort of healthy subjects. In some embodiments, the cohort of healthy subjects consists of between 5 and 50 healthy subjects, between 5 and 100 healthy subjects, between 5 and 1000 healthy subjects, between 5 and 5000 healthy subjects, between 50 and 500 healthy subjects, between 50 and 1000 healthy subjects, between 50 and 5000 healthy subjects, between 100 and 500 healthy subjects, between 100 and 1000 healthy subjects, between 100 and 5000 healthy subjects, between 500 and 1000 healthy subjects, or between 500 and 5000 healthy subjects, or more. In some embodiments, healthy subjects include for instance subjects that are not diagnosed with any disease and/or are not diagnosed with cancer. In some embodiments, the healthy subjects have the same sex and/or age range as the test subject.

In some embodiments, the liquid biopsy sample comprises a body fluid, blood, or plasma.

In some embodiments, the origin of the cancer comprises but is not limited to colorectal cancer (CRC), liver cancer, lung cancer, breast cancer (e.g., block 232 of FIG. 2C), or gastric cancer.

In some embodiments, the subject is a mammal. In some embodiments, the subject is a non-human mammal, such as but not limited to a livestock or a pet (e.g. ovine, bovine, porcine, canine, feline and marine mammals). In some embodiments, the subject is subject is human.

In some embodiments, the disclosed machine learning model is a composite model comprising four attribute models and a combination model, where each respective attribute model in the four attribute models produces an initial categorical classification upon input of a different one of the analyzed sequencing results from (d)(i)-(d)(iv), and where the combination model combines the respective categorical indication of the presence or absence of cancer in the test subject of each attribute model in the four attribute models by a weighted combination of the four attribute models.

In some embodiments, the combination model is a logistic regression combined linear model of the four attribute models, in which each of the four attribute models is independently assigned a different probability weight.

In some embodiments, the disclosed model (e.g., machine learning model) comprises at least 5, at least 10, at least 20, at least 30, at least 40, at least 50, at least 60, at least 70, at least 80, least 90, at least 100, at least 110, at least 120, at least 130, at least 140, at least 150, at least 160, at least 170, at least 180, at least 190, at least 200 or more parameters. In some embodiments, the disclosed machine learning model comprises at least 100 parameters.

In some embodiments, the disclosed machine learning model comprises a logistic regression, a deep neural network, a fully connected neural network, a convolutional neural network, a graph based neural network, or a support vector machine. In some embodiments, the deep neural network specifies a tissue for cancer origin. In some embodiments, the disclosed model comprises machine learning models known in the art including but not limited to supervised algorithms (such as algorithms where the features/classifications in the data set are annotated) using linear regression, logistic regression, decision trees, classification and regression trees, naïve Bayes, nearest neighbour clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

In one aspect, the disclosure provides a method for detecting the presence of a cancer and for identifying the cancer origin in a test subject. The disclosed method comprises a computer system having one or more processors, and memory storing one or more programs for execution by the one or more processors: obtaining, in electronic form, a sequencing data generated from a first sequencing library for (i) a plurality of specific target genomic regions and (ii) a second sequencing library for a genome from a flow through of the first sequencing library; determining a methylation pattern based on the sequencing data from the first sequencing library from the test subject relative to a cohort of healthy subjects, where the methylation pattern comprises a methylation state of each CpG site in a corresponding plurality of CpG sites in 450 cancer specific gene regions; determining a methylation pattern based on the sequencing data from the second sequencing library from the test to a cohort of healthy subjects, where the methylation pattern comprises a methylation state of each CpG site in a corresponding plurality of CpG sites in 2734 bin regions, where each bin region comprises one million nucleotides (one megabase); determining number of copies of cfDNA based on the sequencing data from the second sequencing library from the test subject suffering from cancer relative to a cohort of healthy subjects, where the number of copies of cfDNA comprises measuring of the number of copies of cfDNA in 2734 bin regions, where each bin region comprises one million nucleotides (one megabase), further where the measuring of number of copies of cfDNA identifies bin regions with variation in the number of copies of cfDNA per bin between the test subject and a cohort of healthy subjects; determining size patterns of cfDNA based on the sequencing data from the second sequencing library from the test subject relative to a cohort of healthy subjects, where the size patterns of cfDNA comprises measuring of the number of copies of cfDNA in 588 bin regions, where each bin region comprises five million nucleotides (five megabases), further where the measuring of number of copies of DNA identifies bin regions with variation in the number of copies of DNA per bin between the test subject and a cohort of healthy subjects; and applying a machine learning model for the data set for each of the (b)-(e) to indicate presence or absence of the cancer in the test subject, and in the case where the model determines presence of the cancer in the test subject, identify an origin of the cancer.

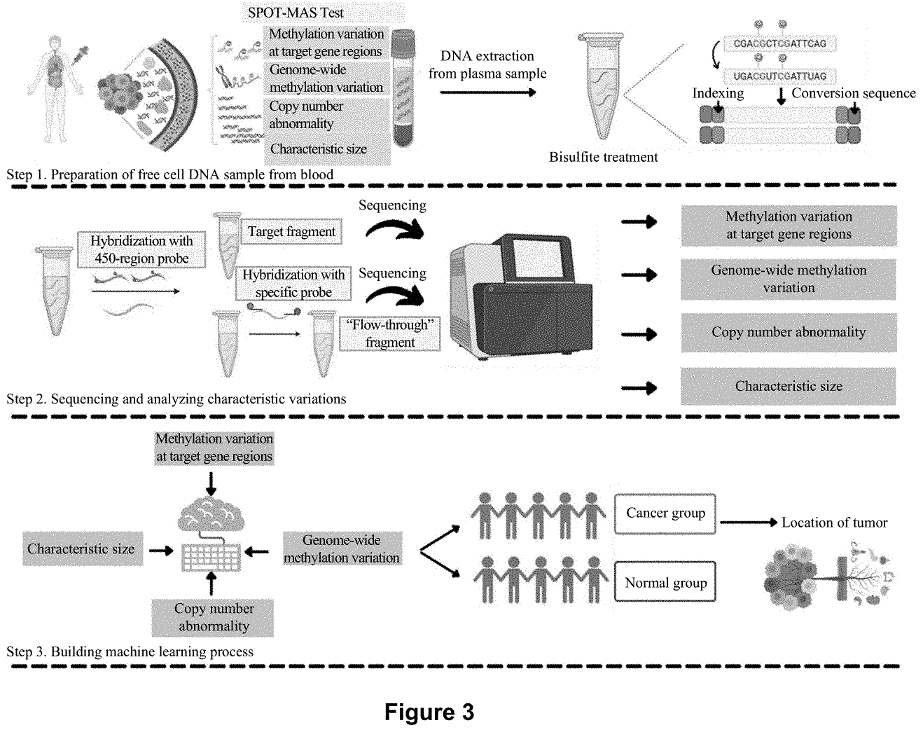

Details of an exemplary system for providing clinical support detecting cancer using a liquid biopsy assay are described in conjunction with FIG. 3 which illustrates the protocol for detecting tumor DNA in peripheral blood using the SPOT-MAS test procedure according to an embodiment of the present disclosure.

Specifically, the present disclosure provides a SPOT-MAS test procedure for detection of tumor DNA in the blood of mammals, comprising:

Element 1: Create a sequencing library of bisulfite-treated cell-free DNA (cfDNA)

Block 204. Referring to block 204 of FIG. 2A, in some embodiments, the first element comprises collecting blood samples and processing blood sample to collect plasma and stratify monocytes. In some embodiments, the cfDNA is extracted from plasma. To perform this extraction of cfDNA, any known commercially available kit can be used, such as but not limited to the MagMAX cell-free DNA extraction kit (supplied by Thermo Fisher, USA) on KingFisher Flex Magnetic 96DW automatic system (supplied by Thermo Fisher, USA).

Block 208. Referring to block 208, in further embodiments, the obtained cfDNA is treated with bisulfite (BS) to convert C nucleotides without methyl moiety (—CH3) into T nucleotides, while the C nucleotides with methyl moiety are preserved (e.g., block 234 of FIG. 2C). In other embodiments, purification, desulfurization and resolution are carried out to recover the bisulfite-treated cfDNA. In some embodiments, the processing of the cfDNAs can use the bisulfite conversion kit EZ_DNA methylation Gold Kit (supplied by Zymo) with the advantages of being able to convert DNA at with low cfDNA input (minimum 500 pg), achieving a conversion efficiency of over 99% and a recovery efficiency of over 75%.

In some embodiments, the cfDNAs, after being treated with bisulfite, is used to create a sequencing library. The process of preparing a sequencing library is known in the art and involves attaching fragments of nucleotide sequences (also known as adapters and indexes that contain sequences that help distinguish different library samples and sequences that pair with primers that help attach to the expository substrate) to the 2 ends of the cfDNA. In some embodiments, the procedure for attaching adapters and indexes to bisulfite-converted cfDNAs can be performed using the Accel-NGS™ Methyl-Seq DNA library kit (supplied by Swift Bioscience, USA). In some embodiments, the generated cfDNA library will be used for 2 purposes: (i) to analyze characteristic variations at 450 target sequence regions (see details in Table 23 provided elsewhere herein) and (ii) across the entire genome.

Start Here Fragmentation of the cfDNA Library for Variation Analysis at 450 Target Sequence Regions:

In some embodiments, the disclosed cfDNA library relates to 450 regions (e.g., containing 18,000 CpG sites) carrying methylation characteristic variations of many recorded types of cancer (Tables 23 and 24), hybrid captured by a probe set consisting of 2250 probes with the size of 120 bp specifically designed to capture these target sequence fragments through the principle of complementary pairing (Table 25). In some embodiments, the disclosed hybrid capture procedure is performed using the xGEN® Lockdown Reagent kit (supplied by Integrated DNA Technologies-IDT, USA). To reduce the rate of nonspecific capture (including adapter fragments and high repeat sequence regions in the genome), locking and preventing probes from binding can be implemented, for example, Human Cot 1 DNA (provided by Invitrogen, USA) and xGen Universal Blockers (provided by IDT, USA) can be used. After locking nonspecific sequences, this cfDNA library is hybridized with a probe set to capture target sequence regions. Next, magnetic beads are used to retain the probes bound to target sequence regions, for example, Dynabead™ streptavidin (provided by Invitrogen, USA). Meanwhile, the remaining sequences that are not captured by magnetic beads (called the “flow through” fragment) are recovered to analyze other markers. In some embodiments, the target sequence regions that have been retained by magnetic beads are then PCR amplified by, for instance, KaPa Hifi hotstart Polymerase enzyme (provided by Roche, Switzerland) with specific primers for 2 adapter fragments at 2 ends of each cfDNA fragment.

Library Fragment for Analysis of Genome-Wide Variations (“Flow Through” Fragment):

In some embodiments, the other cfDNA library fragment (“flow through” fragment) is recovered by hybridization with biotin-bound probes (e.g. a biotin-bound P5/P7 probe assembly provided by Integrated DNA Technologies—IDT, USA). In some embodiments, the cfDNA library fragment is obtained by streptavidin-bound magnetic beads (Dynabeads® M-270 Streptavidin beads—Invitrogen) via this bead's biotin-streptavidin binding. In some embodiments, the cfDNA library fragment is then PCR amplified and purified. PCR amplification can be performed using various suitable polymerases enzymes such as but not limited to KaPa Hifi hotstart Polymerase enzyme (provided by Roche, Switzerland). Purification can be performed using for instance, Kapa Pure Beads (provided by Roche, Switzerland). In some embodiments, the disclosed cfDNA library fragments are further sequenced. Sequencing can be performed via various suitable sequencing techniques known in the art, such as the MGI DNB-G400 system (provided by BGI, China). In some embodiments, after sequencing, the cfDNA library for such fragment (after hybrid capture) can be used to analyze methylation density, copy number abnormalities, and typical size of cfDNA across the whole genome including 22 autosomes.

Element 2: Analyze Different Variation Patterns of cfDNA.

Methylation density analysis at 450 target sequence regions:

In some embodiments, the sequencing data from the disclosed cfDNA library fragment comprises the promoter, the exons, the introns, and specific regions in the whole genome. In some embodiments, the disclosed SPOT-MAS test procedure comprises sequencing at a higher depth which increases the resolution to identify differences of methylation at the threshold level of at least 1%. Thus, the SPOT-MAS test procedure as provided herein improves sensitivity in detecting methyl changes that occur at early stages of cancer cell development.

Genome-Wide Methylation Density Analysis:

In some embodiments, the standard human genome is uniformly subdivided into non-duplicating fragments (bin) of 1 megabase (one million nucleotides) length (e.g., block 224 of FIG. 2B). In some embodiments, the methylation density (MD) per bin is calculated using the following formula:

$$MD = \frac{\sum mC}{( \sum mC + \sum T )} \times 100$$

where Σ mC is the total number of methylated C nucleotides and Σ T is the total number of nucleotides.

In some embodiments, the methylation trend is evaluated based on the Z-score of each bin using the following formula:

$$Zscore = \frac{\begin{matrix}MD in survey bin - \\ Mean MD in corresponding bin of the reference group\end{matrix}}{\begin{matrix}Standard deviation MD in \\ corresponding bin in the reference group\end{matrix}}$$

In some embodiments, if the Zscore of the tested bin region is less than −3 (Zscore<−3), that bin region is less methylated than the bin in the reference group.

In some embodiments, if the Zscore of the tested bin region is between −3 and 3 (−3<Zscore<3), methylation in that bin region is equivalent to the bin in the reference group.

In some embodiments, if the Zscore of the test bin region is more than 3 (Zscore>3), that bin region is more methylated than the bin in the reference group.

The analysis element as disclosed herein, helps selecting bin regions with different methyl variation levels between cancer patients and healthy people.

Analysis of Genome-Wide Copy Number Abnormalities:

In some embodiments, the standard human genome is uniformly subdivided into non-duplicating fragments (bin) of 1 megabase (one million nucleotides) length. In some embodiments, the copy number abnormalities are evaluated using the Zscore value using the formula:

$$Zscore = \frac{\begin{matrix}number of reads in survey bin - Average number of reads \\ in the corresponding bin of the standard reference group\end{matrix}}{\begin{matrix}Standard deviation of the number of reads \\ in the corresponding bin in the reference group\end{matrix}}$$

In some embodiments, if the Zscore of the tested bin region is less than −3 (Zscore<−3), that bin region has fewer copies than the bin in the standard reference group.

In some embodiments, if the Zscore of the tested bin region is between −3 and 3 (−3<Zscore<3), the number of copies that bin region has is equivalent to the bin in the standard reference group.

In some embodiments, if the Zscore of the tested bin region is more than 3 (Zscore>3), that bin region has more copies than the bin in the standard reference group.

In some embodiments, the Zscore value for variation in methyl density and DNA copy number as determined by the SPOT-MAS test helps identifying regions of genetic instability in the tumor genome. This is a prominent advantage of the SPOT-MAS test procedure because these markers contribute to accurate determination of the presence of cancer cells as well as their tissue origin based on the regions carrying these characteristic variations.

Analysis of Variation in cfDNA Size:

In some embodiments, the standard human genome is uniformly subdivided into non-duplicating fragments (bin) of 5 megabase (five million nucleotides) length. In some embodiments, within each of these bins, the ratio of the number of DNA fragments with size<=150 bp to those with size>150 bp is determined and used as a characteristic attribute of cfDNA size. It is known in the art that cancer cells tend to release more cfDNA fragments that are less than 150 bp in size. Thus determining the size difference of DNA fragments via the disclosed SPOT-MAS test procedure allows increasing the chances of tumor DNA being detected.

In one aspect, the disclosed SPOT-MAS test procedure provides generating data on different patterns of variation across the entire cell's DNA and identifying which variations are characteristic of tumor DNA. It is known in the art that methyl or size changes in tumor DNA are also markers to determine the origin of tumor DNA. Thus, incorporating the simultaneous analysis of these features by the disclosed SPOT-MAS test procedure addresses the need of increasing the chance of detecting tumor DNA and identifying its origin.

Element 3: Build a Machine Learning Model that Predicts Samples Carrying Cancer and Tumor Origin

In some embodiments, the machine learning model distinguishes samples with/without cancer.

Build a Machine Learning Model for Each Attribute.

In some embodiments, the process of building a machine learning model for each attribute comprises the following:

Divide dataset: In some embodiments, the dataset is divided into two sets, the training set and the leave-out test set using the 7:3 ratio. For the model training set, the data is further randomly divided several times (with cross-validation) into model training and validation sets.

Model training: In some embodiments, the algorithm model is trained in turn with the models using the training data sets and evaluates the effectiveness of the model after training with the model validation sets using the algorithm combining 1000 basic classification models of the same type called Bagging Ensemble. This model is trained based on classification algorithms including Extreme Gradient Boosting (XGBoost), logistic regression (LR) and support vector machine (SVM) models. Nowadays, LR and SVM classification algorithms are widely applied to perform binary classification. XGBoost is a recently developed boosting algorithm and has been shown to have good speed and performance on many large datasets. For each algorithm, the parameters are adjusted to optimize for the performance (e.g., sensitivity, specificity, accuracy, etc.) of the model using the GridsearchCV algorithm.

Set the cut-off threshold: To set a suitable cut-off threshold for the model, it is necessary to determine the sensitivity, specificity, and accuracy of the model. In some embodiments, sensitivity, specificity and accuracy are calculated using the formula:

$$Accuracy = \frac{( a + d )}{( a + b + c + d )}$$

$$Sensitivity = \frac{( a )}{( a + c )}$$

$$Specificity = \frac{( d )}{( b + d )}$$

where:
a (true positive) is a cancer sample and is classified as cancer by the algorithm.
b (false positive) is a healthy sample and is classified as cancer by the algorithm.
c (false negative) is a cancer sample and is classified as a healthy sample by the algorithm.
d (true negative) is a healthy sample and is classified as a healthy sample by the algorithm.

In some embodiments, the cut-off threshold value is set based on the value of specificity and is surveyed to range from 0 to 1. In some embodiments, for each specificity value, a different set of sensitivity and accuracy values is obtained. From there, the ROC (receiver operating curve) model is built. In some embodiments, based on the ROC curve, a cut-off threshold is selected so that the specificity is at least 80%, at least 85%, at least 90%, at least 91%, at least 92%, at least 93%, at least 94%, at least 95%, at least 96%, at least 97%, at least 98%, or at least 99%. In some embodiments, based on the ROC curve, a cut-off threshold is selected so that the specificity is at least 95%. The area under the ROC curve is then calculated, often called AUC (area under the ROC curve). It is known in the art that the larger the area, the higher the accuracy of the model.

In some embodiments, the weight and number of occurrences of gene or bin regions in each attribute in 1000 times when training the model will be recorded and rated. The larger the weighted bin or gene regions and the higher the frequency of occurrence, the greater the significance of contributing to the model's performance.

In some embodiments, the effectiveness of the model on the leave-out test set is evaluated based on the following: After selecting a model with the best performance, the effectiveness of the selected model will be evaluated on the model evaluation dataset. Like the model training element, the indicators of specificity, sensitivity, accuracy, and AUC values of the model are determined on the model evaluation dataset. The model achieves the best performance when these values are highest and are equivalent to the values obtained in the model training element.

Build a Model that Combines Different Attributes.

In some embodiments, after evaluating the effectiveness of the models built on each attribute, the multi-attribute combination model is built with a strategy of linearly combining the categorical prediction results of each individual attribute.

The prediction result of individual models built on each attribute group of cfDNA is the probability value corresponding to that attribute for each sample. In some embodiments, a new dataset is formed, consisting of four categorical prediction values corresponding to four attribute groups. In some embodiments, the newly built logistic regression combined linear model as disclosed herein allows combining these attributes and determining the weight of each attribute's contribution to the final categorical prediction result. In some embodiments, the final model applied in the disclosed SPOT-MAS test procedure is a stacking model of individual attributes for the first layer and a logistic regression model for the second layer.

Determining the Origin of the Tumor

In some embodiments, after classifying cfDNA as being of tumor origin, the SPOT-MAS test procedure as provided herein further analyzes the source (from which organ in the body) of cfDNA release. The analytical procedure is based on the principle that cfDNA released from which organ will have variations in the methylation level, the size of DNA fragments that is characteristic of that organ. Specifically, the classification of tumor origin is built based on machine learning classification algorithms. In some embodiments, the attributes initially included in the analysis comprise variation in genome-wide methylation density, target methylation density, and size of cfDNA fragments (long fragment, short fragment, size ratio). In some embodiments, for each attribute type, machine learning algorithms are used to classify the tumor origin from different organ types (e.g., liver, lung, colorectal, stomach, and breast) by default to find the most suitable algorithm and attribute for the highest classification efficiency. In some embodiments, the machine learning algorithms to be surveyed include a deep neural network, logistic regression, random forest, and support vector machine. In some embodiments, the machine learning algorithm is a deep neural network.

In some embodiments, four patterns of characteristic variations in tumor DNA include:

Methylation at Specified Sites of Genes Involved in Tumor Growth

Methylation is a epigenetic mechanism known in the art that indicates when cytosine sites (C sites) in CpG islands are linked with CH₃ group. In some embodiments, to detect C sites that are linked with CH₃ group, the DNA is treated with bisulfite chemicals. Under the influence of chemicals, which C sites do not have “protection” of CH₃ group will be converted to T nucleotides while C sites that are linked with CH₃ group will be preserved. In some embodiments, sequencing methods allow determining which C sites are or are not methylated. Based on such determination, the methylation density at these sites can be calculated.

In some embodiments, the relevant genomic regions selected for investigation in the SPOT-MAS procedure are a list of 450 target gene regions containing 18,000 CpG sites that control the expression of tumor suppressor genes (Table 23). In the early stages of cancer, these regions are highly methylated to inhibit the expression of tumor suppressor genes that promote tumor proliferation and transformation. Therefore, based on this feature, it is possible to distinguish the DNA released by cancer cells into sample from the DNA of normal cells.

Genome-Wide Methylation of Tumors

The methylation and determination of genome-wide methylation status of tumor are similar to the methylation at specific sites of genes associated with tumor growth. However, when investigating genome-wide methylation characteristics, many studies demonstrated that the methylation status tends to decrease in many different cancers. This tendency of methylation decrease facilitates the activation of oncogenes, especially in the early stages of tumorigenesis. Thus, when comparing the trend of genome-wide methylation in cancer patients with healthy people, the trend of methylation decrease in cancer patients has been observed. Harnessing this feature allows cancer to be identified at a very early stage.

Genome-Wide Copy Number Abnormalities of Tumor DNA.

The presence of structural abnormalities of the chromosome is a common characteristic found in all types of cancer. These abnormalities often occur very early and accumulate gradually during the formation and growth of the tumor. Abnormalities range from fragment deletions, duplications, and inversions on whole branches of chromosomes to fragment amplifications or deletions located at different sites in the genome. The consequence of these abnormalities is structural rearrangement of genes and instability of the genome, and the resulting proteins are structurally and functionally defective.

Often, the genome in cancer patients will have regions that are amplified many times or lost some regions. By sequencing the whole genome, the number of cfDNA molecules on each bin region of the chromosome will be counted, thereby determining which bin regions increase or decrease the copy number of the entire tumor genome. When comparing the copy number of each bin region of the genome in cancer patients and healthy people, copy number abnormalities were noted. Based on the abnormality of the copy number on the whole genome, it is possible to identify the presence of cancer cells.

Characteristic Size of DNA Released by the Tumor into the Bloodstream

The cfDNA molecules present in the blood are released from cells undergoing the apoptosis. This apoptosis of cancer cells and normal cells is different, resulting in cfDNA released from these two cell types with different lengths. Specifically, the size of cfDNA released from tumors is usually shorter than that of cfDNA released normal cells.

To determine the size of cfDNA, whole-genome sequencing is performed to “measure” the length of the cfDNA fragments. Count the number of cfDNA molecules of the same size and use them to calculate the distribution density on a scale from 0 to 250 nucleotides. The density of cfDNA fragments smaller than 150 nucleotides is usually higher in the blood of cancer patients than in the blood of healthy individuals. Based on the size characteristics of cfDNA, it is possible to identify the presence of cancer cells.

### EXAMPLES

The present disclosure is now described with reference to the following Examples. These Examples are provided for the purpose of illustration only and this disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed systems and methods. The following working examples, therefore, specifically point out the preferred embodiments of the present disclosure, and are not to be construed as limiting in any way the remainder of the disclosure.

In the examples disclosed herein blood tests of a group of patients with colorectal cancer (CRC), liver cancer, lung cancer, breast cancer, gastric cancer and a group of healthy people were conducted using a liquid biopsy procedure (SPOT-MAS test procedure) to detect tumor DNA.

As shown in FIG. 3, the disclosed liquid biopsy procedure (SPOT-MAS test procedure) allows simultaneous detection of four patterns of characteristic variations of tumor DNA including: i) methylation at specific sites of genes related to tumor growth; ii) genome-wide methylation of tumor; iii) genome-wide copy number abnormalities of tumor DNA; and iv) the typical size of DNA released by the tumor into the bloodstream.

The materials and methods employed in the experiments disclosed herein are now described.

Materials and Methods

Element 1: Prepare a sequencing library of bisulfite-treated cell-free DNA (cfDNA)

1.1 Preparing cfDNA Library

Cell-free DNA (cfDNA) is DNA that can be released from cancer cells and normal cells (leukemic cells) into the bloodstream when undergoing the apoptosis or necrosis. For cfDNA collection, blood samples can be collected and stored in a Streck cell-free DNA BCT (218997) anticoagulant test tube. First, plasma and cellular components were separated twice by centrifugation. Then, extract cfDNA from the plasma using extraction kits, for example, the MagMAX cell-free DNA extraction kit (supplied by Thermo Fisher, USA) on the KingFisher Flex Magnetic 96DW automated system (provided by Thermo Fisher, USA) following the manufacturer's instructions. At the end of the program, the resulting cfDNA was recovered and stored in a Lobind tube (Eppendorf AG), kept at −20° C. if not used immediately and the concentration was evaluated using the QuantiFluor dsDNA system (provided by Promega, USA).

1.2 Bisulfite Treatment

The treatment of cfDNA with bisulfite was carried out to convert cytosine (C)-type nucleotides with a methyl moiety (—CH3) to uracil-type (U) nucleotides, while C-type nucleotides without methyl moiety are not converted. Thus, the treatment of cfDNA with bisulfite (BS) helps detecting methylation on cfDNA. Bisulfite conversion was performed on cfDNA using the EZ DNA Methylation-Gold Kit (provided by Zymo Research, USA) following the manufacturer's instructions. The product was then purified and desulfurized on Zymo-Spin™ IC Column. The resulting cfDNA was resolved in 7.5 μL of M-elution buffer.

1.3 Creating cfDNA Sequencing Library

After processing with BS, cfDNA was attached with adapters and indexes. An adapter is a nucleotide sequence attached to two ends of a DNA fragment that enables the DNA to attach to a rack on the surface of a flow cell in a sequencing system and be recognized by primer sequences to be amplified. An index is a nucleotide sequence that is specific to each sample and helps to distinguish different samples when performing simultaneous sequencing of multiple samples. The procedure for attaching adapters and indexes to bisulfite-converted cfDNA is known in the art and can be performed for instance by using the Accel-NGS™ Methyl-Seq DNA library kit (supplied by Swift Bioscience, USA) following the manufacturer's instructions. After attaching adapters and indexes, the cfDNA fragments were called cfDNA library and used for the portions of the pipeline.

Tumor formation and growth is the result of expression changes of many oncogenes and tumor suppressor genes. The expression of these genes is closely controlled through a methylation mechanism that occurs at regulatory regions such as promoters and enhancers regions. These regions often contain CpG islands which are CG sequences that appear with high frequency and the addition of CH₃ group (referred to as methylation) at C sites of CpG islands inhibits gene expression. Methylation at regulatory regions of tumor suppressor genes often occurs during tumor initiation. Therefore, methylation variation in these regions can be used as tumor markers. Based on previous publications and knowledge in the art, a list of 450 target genomic regions containing 18,000 CpG sites carrying characteristic methylation variation of many types of cancer has been established. To investigate the methylation density at 450 target genomic regions (Tables 23 and 24), a probe set consisting of 2250 DNA fragments with the size of 120 bp was specifically designed to capture these target sequences through the principle of complementary pairing (Table 25).

The hybrid capture procedure was performed with the xGEN® Lockdown Reagent kit (provided by Integrated DNA Technologies-IDT, USA) following the manufacturer's instructions. To reduce the rate of nonspecific capture (including adapter fragments and high repeat sequence regions in the genome), locking and preventing probes from binding was implemented, for example by using Human Cot 1 DNA (provided by Invitrogen, USA) and xGen Universal Blockers (provided by IDT, USA). After locking the nonspecific sequences, the disclosed cfDNA library was hybridized with a probe set to capture target sequence regions. Next, Dynabead™ streptavidin magnetic beads (supplied by Invitrogen, USA) were used to retain the probes bound to target sequence regions. Meanwhile, the remaining sequences that were not captured by magnetic beads (called the “flow through” fragment) were recovered for other markers analysis. The target sequence regions that was retained by magnetic beads was subsequently used for PCR amplification by KAPA Hifi hotstart Polymerase enzyme (provided by Roche, Switzerland) with specific primers for 2 adapter fragments at 2 ends of each cfDNA fragment. After PCR, the concentration of cfDNA library product after hybrid capture was quantified using the Quantus system. After the amplification reaction, the cfDNA library fragments was sequenced using paired-end sequencing mode at 100-bp on the MGI DNB-G400 system (provided by BGI, China) with a depth of 20 million reads for 1 sample.

1.4 Collecting and Processing “flow Through” Fragments

After hybrid capture, the remaining cfDNA library fragments (“flow through” fragments) was recovered by hybridization with a P5/P7 probe assembly (provided by Integrated DNA Technologies—IDT, USA). These probes are nucleotide sequences with biotin molecules attached and additionally paired with adapter sequences P5 and P7 at both ends of the cfDNA library. cfDNA in this flow-through fragment, after being specifically attached to the P5/P7 probe, were collected using magnetic beads (Dynabeads® M-270 Streptavidin beads-Invitrogen) through the magnetic beads' biotin-streptavidin binding. Then, the cfDNA library in this flow-through fragment was PCR amplified using the KaPa Hifi hotstart Polymerase enzyme (provided by Roche, Switzerland). After amplification, the product was purified using Kapa Pure Beads (provided by Roche, Switzerland). Amplified product concentration was quantified using the Quantus system. cfDNA sequencing was performed on this flow-through fragment using the MGI DNB G400 system with a depth of 20 million reads per sample as described above.

Element 2: Analyze Different Variation Patterns of cfDNA.

2.1 Analysis of Methylation Variation at 450 Target Gene Regions (Containing 18,000 CpG Sites)

Sequencing data from cfDNA sequencing library fragments was particularly focused on promoters, exon, intron, and intergenic regions of cancer-related genes. The quality of the raw data was checked using FastQC tool (Babraham Institute, version 0.11.9). Poor quality data and adapter sequences were removed using a trimmomatic tool (USADEL lab, version 0.39).

Read sequences were aligned with the standard genome and analyzed to determine methylation percentage using the Bismark aligner tool (Babraham Institute, version 16.0.2). Regions with different methylation percentages between cancer and healthy groups (called DMR: Differentially Methylated Regions) were determined by the methylation percentage per CpG determined using the following formula:

$$Methylation percentage = \frac{{N}_{C , i}}{{N}_{C , i} + {N}_{T , i}} \times 100 \%$$

where:
i: The i^{th} CpG site in the region of interest;
N_{T,i}: Number of T nucleotides observed at the i^{th} CpG site; and
N_{C,i}: Number of C nucleotides observed at the i^{th} CpG site.

The regions with different methylation percentage between the cancer group and the healthy group were determined accordingly. Specifically, the percentage of methylation of the healthy group and the cancer group on each corresponding CpG site were compared by the Wilcoxon ranked sum test (Mann Whitney U test), in order to identify regions with (statistically significant) differences on the methylation density of CpG. The Wilcoxon ranked sum test is suitable when comparing multiple variables simultaneously between 2 groups of independent samples and variables that are not normally distributed (non-parametric test). In addition, the p-value of the statistical test was corrected using the Benjamini Hochberg method to avoid the false-positive situation encountered when the number of variables to be compared is much larger than the number of analyzed samples. The regions with different percentages of methylation between cancer and healthy groups were identified when p-value was less than 0.05 (p-value<0.05).

The methylation fold change between the cancer group and the healthy group was determined. Specifically, the percentage of methylation (between cancer and healthy groups) on each respective CpG site is used to determine how many times the methylation fold change has changed. The methylation fold change was corrected by taking the log to base 2 (|log 2|) of the absolute value of the above percentage. If this value was greater than 1, the methylation fold change has changed more than 2 times between the cancer group and the healthy group.

2.2 Genome-Wide Methylation Density Change Analysis

The quality of the sequencing data of the flow-through library fragments was checked by using FastQC software. Poor quality data and adapter sequences were removed using a trimmomatic tool. Read sequences were aligned against the human reference genome sequence (version hg19) using the BSAligner software in the Methyl pipe analysis package (DOI: 10.1371/journal.pone.0100360). The following parameters were checked: (1) proportion of reads is aligned against the reference genomic sequence in total mappability, (2) depth of sequencing, (3) sequencing coverage of all samples.

Genome-wide methylation variation consisting of 22 chromosomes was determined as follows. The standard human genome was uniformly subdivided into non-duplicating fragments (bin) of 1 megabase (one million nucleotides) length. Analysis of methylation variation was performed on each bin. The methylation density (MD) per bin was calculated using the following formula:

$$MD = \frac{\sum mC}{( \sum mC + \sum T )} \times 100$$

where: ΣmC is the total number of methylated C nucleotides; and ΣT is the total number of T nucleotides. Bins with variation in methylation state were identified. Sequencing data from 19 healthy subjects were randomly selected to determine the reference MD value for each bin. Variation in values of methylation density in each bin was evaluated based on the “Z score” value using the following formula:

$$Zscore = \frac{\begin{matrix}MD in survey bin - \\ Mean MD in corresponding bin of the reference group\end{matrix}}{\begin{matrix}Standard deviation MD in \\ corresponding bin in the reference group\end{matrix}}$$

If Zscore<−3, that bin region was less methylated than the bin in the reference group.

If −3<Zscore<3, methylation in that bin region was equivalent to the bin in the reference group.

If Zscore>3, that bin region was more methylated than the bin in the reference group.

2.3 Genome-Wide DNA Copy Number Abnormalities Analysis

Sequencing data of the flow through library fragments was used for genome-wide DNA copy number abnormalities analysis. Data quality was checked using FastQC software. Poor quality data and adapter sequences were removed using a trimmomatic tool. Read sequences were aligned against the human reference genome sequence (version hg19) using the BSAligner software in the Methyl pipe analysis package (DOI: 10.1371/journal.pone.0100360).

The following parameters were checked: (1) proportion of reads was aligned against the reference genomic sequence in total mappability, (2) depth of sequencing, (3) sequencing coverage of all samples. DNA copy number abnormalities analysis on 22 chromosomes was performed on each bin.

The number of copies of DNA in the bins were determined: Differences in the number of reads between bins can occur due to the influence of the bin region containing many G and C nucleotides (GC-bias) or the presence of repeat sequence regions (tandem repeat). Therefore, after alignment, the number of reads in each bin were corrected using the QDNASeq tool (DOI: 10.1101/gr.175141.114). The median copy number of all bins after correction were calculated. The degree of variation in the number of copies per bin was determined by taking the log to base 2 (|log 2|) of the absolute value of the ratio of the number of reads in that bin to the median of the reads of all bins. If this value was greater than 1, then the degree of variation was more than 2 times between the investigated bin and the whole genome.

The proportion of bins with DNA copy number abnormalities between the cancer group and healthy people was determined.

Sequencing data from 19 healthy subjects were randomly selected to determine the average number of reads for each bin. Variation of gene copy number in each bin was evaluated based on the “Z score” value using the following formula:

$$Zscore = \frac{\begin{matrix}number of reads in survey bin - Average number of reads \\ in the corresponding bin of the standard reference group\end{matrix}}{\begin{matrix}Standard deviation of the number of reads \\ in the corresponding bin in the reference group\end{matrix}}$$

If Zscore<−3, that bin region had fewer copies than the bin in the reference group

If −3<Zscore<3, the number of copies that bin region had was equivalent to the bin in the reference group

If Zscore>3, that bin region had more copies than the bin in the reference group

2.4 Analysis of Variation in cfDNA Size.

The sequencing data of the flow through library fragments was used to analyze variation in cfDNA size. Data quality was checked using FastQC software. Poor quality data and adapter sequences were removed using a trimmomatic tool.

Read sequences were aligned against the human reference genome sequence (version hg19) using the BSAligner software in the Methyl pipe analysis package (DOI: 10.1371/journal.pone.0100360). Check parameters: (1) proportion of reads is aligned against the reference genomic sequence in total mappability, (2) depth of sequencing, (3) sequencing coverage of all samples.

Variation in cfDNA size was determined as follows. The standard human genome was uniformly subdivided into non-duplicating fragments (bin) of 5 megabase (5 million nucleotides) length. Size variation analysis was performed on each bin. After alignment, the length of each cfDNA fragment was calculated using software (bsalign). The size of cfDNA fragment was calculated based on the distance between the starting point of the Watson reading in the standard genome and the end point of the reading in the opposite direction (Crick). The size distribution ratio of cfDNA fragments of cancer and healthy samples in the range of 0 to 250 nucleotides was determined. Fragment ratio (RF) per bin was calculated using the following formula:

$$R F = \frac{( P \leq 1 \; 50 bp )}{( P > 1 \; 50 bp )} \times 100$$

where: P≤150 bp means length of reads is 150 nucleotides or less and P>150 bp means length of reads is over 150 nucleotides.

RF variation on all 22 chromosomes was determined.

Element 3: Build a Machine Learning Model that Predicts Samples Carrying Cancer and Tumor Origin.

Resulting analytical data in sections 2.1, 2.2, 2.3 and 2.4 as provided above herein was converted to quantitative data of 4 different attributes for each cfDNA sample including: methylation density attribute of 450 target regions (2.1); methylation density attribute of genome-wide bins (22 chromosomes) (2.2); DNA copy number attribute of genome-wide bins (22 chromosomes) (2.3); cfDNA size-specific ratio attribute of genome-wide bins (22 chromosomes) (2.4). The machine learning model was built for each individual group of attributes and combination of all attribute groups. The effectiveness of this model was evaluated based on its ability to classify 2 groups of samples as cancer and healthy people or between malignant and benign tumors.

3.1 Machine Learning Model can Distinguish Samples with and without Cancer.

Build a Machine Learning Model for Each Attribute.

The process of building a machine learning model for each attribute comprised the following:

Dividing dataset: The dataset was divided into two sets, the training set and the leave-out test set using 7:3 ratio. For the model training set, the data was further randomly divided several times (with cross-validation) into model training and validation sets.

Model training: The algorithm model was trained in turn with the models using the training data sets and evaluated the effectiveness of this model after training with the model validation sets using the algorithm combining 1000 basic classification models of the same type called Bagging Ensemble. This model was trained based on classification algorithms including Extreme Gradient Boosting (XGBoost), logistic regression (LR) and support vector machine (SVM) models. Nowadays, LR and SVM classification algorithms are widely used in the art to perform binary classification. XGBoost is a recently developed boosting algorithm and was shown to have good speed and performance on many large-sized datasets. For each algorithm, the parameters used in this disclosure were adjusted to optimize the efficiency of the model using the GridsearchCV algorithm.

Set the cut-off threshold: To set a suitable cut-off threshold for the model, it is necessary to determine sensitivity, specificity and accuracy of the model. In the present disclosure the sensitivity, specificity and accuracy were calculated using the formula:

$$Accuracy = \frac{( a + d )}{( a + b + c + d )}$$

$$Sensitivity = \frac{( a )}{( a + c )}$$

$$Specificity = \frac{( d )}{( b + d )}$$

where:
a (true positive) is a cancer sample and is classified as cancer by the algorithm,
b (false positive) is a healthy sample and is classified as cancer by the algorithm,
c (false negative) is a cancer sample and is classified as a healthy sample by the algorithm, and
d (true negative) is a healthy sample and is classified as a healthy sample by the algorithm.

The cut-off threshold value was set based on the value of specificity and it was surveyed to range from 0 to 1. For each specificity value, a different set of sensitivity and accuracy values were obtained. From there, the ROC (receiver operating curve) model was built. From the ROC curve, the cut-off threshold was selected so that the specificity was at least 95%. The area under the ROC curve, often called AUC (area under the ROC curve), was calculated. The larger the area, the higher the accuracy of the model.

The weight and number of occurrences of the gene or bin regions in each attribute in 1000 times when training the model was recorded and rated. The larger the weighted bin or gene regions and the higher the frequency of occurrence, the greater the significance of contributing to the model's performance.

The effectiveness of the model was evaluated on the leave-out test set: After selecting the model with the best performance, the effectiveness of the selected model was evaluated on the model evaluation dataset. Similar to the model training element, the indicators of specificity, sensitivity, accuracy and AUC values of the model were determined on the model evaluation dataset. The model had the best performance when these values were the highest and were equivalent to the values obtained in the model training element.

Build a Model that Combines Different Attributes.

After evaluating the effectiveness of the models built on each attribute, the multi-attribute combination model was built with a strategy of linearly combining the categorical prediction results based on each individual attribute.

The prediction result of individual models built on each attribute group of cfDNA corresponded to the probability value corresponding to that attribute for each sample. Thus, a new dataset was formed, consisting of 4 categorical prediction values corresponding to 4 attribute groups. The newly built logistic regression combined linear model allowed combining these attributes and determining the weight of each attribute's contribution to the final categorical prediction result. The final model applied in the SPOT-MAS test procedure was a stacking model of individual attributes for the first layer and a logistic regression model for the second layer.

3.2 Determining the Origin of the Tumor.

The sequence for building a model to determine the tumor origin included the following selected attributes: methyl region or bin region with methylation, the size of DNA fragments that was characteristically different between five (5) types of cancer:
Each sample had fragment size data of 588 bins, methylation of 2734 bins and 450 regions.
All data from samples in the cancer (5 types) group and healthy group were divided into algorithm training set (7 parts) and algorithm test set (3 parts).
In the algorithm training sample group, the Least Absolute Shrinkage and Selection Operator (LASSO) was used to find bins with characteristically different DNA methylation or fragment sizes between 4 types of cancer.

After selecting useful attributes, a logistic regression machine learning algorithm was used to build a model using a training sample group to help determine the probability value of 5 cancer types of that sample. From there, the organ origin of ctDNA was determined based on the highest probability value of that organ.

After training, the classification algorithm was tested on a test sample set, and for each true or false classification result, the sensitivity, specificity and accuracy of the model were calculated to evaluate the classification effectiveness of the model.

### Example 1: Element 1—Create a Sequencing Library of Bisulfite-Treated Cell-Free DNA (cfDNA)

1.1 Process Blood Samples to Collect Plasma

A 10 ml BD Vacutainer blood collection tube, USA (368589) with anticoagulant (K2-EDTA) was used to collect blood samples from the patients. Process the collected blood samples within no longer than 6 hours at a temperature of about 4° C. Separate the plasma twice by centrifugation as follows:

First centrifugation: Blood tubes were centrifuged at 1,600 g for 10 min at 4° C. The upper plasma layer was gently aspirated into a 2 ml Eppendorf tube without touching the mononuclear cell layer. Then the mononuclear cells were aspirated into a 2 ml Eppendorf tube and freeze at −80° C.

Second centrifugation: The above-mentioned plasma layer was centrifuged at the speed of 16,000 g for 10 minutes, at 4° C. The supernatant was collected into 1.5 ml Eppendorf tubes and the residue at the bottom of the tubes was discarded. The obtained plasma sample was either used immediately for cfDNA extraction or frozen at −80° C.

1.2 Extraction of cfDNA:

cfDNA extraction was performed on KingFisher Flex Magnetic 96DW automated system using the commercial MagMAX cell-free DNA Isolation kit (supplied by ThermoFisher Scientific, USA).

880 uL of plasma was used for cfDNA extraction. The plasma was divided equally between the 2 sample plates. Table 1 below lists the chemicals used for cfDNA extraction corresponding to the elements to perform the cfDNA extraction in the KingFisher Flex Magnetic 96DW with 96 deep well plate process. Be sure to use the standard plate for the 6^{th} position and deep well plates for all other positions.

TABLE 1

| | Plate | | | |
|---|---|---|---|---|
| | position | | Volume | |
| | on the | | of each | |
| Purpose | extractor | Chemicals used | well | |
| Lysing and mixing | 1 | MagMAX ™ Cell Free DNA | 550 | μL |
| sample with | | Lysis/Binding Solution | | |
| magnetic beads | | MagMAX ™ Cell Free DNA | 8 | μL |
| | | Magnetic Beads | | |
| | | Plasma blood sample | 440 | μL |
| Lysing and mixing | 2 | MagMAX ™ Free DNA Cell | 550 | μL |
| sample with | | Lysis/Binding Solution | | |
| magnetic beads | | MagMAX ™ Cell Free DNA | 8 | μL |
| | | Magnetic Beads | | |
| | | Blood sample plasma | 440 | μL |
| 1^{st} wash | 3 | MagMAX ™ Cell Free DNA | l | mL |
| | | Wash Solution | | |
| 2^{nd} wash | 4 | 80% alcohol | 1 | mL |
| 3^{rd} wash | 5 | 80% alcohol | 500 | mL |
| Recover cfDNA | 6 | MagMAX ™ Cell Free DNA | 30 | μL |
| | | Elution Solution | | |

| | |
|---|---|
| 7 | The tip-comb was placed in deep well |
| | plate for lysis |

The attachment, washing and elution of the obtained cfDNA were performed as follows: setting parameter, selecting function for suitable plate position on KingFisher Flex Magnetic 96DW extractor. The chemical plates and samples were paced in suitable positions on the extractor and the extraction was carried out. At the end of the cycle (approximately 47 minutes), the cfDNA recovery plate located at the 6^{th} position on the extractor was removed from the extractor. The cfDNA sample was either used immediately for the next element or transferred to a Lobind tube (Eppendorf AG) for storage at −20° C. for a long-term use.

1.3 Measure cfDNA Concentration Using QuantiFluor dsDNA System.

The concentration of cfDNA was measured with Quantus™ Fluorometer (E6150) measuring system, using QuantiFlour dsDNA system (E2670). This was as follows: Dilute 20×TE buffer 20 times with distilled water to obtain 1× TE buffer. Dilute QuantiFlour dsDNA dye 400 times with 1×TE buffer to obtain a measuring buffer. Aspirate 198 μL of measuring buffer into a 0.5 ml thin-walled PCR tube (Cat. #E4941). Add 2 μL of cfDNA sample to be measured into the PCR tube and incubate at room temperature for 5 minutes, avoiding direct sunlight. Measure sample with Quantus™ Fluorometer meter system and record the obtained cfDNA concentration.

1.4 Bisulfite Treatment (BS).

Bisulfite treatment of cfDNA was performed with 2ng cfDNA using Zymo EZ DNA Gold methylation reagent kit (D5006), including the following:

CT Conversion Reaction.

CT conversion reagent tube was dissolved with 900 μL of H₂O, 300 μL of M-Dilution buffer and 50 μL of M-Dissolving buffer. The tube was placed on a shaker for 10 minutes or until completely dissolved. 20 μL of cfDNA were aspirated into 0.2 mL PCR tube. The amount of H₂O was adjusted so that the volume of cfDNA in the tube reached 2ng. 130 μL of CT conversion reagent were added and mixed by suction and release 10 times. The mixture was placed in a heat cycler and the thermal process followed the settings shown in the Table 2 below.

TABLE 2

| Element | Temperature | Time | |
|---|---|---|---|
| 1 | 98° C. | 10 | minutes |
| 2 | 64° C. | 2.5 | hour |

Kept at 4° C.

Purifying the product after bisulfite modification.

The purification element involved the following: Prepare an M-wash buffer by adding 24 ml of 100% alcohol to 6 ml of concentrated M-wash buffer. Prepare the Zymo-Spin™ IC membrane kit and collection column. Add 600 μL of M-binding buffer into the membrane kit. Aspirate all 150 μL of the CT conversion product mixture in the PCR tube into the collection column and mix well by manually inverting several times. Centrifuge the collection column at 11,000 g for 30 seconds and then discard the solution in the collection column. Add 100 μL of M-wash buffer to the collection column and centrifuge the second time at 11,000 g for 30 seconds. Add 200 μL of M-Desulphonation buffer to the collection column and incubate at room temperature for 15 minutes. Then centrifuge the column for the third time at 11,000 g for 30 seconds. Add another 200 μL of M-wash buffer to the collection column and centrifuge the fourth time at 11,000 g for 30 seconds. Discard the solution in the collection column and continue adding 200 μL of M-wash buffer. Then centrifuge the column for the fifth time at 11,000 g for 30 seconds. Empty the collection column and transfer Zymo-Spin™ IC membrane to a new 1.5 ml Eppendorf tube. Add 7.5 μL of M-elution buffer to the center of the membrane and incubate for 5 minutes at room temperature, centrifuge at maximum speed for 1 minutes to obtain cfDNA sample. This cfDNA sample can be used immediately or stored at −20° C.

1.5 Generating a Sequencing Library for Bisulfite Treated cfDNA.

Attaching adapters and indexes.

Denaturation-separation of cfDNA: After bisulfite treatment, cfDNA product was denatured to separate single-stranded cfDNA by incubation at 95° C. for 2 minutes in a heat cycler. The sample was immediately removed and placed on cold ice for 2 minutes to prevent regurgitation. A reaction mixture was prepared for attaching the adapter 1 to the components as shown in the Table 3 below.

TABLE 3

| Chemicals | Volume (μL) |
|---|---|
| Low TE buffer | 6.75 |
| G1 buffer | 2 |
| G2 chemicals | 2 |
| G3 chemicals | 1.25 |
| G4 yeast | 0.5 |
| G5 yeast | 0.5 |
| G6 yeast | 0.5 |
| Total volume | 13.5 |

13.5 μL of the above reaction mixture was added into 7.5 μL cfDNA sample after the denaturation-separation element. The reaction mixture was mixed well by suction-release 10 times and incubated in a heat cycler with the program set at the temperature and time shown in the Table 4 below.

TABLE 4

| Element | Temperature | Time |
|---|---|---|
| 1 | 37° C. | 15 minutes |
| 2 | 95° C. | 2 minutes |

Kept at 4° C.

Extend strands to create non-Uracil library: The chemical mixture was prepared for strand extension reaction with the components and volumes shown in the Table 5 below.

TABLE 5

| Chemicals | Volume (μL) |
|---|---|
| Y1 chemicals | 1 |
| Y2 yeast | 21 |
| Total volume | 22 |

Right at the end of attaching adapter 1 process, 22 μL of the extension chemical mixture was added. This mixture was mixed well by suction-release 10 times and incubated in a heat cycler with the program parameters as shown in the Table 6 below.

TABLE 6

| Element | Temperature | Time | |
|---|---|---|---|
| 1 | 98° C. | 1 | minute |
| 2 | 62° C. | 2 | minutes |
| 3 | 65° C. | 5 | minutes |

Kept at 4° C.

Purifying the product after strand extension: 50.4 μL of KAPA magnetic beads were added into the tube containing the strand extended product, mixed well by suction-release 10 times and incubated at room temperature for 5 minutes. The sample tube was placed on a magnetic tray to capture magnetic beads until the solution cleared, and then the supernatant was discarded. 200 μL of 80% alcohol solution was added, incubated for 30 seconds and the supernatant was discarded. Add 200 μL of 80% alcohol solution, incubate for 30 seconds and discard the supernatant. The magnetic beads were left to dry naturally for 1 to 3 minutes but without letting them dry too much. The tube from the magnetic tray was removed and 7.5 μL were added of low TE. A magnetic bead suspension was created by suction-release 10 times and incubated at room temperature for 5 minutes. The tube containing the amplified product was placed on the magnetic tray to capture the magnetic beads, until the solution became clear, then the supernatant was transferred into a new 0.2 ml tube to prepare for the next element.

Connecting and attaching the 2^{nd} adapter: The chemical mixture for the coupling reaction and attaching the 2^{nd} adapter with the components and volumes are shown in the Table 7 below.

TABLE 7

| Chemicals | Volume (μL) |
|---|---|
| B1 buffer | 1.5 |
| B2 chemicals | 5 |
| B3 yeast | 1 |
| Total volume | 7.5 |

The connection of the 2^{nd} adapter involved the following: Add 7.5 μL of the above chemical mixture to 7.5 μL of the cfDNA product purified in the previous element. Mix this mixture well by suction-release 10 times. Incubate this mixture in a heat cycler at 25° C. for 15 minutes. To purify the product after connecting and attaching the 2^{nd} adapter, add 18 μL of KAPA magnetic beads into the tube containing the amplified product. Mix well by suction-release 10 times and incubate at room temperature for 5 minutes. Place the sample tube on a magnetic tray to capture magnetic beads, wait for the solution to clear and discard the supernatant. Add another 200 μL of 80% alcohol solution into the sample tube, incubate for 30 seconds and discard the supernatant. Add another 200 μL of 80% alcohol solution, incubate for 30 seconds and discard the supernatant. Let the magnetic beads dry naturally for 1 to 3 minutes and avoid letting them too dry. Remove the tube from the magnetic tray, add another 10 μL of low TE. Create magnetic bead suspension by suction-release 10 times and incubate at room temperature for 5 minutes. Place the tube containing the amplified product on the magnetic tray to capture the magnetic beads, wait for the solution to clear and transfer the supernatant into a new 0.2 ml tube to prepare for the next element.

Amplify and attach indexes: The chemical mixture for amplification reaction was prepared and the index attachment including the components and volumes are shown in the Table 8 below.

TABLE 8

| Chemicals | Volume (μL) |
|---|---|
| Low TE buffer | 5 |
| R1 buffer | 5 |
| R2 chemicals | 2 |
| R3 yeast | 0.5 |
| Total volume | 12.5 |

The amplification and attachment of the indexes involved the following: Add 12.5 μL of the above chemical mixture into a sample tube containing 10 μL of the cfDNA product purified in the previous element. Add another 2.5 μL of different index primer pairs specified for each sample. Mix the mixture well by suction-release 10 times and place the sample tube containing the mixture in the heat cycler. The amplification program followed the parameters shown in Table 9 below.

TABLE 9

| Element | Temperature (° C.) | Time (seconds) |
|---|---|---|
| 1 | 98 | 30 |
| 2 | 98 | 10 |
| 3 | 60 | 30 |
| 4 | 68 | 60 |

Repeat 2-4 for 15 cycles
Kept at 4° C.

After amplification, the purification of the product involved the following: add 20 μL of KAPA magnetic beads into the sample tube containing the above amplified product. Mix well by suction-release 10 times and incubate at room temperature for 5 minutes. Place the sample tube on a magnetic tray to capture magnetic beads, wait for the solution to clear, and discard the supernatant. Add another 200 μL of 80% alcohol solution and incubate for 30 seconds, then discard the supernatant. Add another 200 μL of 80% alcohol solution and incubate for 30 seconds, then discard the supernatant. Let the magnetic beads dry naturally for 1 to 3 minutes and avoid letting them too dry. Remove the tube from the magnetic tray and add 20 μL of TE with less EDTA. Create magnetic bead suspension by suction-release 10 times and incubate at room temperature for 5 minutes. Place the tube containing the amplified product on the magnetic tray to capture the magnetic beads, wait for the solution to clear, and transfer the supernatant into a new 1.5 ml Eppendorf tube. Check concentration of cfDNA library after amplification using Quantus™ Fluorometer meter system.

Fragmentation of the cfDNA Library for Variation Analysis at 450 Target Sequence Regions

Hybrid capture was performed using xGEN® Lockdown reagent kit (1080584) combined with human DNA Cot reagents (1080769) and xGen Universal Blocker-TS key mixture (1075474) to increase the specificity of hybrid capture process. The process of hybrid capture included the following:

Hybrid reaction: 16 libraries of different samples were pooled together in 1 hybrid reaction with an input of 50ng for each sample. A chemical mixture was prepared for nonspecific site-locking reaction including the components shown in the Table 10 below.

TABLE 10

| Component | Volume (μL) |
|---|---|
| Human DNA Cot | 5 |
| xGen Universal Blocker-TS key mixture | 2 |
| Total | 7 |

7 μL of the above key mixture were added into the sample tube containing the pooled libraries. The mixture was mixed and concentrated the sample on a concentrator at 1700 rpm, 65° C. until the solution turns colloidal. The hybrid buffer mixture included the components shown in the Table 11 below.

TABLE 11

| Component | Volume (μL) |
|---|---|
| xGen 2X hybrid buffer | 8.5 |
| xGen hybrid enhancer | 2.7 |
| Target probe | 4 |
| Water | 1.8 |
| Total | 17 |

The sample suspension was reconstituted with 17 μL of the above hybrid buffer mixture. The solution was mixed and incubated at room temperature for 5 to 10 minutes. The entire sample was transferred into a 0.2 ml PCR tube, then placed it in a heat cycler and run the thermal process with the settings shown in the Table 12 below.

TABLE 12

| Element | Temperature | Time | |
|---|---|---|---|
| 1 | 95° C. | 30 | seconds |
| 2 | 65° C. | 4 | hours |

Kept at 65° C.

The wash buffers were diluted and the probe capture reagent were prepared onto magnetic beads. The high-concentration stock buffers were defrosted and if the buffers have crystallized, incubated at 65° C. until completely dissolved. The components were diluted according to the Table 13 below.

TABLE 13

| | Water | Buffer | Total | |
|---|---|---|---|---|
| Component | (μL) | (μL) | (μL) | Storage |
| xGen 2X magnetic beads | 250 | 250 | 500 | Room temperature |
| wash buffer | | | | |
| I xGen 10X wash buffer | 270 | 30 | 300 | Divide into 2 parts: at 65° C. and |
| | | | | room temperature |
| II xGen 10X wash buffer | 180 | 20 | 200 | Room temperature |
| III xGen 10X wash buffer | 180 | 20 | 200 | Room temperature |
| xGen 10X strong wash | 360 | 40 | 400 | At 65° C. |
| buffer | | | | |

The reaction mixture was prepared for probe hybrid capture onto magnetic beads and included the components shown in the Table 14 below.

TABLE 14

| Component | Volume (μL) |
|---|---|
| xGen 2X Hybridization Buffer | 8.5 |
| xGen Hybridization Buffer Enhancer | 2.7 |
| Nuclease-Free Water | 5.8 |
| Total | 17 |

The washing of the streptavidin magnetic beads included the following: Bring Dynabeads M-270 Streptavidin magnetic beads from 4° C. to room temperature at least 30 minutes before use. Create magnetic bead suspension using a shaker for 15 seconds. Aspirate 100 μL of magnetic beads into each 1.5 ml non-stick tube. Add 100 μL of magnetic beads wash buffer into each tube. Create suspension by suction-release 10 times. Place the tube in a magnetic tray, wait until the magnetic beads separate from the supernatant (about 1 minute) and discard the supernatant, making sure that the magnetic beads remain in the tube. Remove the tube from the magnetic tray and perform the washing again with 100 μL of magnetic bead wash buffer. Reconstitute the magnetic bead suspension in 17 μL of the above capture reaction mixture solution. Mix well to ensure that the magnetic beads do not dry on the wall of the tube. Magnetic beads are ready for capture reaction.

After hybridization the library capture followed the protocol as detailed herein: After incubation for 4 hours, end the hybridization program, remove the sample from the PCR machine. Transfer 17 μL of the above-suspended magnetic bead mixture into the tube containing the hybrid sample. Mix well by suction-release 10 times and incubate the sample tube in a heat cycler at 65° C. for 45 minutes. Make sure the cap of the heat cycler is at 70° C. Every 15 minutes, gently create suspension to mix well the magnetic beads. After 45 minutes, remove the sample from the PCR machine and immediately proceed to the washing with annealing.

The 65° C. hot washing involved the following: Use wash buffer I and strong wash solution that has been incubated at 65° C. Transfer 100 μL of wash buffer I into the sample tube and do suction-release 10 times without forming air bubbles. Place the tube on a magnetic tray for 1 minute. Collect the supernatant into a 1.5 ml non-stick tube, used for the flow through the library fragment collection. Remove the tube from the magnetic tray and add 200 μL of strong wash solution to the sample. Suction and release 10 times using a pipet without air bubbles and incubate the sample at 65° C. for 5 minutes. Place the tube on a magnetic tray for 1 minute and discard the supernatant. Remove the tube from the magnetic tray and add 200 μL of strong wash solution to the sample tube. Suction and release 10 times using a pipet without air bubbles and incubate the sample at 65° C. for 5 minutes. Place the tube on a magnetic tray for 1 minute.

The room temperature washing involved the following: Wash buffers I, II and III are placed at room temperature. Discard the supernatant and add another 200 μL of wash buffer I. Create suspension to mix the sample well and incubate for 2 minutes (alternately shake for 30 seconds, rest for 30 seconds). After incubation, quickly centrifuge the sample tube and place it on a magnetic tray for 1 minute. Discard the supernatant and add another 200 μL of wash buffer II. Create suspension to mix the sample well and incubate for 2 minutes (alternately shake for 30 seconds, rest for 30 seconds). After incubation, quickly centrifuge the sample tube and place it on a magnetic tray for 1 minute. Discard the supernatant and add 200 μL of wash buffer III. Create suspension to mix the sample well and incubate for 2 minutes (alternately shake for 30 seconds, rest for 30 seconds). After incubation, quickly centrifuge the sample tube and place it on a magnetic tray for 1 minute. Discard the supernatant and use a suitable aspirator to remove all residual solution, then remove the tube from the magnetic tray. Add another 20 μL of H₂O, magnetic bead suspension by suction-release 10 times. Magnetic beads in the form of suspension are used directly for the next element of the method.

The Post-capture library amplification involved the following: Prepare chemical mixture for amplification reaction (after capture) including the components shown in the Table 15 below.

TABLE 15

| Component | Volume (μL) |
|---|---|
| KAPA HiFi HotStart 2X mixture | 25 |
| P5/P7 primer mixture | 5 |
| Total | 30 |

Add 30 μL of chemical mixture to 20 μL of magnetic beads in the form of suspension in the previous element of the method. Mix the mixture well by suction-release 10 times. Place mixture tube in a heat cycler and run amplification program with the parameters shown in Table 16 below.

TABLE 16

| Element | Temperature | Time |
|---|---|---|
| 1 | 98° C. | 45 seconds |
| 2 | 98° C. | 15 seconds |
| 3 | 60° C. | 30 seconds |
| 4 | 72° C. | 30 seconds |

Repeat 2-4 for 14 cycles (*)

| | | |
|---|---|---|
| 5 | 72° C. | 60 seconds |

Kept at 4° C.

Purifying the product after amplification: Place the tube containing the amplified product on the magnetic tray to capture the magnetic beads, wait for the solution to clear and transfer the supernatant into a tube containing 45 μL of KAPA magnetic beads. Mix the sample well by suction-release 10 times and incubate at room temperature for 5 minutes. Place the sample tube on a magnetic tray to capture magnetic beads, wait for the solution to clear and discard the supernatant. Add 200 μL of 80% alcohol solution and incubate for 30 seconds, then discard the supernatant. Add another 200 μL of 80% alcohol solution and incubate for 30 seconds, then discard the supernatant. Let the magnetic beads dry naturally for 1 to 3 minutes, avoid letting them too dry. Remove the tube from the magnetic tray and add 22 μL of TE 0.1×. Create magnetic bead suspension by suction-release 10 times and incubate at room temperature for 5 minutes. Place the tube containing the amplified product on the magnetic tray to capture the magnetic beads, wait for the solution to clear and transfer the supernatant into a new 1.5 ml tube. Check concentration of cfDNA library after the amplification using Quantus™ Fluorometers meter system.

The collection of library fragments for analysis of genome-wide variation (“flow through” fragment) involved the following:
Prepare chemicals, tools and equipment:
Wash solution I (high salt concentration): NaCl 1M, Tris-HCl 10 mM, Tween-20 0.05%.
Wash solution II (low salt concentration): NaCl 15 mM, Tris-HCl 10 mM.
Dynabeads® M-270 Streptavidin magnetic beads (Cat No. 11205D)
Biotin-bound P5 Probe (12.5 μM) (Integrated DNA Technologies-IDT)
Biotin-bound P7 Probe (12.5 μM) (Integrated DNA Technologies-IDT)
Hybridization buffer
Hybridization enhancer.
KaPa Hifi HotStart Ready mixture (Cat No. KK2601)
P5, P7 Primer mixture (Integrated DNA Technologies-IDT)
Kapa Pure Beads magnetic beads (Cat No. KK8002)
Sample concentrator (Thermo Fisher Scientific SpeedVac system)
Magnetic 1.5 ml and 0.2 ml tube trays (magnetic trays)
Vortexer.
PCR heat cycler

The concentration of library fragments involved the following: Wash solution I sample containing the remaining cfDNA library fragments is evaporated on the sample concentrator system at 1700 rpm at 65° C. Attach P5/P7 probe to Dynabeads® M-270 Streptavidin magnetic beads. Add another 100 μL of magnetic beads to a 1.5 ml Eppendorf tube. Place the tube on a magnetic tray to capture magnetic beads, wait for the solution to clear and discard the supernatant. Remove the tube from the magnetic tray, add 100 μL of wash solution I into the tube. Mix well the mixture for 5 seconds on a vortexer. Place the tube on a magnetic tray to capture magnetic beads, wait for the solution to clear and discard the supernatant. Wash the magnetic beads again with wash solution I for 2 more times. Place the tube on a magnetic tray to capture magnetic beads, wait for the solution to clear, discard the supernatant. Add 16 μL of H₂O into the tube containing washed magnetic beads, mix well and transfer to a 0.2 ml tube. Add 2 μL of P5 probe and 2 μL of P7 probe and mix well, incubate at room temperature for 15 minutes. Place the tube containing the mixture of magnetic beads fitted with P5/P7 probe on a magnetic tray to collect magnetic beads, wait for the solution to clear and discard the supernatant. Add 100 μL of wash solution I and mix well the mixture for 5 seconds. Place the tube on a magnetic tray to capture magnetic beads, wait for the solution to clear and discard the supernatant. Wash the magnetic beads again with wash solution I for 2 more times. Place the tube on a magnetic tray to capture magnetic beads, wait for the solution to clear and discard the supernatant. Add the following components into the library tube (concentrate): 1.8 μL of H₂O; 8.5 μL of hybrid buffer and 2.7 μL of hybrid enhancer. Incubate this mixture at room temperature for 10 minutes. Mix well by suction-release 10 times and transfer the entire mixture to a 0.2 ml tube. Place the tube in a heat cycler and incubate at 95° C. for 10 minutes. Transfer the entire mixture to a tube containing the magnetic bead mixture fitted with P5/P7 probe. Mix well by suction-release 10 times and incubate at room temperature for 30 minutes. Place the sample tube on a magnetic tray to capture magnetic beads, wait for the solution to clear and discard the supernatant. Remove the sample tube from the magnetic tray, add 100 μL of wash solution I into the tube. Mix the mixture well by suction-release 10-20 times. Place the sample tube on a magnetic tray to capture magnetic beads, wait for the solution to clear and discard the supernatant. Wash again with wash solution I for one more time. Then, add 100 μL of wash solution II to the tube and mix the mixture well by suction-release 10-20 times. Place the sample tube on a magnetic tray to capture magnetic beads, wait for the solution to clear and discard the supernatant. Add 20 μL of H₂O into the tube, suspend the magnetic bead evenly by suction-release 10 times. Magnetic beads in the form of suspension are used for the next element of the method.

The amplification of DNA with KAPA HiFi DNA Polymerase yeast involved the following: Transfer 3 μL of the mixture of magnetic beads in form of suspension to a 0.2 ml tube. Place the tube in a heat cycler and incubate at 65° C. for 10 minutes. Place the sample tube on a magnetic tray to capture magnetic beads, wait for the solution to clear and discard the supernatant. Measure the concentration of cfDNA in the supernatant using Quantus™ Fluorometer meter system.

The preparation of the library amplification reaction involved the following: Add another 3 μL of H₂O; 25 μL of KAPA HiFi HotStart Ready Mix and 5 μL of P5/P7 primer mixture into 17 μL of magnetic beads in the form of suspension. Mix the mixture well by suction-release 10-20 times. Place the sample in a heat cycler and run the heat program as shown in Table 17 below.

TABLE 17

| Element | Temperature (° C.) | Time (seconds) |
|---|---|---|
| 1 | 98 | 45 |
| 2 | 98 | 15 |
| 3 | 60 | 30 |
| 4 | 72 | 30 |

Repeat 2-4 for 10 cycles (*)

| | | |
|---|---|---|
| 5 | 72 | 60 |

Kept at 4° C.
(*) number of cycles is adjusted depending on the library concentration before amplification and the amount of library required after the amplification.

The purification of the product after amplified involved the following: Place the tube containing the amplified product on the magnetic tray to capture magnetic beads, wait for the solution to clear, transfer the supernatant into a tube containing 45 μL of KAPA magnetic beads. Mix well by suction-release 10 times and incubate at room temperature for 5 minutes. Place the sample tube on a magnetic tray to capture magnetic beads, wait for the solution to clear and discard the supernatant. Add another 200 μL of 80% alcohol solution, incubate for 30 seconds and discard the supernatant. Add another 200 μL of 80% alcohol solution, incubate for 30 seconds and discard the supernatant. Let the magnetic beads dry naturally for 1-3 minutes and avoid letting them too dry. Remove the tube from the magnetic tray and add 20 μL of TE 0.1×. Mix well by suction-release 10 times and incubate at room temperature for 5 minutes. Place the tube containing the amplified product on the magnetic tray to capture magnetic beads, wait for the solution to clear, transfer the supernatant into a new 1.5 ml Eppendorf tube. Check concentration of cfDNA library after the amplification using Quantus™ Fluorometer meter system.

The Procedure for Library Transformation and Sequencing Using MGI-DNBseq System Involved the Following:

To be sequenced on a DNBseq system, the cfDNA library needed to be converted into DNA library spheres, the process is done with MGI Easy Universal library conversion reagent kit (1000004155). The specific protocol was as follows:

Adapter conversion: The libraries of each sample were mixed with equal amounts of DNA to form a mixture of pooled library. The pooled library was fitted with a suitable adapter for the MGI-DNBseq sequencing system through the AC-PCR reaction amplification. The reaction components included 25 μL of AC-PCR amplification chemical mixture and 3 μL of AC-PCR primer mixture. The PCR reaction was done in a heat cycler with thermal cycling as shown in the Table 18 below.

TABLE 18

| Element | Temperature | Time |
|---|---|---|
| 1 | 98° C. | 3 minutes |
| 2 | 98° C. | 30 seconds |
| 3 | 62° C. | 15 seconds |
| 4 | 72° C. | 30 seconds |

Repeat 2-4 for 5 cycles

| | | |
|---|---|---|
| 5 | 72° C. | 5 minutes |

Kept at 4° C.

After amplification, the purification of the product involved the following: Add 60 μL of KAPA magnetic beads into the tube containing the amplified product. Mix well by suction-release 10 times and incubate at room temperature for 5 minutes. Place the sample tube on a magnetic tray to capture magnetic beads, wait for the solution to clear, and discard the supernatant. Add another 200 μL of 80% alcohol solution, incubate for 30 seconds and discard the supernatant. Add another 200 μL of 80% alcohol solution, incubate for 30 seconds and discard the supernatant. Let the magnetic beads dry naturally for 1-3 minutes, avoid letting them too dry. Remove the tube from the magnetic tray and add 30 μL of TE 0.1×. Create magnetic bead suspension by suction-release 10 times and incubate at room temperature for 5 minutes. Place the tube containing the amplified product on the magnetic tray to capture magnetic beads, wait for the solution to clear and transfer the supernatant into a new 1.5 ml Eppendorf tube. Check concentration of cfDNA library after the amplification using Quantus™ Fluorometer meter system.

Denaturation—separation: The library were denatured to separate into a single strand. Specifically, after AC-PCR, 1 pmol of product was denatured in a heat cycler at 95° C. for 3 minutes and then placed on cold ice immediately to prevent regurgitation of single-stranded DNAs.

Cyclization reaction: The straight single-stranded DNA library was converted to cyclic form by a cyclization reaction. The reaction used 1 short single-stranded DNA fragment (splint Oligo) capable of complementary pairing with 2 adapters attached in the AC-PCR. This splint Oligo fragment acted as a splint to connect 2 ends of single-stranded DNA fragments. The reaction components included: 11.6 μL of splint buffer and 0.5 μL of ligation enzyme, done in a heat cycler at 37° C. for 30 minutes and then immediately place the product on cold ice.

Reaction of cleavage of non-cyclic DNA library fragments: Non-cyclic single-stranded DNA library fragments were enzymatically chopped. The reaction used 4 μL of a mixture of cutting enzymes (including 1.4 μL of cutting buffer and 2.6 μL of cutting yeast). The reaction was incubated at 37° C. for 30 minutes using a heat cycler. After being chopped, DNA fragments were removed using the purification process.

After fragmentation, the purification of DNA product involved the following: Add 170 μL of KAPA magnetic beads into the tube containing chopped product. Mix well by suction-release 10 times and incubate at room temperature for 5 minutes. Place the sample tube on a magnetic tray to capture magnetic beads, wait for the solution to clear, discard the supernatant. Add another 500 μL of 80% alcohol solution, incubate for 30 seconds and discard the supernatant. Add another 500 μL of 80% alcohol solution, incubate for 30 seconds and discard the supernatant. Let the magnetic beads dry naturally for 1-3 minutes, avoid letting them too dry. Remove the tube from the magnetic tray and add 27 μL of TE 0.1×. Mix well by suction-release 10 times and incubate at room temperature for 5 minutes. Place the tube on the magnetic tray to capture magnetic beads, wait for the solution to clear, transfer the supernatant into a new 1.5 ml Eppendorf tube. Check the concentration of cfDNA library after fragmentation using Quantus Fluorometer meter system.

DNA sphere (DNB) generation—circle amplification reaction: A mixture of 20 μL of App-A buffer produced DNB and 60 fmol (equivalent to 9.9ng) of cyclic DNA library. The mixture was placed in a heat cycler using program parameters as shown in Table 19 below.

TABLE 19

| Element | Temperature (° C.) | Time (minutes) |
|---|---|---|
| 1 | 95 | 1 |
| 2 | 65 | 1 |
| 3 | 40 | 1 |

Kept at 4° C.

44 μL of mixture for generation of DNB 2 were added to the element 1 product (kept on cold ice). The mixture was placed in a heat cycler using program parameters as shown in the Table 20 below.

TABLE 20

| Element | Temperature (° C.) | Time (minutes) |
|---|---|---|
| 1 | 30 | 25 |

| | |
|---|---|
| 2 | Kept at 4° C. |

As soon as the temperature reached 4° C., 20 μL of Stop DNB reaction buffer were added. The DNB library mixture was mixed well by suction-release gently with a wide-mouth straw to avoid breaking DNBs. The amount of formed DNB was quantified using the QuBit system.

Load DNB onto a flowcell: The DNB mixture was mixed with 8 μL of DNB II loading buffer and 0.25 μL of DNB II LC yeast mixture. The mixture was mixed well by suction-release using a wide-mouth straw. The flowcell was fitted to the sample feeder. Using a wide-mouth straw, 30 μL of the DNB library mixture was transferred to the sample loading position on the feeder. The DNB library solution automatically flew into the flowcell without being injected.

Preparation the sequencing reagent cartridge: After the sequencing reagent cartridge was defrosted, it was stirred well and wiped dry the outer shell. A pointed tip was used to puncture the membrane of the wells marked with 1, 2, 3, 4, 6, 7 and 8 on the sequencing reagent cartridge. The sample was loaded according to the Table 21 below.

TABLE 21

| | Absorb the liquid that | Add to the solution |
|---|---|---|
| Well | is already inside | mixture |
| 1 | | 1.8 ml of dNTPs mixture |
| | | 1.8 ml of sequencing yeast mixture |
| 2 | | 1.8 ml of dNTPs mixture |
| | | 1.8 ml of sequencing yeast mixture |
| 3 | App-A insertion primer 1 | 2.2 ml of App-A insertion primer 1 (1 μM) |
| 4 | | 2.9 ml of App-A index primer 3 (1 μM) |
| 6 | App-A insertion primer 2 | 2.9 ml of App-A index primer 2 (1 μM) |
| 7 | App-A MDA primer | 3.1 ml of App-A MDA primer (1 μM) |
| 8 | App-A index primer 2 | 3.3 ml of App-A insertion primer 2 (1 μM) |

The sequencing reagent cartridge and flowcell were placed into MGiseq-2000 sequencer, the required information was entered and the sequencing process was started.

### Example 2: Element 2—Analyze Different Variation Patterns of cfDNA

2.1 Analysis of Methylation Variation at 450 Target Regions (Containing 18,000 CpG Sites)

Raw data was quality checked using FastQC tool (Babraham Institute, version 0.11.9). Poor quality data and adapter sequences were removed using a trimmomatic tool. Read sequences were aligned with the standard genome and analyzed to determine methylation percentage using the Bismark aligner tool (Babraham Institute, version 16.0.2).

Regions with different methylation percentages between cancer and healthy groups (called DMR—Differentially Methylated Regions) were determined by the methylation percentage per CpG determined using the following formula:

$$Methylation percentage = \frac{{N}_{C , i}}{{N}_{C , i} + {N}_{T , i}} \times 100 \%$$

where:
i: The i^{th} CpG site in the sequence region of interest,
N_{T,i}: Number of T nucleotides observed at the i^{th} CpG site, and
N_{C,i}: Number of C nucleotides observed at the i^{th} CpG site.

The regions with different methylation percentage between the cancer group and the healthy group were determined. Specifically, the percentage of methylation of the healthy group and the cancer group were compared on each corresponding CpG site by the Wilcoxon rank sum test (Mann Whitney U test), in order to identify regions with differences (statistically significant) on the methylation density of CpG. The Wilcoxon rank sum test is suitable when comparing multiple variables simultaneously between 2 groups of independent samples and variables that are not normally distributed (non-parametric test). In addition, the p-value of the statistical test was corrected using the Benjamini Hochberg method to avoid the false-positive situation encountered when the number of variables to be compared was much larger than the number of analyzed samples. Regions identified with different percentages of methylation between cancer and healthy groups when p-value was less than 0.05 (p-value<0.05).

The methylation fold change was determined between the cancer group and the healthy group. Specifically, the percentage of methylation (between cancer and healthy groups) on each respective CpG site was used to determine how many times the methylation fold change had changed. The methylation fold change was corrected by taking the log to base 2 (|log 2|) of the absolute value of the above percentage. If this value was greater than 1, the methylation fold change had changed more than 2 times between the cancer group and the healthy group. With some of the results depicted in the figures:

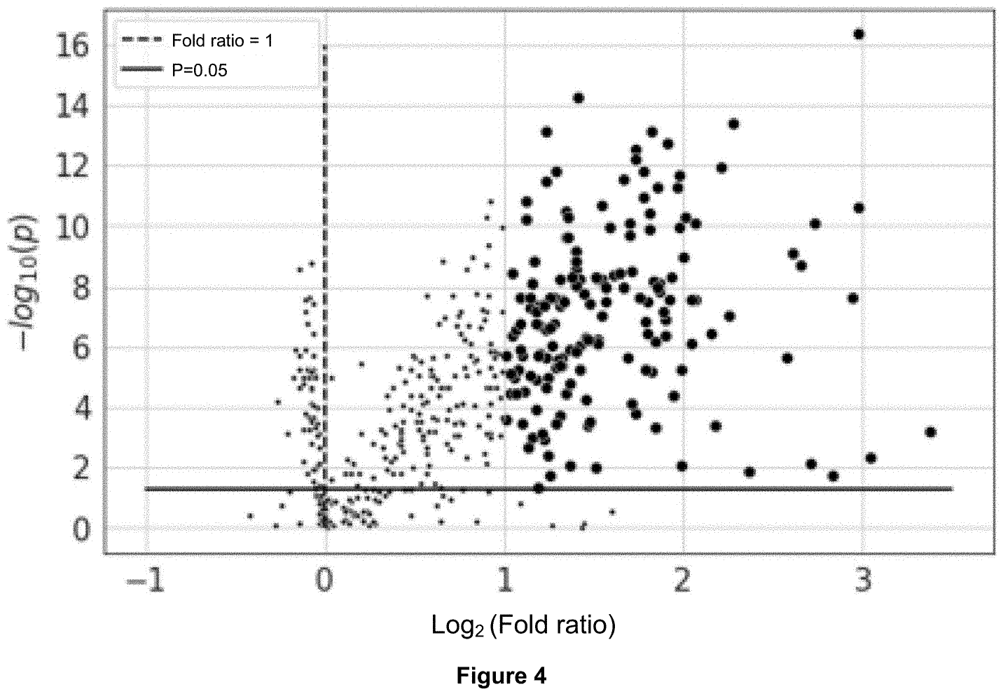

FIG. 4 illustrates 353 sequence regions out of 450 target sequence regions surveyed with statistically significant differences in methylation density (p-value<0.05) between the liver cancer group and the healthy group specified when performing the SPOT-MAS test procedure according to the present invention (as described above). Specifically, in each survey region, the percentage of methylation was compared between the cancer group and the healthy people using the Wilcoxon rank sum test with correction using the Benjamini-hochberg method. It was noted that 353 out of 450 target sequence regions had differences in methylation density (p-value less than 0.05) (including dots above the solid line with value −log 10(p-value)>1.30). In these 353 regions, there were 154 regions with methylation density in liver cancer patients being 2 times that of healthy people (including large dots, located to the right of the dashed line with log 2 value (fold ratio)>1).

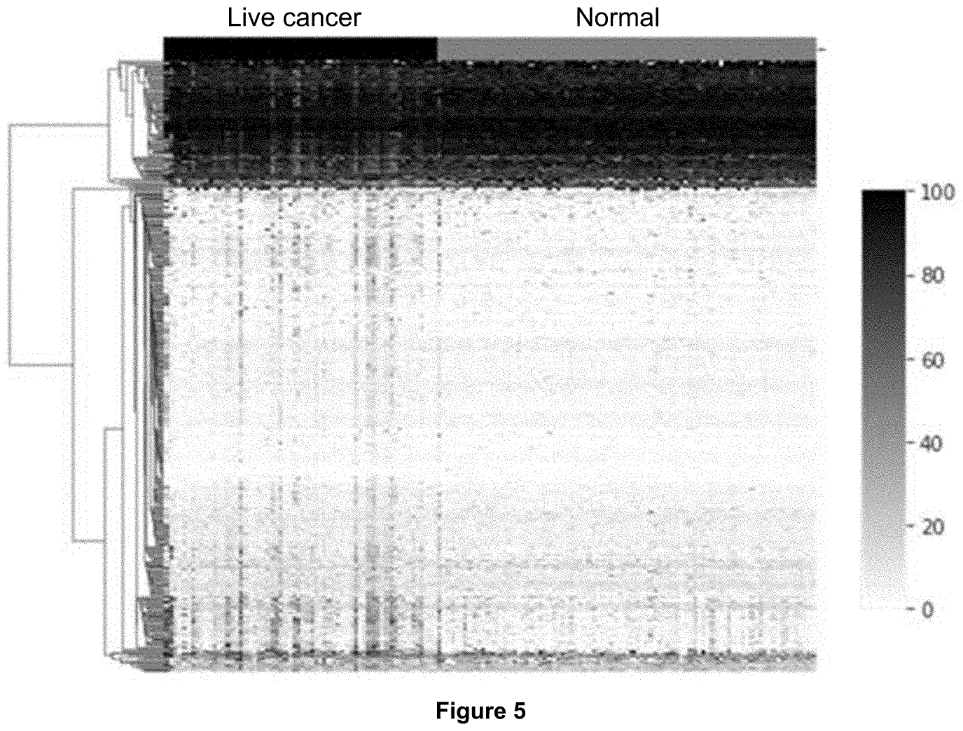

FIG. 5 is a heatmap illustrating the clustering according to the methylation density at target sequence regions between liver cancer patients and healthy subjects obtained after performing the SPOT-MAS test procedure according to the present invention. The lightness on the heatmap represented the degree of change in methylation density (with a scale of 0 to 100, the darker the color indicates the higher the methylation density). Specifically as shown in FIG. 5, from top to bottom, the regions of DNA sequences were grouped according to the descending order of the methylation density. From left to right was the list of analyzed samples, with the left side being the group of liver cancer patients, the right side being the group of healthy people. The results from the heatmap showed that the samples in the liver cancer group with multiple target sequence regions had increased methylation density compared with the healthy control group.

2.2 Methylation density change analysis on 22 Chromosomes

The quality of the sequencing data of the remaining flow through the library fragment was assesses using MultiQC software (https://multiqc.info/). Poor quality data and adapter sequences were removed using a trimmomatic tool. Read sequences were aligned against the human reference genome sequence (version hg19) using the BSAligner software in the (Methyl pipe analysis package, DOI: 10.1371/journal.pone.0100360). Check parameters: (1) proportion of reads was aligned against the reference genomic sequence in total mappability, (2) depth of sequencing, (3) sequencing coverage of all samples.

Genome-wide methylation variation was determined as follows. The standard human genome was uniformly subdivided into non-duplicating fragments (bin) of 1 megabase (one million nucleotides) long. Analysis for methylation variation was performed on each bin. The methylation density (MD) per bin was calculated using the following formula:

$$MD = \frac{\sum mC}{( \sum mC + \sum T )} \times 100$$

where ΣmC is the total number of methylated C nucleotides and ΣT is the total number of nucleotides.

Bins with variation in methylation state were identified. Sequencing data from 19 healthy subjects were randomly selected to determine the reference MD value for each bin. Variation in values of methylation density in each bin was evaluated based on the “Z score” value using the following formula:

$$Zscore = \frac{\begin{matrix}MD in survey bin - \\ Mean MD in corresponding survey bin of the reference group\end{matrix}}{\begin{matrix}Standard deviation MD in \\ corresponding bin in the reference group\end{matrix}}$$

If Zscore<−3, that bin region was less methylated than the bin in the reference group.
If −3<Zscore<3, methylation in that bin region was equivalent to the bin in the reference group.
If Zscore>3, that bin region was more methylated than the bin in the reference group.

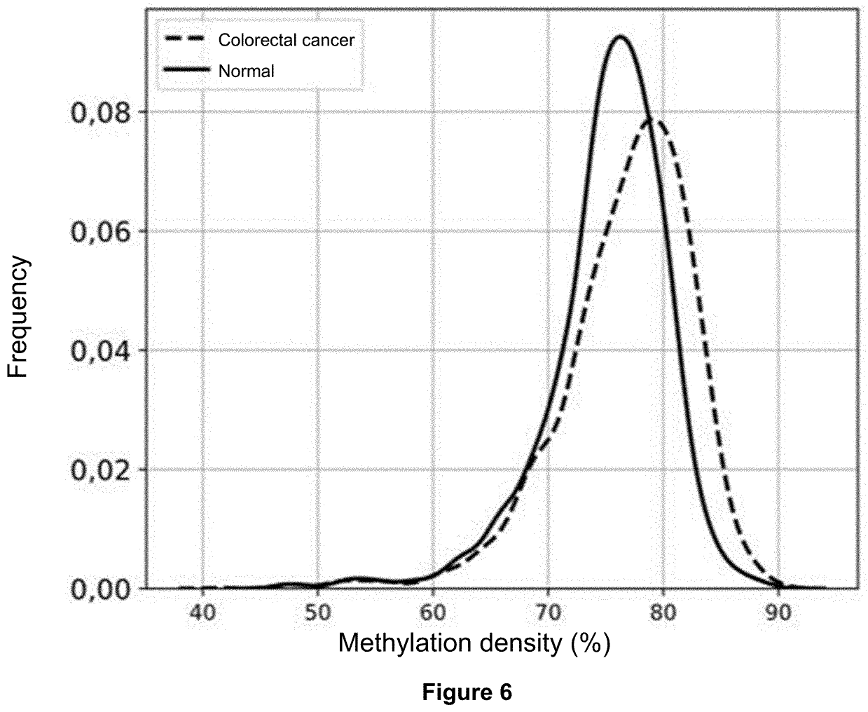

FIG. 6 illustrates the results of analysis of mean values of methylation density on all survey bins belonging to 22 chromosomes of patients with colorectal cancer (CRC) and a group of healthy people who underwent SPOT-MAS test procedure according to the present disclosure (as described above). Specifically, the solid curve represents the distribution of methylation density values of all the survey bins belonging to 22 chromosomes of the group of patients with colorectal cancer. The dotted curve depicts the distribution of methylation density values of all the survey bins of the 22 chromosomes of the healthy group. It can be seen that the distribution of methylation density values in the cancer group was skewed to the left (the tendency to decrease methylation) compared with the healthy group.

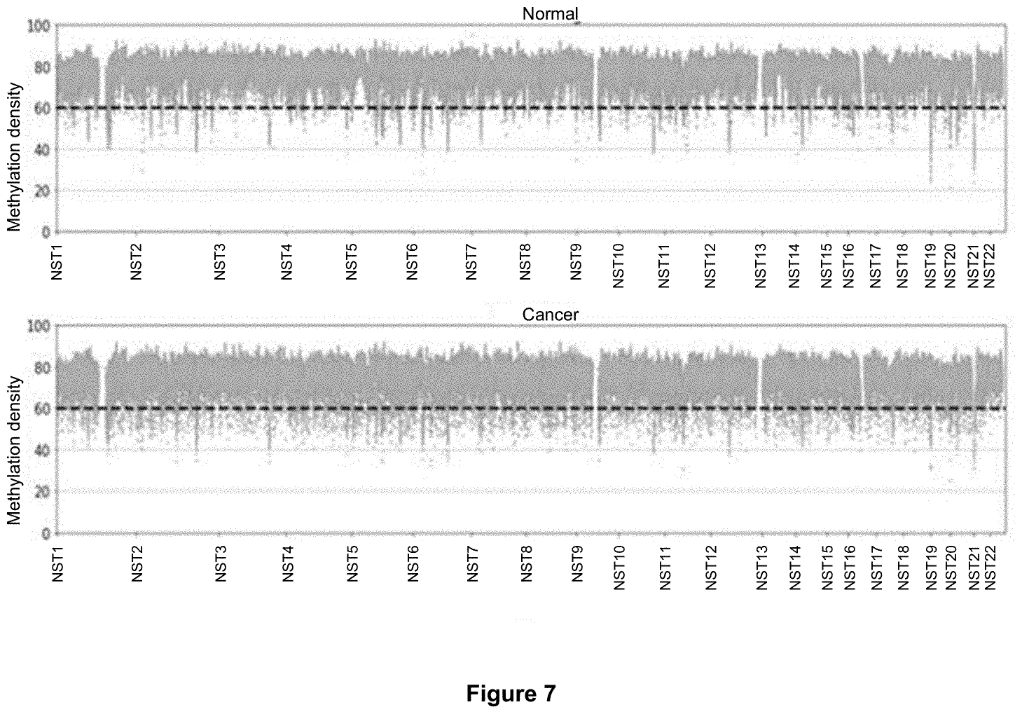

FIG. 7 shows a graph illustrating the decrease in methylation on all the ‘bin’ regions of the 22 chromosomes of the CRC group compared with the healthy group who underwent the SPOT-MAS test according to the invention (as described above). Specifically, the vertical axis represents the values of methylation density and the median represents the list of 22 chromosomes examined in healthy people (top chart) and CRC patients (bottom chart). The methylation density values of each bin are indicated by dots. When setting the benchmark (dotted line at the values of methylation density reaching 60%), it can be seen that the methylation density on some bins in the group of people with colorectal cancer was lower than in the healthy group.

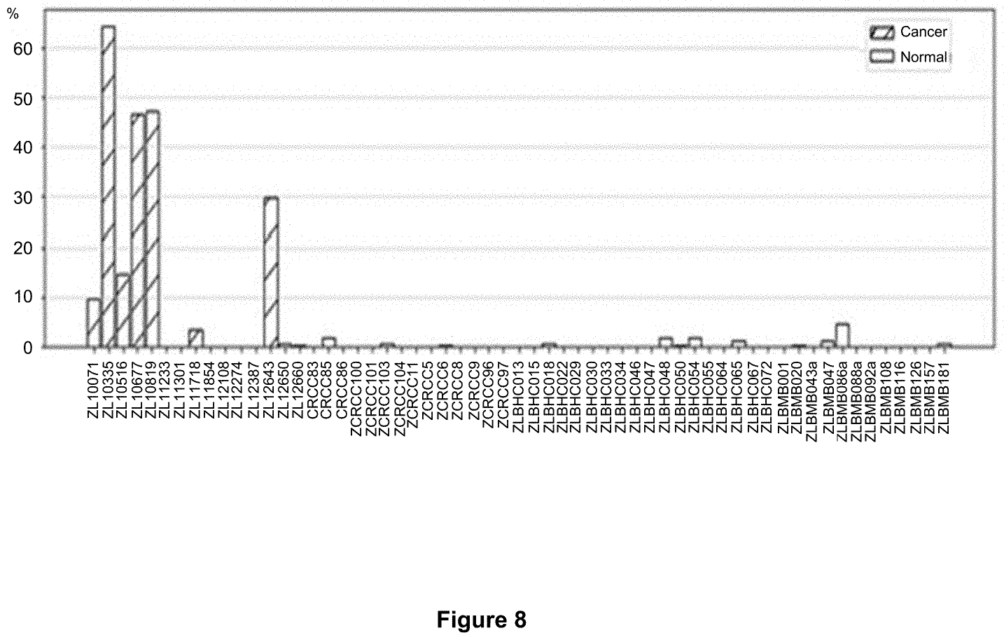

FIG. 8 shows a graph illustrating the percentage of bins that are determined to be less methylated (Zscore<3 according to the analysis described above) between the group of colorectal cancer patients and the group of healthy people who underwent the SPOT-MAS test procedure according to the invention. Accordingly, the vertical axis represents the percentage of bins that were less methylated, and the horizontal axis is the list of analyzed samples (with cancer samples being bars with slashes, and healthy samples being bars without slashes). The percentage of bins less methylated in the total number of bins surveyed was calculated for each sample. The results showed that, 5/15 (ZL10071, ZL10335, ZL10516, ZL0819, ZL12643) colorectal cancer samples had a higher percentage of less methylated bins than the healthy group.

2.3 DNA Copy Number Abnormalities Analysis on 22 Chromosomes

Sequencing data of the remaining flow through library fragments was used for genome-wide DNA copy number abnormalities analysis. Data quality was checked using FastQC software. Poor quality data and adapter sequences were removed using a trimmomatic tool. Read sequences were aligned against the human reference genome sequence (version hg19) using the BSAligner software in the (Methyl pipe analysis package, DOI: 10.1371/journal.pone.0100360).

Check parameters: (1) proportion of reads was aligned against the reference genomic sequence in total mappability, (2) depth of sequencing, (3) sequencing coverage of all samples.

Identifying DNA copy number abnormalities on 22 chromosomes

The standard human genome was uniformly subdivided into non-duplicating fragments (bin) of 1 megabase (one million nucleotides) long. Copy number abnormalities analysis was performed on each bin.

The number of copies of DNA in the bins was determined. Differences in the number of reads between bins can occur due to the influence of the bin region containing many G and C nucleotides (GC-bias) or the presence of repeat sequence regions (tandem repeat). Therefore, after alignment, the number of reads in each bin was corrected using the QDNASeq tool (DOI: 10.1101/gr.175141.114). The median copy number of all bins was calculated after correction. The degree of variation in the number of copies per bin was determined by taking the log to base 2 (|log 2|) of the absolute value of the ratio of the number of reads in that bin to the median of the reads of all bins. If this value was greater than 1, then the degree of variation was more than 2 times between the investigated bin and the whole genome.

The proportion of bins with DNA copy number abnormalities between the cancer group and healthy people was determined. Sequencing data from 19 healthy subjects were randomly selected to determine the average number of reads for each bin. Variation of gene copy number in each bin was evaluated based on the “Z score” value using the following formula:

$$Zscore = \frac{\begin{matrix}Number of reads in survey bin - Average number of reads \\ in the corresponding bin of the standard reference group\end{matrix}}{\begin{matrix}Standard deviation of the number of reads \\ in the corresponding bin in the reference group .\end{matrix}}$$

If Zscore<−3, that bin region had fewer copies than the bin in reference group.
If −3<Zscore<3, the number of copies that bin region had is equivalent to the bin in the references group.
If Zscore>3, that bin region had more copies than the bin in reference group.

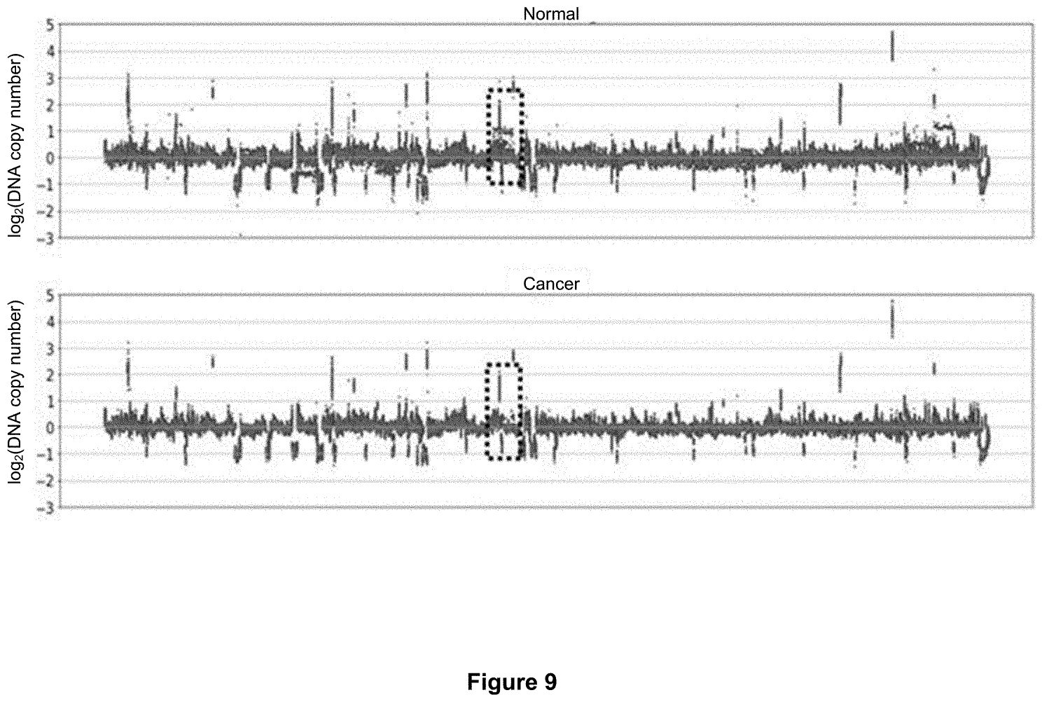

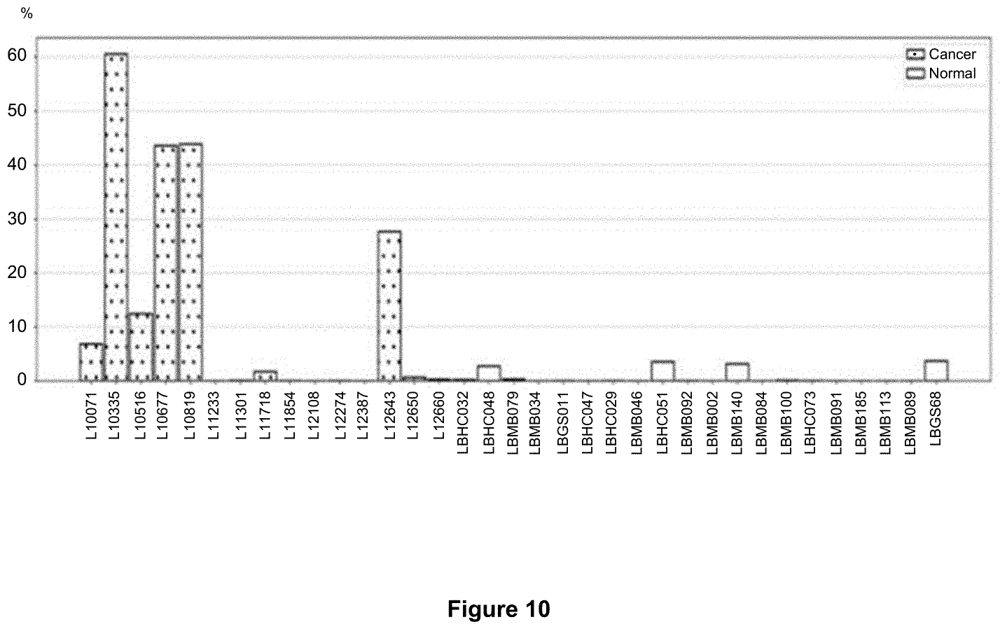

The obtained test results are shown in FIGS. 9 and 10.

FIG. 9 is a chart illustrating DNA copy number variations on all 22 chromosomes of the group of colorectal cancer patients and the group of healthy people who underwent the SPOT-MAS test procedure according to the disclosure, as described above. Specifically, the vertical axis represents the log to base 2 value of the number of DNA copies and the horizontal axis represents the list of chromosomes examined in healthy people (top chart) and CRC patients (bottom chart). The chromosome outlined by the dashed line is the chromosomes with DNA copy number abnormality. This result showed that colorectal cancer patients had copy number abnormalities in peripheral blood compared with the group of people with colorectal cancer.

FIG. 10 is a chart illustrating the percentage of the bins with gene copy number abnormalities in the total number of surveyed bins between the CRC group and the healthy group who underwent the SPOT-MAS test procedure according to the disclosure, as described above. Accordingly, the vertical axis represents the percentage of the bins with copy number abnormalities, and the horizontal axis is the list of analyzed samples (with cancer samples being spotted bars, and healthy samples being non-spotted bars). The percentage of bins with abnormalities (when absolute value of Zscore (|Zscore|)>3) in the surveyed bins was calculated for each sample. The results show that, 6/15 colorectal cancer samples (ZL10071, ZL10516, ZL10335, ZL10672, ZL0819 and ZL12643) that were surveyed had a higher percentage of bins with abnormalities than that of the healthy group. This result demonstrated instability in the DNA copy number in peripheral blood of the colorectal cancer group.

2.4 Analysis of Variation in cfDNA Size

Sequencing data of the remaining flow through library fragments was used to analyze variation in cfDNA size. Data quality was checked using MultiQC software (https://multiqc.info/). Poor quality data and adapter sequences were removed using a trimmomatic tool.

Read sequences were aligned against the human reference genome sequence (version hg19) using the BSAligner software in the (Methyl pipe analysis package, DOI: 10.1371/journal.pone.0100360). The parameters: (1) proportion of reads was aligned against the reference genomic sequence in total mappability, (2) depth of sequencing, and (3) sequencing coverage were checked for all samples.

Variation in cfDNA size was determined as follows.

The standard human genome was uniformly subdivided into non-duplicating fragments (bin) of 5 megabase (5 million nucleotides) long. Size variation analysis was performed on each bin.

After alignment, the length of each cfDNA fragment was calculated using software (bsalign). The size of cfDNA fragment was calculated based on the distance between the starting point of the Watson reading in the standard genome and the end point of the reading in the opposite direction (Crick).

The size distribution ratio of cfDNA fragments of cancer and healthy samples in the range of 0 to 250 nucleotides was determined.

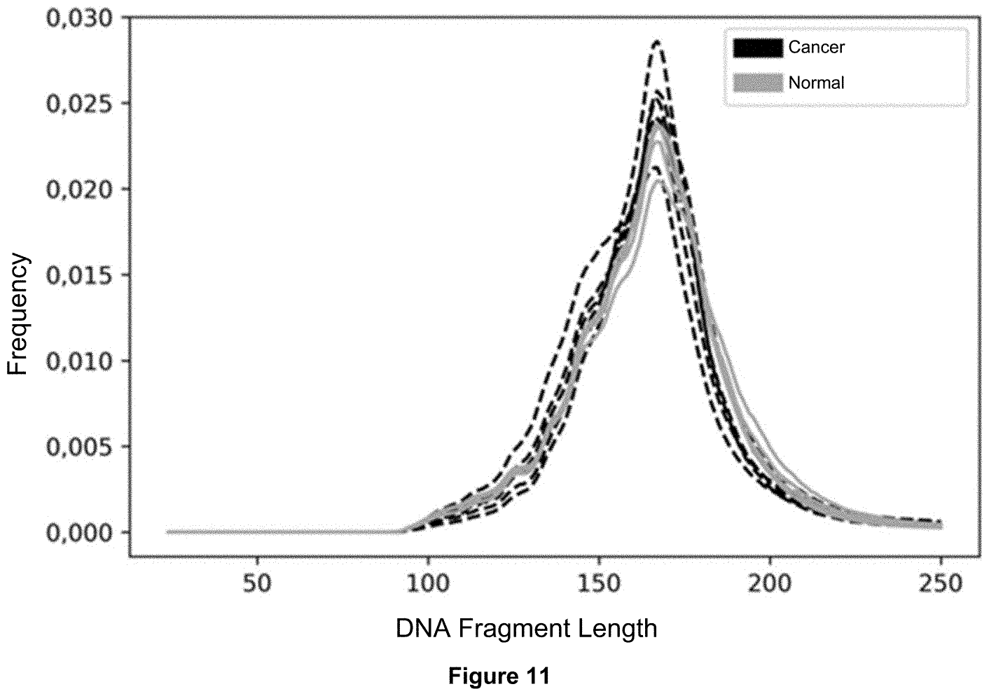

FIG. 11 is a histogram showing the size distribution of cfDNA fragments in colorectal cancer samples and healthy subjects who underwent the SPOT-MAS test procedure according to the disclosure, as described above. Specifically, the horizontal axis of the graph represents the scale of cfDNA size (from 0 to 250 nucleotides) and the vertical axis represents the density of cfDNA fragmentation in the blood. The black dashed line represents the cfDNA size distribution in the blood of CRC patients, while the gray solid line represents the cfDNA size distribution in the blood of the healthy people. The results showed that the density of cfDNA in colorectal cancer samples with cfDNA size<150 bp was higher than in healthy samples. This result suggested that a person's condition can be represented by the distribution of cfDNA lengths found in that person's plasma.

Fragment ratio (RF) per bin was calculated using the following formula:

$$R F = \frac{( P \leq 1 \; 50 bp )}{( P > 1 \; 50 bp )} \times 100$$

where P≤150 bp means length of reads is 150 nucleotides or less and P>150 bp means length of reads is over 150 nucleotides.

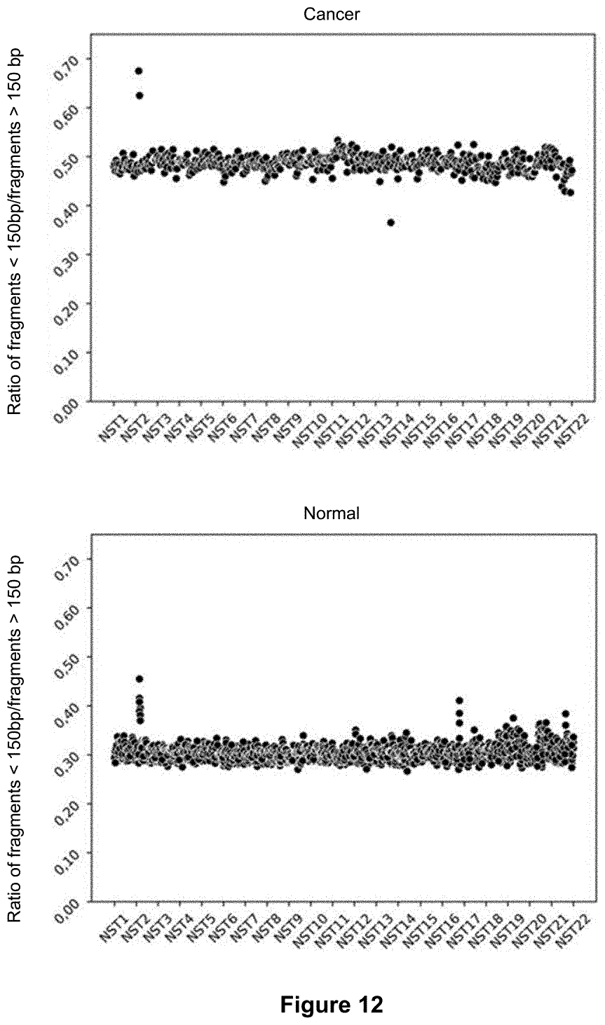

FIG. 12 is a histogram showing the RF ratio variation across all 22 chromosomes as determined by the SPOT-MAS test procedure according to the disclosure, as described above. Specifically, the vertical axis represents the RF ratio and the median represents the list of surveyed chromosomes. Within each region (bin) on the chromosome, the RF ratio is represented as a dot. When comparing patients with colorectal cancer (left graph) and healthy people (right graph), the RF ratio was higher in the colorectal cancer group than in healthy people on the entire surveyed chromosome. This result established that there was a difference in cfDNA size fluctuations in peripheral blood that can help distinguish between cancer and healthy people.

### Example 3: Element 3—Building a Machine Learning Model that Predicts Samples Carrying Cancer and Tumor Origins

The analytical data as provided above in Example 2, sections 2.1, 2.2, 2.3 and 2.4, established the basis of quantitative data of four different attributes for each cfDNA sample: methylation density attribute of 450 target regions (2.1); methylation density attribute of bins in 22 chromosomes (2.2); DNA copy number attribute of bins in 22 chromosomes (2.3); cfDNA size-specific ratio attribute of bins in 22 chromosomes (2.4). The machine learning model was built for each individual group of attributes as well as the combination of all four attribute groups. The effectiveness of this model was evaluated based on its ability to classify 2 groups of samples as cancer and healthy people or between malignant and benign tumors.

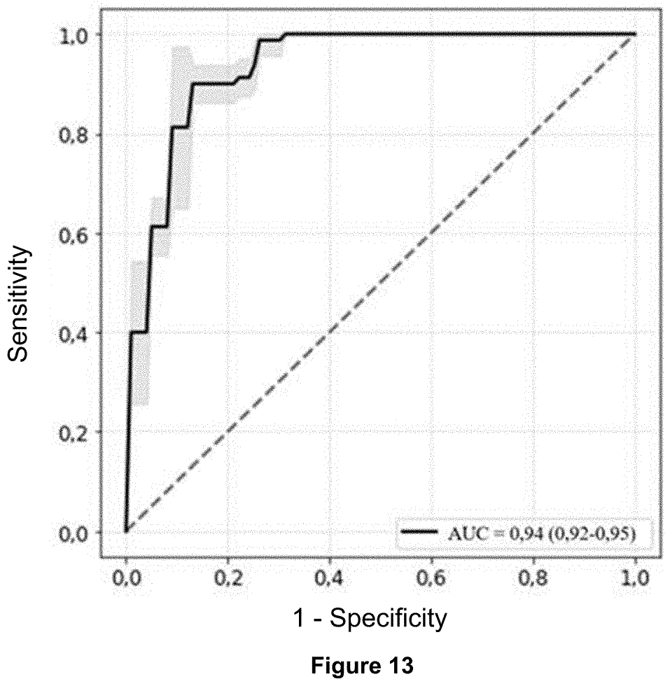

The model applied in the SPOT-MAS test procedure was a stacking model of individual attributes analyzed in element 2. The results of building the accuracy of the model are depicted in FIG. 13.

FIG. 13 is a chart illustrating the results of evaluating the effectiveness of blood sample classification of 4 groups of patients with liver cancer, lung cancer, colorectal cancer, and breast cancer with blood samples of healthy people who underwent SPOT-MAS test procedure according to the invention. Specifically, in the graph, the vertical axis represents the test's sensitivity and the horizontal axis represents the [1-specificity] value (or false-positive rate) of the test. Corresponding to a pair of sensitivity and [1-specificity] values, a point will be plotted on the graph. The changes in value of [1-specificity] from 0 to 1 will create a receiver operating curve (ROC). The area bounded by the ROC curve and the right and bottom sides of the graph is called the area under the ROC curve (or AUC). The larger the area, the higher the accuracy of the model. FIG. 13 showed that the AUC area is 0.94 (with confidence intervals ranging from 0.92 to 0.95), which means that the model's accuracy was up to 94% when classifying cancer samples and healthy samples.

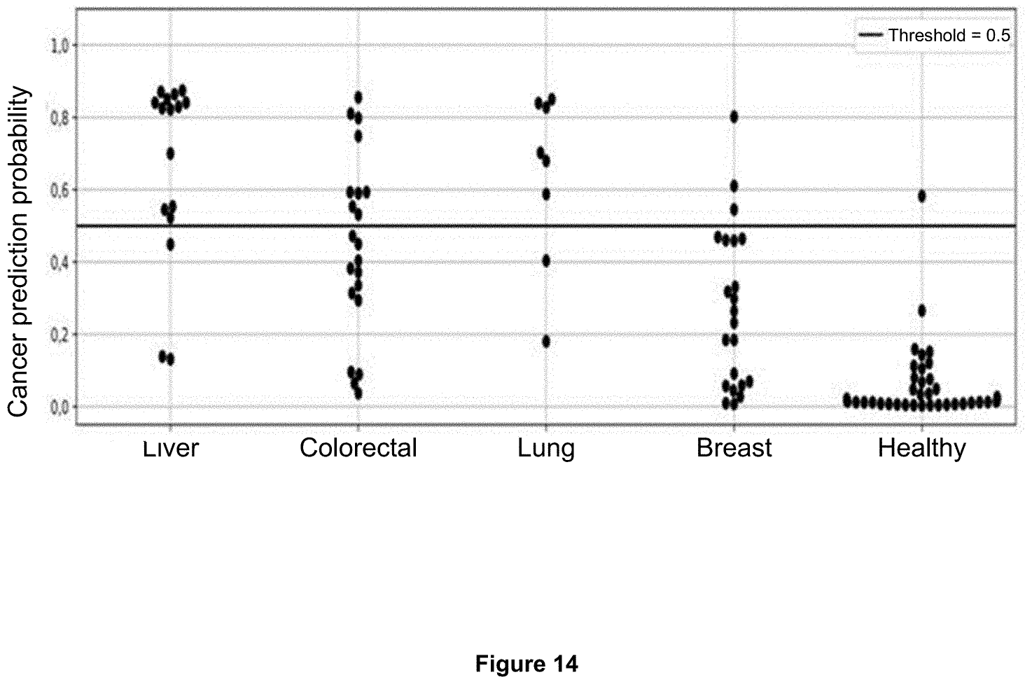

After selecting the model with the best performance, the effectiveness of the selected model was evaluated on the model evaluation dataset. Similar to the model training, the specificity, sensitivity, accuracy and AUC values of the model were determined on the model evaluation dataset. The model has the best performance when these values were the highest and were equivalent to the values obtained in the model training. The model's evaluation results are described in Table 22 and FIG. 14.

TABLE 22

| | Average | Confidence interval |
|---|---|---|
| Sensitivity (%) | 70.00 | 66.90-73.10 |
| Specificity (%) | 89.67 | 87.18-92.16 |

The results when applying the model on the leave-out test set show that the sensitivity of the test reaches 70% (with confidence intervals ranging from 66.90%-73.10%) and the specificity reaches 89.67% (with confidence intervals ranging from 87.18% to 92.16%).

FIG. 14 is a diagram showing the test results of blood samples from patients with liver cancer, lung cancer, colorectal cancer, and breast cancer using the SPOT-MAS test procedure according to the invention. Specifically, the vertical axis represents the probability (likelihood) of cancer prediction of the analyzed sample, and the horizontal axis is the list of surveyed cancers. The classification threshold value from the algorithm was 0.5 (solid line). The samples above the classification line were predicted by the model as cancerous and below this line were considered noncancerous. The results showed that the model was able to correctly predict 13/16 liver cancer samples, 9/21 colorectal cancer samples, 6/8 lung cancer samples and 3/22 breast cancer samples. In the group of healthy people, the model only wrongly predicted 1 case of cancer in a total of 36 surveyed samples. This result demonstrated that the disclosed SPOT-MAS classification model achieved different detection efficiency for different cancer groups. The model delivers good results for the group of healthy, liver cancer and lung cancer samples while the effectiveness is lower for the group of colorectal cancer and especially breast cancer samples.

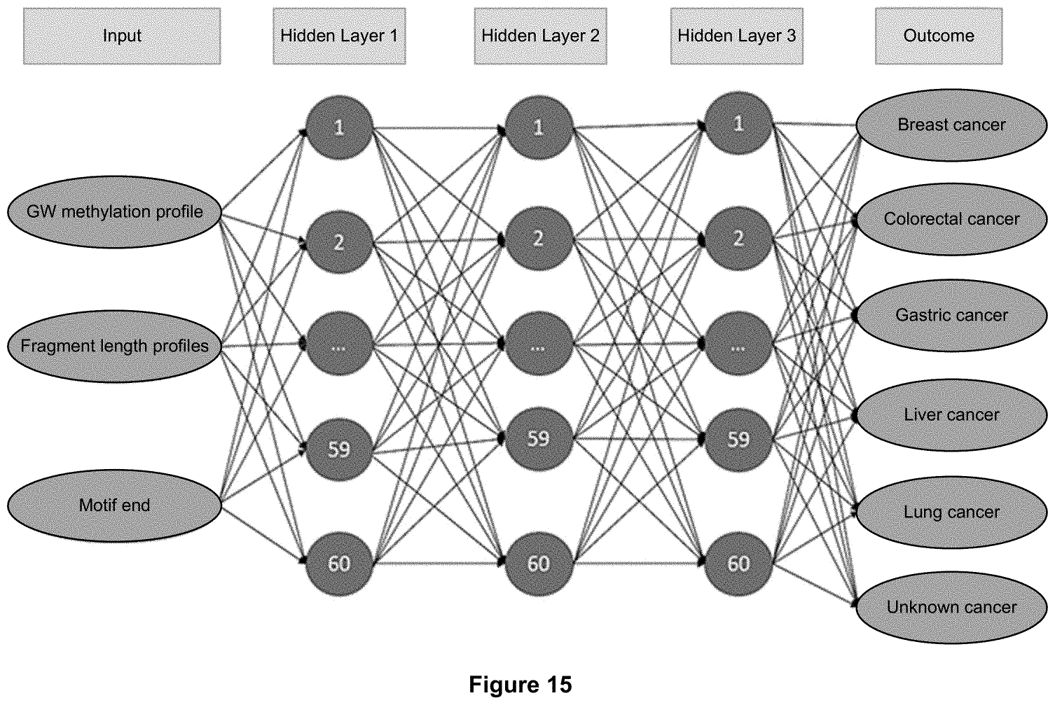

cfDNA released from different organs have variations in epigenetic marks including the methylation, fragment length and motif-end profiles that can differentiate one cancer type from other cancer types. To determine the tumor tissue origin, a deep neural networks (DNN) model was built from such epigenetic signatures (FIG. 15) as inputs. Structural for deep neural networks model was based on the multi-layer feedforward artificial neural network that was trained with stochastic gradient descent using back-propagation. A random grid search in H₂O platform was used to select the hyperparameter for of the deep neural networks. The model was built from epigenetic signatures such as GC methylation, fragment length and motif end. The hyperparameters included for instance (1) three hidden layer with 60 nodes in a layer; (2) activation function: Rectifier With Dropout; (3) Input layer dropout ratio: 0.01; (4) Loss function: Cross Entropy; (5) Rate annealing: 1e-06; (6) L1 regularization: 0; (7) L2 regularization: 0.

The disclosed DNN model returned probability scores of five (5) cancer types (breast cancer, gastric cancer, colorectal cancer, liver cancer and lung cancer) and probability scores of unknown cancer. The DNN model had 3 hidden layers and 60 nodes in each layer.

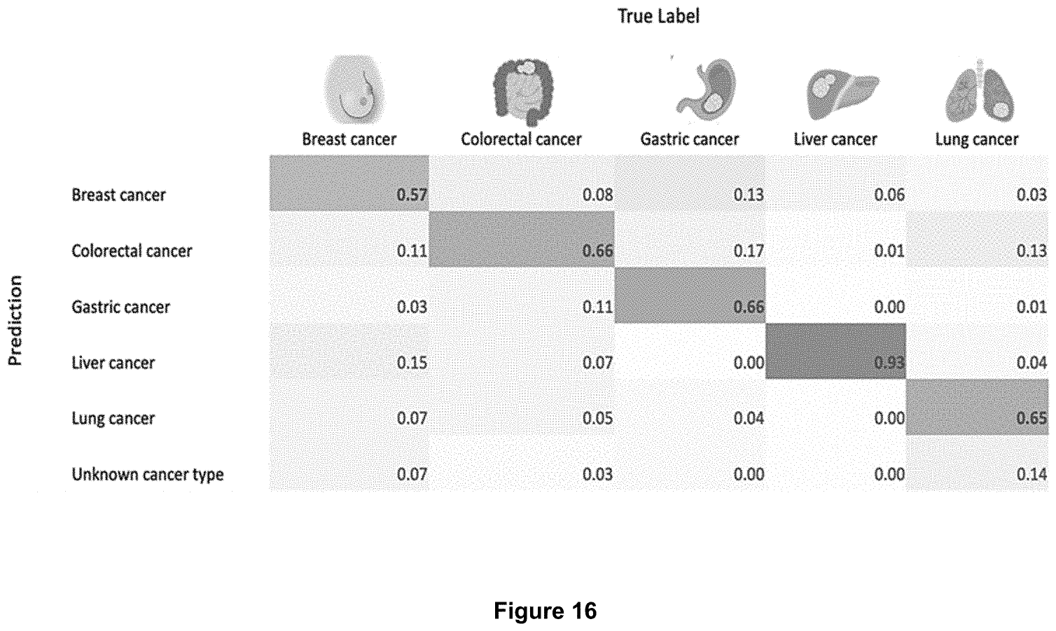

The performance of deep neural networks with hyperparameter was tested using leave-one-out cross validation (train in (n-1) sample of data, leave one sample to test the model). The result for the leave-one-out cross validation was shown in FIG. 16. The model achieved a mean accuracy for five (5) cancer types of 0.69 (95% CI: 0.68-0.76). Of the five cancer types, liver cancer can be effectively differentiated from others with the highest accuracy of 0.93 while breast cancer showed lowest accuracy of 0.57. The accuracy for identifying colorectal, gastric and lung cancer were of 0.66, 0.66 and 0.65, respectively.

### Example 4: Effectiveness of the Systems and Methods of the Present Disclosure

Due to the combination of simultaneously identifying four attributes carrying characteristic variations occurring in the entire tumor genome, the SPOT-MAS test procedure according to the systems and methods of the present disclosure provides higher accuracy (sensitivity and specificity) than published tests that rely solely on one or two attributes. Therefore, the SPOT-MAS test is effective in detecting benign tumor DNA in the following cases:
Early stage cancer with low tumor cfDNA level in the blood.
Certain types of cancer tend to release less tumor cfDNA.
Tumor recurrence after treatment.

Using a single cfDNA library preparation procedure (bisulfite treatment) for simultaneous analysis of four tumor DNA markers also helped reducing the cost of the disclosed SPOT-MAS test as compared with similar tests that need to take blood samples and multiple independent cfDNA processing procedures. Therefore, the SPOT-MAS test allow increasing the patient's chance of accessing a cancer screening test.

The disclosures of each and every patent, patent application, and publication cited herein are hereby incorporated herein by reference in their entirety.

While this disclosure was provided with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

TABLE 23
List of target sequence regions of interest

| | | SEQ |
|---|---|---|
| Gene Name | Sequence (5′-3′) | ID NO: |
| C1orf159_TTLL10 | CCGAGAGGGGTCACGTTCTTGCCGCCTACCTGACAGCAGGCCTTCTAGAAAGTTCTC | 1 |
| | TCCAGAAGCAGCCACCGCCGTCCTGAGGCACTTTGTGCGGAGACGGGAAGCTGTC | |
| | GCCTCAGAGGTGGGTGCGTAGAAGGGTTTGGCCGGGTGCGAGGATGACCGCGTCT | |
| | CCCTTGGGCTCTGGAGTCTGCGGTGGGAAGGGCTTGGTTTCAGCACCCTCTGGTCA | |
| | GAGGCCGGCCG | |
| PEX10_PLCH2 | CCGCTGACTGCGCCTCCCGGCCCGCAGCCCCCGCCCCCGCCGCCCTCGCTGCCCTCG | 2 |
| | CTGCAGCCGCCACGGAGACAATGGACGCGGGAGCCGCCCCGCAGAAGCACAGTAG | |
| | GTGCCGCTCCTGCCGCTGCGCCGCTGCCAACCGGGATGCGCGGGTGGACGCGCGG | |
| | GGGCGCCGCAGCCCTGGTGCGGGTCGGGGCTGAGCCGCCTGGGCTTCAGACTCGG | |
| | GAGCGGAGGCTCGGATCGCGGTGGCACGGGCAGGGGTGCGGGCGCGGGACTGTG | |
| | GGCGGGACGGGCGGAGCGGTCTTGAGCTCTCCGGATGGCCTCAGGTGCGGGGTG | |
| | AGGGATCTGGGGGCCGCCCCTCGGCAAACTTTCCTTCCCCGGGCTTCTGCG | |
| ACTRT2_MMEL1 | CCGGACTGCGGCCCGGTCGATGGAAGCAGCGGAGCTAGACCTGCCTCGGGTGCTT | 3 |
| | TGGGAAGTCACCAGCCACTGCCTCCGTTCATTCCTTTGTAAAATAGGAGGAAACACA | |
| | TCCGTCGCTACCTCGAAGGAGACCCGCAGGAAGCAGCGGCCCCAGCGTGCCCGGG | |
| | CGGGTCCTCACCCCTCCTGCGTGGTGGGGCCGCCCGTCTCTGCGGCCTCCCTCCGGC | |
| | CCTGCGCTCTGGACGGCCCGGCGCGTGGAGATCGCTGCAGCATCCCACGGGCCTCC | |
| | TCCCG | |
| ACTRT2_MMEL1 | ACGCGCTGCCCGCCAGCACCCGCAGCAGTCCCCGGCCGCACAGCGCGCGCACACA | 4 |
| | GCCCCCCGGGTGCGGCGCCCCCTGCTCCACTACCGTCTGGAAGTCCTCCATGGCGC | |
| | GCCGCGAGTCCCCGGCGTGCAGCGCGCAGAAGCCGCGCAGAGCCAGGAGAGGTG | |
| | CGCGGTCCCCTGCACGGTCCCCCGCAGGGTCCTCAGGGCGTGCGGGCCGCAGCAG | |
| | GCGCTCGCACATGGCCCGGGCGCCCGCCGCGTCCCCTGCCAGCAGCAGGCACTCGG | |
| | CGAGGCGGGCTCCCAGCGCGGGTCGCGCCCCGGCAGGCGCCAGGCGAAGCAGGA | |
| | CGCGGAGCGCGCGGGTTACCGGGGCCAGCAGCTCGCGGCCCGCATCCTCGCGGAG | |
| | CTCAGGGTGGTTCCGCACG | |
| KCNAB2_CHD5 | CCGCGTTTCCTTCCTTGGGCCATCTGTGTCATAACCATCAAGACGCAGTGGCTTCTTC | 5 |
| | ACATTTCTGGTGATGTTGCTTCTCCATGTGCCAATCCCCCAGCGGATACCCCACTCTC | |
| | CGGAGGGAGAACCCCAAGCAGGTGCCGCTGGGCATGCGCCAGGGAGGCTGTGAC | |
| | CGGAGCAAGCACTGCCTTGCTTGGAGCTGGCTGCCTACAAGCTCAGACATCCAGCC | |
| | CGCAGAGTCCACCCTGGCTGCAGCG | |
| VAMP3_CAMTA1 | GCGGCGGTTTCCATGGAGAAGGTCCTGATGTTCTCCAGTAATTTCTGCAGTTCTTTG | 6 |
| | TTCCCGGCAGCAGCCCCAGCCTCATGCTAGCAGCTGTTGATTGCG | |
| VAMP3_CAMTA1 | CCGCCG | 7 |
| SLC25A33_SPSB1 | GCGGGTCGCTTTGGTGGGAGTTTCTTGCTTCCTTGGCACACCATTCGCTCCGCGAGT | 8 |
| | TTGTTAAGGGCCCCTGTGTGCCAGGCTCGGCCCGAGCATCTGTGGAACCAGAGGAA | |
| | GCTGGGTGGACAGTCGCAGGTTTGGTGACGTGCCAGGTGGGGAGAGGAAGCAGC | |
| | TGCACTCATTCCCCTTTCCGGGCAGGTTGGGGAAACGCAGCGATTGTTCTGGGAAG | |
| | CTGCAGCTTAGGGAGAGATGACGTTCCCTGTGGCCCAGTGAGGGTGGGGCCCTGG | |
| | GGTCTGGGCTGACAGCAGGCAGTGGGGGAAGGTGGGTGTGGGCACCCGGAGGCC | |
| | CATGATGCCCCCAGATCCTCCACCACG | |
| EFHD2_TMEM51 | CCGTCCCAGCATGGATGCCTCAGGCCGACAGAAAGTTTTCCCTTTAGGTTGAGTTGT | 9 |
| | GTCAAACTCTTACGCCCCGGAGGAGTATCAGTCCTCCGCCCTCCCCTGCGCTCCCAC | |
| | AAGATACATCTACTTCCTCTTCCACATGATGACTCAGATGTGTGAAAACAGGGGCGC | |
| | CCGCACCCTGTGTCTGCTCCTCCCCGGGCCCAAGCGCCCTTGTTCCTCAGGTCCCTCA | |
| | CAGGACTAGAGCCTGGCCCTGGCTGCCTCCTGTGGCCTGTGCTGCTCTCCAGAAGT | |
| | CACAGACTGGTAGCTCAGCG | |
| PTPRU | GCGCACAGCGTCCCGGCCCTCCCCTAGCTCTGCTCTGCGCTTTCTTGGGTCCCCCATT | 10 |
| | CCCCCAGGTTAGAGCGCGGCTCCAGGAACCTATGTCCGCGCGGTGTAGTAGGGAC | |
| | GGCTAAATGGGGCCCGGGTCAGAGCGAGATCGGGACCCCTCGCTCCGAGGCGCCC | |
| | CTGACCCCCTCACTCTCTTCCCTGCAGCGGCAGAGCGGGGCGCTGGTGCCGGCGGC | |
| | GGGCGTGCGGCACATCAGCCACCGGCGCTTCCTGGCCACTTTCCCGCTGGCTGCCG | |
| | TGAGCCGCGCCGAGCAGGACCTGTACCGCTGTGTGTCCCAGGCCCCGCGCGGCGC | |
| | GGGCGTCTCTAACTTCGCGGAGCTCATCGTCAAGGGTCAGCTGGTGGACGCCGGG | |
| | GAGCGCCGGGACCTCACCCTCGAGGGGCGGGGCCGGCGACGGGGGCGGGCTCTG | |
| | CCCGGGGGCGTGGCCG | |
| ZC3H12A_MEAF6 | CCGTCAGGGCACCCCAAGGCCGGGTCAAGAGCTGGCCGCTGAGGAGGCCTCGGCC | 11 |
| | CTGGAACTGCAGATGAAGGTGGACTTCTTCCGGAAGCTGGGCTATTCATCCACGGA | |
| | GATCCACAGCGTCCTGCAGAAGCTGGGCGTCCAGGCAGACACCAACACGGTGCTG | |
| | GGTGAGCTGGTGAAACACGGGACAGCCACCGAGCGGGAGCGCCAGACCTCACCG | |
| KDM4A_PTPRF | GCGGGTGGAGGTGGATTGGAGGGAAGCGGAGGGCGAGGCCTGGTTGAGGGGCG | 12 |
| | GGGCCTGCCTGTCTGGTCCCCCGGGCTGCCTTGGGCCAGCTTGGCCTAGTCTGTTG | |
| | GGTGGGCGGGCAGGGTGCAGGCTCCTCTCCAGCCTCCAAGGGAGGGGAGTTGTTC | |
| | TGCCTCCTCGATAGCCCCAGGCCTTGGGCACAGCCCAGCCTCCCACG | |
| FOXD2_TRABD2B | TCGCGGGCAAAGATCCGATGAGAGAGAGGCAGAGAAAATGAGAGGCAGAGACAG | 13 |
| | AGGCAAAGGCACAGCGAGACACCGGGGAAACGGGGAAGCAGGTCAGAGAGGAA | |
| | GAGAGAGACAGGCCGGAAGAGACTGTGCCCAGGAGCCTGGACAAGGGATGCCGT | |
| | GCCCAGCAGCCTGGACAAGGGATGCCG | |
| DMRTA2_ELAVL4 | GCGGTGGGGCAGAGGACGGGGATGAGGCGGCCGGAACCGCCCTACGAGGAGAG | 14 |
| | GCTGGGAGGCTCCGAAAACCTGGGGTAGGGGGAGCGCACCGGGGCTTTAGAGGG | |
| | CGCAGCGGCCAAGGGCAAGAAAGTTTACACTCCCAGAAGCTTCCGCACGCTTTCTC | |
| | CCG | |
| DMRTA2_ELAVL4 | CCGCGGAGTAGGCCAGGCGCAGGGGGCTGAGGCCGAGCGGCGCGCCCAGCGGGT | 15 |
| | AGGCGCCCGCGTCGGCACCGAAGTGACTGGCGTTGGGCTGCAGCGGCGAGAAGG | |
| | CCGAGCGGCTGCTCAGCGAGCCCAGCGCCCCAGGCGCCATGGCGCCGGCCAGCAA | |
| | GGGTCTGTGGTGCGGAGGTGCGGCGGGCCCCGCCTGCAGCGGCGCAGGCAGCCC | |
| | AGGCCCCCCGGCGGCGGCGGCGGCGGCGGCGGCGGCGTCGACGCGGCTGGGCCA | |
| | CGCGTCGTCTGCAGCTGCTGCAGCACCCACGGCGGCCTTATCTGGGGGCGCCGCAG | |
| | GGCCCAGGCCGGCCGCCAGGCCCCCACGGTGGTGGTTCAGCACCTGCTCGATGGCC | |
| | TGCACCACGTCGCCGCCGCAGCCCTGCAACACCAGCTCCAGGACGCCTCGCCGGTG | |
| | GCCTGGGAACACGCGTGTCAAGATATCCAGCGGCGTCCGCTGCCGTGGACCCGAG | |
| | CCTCCGCCCAGCCCTGGCGCCGGCGCGGCCTCACCCTCTTCTTTGTCAGCCTCTGAA | |
| | CCGGATTCAGAGCCCAGAGGGCTAGCGGAGCCCGGGCTGTCCTCCTCGCCG | |
| DMRTA2_ELAVL4 | GCGGGCTGCCCGGGCGGCCTGCCTGCAGCAGCGTCTTAGGAAACAGGTCAAACTTC | 16 |
| | TGCAACTTGGCCTCTGGGAGGGGAGAAAACGTGTCGTGAGGAGCGGTTAGCTAGA | |
| | AGACAGCAGTCACAGCACCTCG | |
| FOXD3 | CCGGGGAATGGACGGATCAGGCTGGGCCGTGGCAGAGGGAGGGTAGGAGGCAG | 17 |
| | CGACCAGCAGCGTGGAGGGAGTCCAGAGAGCTAGCCTCTGCGGACGGCGGAATCG | |
| | AAATTAGGCTCATTTGGAGACTACTTCGAGACCGGTGAGGGGAGCCCTGTAGCCAC | |
| | CATCCTCCGGCGCGCATCCACACATACTAGTCCACGCGGGCCCAGCCACCAAGGCC | |
| | GCGGCAGGGCCAGCGCTGCGCCCCG | |
| SERBP1_GADD45A | TCGCTGCTTGTTAGGCTTTTTGTGCTTTGATGCCAAGAGCCTCAGTCTCACACGCCCC | 18 |
| | TCTGGCCGTCCCTGCCTGGGACACCGAGTTGAATTTCCCCACCCTGCGTCTGGGTCC | |
| | TCACTCCCGCGCTCCGGGCGTCCAGCTCACGCCTGTCTGGTGGATCTTCTAGTCTCT | |
| | GCGTTGGCTCTCTCTGACCG | |
| BARHL2 | GCGGCGGAGACGCGATGCCGGGCGACTCCGGCCGCTGCCGGGCGCGTTCGCTTGT | 19 |
| | AATCCGGCTGCTGGCGGGCGGCGCCGACCCCCTCCCGTGACGTCACGGCCACTACC | |
| | GCCGCTCCCCGCGCCGCGCCGCGCCGGGCCCGCG | |
| CSF1_EPS8L3 | ACGGAGAAGCATGTTCGCTGCCGGCAGAGGCTGCTGAGAGACCAGCCTGTTTGCAT | 20 |
| | GGCTGGAGCG | |
| CSF1_EPS8L3 | GCGTCCTGGCCCCACAGGACAACTGGAGCCG | 21 |
| ALX3 | CCGCAGTCCCCAGCCGACCCCGATTTGACCACTCTAGGTTGAGGCCCAGCCTCAGG | 22 |
| | GCCCTCAAAGGGCGCCAGACACAAAAGCCGCGCTTCTTCGTCAGGTCTCAGTGTGG | |
| | CTCCACAGCCCTCGGCCGGGTCTGGGCTTCAGGGTAGGTGGCAGTTCCAGTCCAAC | |
| | TTCGGCAGAGCATGCTCTCTCCTTCCCAGGTCCAACTGCTTTCGGGCCCCGACTGGA | |
| | CTCCGGGCCGTCGCCACTGCACCTTCCCTCGACCTCCCGCCTTCCATTCCCGCCGCCG | |
| | AGGAACGGTGGTTCACCCTCCCGCCCCACACTGGCCTTTGCCTGGCCCGGGCCAGC | |
| | GCCAACCCGGCTTCCG | |
| UBL4B_ALX3 | CCGCTTGGGGAGGATCTGGCTGGTTTAATGGTGATTCGATGCAAAAACCGTTGATT | 23 |
| | CCATTCTGATGTACTCAAGAACAGAGATGGCTGGAGACAGAGACAAGGAGAGTCA | |
| | GAAAGCGACAGAAAGTAAGTCTCTCCGGGCCTCTCCACCCAGCCAATGACAGTATC | |
| | ACTTCAGGAAGAGACACTCCCTGTTCCCCAACTTCGGTTCCCCCTCCGCCAAAACCG | |
| CHIA_CHI3L2 | GCGGTGACCCACCGGTGAGTCCCGGGTGGCCTAGGGTAAGGCGGACCGGGAGCC | 24 |
| | ACCTCACACCCACACAGCCTGCGGGAAGGATCCGACAAGGTGAGGGTAGCCCCGC | |
| | GCGGGGCCGCAACAGCCTATTCCTCCCGTGTGTGACGACCCCAGCCAGAGAGAACC | |
| | CAACCTGAGTGCCAGCGAGAGCCTGTCCTTGGTCGCTCCGACCCCTCG | |
| CHIA_CHI3L2 | GCGGGGCAAGGAAGCGGATCTTCATCCATGTCCCTGGATGGAGTAAGGCACACTCT | 25 |
| | GGAGGTAGCAGCGAGTTTGAAGTGTCTAAGAAAAAGGCCTTCTGCAATTCACAATT | |
| | CTTATGGCTACCTGCACCTTTCATTTACCCACTCAAAGCTAAAGGTAGCCGACG | |
| SPAG17_TBX15 | CCGCCATCCCTCAGGGTTCCGGGTCCCGGGTTTCCAGGGTCCCGGGTTTCCAAGGC | 26 |
| | CCCGCGATAACCCCGGGCGCACGCGGCGCGATGCGGCGAGGCGAGGCGAGGCGG | |
| | TGGGGCCAGCGCGGAGCCCCAGGCGCGAGAACAGGAACTCGGGCTGGCACACCG | |
| | AGGCCTCGCAGCCAAGCCG | |
| SPAG17_TBX15 | TCGCTCCGCGGGAGACCCGGCTTCGGCAGCACTTAGCAGAAGATTTTGGCGGGAA | 27 |
| | AGGCCCAAGCCCTAGCTGAGGACTCCGGGTGGAGCAGGGGCTGAGGTCCGAGCGC | |
| | AGATGGCGCCGCCGAGCGCCTGAAATATACTTGCAAGGCCGCAGCAATATACTTGC | |
| | AAGGCCGCAGCCGGAGCAGCTGTTCCAGCCGATCCTAGCTCGAAAGTTCCTCTGTT | |
| | GCTCTGGGAGAGGGCGGGGGAGAGCAGGCTCGAGAGCCAGGCTCCTCCG | |
| TBX15 | ACGAACATGAACTCTGGGGAGCTGGAAGCAGGGTACTGGTCCCCGCCTCCTGCAGC | 28 |
| | TCTGCCCAGAGGACTTGGGGAGCCCGGATGGAGAGGCGCAGGATCTCCCACTTCA | |
| | GTCAGCATTTGGCGTTGCTTCCAGGAGTCGTCGCTGAAAGTCAGCGCGCATTCACT | |
| | GCTACCGGGCTTCAGCAGAGAAGCTGGAGACAAGGCAGACGGGAACCCGCAATTT | |
| | CCTTCCCCAGCGGCTGGGGCCTCTCTCTCACCTCCCAACTCTGGTGTCGCCCGGCGT | |
| | TTTCCGCCTGCG | |
| TBX15 | TCGCCTTCGGCCGCCGCGGTGTGGCCGGCAGAGCCGGGGCCGGCGGGCCGCAAAA | 29 |
| | TTGCGCGATTGTTCGCTGACTTCGGTCTGCGCAGGAGCAGGGCCCCTCCACAAAGG | |
| | GAGCCTTGTGTGGCCAGGCCGGAGCGGCCGCGCCCAAGAGGTGAGGAAATCCTGT | |
| | TCCCCCAGGCCCAGCTTCTCTTTCCCCACGGCGTTTCGTGCAACGCCGCAGCCCGAC | |
| | CTTCG | |
| ENSG00000255168_ | GCGCCCTGGCAGTCCCGGAAAACACCAGGAAAACAAGCAGGAACCGTAGCTAGGA | 30 |
| PDE4DIP | CTGGGGTGGCCAGGCCCAGGAAATCCATGAAGGGCACAGACAGCGGGTCCTGCTG | |
| | CCGCCGCCGATGCGACTTTGGCTGCTGCTGTCGCGCGTCCCGCCGGGCTCACTACA | |
| | CGCCTTACCGGTCCGGGGACGCG | |
| PIAS3_ITGA10 | TCGGCAAGCCCCAATGAGATGCTCCATCTTCTCTTTCAGCAGCTCTGCCGTTTTCTCA | 31 |
| | AACTGCTCGGAGCGCCCCCGCATCTCGCTGGCCTGGCGCTCTTGTTCGGCTGCCTGA | |
| | GCGGCCAGGTCCCCGCTCCGGCGGCGCTCCTCGGCCACCGCCTCCCGCAGCCGACC | |
| | CACTTCCCGGCAGGCCGTATTATACTTTTCCAACAGAGCCTTTAGCTCCTGGCCCCAA | |
| | GCCTGAGCCTGGGCCACCAGCGAACCCCGAGTTTTCTCGTGTTGCCGTAGGCTGGC | |
| | TGCCTCCCG | |
| PKLR_HCN3 | ACGGTGTTCGCGTTCCCCCGCGTCCGGAACGCGGGGTCCACAGTCACCAGCACCTG | 32 |
| | GGAGCCCTTCACCAGCTCCACTTCCGACTCTGGACCCTAAGGAGGGAGCCAGAGGA | |
| | GATGTGAGTTCTGAGCCCCGGAGTCCGGGACCCGCCCCTGCCCACGCCTGGGCCCA | |
| | ACCCTACAGGCGCCGCCTTTCCGGCCCTGGCCCAGCGAGTCCCAGCCCCACTGCTCA | |
| | CCCCCTGCAGGATCCCAGTGCGGATCTCCGGTCCCTTGGTGTCCAGGGCGATGGCC | |
| | ACGGGCCGGTAGCTGAGTGGGGAACCTGCAAAGCTCTCCACCGCCTCCCGGACGTT | |
| | GGCGATGGACTCAGCATGGTACTGGGGGAGGGAGCGGAGCGAGGGTTTCAGGGG | |
| | AAGGTGGCCAGGACCTCGAGGCATCCTCCTGCCCCACCCACTGCCCGGCGGCCCGT | |
| | CCCGCACCTCGTGGGAGCCGTGGGAGAAGTTGAGTCGCG | |
| SEMA4A_LMNA | TCGTTTTCGATGCCTCTCCCTTCTGGACGGTGGAAAGGGCTGTGTCATAGAGTAGG | 33 |
| | AACGGGAGATGCGGCACAGGAATGGCTCCCATTGACCCGGGTTGGGGGCTAGGGC | |
| | GAAGGCCTAGGAGAGGCAGAACTGTTACCTTAGAGCTGGCCAGGATTAGAGAACA | |
| | GTGCCTGGAACCGGGGGGAGGGGCACGGTGACCTTGGGCTGCCCACCTTCTACCCT | |
| | TCCAGCACCCATACTGGCTCCCCCAACCTGCG | |
| C1orf61_MEF2D | CCGGGGAGAGCGGGAAGCCTGGCAAGCCAGGGAAAGGGAAGATGAGACAGAGA | 34 |
| | GACATAGAGAGACAGGGACAGAGGGAGACAGAGAGGGGGCTAAGAGCGACGCG | |
| | GGCGAGAGAGGAAGAAAGGCTGGGGAGAAGGAAAAATGAGATAAATAAAGGAA | |
| | AAAAGAGAAGCGAAGGGCGGTGGGAGAGGCAGCCGGGCCTCTCTGGGAGCTTAG | |
| | CCAGAGGCGCCCG | |
| BCAN | CCGGGGAGGGCGGGGCAGGGGCGGGGGGAAGAAAGGGGGTTTTGTGCTGCGCC | 35 |
| | GGGAGGGCCGGCGCCCTCTTCCGAATGTCCTGCGGCCCCAGCCTCTCCTCACGCTC | |
| | GCGCAGTCTCCGCCGCAGTCTCAGCTGCAGCTGCAGGACTGAGCCGTGCACCCGGA | |
| | GGAGACCCCCGGAGGAGGCGACAAACTTCGCAGTGCCGCGACCCAACCCCAGCCC | |
| | TGGGTAGGTGAGTGCCTCCGCAGCCCCGCCGCCCGCCG | |
| ARHGAP30 | TCGCCTCACCCTCCCTCTCCTGTTCCCAGTCACCTGCCCGCTGTTTCATCCACTCCTCC | 36 |
| | TCG | |
| TADA1_ILDR2 | CCGTAGTACTCCTCCAAGGAGTCGTCCTGGTAGAAGCCGCTGTGCGCCCGCGACTC | 37 |
| | CGAGCGCTCGAAGCGGCTCCCGCCCCGCGCCTCGTGACTGTTGCCGTCTGCCCGGC | |
| | GGGGCCGCTGGCCGTAGGAGTCAGCGAAGGCCGCCAGCTCGTCCATGGAAACGGC | |
| | CGGCACCCCCGTGGCGAAGTTCTTCCGCGACAGCATCTCCGACTTGGAGCGCG | |
| HLX | GCGGATTTGCGTCACCCGAGCAACTTGCCGGTGGAGATAAAGTTGCACAAATATTG | 38 |
| | AAAGGGGAAGTGCTAGGAGTCATTATAGAGTTTTTCTCCGGAAGAAATAAGGATTT | |
| | CTGCAGTATCCTAAAATACTAAGGCCGCTTCTATTTTGAGACCAATCTCGCAGGCAC | |
| | ATCCG | |
| HLX | GCGGGAGTCTGCGGGCTCAGAACTCGGCGAGGGGCCTGCAGGGGCCAGGCTTGG | 39 |
| | GCCTGGGGAAGGGGTAGAGGGGGCGGCGGGGGTCGCTCCAAAGACTTGTATTTC | |
| | GCGTTTGCCTCCGGGAGCTGGGAGTAAGGCCTTGGATGGCGCCGACGCGGTTGCG | |
| | AGGAAGCTGAGGCCTGGGAGAGCAAGGGGCGCGCAGGCGAAGTTGCAACTTGCA | |
| | CTCCAGCCGCGGGCCTGGCG | |
| RYR2 | GCGAGCGCGGCTGGGCTGCGGGGCTGCTTCCCCGCGTCCTCCGGGCCCGGGCCGC | 40 |
| | CCTCCTCCCGCACAGTGCGGAGCAGGGAGGCCCCGCGCCTCGACCACCCGCGCCCG | |
| | AGCGTCCGCGCCTCCTCCTCCGCTCTGCAGGCGGGGACCGCCCGGCGCTCGGCACC | |
| | CGGCAGCGCGGCCCCCTCCAGCCCCCGGCTCCCG | |
| RYR2 | GCGTCAGGGCATCCACTAGCGGGGTCCGGGCAGAGTGACAGCGGGCAGCGGGGA | 41 |
| | CTCGCGGGCGGGGCGAGGGGGTGCCCCCTGAGGATGCGGGAGGAGCGGGCATCA | |
| | CCAAGTGTGTGCAGGTGTGCGTGTTGGGGCGAGGGAAGGCAAGGGCGCGTGTCT | |
| | GTGCGCGCGTGTGGAAAGCTAGAGGATGGAGCGCGGCTAGCCGGCGGCAGGCGC | |
| | CCGGGCTCGGACCCGGGGCACCGGGGACAGGAGCGTCGGAGCTGCGGGAACCGG | |
| | GAGAGGAGGGGACGGCCGGTCCGGCCTGCCTGGTGGCACGGCTGGGACCTCCCG | |
| | GGCG | |
| FMN2_CHRM3 | GCGCGCCCCGTCGGGGACCGGGCGGGGACGGGAGAAGGAAAAGGGCCCCTGGCT | 42 |
| | CCGGGACCAGGGCTCCGGAGGGTGCCGGGCGGGGAGCGGAACAGGGAACGGGC | |
| | TGGTGGCGGCCCCAAGCGGGAGGGACGGACCGACACGCGGCCCCCTGGCGGCCTT | |
| | GCG | |
| FMN2_CHRM3 | ACGGTCGCCGCGGGCAAGGACCGCGAGGTTGCGGCCCTGCTCCGAATCCCGGCTG | 43 |
| | CGCTGGCCACGCTCCTCCACGCGCGGGGCGGCCGCTCCGCCACCCGCACGGCGCCC | |
| | CGCAGCTGCTCCGGCTGGGGATTCG | |
| TRIM58 | GCGCCGCCCGGGGAGCGGCTGCGCGAGGATGCGCGGTGCCCGGTGTGCCTGGATT | 44 |
| | TCCTGCAGGAGCCGGTCAGCGTGGACTGCGGCCACAGCTTCTGCCTCAGGTGCATC | |
| | TCCGAGTTCTGCGAGAAGTCGGACGGCGCGCAGGGCGGCGTCTACGCCTGTCCGC | |
| | AGTGCCGGGGCCCCTTCCGGCCCTCGGGCTTTCGCCCCAACCGGCAGCTGGCGGGC | |
| | CTGGTGGAGAGCGTGCGGCGGCTGGGGTTGGGCGCGGGGCCCGGGGCGCGGCG | |
| | ATGCGCGCGGCACGGCGAGGACCTGAGCCGCTTCTGCGAGGAGGACGAGGCGGC | |
| | GCTGTGCTGGGTGTGCGACGCCGGCCCCGAGCACAGGACGCACCGCACGGCGCCG | |
| | CTGCAGGAGGCCGCCGGCAGCTACCAGGTGAGGCGCCCCCCGGCGGGGGCTGCG | |
| DIP2C_ZMYND11 | CCGCGCTGCTCCCCCTCCCACCCCGAGGCAGCTCCAGATGGACACAGCAGGTCGGA | 45 |
| | ACATCCCACACCCCAAAGACAGACTACGGAGCAGAGCCGGCTTCCGCAGCG | |
| PITRM1_KLF6 | CCGGCAGGTTCGGGAAGTCCTCCCGTATTCGAGGTACCAGGAGCCATAAATCCATA | 46 |
| | TTTAATTAGCTTTGAACG | |
| PRKCQ_SFMBT2 | GCGTCGTCCCGGGATTCTCGGACACCACAAACGCCATCAACCACGAGCACCGGTGT | 47 |
| | CCGTGGCTATTGCCCCGAATGGTCCCCATCCGCGTCCCCGGGAACTCCCTCGGCTTT | |
| | TCGCGCATCCAGGTCCCCAGCCCCAGCTACTGGTGCGCCCCGAGCCCCTAGGTGCC | |
| | AGAGCGGTGGTCGGCCGGGCTCCTGCCCAGTCTCG | |
| SFMBT2 | CCGCGCTGCGCCTACCCAGTGGCCCTGGCCCCGCAGGGCGACAGCGGCTGCTCCCT | 48 |
| | CCCATTTGCGTCCCAGACCGCGCGGCCTCGCTTAGCTCCCGGGAGCCGACAGGCGC | |
| | TTGCCCTGGTGCCAGCGCAGGGCTTCCCG | |
| GATA3 | TCGAGATCTTTTATTTTTCTAAAGGTGGGGGTTGCCCTTCTCCATCCCCGGCCAGTCC | 49 |
| | GACTTGGTGCTCGCGATTGAATTTAAACGAATAATCCCTACTTCCCCATCCAAAATTA | |
| | GCGGATAGGCGCCCTTGCACCG | |
| PTF1A | CCGGATCACCTTCCAATGACACCCGCATATACTCTGCAAACTGTGCAAAAGCCCTTG | 50 |
| | AAAAGTCCAGAGATGGGACAGAAGCCCCCAGCAGAACCCAGGCCGGAGCCCCGCG | |
| | CACCTCGGATAAGGGGGTGGCGGAATGCACCCACCTGGTCCCTGAGGGCAGCACC | |
| | CTTAGATTGCCCAGGCTGCCGCGGAGGAGGACGATCGCCGCGCGGGCTCCGCTCTC | |
| | GCCGTCTGGGCCACCGGCGCG | |
| MKX | CCGCGCGCGGCCACCCGCGCCTCTTCTCAAATCACTTACCCCGATTCACTCCAGACT | 51 |
| | GTGGCCGGGGAGGTCACTCCCTGCAGAAGTGTCCCCCTCCCCCAACGCCGGCGAAT | |
| | AATTTTAAAGCAAAGGAGGCGCGGCCAGGTGGGCTCCCAAGCTCCGCGCAGACCC | |
| | TTGGGCCAGCCTTGGCCGCTACCCGAGCG | |
| MKX | GCGGGGCCGACGGCCGGCTGCAGGGCGGCTGGCTCTCCCGCCTCGAGACTAGGCG | 52 |
| | CACTCCCATCCCCGCCGCATGTTCTCCACGCGGGCTCCAGCGCGCTCACCACCGCCA | |
| | CCGCCGTCGTCTCGGCTTTATTTACCCAGCCCGGCGCGCGCCGCCCGGGAACAGGA | |
| | ATAGCGAGGCCTTCTCATGTTTCCTGACTGCCGGTCCCAGCCGGCG | |
| PRF1_PALD1 | ACGCGCTCGGCCCGCAGGTGGCACTCAGTAGACCCTGACGCACGTGTTCTGCTTGT | 53 |
| | GTGGTAGCCTGGGGAGGCTCCCCAGCCCTGCCTCAGTGGGCCTCTCCCTGGTGGCC | |
| | CGGCAAAGAGCAGAGCTTCATGAGAGCCCCTGCTGGCACTGCTGGGCTGCCTCGAT | |
| | GCCAGCCAGGCCGGAGGCTTGAGATGCCCGAAGTACCCAGTGCCCCGGCCACCTCT | |
| | CCTGGCCCTCTTCTATTTTAGGGCTCAGTCCAATGGATGAGGAAGCCTTGTCCGGCT | |
| | CCACCACAGCTAATGACAGCCTGGCAGGCCG | |
| DNAJB12_DDIT4 | TCGACCTTTCAGCCCGGTGGAGAAAGCAACTTCG | 54 |
| DNAJB12_MICU1 | GCGAATGGAGGTGACTGAAGGTATCAGTGCCAAACAGGTTCTTTTCTGCTTCATAC | 55 |
| | ACATTCCG | |
| EXOC6_HHEX | TCGGTGGGAACGTGTTAGGTCCACGTGCCGGTGGGTGTATGTGAATGTGTCTGGTT | 56 |
| | GGGTGGCCTCCTGGCCTACCTTTGTCATCCCTGGGGCCCGACAGCTCTGGGGTCTG | |
| | GCCAGGCCGCTCCAGGGCAGTGGGTGAGCGCCGCTCTTCCCGCTCG | |
| CYP26A1_CYP26C1 | GCGAAAGCAAAAGCCAGGAAGTTTAGGTCTGGGCCGCTTGGAAGAGGGAGAAAG | 57 |
| | GACCGGAACTGGCCTTCTGGCTACTCCGGAATCGCCAAGCAGATGAGGCCAGACCG | |
| | CCGCCAGCGCTGATCACGCGCGCTCCCACAGGTCCTGGCGCGCGTGTTCAGCCGCG | |
| | CCGCGCTGGAGCGCTACGTGCCGCGCCTGCAGGGGGCGCTGCGGCATGAGGTGCG | |
| | CTCCTGGTGCGCGGCGGGCGGGCCGGTCTCAGTCTACGACGCCTCCAAAGCGCTCA | |
| | CCTTCCGCATGGCCGCGCGCATCCTGCTGGGGTTGCGGCTGGACGAGGCGCAGTG | |
| | CGCCACGCTGGCCCGGACCTTCGAGCAGCTCGTGGAGAACCTCTTCTCACTGCCTCT | |
| | GGACGTTCCCTTCAGTGGCCTACGCAAGGTACGGCCGCCCCG | |
| CYP26A1_CYP26C1 | GCGTGATGTATAGCATCCGGGACACGCACGAGACGGCTGCGGTGTACCGCAGCCC | 58 |
| | TCCCGAAGGCTTCGATCCAGAGCGCTTCGGCGCAGCGCGCGAAGATTCCCGGGGC | |
| | GCCTCCAGCCGCTTCCATTACATCCCGTTCGGCGGCGGTGCGCGCAGCTGCCTCGG | |
| | CCAGGAGCTGGCGCAAGCCGTGCTCCAGCTGCTAGCTGTGGAGCTAGTGCGCACC | |
| | GCGCGCTGGGAACTGGCCACACCCGCCTTCCCCGCCATGCAGACGGTGCCCATCG | |
| FRAT1_FRAT2 | ACGCACTGGGTTGCGGGACAGAGTAGCCAGGTTCTGCCGGTGCTCGGAGAAGAGC | 59 |
| | GCAGTGTTTTGCAAGTGCTGGAGTCTCCTGAGGACACGCGCGTCGCCGCCACCGCG | |
| | GGTGTGGGAAAGCGCGGACGTGCTGGGCGGCTGTGCTTCGGTAGGCGACCACCGC | |
| | CCCTGGCCGCGCTCCGGGCTTTCACGGAAACTCCCGAGACCGGGCCCTGGGTTCCT | |
| | CCTCTCCTACTCG | |
| TLX1_LBX1 | CCGCGGAGAGCACATGCAGGCCGGAGCCCTCAGCCCGGCAGCTCTCGGACCCTGC | 60 |
| | CCAGCTCGACGCGGACTCATGCAGAAGAGGACATTCCGCAGGTAGGTACAATCCCA | |
| | GCGCTGGGGCCTGGGGCGTCCGGGGGGCGGCCTTTGAGCTTCCCGGATACCGCTC | |
| | GCCTGCTCCCGGAGCTGTTCGGCCGCCGGCTGCCCGGGTCGTGCACTTTCAGTAGG | |
| | GCCCCGCTGACTCTCCTGCCCTTGGGCTAGGCCTCCCGGGGATGCCAGACTCCTGG | |
| | GGACGCTGGGACCCGCGGCGCGGCGGGACACGCAGGACTCCCG | |
| BTRC_LBX1 | CCGCGCGCAGCTGGAGCCCGGCGAGAGGGCCGCGGAAGGGGGGTGCGAACCGG | 61 |
| | GGCCGGACCCCGGGGAGGAGCCGGGAGGCGAGCGGCGAGGGGCACTGCGCGGC | |
| | TGGGTCTGCCCCGGGGTTTCGCACTGCGCCGCGGGTCGAAGTACCGCGAGTTGGCC | |
| | CTGACTGTCTGCAGGATGAGGGTGTCGAGGAGGGTTCCAGGCCAGCGTGCCTGCC | |
| | TCGCCTCCAGCCCGGGGTAAGGAGATCCACGGAGGCCTCTGCGCCTAAACTCAGGT | |
| | GGCCAGACAGAGTTGGGGCGGGAGGCGGGTATACG | |
| SORCS1 | CCGTCAGCGCAAACGTGGTGCTGGTCAGTCTCAGCTCCTCCATCCGGAAGCGGGTG | 62 |
| | GCTTTGTCCGGGTCCCGCTCCCGAGTCCCAGGCTCCTGCTGCCCTCCATCTCTTAGCA | |
| | CTCCCCGGGGGCTCCGACTCGCGCCCTCTCCCCGTTCTGCCTTCTCCTGATCCGCTCC | |
| | GCTCCGTCTCCTCCGGCCGGAGCGTGCAGCAACCGCCATGGATGCCCCAGTGCCCC | |
| | GAGCCCGCTCCAGGGATAGCGCTCGGTCCCCGGGGGCCACTGAGAACAGGGGACG | |
| | CACTACGAGGGGCAGGGGCGTGGCAGGAGCCCTGCCTGGCCGCCCCTGGTGGGAA | |
| | AAGCCCCTAGGGGTCGAGGCCGAGCGTGGAGCGGAGCTGGGGTGCGGCGAGGGG | |
| | CAGCAGGAGCCGCCGCCGCAGACGCCCGGGGCGCAGAGGATCAAGAGCCCCGCG | |
| | CCGGCGAGGAGCGCGCTCAGCCGGGCTTGGGAGCCGCCGCCGGCGCCAACTTTTC | |
| | CCATCGCGGGAGCGAAGAGCAGCG | |
| NONE | GCGGGCTGGCTGCCTGGGCAGCACAGGACTTGAGGGAGCTGCGGGGACTCCTGGA | 63 |
| | GTCTCATCAGGCCTTCCAGTCGCTGTGGGGACCCCGGCTGCGCGCGGATCGCCTGC | |
| | GCCACTGTCCCCACTGACCCGCCCGCCGGGTTTGCCAATTACCAGCGCCACCTGGTC | |
| | CCG | |
| PLEKHA1_TACC2 | TCGGACCACACCGGCGCTCACGCTCATACCCGCACGCCCCGGGCAGAGCCGCGCAC | 64 |
| | GCCGGCCACACTCGGGCGCGCGCCGGCCACACTCGCGCGCACACATACGCGGCGC | |
| | TCGCCCCCCGGCCCCCGGCTCGGGCCGCGAGTCGCAGCTCCCTGCCGCCGCTCCCG | |
| | CCGCCACGGATGCCCGCAGCTGCTCCCCTCTGCAGTGCAGCAACCCCGGCCGCCGG | |
| | CCGGCTCGCCCCGGCTCCCG | |
| PLEKHA1_TACC2 | TCGCTCAGCAGTGGGTGCATGGCTGGGGGGCTTCTCCTGCCGTCAGCATCTTTCCTC | 65 |
| | TGCACCCCCGGCACAGTGGTATTTCCTGCAAGGGAACAGCCAGGCATCAGCGACTG | |
| | CCTCCTCCTAGGAAGAACCCATGAGCGTGGCAGCTCCGTGCCCGGGGCGACAGCCC | |
| | AGTTTCCGGGCAGCTGCGCTTGTGGCTGGGCAGATGGCGTGGTGCGCTCTGGTGG | |
| | ACGTTCCGTCTAGTTAGCCTAAGCATCATCCACATACTCTGGTGAACACTCGAGGAC | |
| | AAGGCCGCTTGCTATTATTAGTAAAGGGCCGAACCGTCCTGTCATTGGTGGAGGCA | |
| | GTGCTTGACTGTGCATCGATCCAGGAATCCGATCTTTTCTCTCAACCACAGAGCTAA | |
| | CGTGCTCAGAAGTGGCCTTTATCCTGGCCGAGTGTTTATTAGAATTCACG | |
| HMX2 | CCGCACGACATATTTACAGTTCAGGAAGGTTCGACCAACTTTCCCTGCCTGCCCCCA | 66 |
| | GCTTTCTTCCCCAGCGGGGTGGCTGGCACTGCTCCCCGAGTTAGCTGGCCAGTTCCC | |
| | CTCGGGGCTGCCTTGACCCTGGCTCCGGAGGCAGCGCCTAGCTCAGGATGTCTGCG | |
| | AGAAGCGGATGGTTAGTGAGAATCCGACGATTCTTTCGCTGAACCTCCCGCGTACC | |
| | CCCCAACAGCGCGGGAGCACGCGGGACCCGCTGCGACGTGGCCCAGGAGCCTGCG | |
| | CCGCCGCGGCGCAGAGGAGAACGCACAAATTGTATTTCAGCGCCAGGTCCTTCCGG | |
| | GTTAATGAGCTGACACCATGATTAAAGCTGACCATTTGTAATGTGTCTCGACCCTGC | |
| | CGCTGAGCCCTGAAGAGGTTAATGCGGTGACGGAGGCCGGCACCTGCCCCTCGCT | |
| | GGCCTCCCGGGCCGCTGCGCGCACCCCCTGGCCCCCGCCCCCTCGCCTGCCCCTGCC | |
| | CCGGCTGCGCGGCCGACTCCTAATCAATTAGCCCATTAACGAGCCCCTCGAGGAGT | |
| | TAAGTAGGGAAGAGTTCTGCCACGGGCAGGGCCGCAGTCGGTAACTCACCGCGGC | |
| | TAATGATATTATAAGCG | |
| BUB3 | GCGCGGAGAGGGAACTGGGCGCGGTGAGGCAGTTCTGCGGCTCAGGAGAGATCC | 67 |
| | GAGGCCCGGGACCAGGCAAAGAAGGTGAGGGAGGCAAAGGCGCTTCCCTACACTC | |
| | TTTTGTTGTTAATAGTTTGCATTGGTTCAGCGTGTGGCTGGATCACCGGCTAGCACG | |
| | CGGCCGCTTGCTCTGAATGGAACCTTGACGCGCGGCGGGGGCGCCCACGGACTTCC | |
| | TCGCCCTGACACCTGCGGCCGCG | |
| OAT_NKX1-2 | GCGAAAGAGGGGCCAGGGGGCTCCGGATTCATAGACGCGGGGCGTAGAAGGGGG | 68 |
| | TCAGGTAGGAAGGCCCAAGGAACGGCGCGAAAGGGCTCCCGGGGGCGGCAGCCG | |
| | TCAGCGGGAAGGAGGCGGCGGACGGGAAGAGGACATTGGCCGCGGAGTAGGAG | |
| | GGGAAAGTCTGGAAGTGCAGAGCGCCGGTGCCG | |
| FOXI2 | GCGCGGAGAAACCTGGCGGGGCCCCGGACTCCCCGGCTTGGGAAAAGCGATGACT | 69 |
| | GCCCTGAACTGCTGGGGCGTTCGAAATTTCCAGGGTCCCGACCCTCCGTGGGGTAC | |
| | GCGCGACTTCGGCGCAGATGTCAGTCCGCTGCCTTCCGGGTTGAGGGAGCGAGGA | |
| | CTCCAGACGACCCCAGGGCCGCTGTCCAGGCCCAGCCCCGCG | |
| MKI67_MGMT | CCGGCAGTGGGGAGCACCAGCTGGAGAGTGGGTGTGAGGCCACCACATCCCCCCT | 70 |
| | GCAGCTCCCAGCGCCATTTGAATACTTTGAGGAAAGATCTCAGCTCCTGCCGGGAA | |
| | GGCCCCTGCACAGGCTGATGACCCTGCTCTCCTGACTCTTTCTGACTCTTTTTCCGGC | |
| | GAACCCTGCCACCTCCTCCTTCAGGCCTGGGCCG | |
| MGMT | ACGGATGCATTCCGTAAGCAACTGGAAACCCCAGTACAAATAGTCCAACTTTAGAC | 71 |
| | AGTAGGACGGAGTAGAAGACAGGGTTCTGCTGAAAAAAAAATAAATGCTTTTCTAA | |
| | GGTTAACGCCGGGAAAAGTCCGGGGCCTCCCGAATTCCACTCCAGTGCTCTTTAGTC | |
| | ACCGGGCCACTTGCCTTGTCAAATGTGCGGCTGGGTTTCATCTCTGCACTGATGACA | |
| | ACGAAGGCCGTGGCAGCTATTAATCTTCACTATGGTCCTCATGAACTAGTTAAGCAT | |
| | GAAGGGTGACAGCCCTGAGCCCCAGGGGCCTTGACAACTGCG | |
| MGMT | CCGAAGAGCTGGCGGAGAGAAGCGGCTCCCAGTGCTTAGCCGGCCTGTCGGAGCT | 72 |
| | TCCTCTGCCTGTCAGCGCCCTCGCCTCTTAGCACATGTTTTCAAGGTCATCTCCTAAC | |
| | ACCGGCTGCCAGTTGCCCAATCGATAGAAGCAACATCACACTCCTTCCTTAAAAAGG | |
| | GAAAAACAAAGCTGCTTTCGATAAAGCCTCATCATCCTATAGCTTCTCCG | |
| VIM | TCGCTCCGAGGTCCCCGCGCCAGAGACGCAGCCGCGCTCCCACCACCCACACCCAC | 73 |
| | CGCGCCCTCGTTCGCCTCTTCTCCGGGAGCCAGTCCGCGCCACCGCCGCCGCCCAGG | |
| | CCATCGCCACCCTCCGCAGCCATGTCCACCAGGTCCGTGTCCTCGTCCTCCTACCGCA | |
| | GGATGTTCGGCGGCCCGGGCACCGCGAGCCGGCCGAGCTCCAGCCG | |
| MGMT | TCCCGACGCCCGCAGGTCCTCGCGGTGCGCACCGTTTGCGACTTGGTGAGTGTCTG | 74 |
| | GGTCGCCTCGCTCCCGGAAGAGTGC | |
| PPP1R3C | CCTGGGACCAATCGCCGGGCCTCGAGCCCCAGGGCGCGACCAACCAGCGCCCAGC | 75 |
| | TGGGGCGCCAGCCCTCGCCCCGGCAACGTGATCGCCCCGGGGCGA | |
| BMPR1A | TTTATGATAGTTTGTCCTGTGTCCTTAGTGATGTGTGTGTGTCTCCATGCACATGCAC | 76 |
| | GCCGGGATTCCTCTGCTGCCATTTGAATTAGAAGAAAATAATTTATATGCATGCACA | |
| | GGAAGAT | |
| ST8SIA6 | TCTCGCACTCCCCGGCTCCCAGGCCAGGTCCCCAGCCCCAGAGTTGGAAGAGCCTT | 77 |
| | AGGGCGGGAAGGAAGAGACAGCAAGGACCAGAATGGGGAGCATGAGATCCTGAT | |
| | GCGGAACCCGAC | |
| ST8SIA6 | TCACCTGAAGGTTGGGGCGCGGAAGCTCAACTCCGTGCTGATTGGGCTCCAAGTTT | 78 |
| | TCTGCGCCCTCGCCTCGTCCCGAGTGCCCGCGAATCCCCCGGACGCCCACGCAGACC | |
| | ACCCAGCCACACCACAACTCTGCCTGCGGAGAGAGGAGAGGAGAAAAAGGGGCC | |
| ATHL1_NLRP6 | TCGGTCGGGACCTGTCGCGCACGTCCAAGACCACCACGTCAGTGTACCTGCTTTTCA | 79 |
| | TCACCAGCGTTCTGAGCTCGGCTCCGGTAGCCGACGGGCCCCG | |
| ATHL1_NLRP6 | ACGGCGGGGTGCCCAGGACCGCGGCTGGCGGCGTTGGGACACTCCTGCGTGGGG | 80 |
| | ACGCCCAGCCGCACAGCCACTTGGTGCTCACCACGCGCTTCCTCTTCGGACTGCTGA | |
| | GCGCGGAGCGGATGCGCGACATCGAGCGCCACTTCGGCTGCATGGTTTCAGAGCG | |
| | TGTGAAGCAGGAGGCCCTGCGGTGGGTGCAGGGACAGGGACAGGGCTGCCCCG | |
| DRD4 | GCGTCTGGCGGAACGGGCCTGGGAGGGAGGTTTTGCCAGATACCAGGTGGACTAG | 81 |
| | GGTGAGCGCCCGAGGGCCGGGACGCACGCACGGGCCGGGTAGGATGGCGCTGGC | |
| | GTCGATGCCCGCGCGCTTCAGGGCCTGGTCTGGCCGCCCCTCCATCCTTGTCGGTTT | |
| | CTCGGGTCGCGGACCCCGCGCGGCGCCGGGCGATGCTGGCCTGCCCGTGGCCACC | |
| | ACCTCGCTTCATTCCCGTCTCTTTGGGCCGCCGCATTCGTCCACGTGCCCGTCTCTCC | |
| | CTGCGCAAAATTCCAAGATGAGCAAATACTGGGCTCACGGTGGAGCGCCGCGGGG | |
| | GCCCCCCTGAGCCGGGGCGGGTCG | |
| TOLLIP | GCGGAGGACAGGCGTTATGCAAAGATTGGCAATCCTTTGACGAGCCCAGGTAGTA | 82 |
| | CAGCACGTCTCCCCCGTGATGTTTTTTGGCTTTTATCTTACATATAAACAAGCGTACC | |
| | CAGGTGGACGCCTTCCTCCTCG | |
| TOLLIP | ACGAATCCTCTTTTGGGGTCTGGATCAGGACCCTTTTCCG | 83 |
| KRTAP5-6_KRTAP5-5 | GCGCCCGTGGCTTCCTGCATCTGCCGACACCACCCGAGGCTGCCAGGCCACAACAT | 84 |
| | GAAGTCAGCTGTGCCAGGAAATCCCAAGCCTCGCCCACACCTGGCCCCG | |
| PAX6_ELP4 | TCGGCGCTTTTCGTCACTTCCTAACCCAGTCTCACAGAGGGTGACTTCCAAACCTGG | 85 |
| | CTAGCGGGGAAAACCGCTGCCCGGGGGACAGAGGGGCTGACAGGAACTGCGGGT | |
| | TGGCTCAGCCGAATGCGGCCGGGGAGAATTTAAGAATTCTCAGCCCGCGCGGCCC | |
| | GATGCCTCTGATTCCTCACGAGAGGAAAGGGAATGAAAAATGAAGCAACAAATGA | |
| | CACCACCCAGGCTGGCAGCCCTCGTTCCCGGCCAGACCCCGCTCCTCAGGCCCGGCT | |
| | CTGGCGCCGGGTGGCGTCCAGCCCCTGCACGCGCGGCGCGGCCCGCGGGAAAGTT | |
| | TGTGCAGCGAGAGTGACTGTCCTTCCGCCTCGCGCGCGCTGCCCCCTTCTGCCCCGG | |
| | AGGGGCGTTGGGTTCCCTTCGGTTTTCCTTTCCAATTCTAAAATAAATAAATAAACTC | |
| | CG | |
| GLYATL2_GLYATL1 | CCGCTGGATCCCGCCTGGATGCACGTCCCGCCACCGCCGCCGACCCATCAGCGGCA | 86 |
| | GAAGGGCAGCAATGGCCACACACCGAAGCACCTTGGCGGGCTATTCCCCTTGCAGC | |
| | TCTCCTCAGCGCGCTGCTCCCACTCGCAATCAAAAGGCGGAAAAAGCGCGAAACCG | |
| | CCAGGCATCTCCCATACCCACCCGGCTGCCG | |
| MYRF_TMEM258 | GCGGCTGCCCAACGGGCTGAGATTATCGCTGGTCAAATACTCCCTGGCGCTTGGCT | 87 |
| | ATTGTTTCCCCACGGGCGGGTGGGGAGCCTGGCCCTGCCTCTGAGCAAGTATCCCC | |
| | GCGGTGATGCCACCCGCCTGCCCGCCTGCGCCATCATGGACGCACCCTTCGGCGGT | |
| | AAGTGGGTGGCTGGGGAAGGCCGTGGGTGCAGCCTGGGTGCAGGCTTCCCAGGC | |
| | CGGGCCCACCTCACCTTAGAGGGTGCTCAGGGGTGCCCTGGCCCCCAGGTGGCCAA | |
| | GAGCAGAACCACCGCGGGAGCAGGCTCCCCG | |
| SCGB1A1_AHNAK | CCGGCCTCTGCCACAGCTGGGTGGGTGCCCAGCCAAGGAAGCTTGTGCCCCATCAT | 88 |
| | TCAGGGCATTGTTCTCCCTTAGAAGAGGATCTCGAAAGCAGAAGGAAATTAGAAAC | |
| | AACCGCACAATGAATACCAGATTCTGCTTTCTCTCAGCTCTGTCTGCCAGGAGATTA | |
| | GGCAGGGTTGGCTGACAGCGTGCCCCGCCCGGCAGCTGCTCGCCCTCCAGGATGTC | |
| | CGCGCCGTGGGGAAGCGGGGGTCCCGCTGGCCTTCTAGCTCTCTATTTATCTCCAA | |
| | AGTGTCCGGTTTTCTTTCTCCTGCTAGATGCG | |
| RCOR2 | GCGGAAGGGGCCAAGGAAGCTGGGCAGCGCGGCCGAGAACCCGGGGCCCTCACC | 89 |
| | TACCCGAGCTACCTCCGAGCTTGGCGCGAGCCGGAGGGCTCCCGGGAATGCCCTCC | |
| | CCGCCATTTTCGCCGATGAGCTCGGGCTCACCCTTCCACTGGAAGCGACAGCGCCTT | |
| | CTTTTCGAGGGCTGCAGGCCAGGACGCAGGCCGCCTGGAAGCAAGTGTGATCAGG | |
| | GCACATTTATTTCCTACG | |
| WNT11_PRKRIR | TCGGGAATATTTGTGGGCTGCCGGCGGGGCAGGCGGGGTGGGGGAGGCTGCCCG | 90 |
| | GCGGGCGGGAAGCCCCGCGCACTCGGGTCCCCTGCGGTCCCCGGCGGGGGTCGGC | |
| | GCGTGCGGAAAGCGGCCCGAGCCCCCAACCTCGGCCCGTCCGCAACCGAAGAGGA | |
| | GGCGACCGCAGCCTGGAAAAGAAGAGCCCCCAGCTGTTTCCTTCCACCCGGGCGG | |
| | GCGGGACGGAGAAGGGAGGGAGCCTGGGAGAGACGCAGGTGTGGCGCTCGCCTG | |
| | TGCTGGCGGGGTGGCAGCCGGGGCGTGGCACCCTCGGAGTCTCG | |
| CAPN5_B3GNT6 | GCGTGAGTTTCTTAGCACTGCAGCAGTGGTTCCTCCAGGCGCCAAGGTCCCCGCGG | 91 |
| | GAGGAGAGGTCCCCGCAGGAGGAGACGCCAGAGGGTCCCACCGACGCTCCCGCG | |
| | GCTGACGAGCCGCCCTCGGAGCTCGTCCCCGGGCCCCCGTGCGTGGCGAACGCCTC | |
| | GGCGAACGCCACGGCCGACTTCGAGCAGCTGCCCGCGCGCATCCAGGACTTCCTGC | |
| | GGTACCGCCACTGCCGCCACTTCCCGCTGCTTTGGGACGCACCGGCCAAGTGCGCC | |
| | GGCGGCCGAGGCG | |
| AMOTL1_CWC15 | ACGTGCAGCCAGGCAGGCATCTCTGGTGTCTGTGCCCGTATGCCCCAGGACCTGGC | 92 |
| | ATGTCTAAACCAGGCCTGGGAGCCGGAGGACTTGTTTGAAGGAAGAGCTGCTGTG | |
| | TTCCCTGCACTGATATTCCTCCTCATTGTTGTCATTGGTGTCCACG | |
| PKNOX2_FEZ1 | TCGCGGGGCTGGGAGTGGATCTGAGGTCCCGACCCAGGCGGCTCGGAGTGCTCCA | 93 |
| | GGAGCCACCTGGGTCTGCGGGCGCAGCGCGGCGGGGCGGGAGCGGTGGCCCGCA | |
| | GGGGCCGCGGCCTGCGATGAAGGCCGGGGGGCAGCGCTAGCAGCGAGGTGCCAC | |
| | AGTGGGCCGAGGAGTCTGGGCTGTGGCCCAGGGTAGGACCGGCTCAAACTCCAGT | |
| | GCCCTGATTGGAGCCGCTTCCTGTGCTTACCCGCGCCG | |
| MPPED2Ã⁻Â¿Â1/2 | GGCCTCGGGCCGCCGCGGGAGCCCGGGGATCGGGCCAACACAATGCACCCAGGCC | 94 |
| | TAGGCCGGGGCGGCTCGAACACATCACCCCGGGACTTTCTAGTAAACAGCTCGCTG | |
| | AGCCCTCGTCC | |
| OPCML | CGCTCCGAGGCGGCACCGGGAGAAAGTGGCGGTCAGGGATGGAGCTGCTGCCAT | 95 |
| | GACAACCCCGGCGGTCGG | |
| ANO2_VWF | CCGCACATACGTGACACAGCCCCGAAGCACCCTAAGGGACACCACCCAGGACAGAC | 96 |
| | CGTTCATCCCCGGCAGGGCAGGACGGGGCAGGGGGCCGACTTACTGCACGCGCTG | |
| | TGGTCGGTCCAGCCGTACAGCACCATTCCCTCCTGGGCACAGGTCCGGGCGTACTC | |
| | CAGGAGGGCAGGGCAGGCGCACTCCAGCCCCCCAGCACACTCACACAAAGTCTTCT | |
| | CACACAGGGCCACAAAAGGCTCGGGGTCCACCAGAGGGTGGCAGCGGGCAAACA | |
| | CCG | |
| IFF01 | TCGGAACCCACACCAACTCGCGGCCCGTTGTGAGTGGTATGACACAGAGAGACCTG | 97 |
| | TCCCCCTTTCCCAATCCCTACCTCCGCTTGTACTCGTCCCGCTCCCGCTTCACTTTGGC | |
| | CAGCACGTTGTAGAGAGCGCGGATCTCGGGCGTGATGGTGTCGATCTGGACGCCC | |
| | ACCCCATCCGGGTGCACCCACG | |
| IFF01 | ACGAAGCCGGTCTGCACTGCCTGGTCGCGACGACCCAGGCCCCGCCGGCCCTGCTT | 98 |
| | ACCCTCCTCCAGCGCTTGCTGCAGTTGCTTCTCCAACAGCCGGTTCCGGCG | |
| IFF01 | CCGGCCGGCGAGAGAGGCGCCGGGGGCAAGTCTCCTCCCCCGGCGAAGTGGTCGC | 99 |
| | CTCCCAGTGAGTCCCCCAGTGGCCCGGCCAGGCCCTGCTGCTCCTGCTGCAGGAGG | |
| | AAGAGGTTGGGGCCGAATAACGGATTCATGGCTGCGCCTTCTGCTGGGAGATGCA | |
| | GACCGGTGCAGGAGCAGGGATGGAAGGCGAGCCAGAAGAGCCAATGCGGCGCCG | |
| | GCGGGACAGAGCCGACCAATCAGGCGGCTCGGCAGCGGGGCAGAGGTCAGGGGG | |
| | CGGGCCGAGGGGAAGCCAATGACAGGCTCCAATTGGAGGCCGGACCCTGGACCTT | |
| | TCCGGGTCTGAGGCCGAGCCCTGTGATGAGGGGAGCCACCGCCTGGACTCCAGCC | |
| | GGGGTGGCGTAAAGCCCAGGACCTCCAGTACCCCATGGGTTCTGGTGGCAAGCCC | |
| | ATCTCCCCTACACGACTTTTTTTTTTTTTGAGACCG | |
| PHB2_PTPN6 | CCGGTGACAGGTAAAGGCCACCAGGGGAGAGGTCCTGGGCTGAGCTTGGGACTGC | 100 |
| | AGAGGGGGGATGAGGGTGGGTAAATCGGTGTGTGTCGCGGGTCGGGAAAGGCTG | |
| | CCGGGGGTAGGGGAAGGTGGCTCAGAGGCGGCGGGCCGACGGTCGAGGGGCTTC | |
| | GGAGGGCCTGCTTGGACTGCAACCTGGGCCTCG | |
| BCAT1 | GCGAGCTACCGAGACCCGGGTTCCAATCCTCCCCCCTTCCGCAAACGCCCGGGTTCG | 101 |
| | AGGTACCTGGCGGGCAAGGGCCGCAGCGGAGCGAAGCGGGCTGGCCATGGGGAG | |
| | GCTGCGGGGACGCGGGGCTGCAGAGAGCGGCAGTGGCACGGAGCGCGCGGCTGG | |
| | AAGCGAAAGCAGGCGGTGTGGCCAAGCCCCGGCGCACGGCCCATAGGGCGCTGG | |
| | GTACCACGACCTGGGGCCGCGCGCCAGGGCCAGGCGCAGGGTACGACGCAACCCC | |
| | TCCAGCATCCCTTGGGGAGGAGCCTCCAACCGTCTCGTCCCAGTCTGTCTGCAGTCG | |
| | CTAAAACCGAAGCGGTTGTCCCTGTCACCGGGGTCGCTTGCGGAGGCCCGAGAATG | |
| | CGCGCCACGAACGAGCGCCTTTCCAAGCGCAGATATTTCGCGAGCATCCTTGTTTAT | |
| | TAAACAACCTCTAGGTGAATGGCCGGGAAGCGCCCCTCGGTCAAGGCTAAGGAAA | |
| | CCTCGGAGAAACTACATTAGGGCAGCTTTTCCACCGACTCCAAATCCAACTGACAAA | |
| | AAGCAGTTTCTGCCCTCG | |
| SYT10 | CCGCCCTGGCTGCCCCTGTCCCGAGGGAAGATGCCCGAGCACTTCTCCCACTCCACC | 102 |
| | TGGCCGGCGAAGCACAGCTCGGTGACGATGTGCAGAGCCTTCTGGCACAGACTGTT | |
| | CACTCCGTCCTCCTTGTGGAAACTCATCGTTTGGCTTTTCTTTCGTTTTCTCTTTTTTTC | |
| | CCAGTTAGCCGTCTTTTCCTCTTCCCGTACCTCTAACCCCTCTGGCG | |
| SYT10 | CCGTAAAAAAGCCAAAGCAAGCCCTCGACTCGCAAGCACGCCCCCCTCCTCTCCCCA | 103 |
| | GCGCACTGGTGTTTCTGGCGGGTGCCTGGCGGCGACGCGTCCAATCGCAGCCCGG | |
| | CGCGGGCGCTAGGTGACAGGCGGCGGAGCGCGCAGACCCGGCTCCCCGCGTCCTC | |
| | TGAAGAAGGGACTCG | |
| HOXC4_HOXC5 | CCGCCGGGAGGACTCGGAAATACACAAAAGGAGCCGAAAGATTTAAACAGTCGGA | 104 |
| | GGCAGAGGCGTCCCGAGGCGGCCAAAGCGGAAATCAATCACGTAATTAAAACAGG | |
| | GAGGGGACGAAGCCCAAGGCTGGGGGTCCCGGGTTCGGAGGAGGCGGCCAAGGT | |
| | GCAGGCCGAGGCTGGCGAGCGGCTTAGGGACGTGGCTCGCCCGCCAGGACCAGA | |
| | GCG | |
| SLC26A10 | TCGGGCTGTGGAGGCTGCGGGCTCGCGCTTGTTCCGGGACAGGGGCGTGGCGCCT | 105 |
| | GCTGCTGGCTCGGCTGCCCGCGCTGCACTGGCTGCCCCATTACCGCTGGCGGGCCT | |
| | GGCTGCTCGGAGATGCGGTGGCCGGAGTGACCGTGGGCATCGTGCACGTGCCCCA | |
| | GGGTGAGAGGCCCTAACAGCAGCCTGTCGGGAGCACAAGCTCTAGAGGGCTTCCG | |
| | GGAGGAGGCTTAGGGAGCTGGGAATCCG | |
| AVPR1A | ACGGCGATCTCCAGTTTGGCCAGCTCCTCGTTGCGCACGTCCCTCGGTGGGCCGTTG | 106 |
| | CCCTCCCCGAGGGCTTCGGCCTCCCGGCTTGTGTTGCCAGCGCCGGTGGCCAGAGG | |
| | CCACCATGGGCTGGAGTTGCCCGAGGGCCCCGCGTCGGGACCGGCGGAGAGACGC | |
| | ATGCTGTCCATGCAGCTCCTACTCGGCCCTCTTCGGAGCTCCAGCCCTCGCGGGCCG | |
| | CTCCCTCCCCGTCTCGGAGGACTTGGGCTCCTCGTCCGAAGCGCAGGGTCTTTGGC | |
| | GCGCTCGCAGCTTGCCGGGCTCTGCGATCCCTCCAGTGGGCGTCTCCCGGAGCAGC | |
| | GTCCCGCCTGCCCACTGAGCAGCTCTCAGCAGGGTGAGCTGGCCCCTCTCCCTGCTC | |
| | TGCCTTTTTTCAACTTCGGCGAGGTCGGGAAGGTGAGCTCCG | |
| HMGA2_ | GCGGCGAGGTCTTGCGGGCTGGCCTTTCTGCTGCTGGTAGGAGGATCATGTGCTGC | 107 |
| ENSG00000228144 | TATTTCGGAGGCTCCTGCCAGTTGGCCCCTGCCCACCTTTTCTGTTCATACTGAAGCA | |
| | GCCAGGAACTGAGAGAAAGAGGAAGCCTCGGCTGTGCTCCGGGCTGCGCTGCCAG | |
| | GGTTGCG | |
| LRRC10_BEST3 | ACGTCGTTCCTCATGTTTATGAATAAAACATGGATGACTGAGATGATTAACTGGCTG | 108 |
| | AATGTCCTGGGACGGCGTTCGATTACAACCTTGTGCTGTTTTTCTAAAGCCTCAGCA | |
| | GCGCCCTTGGCTACCAGATAGCCTTCTGACCCACCCTCCACTGTGTGAGGGTCAGAT | |
| | TCTATTACATCG | |
| LIN7A_MYF5 | TCGTTAAGGAATGCATGCCGGTAGTTGCTGAGATGTACAAATAAGCACCAAAAATT | 109 |
| | AACCACG | |
| NT5DC3_STAB2 | ACGAAACAACAGACTGAATAGTACAGGAAATGTCACG | 110 |
| NT5DC3_STAB2 | ACGATATCATTTATGTTTTGATATGTAACGTTAACAAAAAGATCACTTCAACCTCTTT | 111 |
| | CCTCCCG | |
| LHX5_SDSL | TCGGCCGGGGACTGCGCCTGCGAAGGCGGGCCGTGCGCGAAGAAGTCGTAGTTGC | 112 |
| | TTCCCGGCGCGTAGTAGTCGCCTTGGTAGTCTGCGGAGGGGGAGCGGGAAGGAGA | |
| | CAGGGCGCGGTGAGAGAAGGCGAAGTAGGCGGGGGACCCG | |
| LHX5_SDSL | GCGGTTAGAGACACGCGTGGAAACCCCCGGGGGCG | 113 |
| LHX5_SDSL | GCGGAGGCTGACAGGCCCGGGGAGAGGAACCGGGCAGGGACAAACCAGCGGAC | 114 |
| | AGAGCAGAGCGCGAAATGGTTGAGACCGGGAAGCGACCTGGCCGGGGGAAACTG | |
| | GATCCGGGCCGCGGCAGGAGCGACTGGTGGGTTGGGCCGGGCGGGGCGGCCTTG | |
| | GCGCCCTAAACTCGGTCCCTGCGCCCTACCAACCCAGTCCAAGTCCTTCGCCTCGCC | |
| | AAGTACG | |
| LHX5_RBM19 | ACGCTTTTTCTGGCGAAACGGAGAAAAAACGCCGCGGAAACGGTGCGCAGGGTTG | 115 |
| | GGGAGTATAGGTTCTGATTGCAACATAATTCCGCAAGCTTTTTTATTTTTTATTTTTC | |
| | CCGGGACGCGGTTGCGTCGGAAGAAACGCTTTCTAATCTTTCTAGCTCCCTGGATTT | |
| | GAAGTTGCGGGTCTTGGGGCGAGGCTTAGCTGGTCTGGGGGTCCTTGCGTGTCCAC | |
| | AGCCCCGGATACGCACCCGCGAAACGTTCGACATCGCCGCTTTTTTGTTTTGTTTTG | |
| | CTTTGTTTTTTTAGTCG | |
| RBM19_TBX5 | CCGTTTCACCCCATGTGACACCTTATTTAAAAATTACCAGGATCTACTGAGGGGCCG | 116 |
| | ACTTGAGCGCCCAGTGCGTCCTGGGTTTTGGGCGCAGAGCGCAAGGTGAGGCTCCT | |
| | CCCTCTGCCTGGGCCCAGGTTGTAGCCTGGCGAACCCGAGGCTCCTGGTGCCCTCC | |
| | GGGCAGAGCTCTGTGCGCTCCCAGCGGCCGGTGATGGCGCGCCAGCCAGCCAGGC | |
| | CCCGACCGCAAGACAAATGGTGCGGCGCGCGGGTCTAGTCGGCGGCGCGGAGGA | |
| | GGCAGGAGGAGGCAGGAGGAGGCGGGAGGAGGCGAAGGCTACGGAAGATCAGA | |
| | AGAGGGGTCAAGCCATCGCTCATGCCGGCCTGAATCGGCCGCTGACCTGGCCCTTA | |
| | TTAAGATGCTGGGGGCCGATTCTACACATAGTGCAGAGGGAAAGGAATTATCTAG | |
| | GCCATTGTTAGCTGACCCCAAACGGCCGGATAATTGAGATTTCTCGAACAATTTAAA | |
| | TAGATTTCAAAAATCCTTTGGCCGTAAAGATAACCG | |
| TBX5_TBX3 | GCGCGCGCGCACCACGGCGCGAACTGCTCCATCAAGCATCCACTGGCCTCCAGCCG | 117 |
| | CGTTTCCGGTTGTAGCACTGGGCGCCCCCAGAGTGGACCCGATAAGCTATCGGCGC | |
| | GGCCCAGGAGGGGCGGTCAGCGGCGAGTCAGGGCACCTCGGACCGGCTCCCGGCT | |
| | CCCGGTCCGGCTGCCTGCCAGCGGCCGCTCAGGACAGAAGCGAGATGCCTGCCTA | |
| | GGCGTTTCTGGTTACAATCACCTCACACACCGGCCTGCATTCCG | |
| MLXIP_BCL7A | ACGTGTGCGCACACACATGATCTGGTGACTTGGTTTCTGCTCCATTTTCCCCTGCAG | 118 |
| | AAAAACAAGAATAAGAAAAAAGGCAAGGACGAGAAGTGTGGCTCAGAGGTGACC | |
| | ACTCCGGAGAACAGTTCCTCCCCAGGGATGATGGACATGCATGGTGAGTGCCCATG | |
| | GCCTGCCAGCCTCTCCTGCCCAGCCCGGGGCCTTGGCCAAGCACTCGGTCATGTTTT | |
| | TGTTTCTCCAGCAGGTTTGTTCACATTCCAGGCAAGGGGTAGGAGGGCTGGGCAGG | |
| | GCCCG | |
| NCOR2_ZNF664 | CCGATGTGCAGCTTCAGCCTTCTTCTGCAGGGTGATGGCGAAGAGGAGGAATTTTT | 119 |
| | TTAAAAAACAAAAAAACACAGATTATAAATAGAGGCTTCCCGGAGCAGCGGGCACC | |
| | TGCCCAGCCCAGTCCAGCATGCTGATCCTCAGCACGGGGGAGGGAGGCCCGGGGC | |
| | CCCCTGCAGGCCCTCCCCACGCTGGAAAAAAACACAGAGGAGCCTCAATACCCCCA | |
| | CAGCGGCCCCAGCAAGCCAGCCAAGTTTCGATTTTAGCAAATGCGCCGGGTCCACT | |
| | GAAGCCTGCTCCCCGGCAGGCGCGCAGGCCTCGCTCCCCCAGGGCCCAGCGACGT | |
| | GGGCACCGCTCCCCACCAGCCCAGCCG | |
| MMP17_SFSWAP | GCGAGGCCTTGAGGAGCTTACCAGAATAGTGAGGGCCCACGAGGGCCAAAGACCC | 120 |
| | ACAAGTGGTAAAGGACAGGTGGCCCCACTCAGGAAGACACTTTCTCAGGCAGAAC | |
| | CGGAATGACAATGGGAGGCCAGTTGTGGAGAGCCTGGGACGCCAGAATAAGTGA | |
| | GCACGAGAGACCGACAGGATGAGAGCCGCATTTCCG | |
| CHFR_ZNF605 | GCGAGAGCCACCGCGCCCGGCCTATAAAAACATTTTTAAAAAAGGACAATGACTCT | 121 |
| | AGAGATTCCCCGGCAGAGTTCCTCTGGGAAGCTTTTCCTCACCGAAGACGCGGCCT | |
| | CAAGTCATCCCCAAGCCGGGGCTCCTGGGTGGCTTCTCAGGAAGCCAAGCTCCCTC | |
| | ACCCTGTGGCGACGCCGCGGGCGGAATGCGCATGCGCGCCACGAGCCACAATCGT | |
| | AGGGTTGGGCGCGCCCTGCCGGCCACCAGGGGCAGCGCAGGAGCTGAGCGCACCC | |
| | CATCAGCGAAAGAAGCGCGCCTCCCCGCTCTTTTCTGAACCGTATCTCCTAAACTAT | |
| | AATTTTGGAGATCAAAAGTGCG | |
| BCAT1 | CAGTGCCCGAGGCGGCGGCGAGTACACGTGGCGGGCTGGATTGCAGACCGGCCCT | 122 |
| | CTCGCGGCGGAGACTCGCGACCTAGCGGATTGCATCAGCAGGAAGAC | |
| WIF1 | CTGGCGAGGCCAGCAGTCAGCGGGGCAAATAGAGCGAGAACAGAAGAGCGGGAA | 123 |
| | GGGCTGGCGCGAGCGAGGTGCGAGCGAGGAGTGGGGCCCGCGAGGCCTGGGCG | |
| | GCCGCCACTTGGGGGCGCTGTGGGGCCCCCCCGGGGGCGGGGCCGCGAGGGACC | |
| | CCCGAGGCTGCATTCACAGTGCGGTGCGCCCAGTGGAGCGCC | |
| XPO4_LATS2 | ACGGTGGAGAGACGGGGAGGGCTCCGGAAAACTGCGTTCTCACAAGACCAAAGG | 124 |
| | GAGGGGAGGGAGGGGGAGATGTGGCTGCAAGTGCAGTTGGAGAGGGTGTGAAG | |
| | AGATCGGGAGTCCTCTGCGAGGCTCTGGAGCACCCGGCGCCTAAGAGGCTAGTGC | |
| | GCCCCGTGCCGCTGCGGTAGGACCTGGCGGTCCG | |
| RNF17_ATP12A | GCGCCCGCAGGGCCCGCCCACCGCTTTGCTTACGCCGCTGCCCGTGGGCCACCCCG | 125 |
| | GCGCGCAGGGTCCCCAGCCCGCGCCTCCGCCACAGCCGGCTTTCCCGCGCAGCCAC | |
| | GGACTGCACTGCCGCCACGCCGGCAAGGGCTCCAGCTGGACGGAGGGGGCCTTCC | |
| | TCGCTCCGGGATCCCTGTCCCACTGTGTGGCTTCCCGAGGCCTCCCCTTCCTGCG | |
| RNF17_ATP12A | CCGGCTGAGATTAGAGAGGCCTGGCGAGGTGTGGGGGTGCGCAGGGAGAATGGG | 126 |
| | CTGTGGTCGCCATGGTGCGTGTTGGTCTTGTGGAGATGGATGCTCCTCCGGGTCAA | |
| | TCTCTGCCTTCTCGGGGTCGCCCTCAGTGTCGCTGCTGAAAAGGCCTCCGTCCTCCT | |
| | GGTCCTTGCTGTGCGCTCCCCACGTCACCGCGTTCTCCTTGAGGGGCCGGCGGGCG | |
| | TTGGCGAAGGTGGTGGGGACTGTCGTGAGGATCATCATGGGCAGGGAAGGGCGC | |
| | GCG | |
| RNASEH2B_DLEU1 | TCGGCGCCCCCCTCAGCGCCTCGCACTACCTCCTCCTCTGGGGAGTTCGCCCGCGCC | 127 |
| | GCGGTCCGCCGACTCCTGGTCCCCACGCCCCCGCCCCGCTCCTCGCGCCCGGGCCCC | |
| | GGCCGGGCCCGCGGCGGGCCTGAGCGACGGGCTGGAGCGGTGGACACGTGGTCT | |
| | GGGTCCCGCGGGTTCCCGGGGGCGACTGGACCG | |
| LECT1 | TCGGGCGGGAAACAGCTCGCCCGGGCTCCTACGGGTGCCCCTTTCGCCGCGCTCCC | 128 |
| | TCCCGAGGGTCCTTTGCAGTCGGGCGTGGAAGTGGGATGAGCAAACCCCGCAGCA | |
| | CAGGGCCTTCGCCCCAGGACCTGCACCCTCTACCGGCCACGGGACGTCCCTCCGCA | |
| | CCCGCCTGTGGATGCCGTGACCCCTGCACACTCATACGCGTGGGGCG | |
| ZIC5_CLYBL | GCGGAAATCGGGGCCGGGGCAAGGACGCAGGGGCGTGTCGCCCACGTTTCTGGCC | 129 |
| | CGGCTAGCCGCAACTCCTTGGATGTAAACGAGATTTGGCCGGCGCTGCGGCGTGTG | |
| | GGGAAAGATGATTACACTCGAAAGGAATCACGACTCCTTGCGGAGCCATTACTCGT | |
| | GCCGCTCCGCACGCGCAGGTTCTGGCCCGGCTTTCAGCAACTCCCCGCTCCTCGCTA | |
| | ACCACTCGCTCGTAATTTGTGGGCCGCAGTGGAGCTGCGCCCG | |
| PCCA_ZIC2 | CCGCGAGGTCCCGGGTTTCGCCATCCTGAGACCCCCGCGCGGATGGCCCAGGAGG | 130 |
| | GGCGCGGCGGCCCTGAGTCAAGGTGGGCGGGGGCAGGTGCTTCCCTCCACCGCGT | |
| | TGTCCTATGCCGGCGCGGTCCCCACCGCCCGACCTAGCCCGGCGCCGGCCGAGCAC | |
| | GGCGGCCGCGCTTCGCACTCCTTCCTCCCACCGGGTCCGCAGGCCCGGCTTCACGAT | |
| | TCCCGGGCCCTCGGGCATGTGAGGGACTTGAGTGAATGCAGCTCCCTCAACTCACT | |
| | CCCG | |
| MYO16_TNFSF13B | GCGCGCGGGGAGGGGAGAGGCGGGGCCGGCGGGGACTGTGTCGCCGCCGACGC | 131 |
| | CGCGGCTGCGGGTCGCAGAGGCGGGCAGAGAGAGCCGCCGCCGAGCGGGTGGCG | |
| | GAGCAGTCCCCAGCCTCCAGCCGGCCTGGCTGCGCGCAACCGCGCCGGCCCCGGG | |
| | CACAGGGGCAACTGCCGACCCCTCTCACCCG | |
| MYO16_TNFSF13B | ACGCGGCGGGGCAGCCTCTCCGAGTCTGGAGGTACGCGGGGCGCAGAGGCTGTTC | 132 |
| | TGCACCGCCGGGCTGGGGACGCCGGGAGGGTGCCCCGGGTCGGACTTGCGGCGCT | |
| | GGGTCCCCACCCAGAGTTCCCGCACGGTGAGGGTTGGACGCG | |
| RAB20_COL4A2 | ACGCTCCTGGTGATGCATTTGTTTCAATCACCAACAAGCAAACCCCAAGTGAGATCT | 133 |
| | TCCAACCACAAAGCACCTGCTCCCAACCACACCTGCCGGGGGCACGCTTTCGAAGA | |
| | GGAATGAGACTGAGACCTGTGCTCAGACG | |
| SOX1_TEX29 | GCGTCCGGGAGGGGATCACATTCCTGCGCAGTTGCGCTGCTGGCGGAAGTGACTT | 134 |
| | GTTTTCTAACGACCCTCGTGACAGCCAGAGAATGTCCGTTTCTCGGAGCGCAGCACA | |
| | GCCTGTCCCATCGAGAAGCCTCGGGTGAGGGGCCCGGTGGGCGCCCGGAGGCCGC | |
| | TGGAGGGCTGTGGGAGGGACGGTGGCTCCCCACTCCCGTGGCGAAGGGCAGGCA | |
| | AACCAGAAGCCTCTTTTGAGAGCCGTTTGGGATTGAGACGAGTAAGCCACAGCGAG | |
| | TGGTTAGAAGTAGGTTAGGAAGAAGGGGAGGTAAGAAAGCCGAGTAGGGTTCTG | |
| | GGCCGGAGCCGTTCACTGAGACAGGAACCCTGGGGGAGATGCGCTGTCTCCCTGG | |
| | CGTCTCGGTGCAAATGCCCAGAGAGCG | |
| SOX1 | CCGGGCCAGGGCGCAGATGATGGACTCAGAGCGCCCAGGGACCCTAGAGAGAGG | 135 |
| | AGCACTCCTCAAGAGCCCCCTGGCCATCACCCGAGCGCCCTGGAGCGCCATCACCC | |
| | GAACGCGCGCTCCAGGCCCTCGAACAAGGCCTCTGGCTGCCAGAGCGAGTGAGGG | |
| | GCGCAGAGGCGGCAGAGAGCGGAGAGCCCCGGTGTCTCCGCGAGGGCGGCGGCG | |
| | GCCAGCAGACGGCGATCGAGGCGCGCGCCACGGCACGGCCAGCGCAGACACGCC | |
| | GCGGGGTCTCGGGCCGGAGCCGTGCAGCCGGGCCCGCTGCCTCTTTGCCCCTCATG | |
| | GCTCCGCGCGGGAGGAAACCGGGCCTTCTCCGCCCGCCCTCCTCTCGCTGCGGTGT | |
| | CCCCAGCACCCCCG | |
| MCF2L_ATP11A | ACGTCTGCTCGCCGGTGTTGAGACTTTGGAGTGGGCTTCATCCATTCATCCTGATCG | 136 |
| | TTCCTCCATGAGACAGGGTCCCTTTGTTGCTGGCTGGAAGCGGCCGGGAAGCGTGG | |
| | GCTCGCTGTGGCATGGGCAATGCCACACGGCTCCAGGGAAGCGTTCAGCTTTCCAA | |
| | ACCAGTGTCTGGGCTCGTGGCCACTCCTGAAATTCAGTTGCCGTCTTTGAAGCTTCG | |
| CDX2 | GGTAACCGCCGTAGTCCGGGTACTGCGGGGGGCTGACGAAGTTCTGCGGCGCCAG | 137 |
| | GTTGAGGCCGCCAGAGTGGCGCACGGAGC | |
| SPG20 | GCCTCGCTCCCGCCACAGAGCCCGCAGCACGCCGCCGCCGCAGCCTAGGTCACGTG | 138 |
| | AGTACCCACGCGCGCGTCTTGCCAGCGGATTCATCACC | |
| RNASE12_OR6S1 | CCGGATTACACAGCATCAGTTCCTCTGAATTCTGCATTCGTAATTAAAATCCTGATTT | 139 |
| | CCAATTGGCATTTCTTTCGGTTAGGCAGGGAGGCCTTCTCGCTCGCGGTCTCCTACT | |
| | TTATCCGTTGTACTGACTCTCTGGACCCCAGTTTTTGCACTGCACCATTTGGGTTCCC | |
| | GCAATCAGGAAAGCTCAGTTCTCATCTAAAATACACG | |
| GCH1_SAMD4A | GCGGCTCTGCTCTCCACCCCAGTGGGGCTGAACTAACAAGTTCCCCTTTTGCTTTTCT | 140 |
| | CACCAGAACCTGTGGTTTGCCAACCCCGGGGGCAGCAATAGCATGCCAAGCCGCAC | |
| | CCACAGCTCAGTCCAGAGGACCCGCTCGCTGCCCGTGCACACTTCCCCACAGAACAT | |
| | GCTGATGTTCCAGCAGCCAGGTAGGGCCCGGCGCTTCATGTCCCCTTGACACAGAG | |
| | GGGAGGCCAAAATAGATGCCCTAGCAAACCCAGCCAGAAAGTGCTTAGCCTCGACT | |
| | GTCACCGTGCATTCTTTGGAGCTTATAGAAGCCTTTCCTTTTTTAAACTGTGCCTTGC | |
| | CAGCATGAATAGCGGCG | |
| TMEM260_PELI2 | ACGAAGCTTGTATCTAAAAGCCAGGTGAGTGGCAGATTCCGGGCCCACG | 141 |
| OTX2_TMEM260 | CCGAGGCCGACCCGACCCCTGCACTCCGCCAGGCCGCGAGGTTTCCCAGCGACCGG | 142 |
| | CGCCCCGGCCCGCGGCCGACCTGGAGGCCTGACTGCAGGGCTCGGGCGGGGCCCT | |
| | CTCTCGGCTCTGGCTGGCGGCCCACTCCCGCGGGCGTACAGGCCTCGCCACCGGGC | |
| | CTCGGCCTTGCCGCGGCCCACAGCGCCCTGGGACCGGCGCCCCCGAGGCCTGAGA | |
| | ACTACGCCCGGGGGGCGCGGGCTGAGGCTCAAGAGAGGTCCTAGGTGCGGGCCA | |
| | GGGATGGAGCCAGCCCAGAGAGAAAGGGGAAAACCCGGCAAGGCAAGAGCCTCA | |
| | GTCTCGCCCCTGCCTGGCCCGCCAGGCTGTGAGTGGGGCCCATTGGGCAGCGCCAA | |
| | CCTGGGGAGTCCGGCGTCTGCCCCAGCTGGGGGCCCTCGGGGCAGAGATGTGAGT | |
| | GCTGTTCCCAGGTAACTCCGACTGGGCACTGGGGAGTTAGAAAAGCCAGCTCTTTA | |
| | GCCAGAGCGCCTAGGGCGCGGCGGAGAGCGGGCCGCCCGGCACCACGTTCCTTCT | |
| | GGCAGTTCCGCCCCAGCCTCCCAGCGTCTTGCGCCTGTGGCGGCGGCAGTACG | |
| OTX2_TMEM260 | CCGCCCGGCCCCGAGCCACGACACCTCATTGTCCTGGAGCCTGGGAAGGGGGTGC | 143 |
| | GCGAGCGCGCGGGCGAGCCCTGCCTCTCCCCGCCAGAGAACAGCTGAGGGGCCGC | |
| | GGTCCCAGCGGGAGGATTCCGGTCCCTGGCCCGGCCGCGGCCTTGGGCGGAGCAG | |
| | GGGCCACTAGCTGCCACTTCTGCCCGCCCCAGGTGCGCGCGGAGGGCTACGTGGG | |
| | GCGGGCCGCGACCCGGCAAAGTCATGTTGAAAAAACACTCTTCACGTTCGCTCG | |
| RTN1_JKAMP | ACGCTGCTATTAGGACTCCCTTTGTTCCTGGCTCATTCTCCATGCAGCATCCGGAAG | 144 |
| | GATCAATTGAAAACCCAAGTCTGATCCTGCCACACTTCCCTTCCTTCCCCCTCCTCCA | |
| | GCCAAGCCG | |
| IRF2BPL_VASH1 | ACGAAATGCGTATCCTCCAAACGTTCGTGGCAAATGGTGCAGCAGAGGGGTCCGCT | 145 |
| | GTTGGCCATGGGGGAATCCGGAATGTTTTGGGGGTGCACTTGGTCCATGCCCGGGT | |
| | GGGCGCTAGGCGGCGGGGGCGCCACCTGTAAATTCAGGTCCCCGTTACGTGATGC | |
| | CAAGCGGCGCTGCCCCGGCACGGAGGCCGGCGAGACTGGGCTGCTGCTGTTTCGC | |
| | CGCGCCGACGCAGTGGTAGAGTGCACGGAACTGCCATCCTTGGGCGAGTGCGCTG | |
| | TGCCCAGAGTATCTGCCACCGACATGAGAGCGGCCATAGGGGACGGACCGTTCTG | |
| | GGGGGCTGACTCAGGTGGGGTGGTCCG | |
| LGMN_RIN3 | CCGGGGCTCGACGAATCAAGGCCACACAGGCAGTGGGAGCAAAGGCAAAGCCCG | 146 |
| | GCAGGTGTGGGGCTGGGTCCCTAGGGGTGGAGGACGGCGGGCGGGCGCCCTGCT | |
| | CGTGCTGCGAGTGCCCAGCCCCAGCCCGCAGGCGTCGCCTCGCCTTGCCCGCCCTG | |
| | CTCATGCCGGGCCTTCCCCACCCGACTGCGCCCAGCCTCCTTCACCCGGTCCCCTCCC | |
| | GCTTTACCAATCCCTGCCCCACGCAGCTCCTCAGAGCCCCAGGGCTCTTGCAGCCCT | |
| | AAGGGGCTGGACTGTGTTGCCCGCCCGCACTATGGGATGCCCG | |
| LGMN_RIN3 | TCGGCCCAGCTGGGAAGCCAGGCAGGGAGGGGGACGGGCCCCCCGCAGGCTCGC | 147 |
| | GGCAGAGACGGGAAAGGCGCAGGTGCCGGACTCGCAGACAGCTTGGCGCCCGCC | |
| | ACCCGCTATCCATCCAGGGAGGGGCCTGGGCCGGGAGAGGGCGCCTGAGGAGAC | |
| | AGGGCCCCGCCGTGACCACAGGCCCCTCGCGTCTCCGCAGGACTTCATCTGCGTGT | |
| | CGTACCTGGAGCCCGAGCAGCAGGCGCGGACGCTGGCGTCGCGGGCG | |
| ITPK1_CHGA | CCG | 148 |
| VRK1 | CCGAGCAGCAGCCACCTCAGGGCCAGGGAGCCCGAGCTGCGGGATCCGCCGCCCC | 149 |
| | GGGGCCGCAGCAGCTTCAGCTCCTTGGCGTCTGCGCCGGGGTCCTCGCGGCCGCCG | |
| | CGAACCGCTCCTTCAGTTTCGCTATGCGGAGCGGGCGCGGGACCCCAGCAGGTGA | |
| | GGGCCCAGGGCAGGTGCCTTCCCTCGCCCCGGCTCCCGCCCCAGCTCCTGGCCGGC | |
| | CCAGCGCGTCCTGCTCCCGCTCTCGCCGTGCTCTCGGCGCTGCATGTCCCCGGGGCG | |
| | CGGCGCAGCAGCTGGTGCCGCGGTGGGCATCTGTTCGGCCTCCTCTGTCCCCACGC | |
| | GTGACCTGATCGCTGCGACAGCGGAATCCCACGGTGCAGGCCCAGAGCTGCGCCG | |
| | AGAGCCGCGCGTCCAGCTCCTCCCGGGCCTGGGTTTAGGGTCCACAGCTCTTGCCA | |
| | AATTCCAGAGGCTGGAAGGGACGCGAAGTTCTTCGTGACCCCAGCTTCTCAGGCAG | |
| | CG | |
| WARS_BEGAIN | CCGGCACATCTTTTCCCACCAGTGTGCAGATCTGTGCCGCTCTTTTTGGGGGCTGTG | 150 |
| | TAGCGCTCAGTGTCTGACACACACCATCATTTATGCGTAAAAGTGGCTGCGTCTTCT | |
| | CACCCTCCACAGCGGCAGAATTATTACCTTTTGAAAATGTCTGCTAATTTAATGGTGT | |
| | CTTGTTTGAGAACCAAGTGAGTTCATTTACGTACAGCTCTTTTAGAACGGGCCGGCA | |
| | CTTCG | |
| PACS2_BTBD6 | GCGCTGCACCCGCTTCCTGCAGGAAACGCATTCAAGCGCCCAACACACATGCACGT | 151 |
| | CCACAAAACTGGCCTTCCACCCGGCCACGGCTCGAAGCATTTCCGAAGACTGAAAT | |
| | CACACAGAGGGTGCTCTCTACTGCAGAAGAATCACACCGGCAGTCAGGAAGAAAG | |
| | GCGCTGACTATACTCCTCTACTAGTAAGTCCACAGCAGGACAAGGAAAAAAGCACA | |
| | AGGGAAGCG | |
| TMEM121 | ACGGTGACCAGGGTTCCCTGGCCCCAGTAGTCAAAGTAGTCACATTGTGGGAGGCC | 152 |
| | CCATTAAGGGGTGCACAAAAACCTGACTCTCCGACTGTCCCGGGCCGGCCG | |
| PRIMA1 | CGGCTGCCCGGGGCACTGGGGTGCCCGAGCTCTCTACTACCCTCACGCTGGCCCGC | 153 |
| | GAGAGGCAGCGGCGGGAGGCGCCGGCAGGGAGCTCCCGCTGGGG | |
| CYFIP1_NIPA2 | CCGGCAGCCCTGCCAGCAGACTCCGCAGCCTGGAAGGCAGGAAGCAGCCTCCAGC | 154 |
| | CCCAGCAAGAAGGCAGGTCTTGGCCTTTGGCTGACCTCGGCCACGGTGCCCCAGGC | |
| | CAGCAGGGCAGTTTCCCCTGCCCGGCAGCTCCCCG | |
| NDNL2_APBA2 | CCGCAGGGTGGTCCTGCCAGCAACAGCAGCCTCCTCTTCCCCACCTCTCCAGCGCCT | 155 |
| | GCAGGCTCTGCCCACAGCCCACTTGCAGGAGGCCGCTTGAGCCCTGAGGTGGGGC | |
| | CTGGGCTGGGCTCCTGGACTCACAGCAGTGAACGCCCACAGGCTTGGCTGCGAGTT | |
| | GGGGCCGGCAGGGCGACCCCTTCTCTGAAGCGCCAGCCGCAGAGAGAGCCCCCTG | |
| | AACCCCACACCTCCCAGGAGGCAGCCG | |
| ITPKA_LTK | CCGGACCCAGGATCGTTTCTGGGGTAACCCTTGCCTAGGTCGGGGGGCGGATGCC | 156 |
| | GGGGCTTCCCAGGATGTGGAGTGTGGGGCAGTGAGAGGCCCCCCGCCCCGCCTCT | |
| | TCGGAAAAGCCTGAGCAGCAGCTCCCGGGGCGCGGAAGCTCTGACACCTGAGAGC | |
| | CGGTGCAGGCGAAAGGGCGCGAAGCGCGGGCGCGTCCCGCTTCCCTCTTCCGCCC | |
| | GCAGGGACTCGGCGAAGTGCCTGGGAGAGGGAGTGCGCTAGGAGGAGGTCCTGC | |
| | GGCCCAAGCCTGGGTGTAGAGACCGCCCCGGCTAAGGTCAAGCCTCGGGGACCTG | |
| | GGCGACCCCGCCGCCCTCCGAGCCGTCGGGAGCCGGTGCAAATCGCCGCTGAGGG | |
| | CCCTTCCAGCTCCAAGGCTGCGGCTTCCAGGCCTTCCCCACCCCCAGGCCCGCCGGG | |
| | GCCTCCCCGAAGTCAAACAGCCACAGCGGCG | |
| ITPKA_LTK | GCGGTAGGCCTGATAATCTGCAATTTTTAACAAGGGTGACCATCAGGTAATTCCGAT | 157 |
| | GTTCACAACAGTTCAAAAACCTCGACAGAGCATTTTCGTAACCTGCCCACGCGTTCT | |
| | TCAGTGGCAGAGCTGGAGCGCAAACCGGGGGCTTCAGATGCTAAGTCCAGGCTCTT | |
| | GACAGCTCACTGGAGACGCTGGAATCACCTTCACTGCGCCTGTATCAGCACCCGCC | |
| | ACACAGGCG | |
| ITPKA_LTK | CCGCCTGCAGCAGATCCGGGACACCCTGGAGGTATCCGAGTTCTTCAGGAGGCACG | 158 |
| | AGGTAAGCGGCGGCTGCCCGGGTGCCCGGGCCGCGAGGGCTAGGGCGGGAACCC | |
| | GGCAAGGGCGTCTCTGGGCAGGGCCGCGGCCTGACGGTGCGGGGCTCGCAGGTG | |
| | ATCGGCAGCTCGCTCCTCTTTGTGCACGATCACTGCCATCGCGCCGGCGTGTGGCTC | |
| | ATCGACTTCGGCAAGACCACGCCCCTCCCCG | |
| DUOX1_SHF | ACGTGCTTTCAGACCTGGTGAGCGTGGAAACTCCCGGCTGCCCCGCCGAGTTCCTC | 159 |
| | AACATTCGCATCCCGCCCGGAGACCCCATGTTCGACCCCGACCAGCGCGGGGACGT | |
| | GGTGCTGCCCTTCCAGAGAAGCCGCTGGGACCCCGAGACCGGACGGAGTCCCAGC | |
| | AATCCCCG | |
| ONECUT1 | CCGGGTGCTGGTGGGGCCGTGGAGGCTCGGGCCGTCCCTGCGGTTACTCCCAAGG | 160 |
| | CCCTCCTGCTAAAGCACCCGGAGGCGGTTGCTTTCCAGAAGTACTGACGCAGACAG | |
| | GGTGGACGCCGGCGCGCGGGTCTCCGCTTGGCCCCTAGGGACGCCCTTTTCCCGGC | |
| | GTCCCCGAGAGACGCCTCCAGATTTGAAAATCAATTCAGCTTCGGGAGTAATTTCGC | |
| | CCTTCCCACAGTCACG | |
| PIAS1_SKOR1 | CCGGTAGCCCGAGGGAAAAACGAGGCGAGAGGGGAGAAGGCGACCCCGCGCTGC | 161 |
| | TACCCGCGGAAGATTTATGGCGCCTCCCGGGTTCCAAGGACAGGCTGCGTTCGTCG | |
| | CTGCTGCCACCGCCGGTAGTCGCCGTGGCCGCTGCGCCCCCTGCCCAGGCGGCCCG | |
| | TCGCG | |
| ISL2_SCAPER | CCGTCCCTCTGGCTTGGAGCTGCGGGTCCCCGCCCTCGAGCCGGAGCGCCGCGCTG | 162 |
| | GACACCCGCGGGGTGGGGGCTCGGCTGGGCTGAGCCACGGAGACGCCAGGGTCC | |
| | CGCGGTGGCGGGGGCGCCGATCG | |
| SOCS1_CIITA | ACGGGGAGGGGAGGGCAGTAAGAGCCGCCACAGAAAACAGGAATTCATGGGGGG | 163 |
| | AGTGGGGTTGAGGATTAACGTTGAGTTTCAAGACATCCCTCGCTCCAGCCCACTCTG | |
| | TGAGCTGTCTGGGGCTCCGCCTACACACAGCTCCTCACCCTGAAGCTGCTGGGTTCC | |
| | CCTGCATCACACG | |
| SOCS1_CIITA | GCGGCTGCCGGGTGCGAGCGGGCTCAGGCCTGTGGCCCTGCCTGACGTTGGTCCC | 164 |
| | CATCAAGCCATGTGACGAGACCAGGCCACAAGAAAGAGGTTTCAACAAGCGTTATC | |
| | GTTTCCTGGAACTCCAACTCGGCGACTTCCCCGAAGACCGGCTGTGCCTGGCGGGC | |
| | GGGCTGCGCACAGCGGGGACAAGGCTGCCCCCTTCCTCCTCCGCTGCCTCCGCGGC | |
| | CG | |
| HS3ST2 | TCGGGCGCTGGGCGCGCTCCGAACCCGGCGCACGTAAGAGCCTGGGAGCGCCCGA | 165 |
| | GCCGCCCGGCTGCCCGGAGCCCCATCGCCTAGGACCGGGAGATGCTGGAAATGCA | |
| | ACCGCCTGTTCCCCGAGGAGCCGCTGCCCCCGGGACCCCCTGGCACTGTGCGCACC | |
| | CTGGTCAGCAGCCCCCGGAGAAGACGGCGCCCCCAACGCCCGACCCGCGTGGCCG | |
| | TGGCAGCGCCACGCGAGCCCTCTAGGCGACCGCAGGGCCACAGCAGCTCAGCCGC | |
| | CGGTGCCCCCTCGGAAACCATGACCCCCGGCGCGGGCCCATGGAGCCATGGCCTAT | |
| | AGGGTCCTGGGCCGCGCGGGGCCACCTCAGCCGCGGAGGGCGCGCAGGCTGCTCT | |
| | TCGCCTTCACGCTCTCGCTCTCCTGCACTTACCTGTGTTACAGCTTCCTGTGCTGCTG | |
| | CGACGACCTGGGTCGGAGCCGCCTCCTCGGCGCGCCTCGCTGCCTCCGCGGCCCCA | |
| | GCGCGGGCGGCCAGAAACTTCTCCAGAAGTCCCGCCCCTGTGATCCCTCCGGGCCG | |
| | ACGCCCAGCGAGCCCAGCGCTCCCAGCGCGCCCGCCG | |
| KDM8_NSMCE1 | ACGCACTCGCTACCGAACAAGCCTGGCCCTGTCACTCCCAACTCACCCCCACCCCAG | 166 |
| | GGCTTCCCACCACCCTTAGGTCCAAGAGCCAAGCCCCTAATACGCGTATCTCCCGGG | |
| | CTGCCCTCCGTCTGCTCGCCTCGCAATCTTTGTGCTCAGATGGCCCTGGCCTTAGCTT | |
| | CTTGAGTGCACCTGCTGGCCACAGGGCCACTGCCG | |
| SALL1 | ACGCAGGTTTTTGGGGGAACTCCCGCCGCCCGCCACCAAGGGCTATCTCCAGACGG | 167 |
| | GCGCCGGGTGCAGCGCCGTGACCGGGCGCCCTGGCGCCGGCTCGGGCGCGAAATT | |
| | CAGCGGTGGCAAGCGGAGGGTGGGCTTGGTAACCACCCGCGCGCGCCCGAGCCAA | |
| | GAGTCGCGTACTGTCTGCCCGCGGCAAAGTTCGTCTTTCTCCGCTTGGAGGGCTGTT | |
| | CCTACACCGGTATTAAGAAACCGACTTCGCTAGCGACTGCAAGTGCTTGCGATTTTG | |
| | ACTTTCCGTCCACAGTTGAGCGTCTTGCACTTAAATTCACTGCGCCCCGCATGCAAC | |
| | AGTGCCTCG | |
| GPR56_GPR114 | GCGTCTCTCAGTGGAGGCCCTGGCTGTTCTGGGGTTACCCCTTGCAGTGCACAGCA | 168 |
| | TGGCCGGGCATGCTGGCATGGTGGTCATCCTAGCACCGGGAAGCTGGCAGGTGTG | |
| | AGGTGTGTTCCCGGTGTCCAACGGACACTGCAGGACGCAGGGCAAGGGTGACGCC | |
| | GCGGAGCCTGAGCATGGACGGGAGGCAGGCGGCAGGACCTGAAGTCTCCTGCCTG | |
| | CTTTCCGCAGCGCCCTGAGCAGCTTCCTCCTGGGATCCCACGGAAACCGGTTTGGG | |
| | AGCAGGTTGGCCCAGGTCGTTTGACTTTTGACTGGGGAGGAGAAGGCAGCCTCCCT | |
| | TAGCG | |
| MTSS1L_VAC14 | GCGGCCGGGGAGCCAGCCCTGCAGATGTTACTAAGTGAAACCTGATGTGGTGACA | 169 |
| | TGAGAATCCACAGAACGTCTCACAAACAACCTGCCCCGGGATGTTTTGGATTGAGTT | |
| | TTGTGGTTATGACGTGAAGAAACCTCACATGTCAGGATAAAAATAACCCTGGCTTCA | |
| | GTACATAACGCGAGTTACAGTTCAACAGAACCAGATGTGAAAACGTCAGCCACCCA | |
| | GTTCAGGCCCAGCAGGGTCCCTGCTCCACTCCG | |
| FOXF1_IRF8 | ACGCTGAAGATCACCTTGTAAAGGTGGAGTTCCTCAGGCTTTACTCCGGGAGCCCTC | 170 |
| | CCTGGGGAGCAAGAGAAGGCAGGGTCAGTGCTGAGCCATCCCGGGTGTGTGGACC | |
| | TGCTACGCTAGGTCTGGTCTGGACGGTGCTGATGGGACCGGGGATGACAGAGCCA | |
| | GGAGGGGCCAGAATGAAAGTCGCAGAAAACCAGAAACAGGCTACAAACTTCTCCA | |
| | GTCTGCCCACCCTCCCCTTCCGTTTGTTTCATGAAAACCCATTTCCAATCAGAGGACC | |
| | ACAGGCCAGGGAACATGGTGAGCCCAGCCAAAGACACTTTCAGGACAGATGGTAT | |
| | AGAAACG | |
| FOXL1 | CCGCCTCGCCCATGCTGTATCTGTACGGTCCCGAGAGACCCGGCCTCCCTCTGGCCT | 171 |
| | TCGCCCCCGCGGCTGCTCTAGCTGCCTCGGGCCGGGCCGAGACCCCGCAGAAGCCT | |
| | CCCTACAGCTACATCGCGCTCATCGCCATGGCGATCCAGGACGCGCCCGAGCAGAG | |
| | GGTCACGCTCAACGGCATCTACCAGTTCATCATGGACCGCTTCCCCTTCTACCACGA | |
| | CAACCG | |
| FOXL1 | ACGGCCCCTCTCCGCCGGCGCCCCTCCACTGGCCGGGGACCGCGTCCCCGAACGAG | 172 |
| | GACGCTGGTGACGCTGCCCAGGGCGCAGCGGCCGTGGCGGTCGGCCAGGCAGCG | |
| | CGCACAGGGGACGGCCCGGGGTCCCCTCTGCG | |
| FOXL1_FBXO31 | CCGGCGGCCGTCTGGGTGCCTCGCTCCTGGCCGCCTCCTCCAGCCTCCGTCCGCCTT | 173 |
| | TCAACGCTTCCCTGATGCTCGACCCGCATGTCCAGGGCGGCTTTTACCAGCTCGGGA | |
| | TCCCCTTCCTCTCTTATTTCCCCCTGCAGGTTCCCGACACG | |
| CTU2_RNF166 | TCGTCCTCCCCGGAAGGACTCAGGAAAGACACAAGAGGGAACCCAGCCCGACTGG | 174 |
| | CAGGGCGGCTGGGCCCGAGGAGCAGGAGGCAGAACGAGGCACCCACAGGGTGG | |
| | GTGCTCTATCGGCCTAGTTTCCAGTGACTGCCAGCCTGGTGTTCAGAGAGCCAGCA | |
| | GCCGGGAGTAGTGCCCGCTTCCCCCACAGGAAGTTCCTGTCTGCGCCCACCCAGGG | |
| | GCTGGTGCTGAGCAGCTTCTCAGCTGAAGGAAGTGGCTGAGGGCGATGGGTGTGG | |
| | GGGCGTCG | |
| NDRG4 | CGGTCCCCGCTCGCCCTCCCGCCCGCCCACCGGGCACCCCAGCCGCGCAGAAGGCG | 175 |
| | GAAGCCAC | |
| LGALS9_KSR1 | GCGGGGAGGTTGTCTCTACACAAATGTAAAAGCCTGGCAGCTTCCCCAGGAGAGTG | 176 |
| | CGGGTATGGGCCGGGCCGGGAGAGGGCTGGCTGTTGCG | |
| LGALS9_KSR1 | CCGTGGGCG | 177 |
| LHX1_MRM1 | CCGCGCGGGTGCTCCAGAGCATCCAACTTCATTTCCACTTCAATTTTATCAGCGGCC | 178 |
| | GGGGAGCCGGGCGGGAGATAGGAGGCCGGCCCTGACACGAATTAGCCCGGAGAT | |
| | TGTCCGATACGCCTTGGCCAGGGCGCCGGCGCCGCGCGCTCGCCTCCCTCGCCTCTC | |
| | CTTTGTGTCCGCCTCGCCTCGCCTCTCGGCCTCGCCGCGCTCCATTCCCGCGGCGCT | |
| | GGCCCGGGCCGAGCGAACTGCTTTGCCTTTGGCCACGTTGAGCGCGCCGAGGCAG | |
| | CCGGGGGCGCGGGGCTCCAGGACCCGTCTGCTCCTGGTGCCCCCAGCTCCTCAGGG | |
| | TCCGGCCGGGTCACCTGGGCCG | |
| AATF_LHX1 | GCGAGTAGGGAGAAGGCTGGGAGTAAATCAAGGGGAGGCGGCGAGACCGAGGA | 179 |
| | CCCAATTCACGGCCCTGAATAACGGGGGTAGCTGGTAAGGGGCAGCTCCCGGGCTT | |
| | GCGCCCAGCCTCCTCCCTGCACCCAGGCCCGCGAGGGCTCCCCGCGATCCGCGAGT | |
| | TCCCCGCGCGGCCTTCCTCAGCCCGCCGAGGTCGCGTCTTCCCTCCCTTTCG | |
| PLXDC1_ARL5C | CCGGGGCGCTTCGGGGCTTGCCAAGAGACGGTGTTTAGAGAAAGAGCATAACGCG | 180 |
| | AAGTCACAATCGCAGGAAACTCGCAGCAGCCCCCCATCCCCGCCGCTGGCTCCGTTT | |
| | AGCGGGGAGAAAGGAGGGTCGCCCAGCTTTGCGTCCTGGGGCGCACCGAAGCGCC | |
| | GGGACCCAAGAGGAGCAGGCAGGGACG | |
| HOXB1_HOXB2 | ACGCTGTTAGCGGCCAGGCCTGAACCCCAGTGGGATATTCTACTTCCCCATCCCAGG | 181 |
| | AATGGAGGGGGTAAGGAACCCCAACAGGCTCGCCACCATTTTTTTTAAACCTCCTTC | |
| | CACTGCTTTTTCTCCCCCTCTTCTAGCTGCCCCTCACCCCACCCCCACCACGCTTACCG | |
| HOXB1_HOXB2 | CCGGGCTGGAGGCTGGGGAAGGTTTGCTCGAAAGGAGGAGGAGGAGGAATTAAT | 182 |
| | GTCGACTCCTTGATTGATGAAGTTTGAAATGTCTCCAAGACAGCGGGGAAGGAAGT | |
| | CAGACACTCGGCGAGCGACG | |
| HOXB13_TTLL6 | CCGGTCCTGCTTCTTCCAGCCTCTGCTGGATTTCTCTCCGACCCCTCTGGAGCGAAGC | 183 |
| | CCTTTGGCCCTGCGTTGCATGCGGCACGGTGCGGGTTCGGGCTCTGCGCTGGAGCC | |
| | GGGATGCCCTCCGGCGGAGGGTGCGCGTAGGCGGCGCCTGGGCGTGAGCCCCGC | |
| | CTGCAAGGCTCAGCGTCGGGGAAGCACTTTTCTCGTCGACCCGGGGTCTTTTTCCGC | |
| | CAAGGAGCTCGGGGCTCAAGAACTCGGGACTGGGCTGTGGGCGGGGCATGGTTTT | |
| | CCTCTCTGGGCG | |
| CHAD | ACGCTCGGCCGGGTGCCCTGGATGCGAGGCGGGAGGAAGCGGGGCCGGACAGCT | 184 |
| | GGATGCGTCTCCCTGCGGTGGGCCAGCTGCCTGCGCTTTAAAGGGGCGCTTGTGCG | |
| | GCGCCTGCCGAGCGTGAGAGCCGCCCCGGCGTCGGTCTCCCACTTCAGACTCGACG | |
| | CGCCGAAGCTGGCCCTGGGTAGACCCGAGCTCCTTCCCCACCCTCGGGCGCGCCCC | |
| | CACCCCTCTCTTCCAACCCCGCTTGCG | |
| MSI2_ | TCGGCCTTGGGTAAAGGGAGTGGGGGGCCATGTGTGGAGCCCTCTGGAAGGTCTG | 185 |
| ENSG00000166329 | GACTCCTGCTTTTCCTTGGCTCTTCTCGTTCTCCAACCACCCCCAAGGTTCAGCAGAG | |
| | TCTTGGGCGCGTCTCCTCCGTTTGTGCCGCGTGTTTGTGGCAGCAGCTGTTGGTGCT | |
| | GACTAATAGGACTTCCTGGCAGCTGTGCCGGGCACACGTGGCACCGGCAGGAACT | |
| | GCCTCTCCTCG | |
| BZRAP1 | CCGTCTGTCGCAACCCCTCAGCCCGGCCAGAGGCTTCAGGAGCTGCTGGGGGTGAT | 186 |
| | CCCCAGTGGTCCGCTGTGGTCCTTTATCTCCGGCTCTGCTCTCTGCTGCTGCTCTTTC | |
| | GCTTGCTGGGTGGCTGGGCTGGTCCTAAGGAGGCCTGGGTCG | |
| TBX4_TBX2 | GCGGCGGGGGGTCCTCAGGTCGCTGGGCTGGTCTTTTGCTGAGCCACCCGCTAACC | 187 |
| | TGAAAGGCCAGGAAGGAAACGTCGGCGAGTGTCTGGGATGGGGTTTCCGTCCCGG | |
| | GACTCCCCTACGAGGGCGGTCCCCGGTAGCCAGAAGATCCGGCCGGACTCCGAGC | |
| | CTGGCCCCTTGGGCGCCG | |
| TBX4 | GCGGGTGAGCAGAAGGGCCGTGCCCAGGGCCTGGAAGTGCAAGGCCGCGTGGTG | 188 |
| | GGCATGGTAGGGAAGCGGAGCGTGGGCCTGTGAGGCGCGTGTGCGCCTGCGACC | |
| | TCGGGACCGGGGCTCCCAAATGAACAGCGCGCACAGCTGGGAGCAGGGCTTGGG | |
| | GAGCGGGGCTCTGCGGCCGGGGATCCGTAGAAGCCG | |
| TBX4 | GCGCGTAGGACTGAGAGCGCAGGGCGCGAGCCGCAGGGCTCCGCTGCACGGCTCC | 189 |
| | GGGTGTGACAAGAGCCCAGCAGAGGACCCCATGGCCATGCGGGCCAAGCGCGAG | |
| | ACGGCCCCTCCTTGCGACCCCGCAGGCCGCCACATCTGGGACCAGCGGATCGCTTG | |
| | GTCGCTGGAGCCGATCCCGCCG | |
| SMURF2_LRRC37A3 | CCGCTCCCCGGGCCCTGTCCCGCCTGGACGCCTCCCTCCAGGAGCCTGCGCCCCGG | 190 |
| | CCCCGGGGTCAGGGTTGGGATGCGGGCTCTGCAGGCGCCCCGGCGAACAGCTCTA | |
| | CCTGGAGGCTGTCCCTGCCCCGCTTAGTCCAAGGGCCTTGGTGTGGGGGCCTCCGC | |
| | TGTCAAGGCGGGGGAACCGGTTCTCTCGGTTTCTCTCCCCTTCCCCAGCGGCTTCAA | |
| | CG | |
| CASKIN2_KIAA0195 | CCG | 191 |
| CASKIN2_KIAA0195 | GCGCCATCCTGGTCCTTGCACTGGGCCTACAGAGACGGACACCTGGTCAACCTGCC | 192 |
| | AGTCAGCCTGCTGGTTGAAGGAGACATCATAGCTTTGAGGCCTGGCCAGGAATCG | |
| SMIM6_SMIM5 | GCGGGCTGCGGATGGGTGCGAGGGTGGAATCTCGGTGCTGCGACGAGTGTGGGG | 193 |
| | CCAGCCGTGGAGGCTCCAGGTGTTCTCTCTGCCCCAGCAGAGCCCGGCAGGAGCCC | |
| | CAACAGGAAGCCAGCGCGGCATGGCTGCCACCGACTTCGTGCAGGAGATGCGCGC | |
| | CGTGGGCG | |
| GALK1_ITGB4 | GCGGGTCCGGGGGTCTCTCCTCCCCAGCTGTGCCGAGGCTGCACTCGCTCATCTGG | 194 |
| | AAAGGCTTCAGCCGCGCAAGGGTTTCACCTGCCGCGGCCTTCCCGCTCCGGCCGTG | |
| | CGCATCTACCCCCGCCCCCAACACACACCCCGGGATCCCGGGAGCTGGAGACGGGC | |
| | TCCCCTCGCAGAGCCTACGGCCTTCCCCCGCCTGGCCCTGCTCGGCCCGGCG | |
| TNRC6C_SEPT9 | CCGGGCCCCGCCGGGGGCGCTTCCTCGCCGCTGCCCTCCGCGCGACCCGCTGCCCA | 195 |
| | CCAGCCATCATGTCGGACCCCGCGGTCAACGCGCAGCTGGATGGGATCATTTCGGA | |
| | CTTCGAAGGTGGGTGCTGGGCTGGCTGCTGCGGCCGCGGACGTGCTGGAGAGGAC | |
| | CCTGCGGGTGGGCCTGGCGCGGGACGGGGGTGCGCTGAGGGGAGACGGGAGTG | |
| | CGCTGAGGGGAGACGGGACCCCTAATCCAGGCGCCCTCCCGCTGAGAGCGCCGCG | |
| | CGCCCCCGGCCCCGTGCCCGCGCCGCCTACGTGGGGGACCCTGTTAGGGGCACCCG | |
| | CGTAGACCCTGCGCG | |
| RBFOX3_ENGASE | CCGCCGGGTCTCCGCAGCCTCCGGGTCTCCGCAGCCTCCGGGTCTCCGTAGCCAGC | 196 |
| | CACCCGGCCGAGGGGCTGGGTCCACAGAGGAGGACCAGCAGCAGTGAAGGGCAA | |
| | GTCCACAGAGTTCTGAGGTGTCCAACCTCCGGGACG | |
| CBX8_CBX4 | TCGTGCGTGGCCGCCGGGCTGCCGTCTCGGCCCCTGTGCGGGTCTGCGCTTTGGCG | 197 |
| | GCCGCCGAGCCGAGGGGAGAAAATGGCCGGTGGCGCGGGGCCCGGCCGAGGGTC | |
| | GCGGGAGGGCTGGCAGGCGCGGCCGCTGGAGGGGCGCCGCTCTCAGGGCTCGGT | |
| | CAGGCG | |
| BAIAP2_CHMP6 | TCGAGCTTAACACTCAAATCATGTTTTCTCGAAATCATGTTACTTTCTGGCCAAGTAT | 198 |
| | GCCGGCGAAGCCACTGAGACACGCTCCGCACATCTTTAGAACATAAAGGCCCTGGC | |
| | AGTAGCTTGCGGCGCTCTTTGGAAAACTGCTTGGCTCTCACTGGAAACACAGCCAC | |
| | GCCTCCTCTGGGCCCCG | |
| ZNF750_B3GNTL1 | CCGTGGGTGCACTTTGCTGGGTCTTCCTGGGACACTGAAGTCTCCTGTGTCTCCAGC | 199 |
| | CCTGAGAACTCGGAGCCCGGGTGCTTTTGGGAAGGACGGGGCACCAGCTGGTGAC | |
| | ACATGGGAAGGGAGGTGTGGTTGTCACCTTGCCCAGGTAACCTGCTCTGCCTGGTC | |
| | GGTGCG | |
| ZNF750_B3GNTL1 | CCGGCCCTGGGACTCGGCCTGGAGAGCCTATTGACACCGTGCCATGGGTGCGGGC | 200 |
| | AGGGCGCCCTCCCTGGAGGGCGGCACGTGGTGCCAGTTGGTGACCATGAGCTGCC | |
| | TCACTCCTGAGGAAGAGTGTTCG | |
| ADCYAP1 | TCGATGCAAACTCCAGGGCAGCAGCCAGACTGGCATATGTAGGGCTCTCCGGTTAC | 201 |
| | TTTCTCTGTATGTCGCGGGTGAGAGGAACAGCGAGGACAATTTAGCGCAAACACAC | |
| | GAAGGGTCGGATCTCAAGGGGGCAGCGCTGGGAGAAAGGTTAGGCTTGAAGCGC | |
| | GCGTCGCCTGCCCGGATCTTATCCCGGGCCCCCTCCG | |
| CCDC11 | CCGGTGGGTGACTGTGGCTGGGAACTACGGGCTTTCTCGCCCCGGCGCCCCCTGGC | 202 |
| | GGACCCACCAGCAGGTTGAAGGTGTCCGGCCAGTGCTGAGCACCAAGAGCCTCAG | |
| | CCTTCAGCCAACCCCCCGCCCCCGCGGCCTAGGTAAGTGAATCG | |
| SALL3 | ACGCGAGGACACAACCCGGAAGAGTCCTCCCCGGAGCGGCACTGTGCCGGCCCCC | 203 |
| | GGTCTCGGACCTCCAGCCCCAGAGTGCTGGAGAATAAAGGCCCGTTGCTCATGAGC | |
| | CACTCTGCCTATGCATTTTGTTACAACAGCCTCACCGGAGTCCAACACCAACATCCA | |
| | GGTGAAACTGACG | |
| FGF22_RNF126 | TCGGGCTGGGAGGCTGCCCCGAGGAGCTTTCACTTTGACAGGGAGCTGGCCGGGC | 204 |
| | ACGCAGGGAACTGTACACCCAGCTGACAAAGCGGCAGACACCCAGGCCGGGGTGA | |
| | GCGAGTGTGGGTGAGGAGTGGCGGCTGGCCCCAGGGTCCTTGCTGGACAAGACAC | |
| | TTCAGCTCAGGGTGGGGCAGGGCTCACCCAGGGCTACCCACAGACGATGGCG | |
| STK11_C19orf26 | GCGCTGCAGGGAAAAAGCCTCCTTTGTGTGTGGGAAGTTTAATAAACTCCGCTCAG | 205 |
| | ATTGTGTCTCGCAGCGAGTGTCTGGAACCTTCCAGACAAGCCTCAGGCGTCCGGTC | |
| | CTCCAGTTGGTGTGGAAAGCGTGGGCGATCACCAAGGGGGGTGGGTTGGGGCAG | |
| | ATGGAGCCGGCGTGAGTCCCGTCTCTTCCCTTCCTTCCCAGAAAGGCAGCCCTGGA | |
| | GTCCATGCCTTGTCCCGCTCTCACCGGCAAAAAGTATAATCTTATTAGAAATAGGAA | |
| | AGTTCCAAAAAGCATCAATGAGTTAAAAAGAGGGCTGGGCATGTTCG | |
| C19orf25_APC2 | GCGCACATCGGCCATCCCTCGCGCTTTTACGCGGGAGCGTCCGCAGGGCCGGAAG | 206 |
| | GAGGCCCCTGCCCCGTCCAAGGCTGCACCAGCTGCCCCGCCGCCCGCCCGGACCCA | |
| | GCCCAGCCTCATTGCTGACGAGACCCCGCCCTGCTACTCCCTGAGCTCCTCCGCCAG | |
| | CTCCCTCAGCGAGCCCGAGCCCTCG | |
| CACTIN_PIP5K1C | GCGTGGCCAGCCCGCAGGTGGCGGGGCCGACGGGATGGGTCAGGGTGCACAGAG | 207 |
| | CACACGCCAGCCCCTGGGGGAAGCCCGGCCCGTGCGGGCTGCGGGAGATCCTGAT | |
| | GGGCCCCGAGCTGAGGCTCCCGCAGCCAGGGTCTGCGCGTGGTCCCCACCTCCTTG | |
| | CGCGCTCCGTCTCCAGCACAGCAGAGGTGGACGCCCCTCGCGGCTGGCTCCCCAGC | |
| | GTCCCTGTCCTCCAGGGGCG | |
| PTPRS_KDM4B | CCGTGGCGTTGAGCGCCTCCGCCTCCACCTTCCGCGGCGGCGCGCTGGGCACTGGC | 208 |
| | GGGCGGGAGGGGAGGGGAGGGGCGGGCGGAGCCGTTACCAGGGCGCCCGGCCC | |
| | TGCCCCGGGCAGTGCCACTGTCCGATTCCAGGATGCCGAGTGGCTGCCGGTGAATA | |
| | ACTGGGCGCTCTTAGCGCTCACCACCGGGCGGGAGGACATGGCCTCCTGCACACCC | |
| | CCCACAGCCCTGGGAGGGGCCCCTGAAGGTGCG | |
| CARM1_YIPF2 | CCGTGGGGTGGGTGCAGGGCTTGTTCTGGGAGATTCCAAGCTGAGGAAAGCAGGG | 209 |
| | CTGTCCG | |
| CARM1_YIPF2 | CCGGCCTGCCCACTCTAGGGAGGGGCCCAGATAACTTGCGTAGACGCCGGCCCTCC | 210 |
| | CGCCCCCAGCCTTCG | |
| ILVBL_NOTCH3 | CCGCCCACCTGGGGCTGCAGTCGGGCAGGTCCTGTTCGCAGTGGAAGCCTCCGTAG | 211 |
| | CCTGGCGGGCAGGTGCAGGTGAAGGAGGCCACGTGGTCGGTACAGGTGCCCGGG | |
| | CCGCAGGGGTTGCTCAGGCACTCATCCACATCGCGGGCGCATCGTGGGCCGGCGA | |
| | AACCAGGGAGGCAGGAGCAGGAAAAGGAGCCCACGCCGTCTTGGCACGAGCCAC | |
| | CGTTCAGGCATGGGTCTGCGGACAGGAGGAAGGCG | |
| IFNL2 | CCGGACGCCCCCCAGGGGACAGTGGCCGGCAGCACCTGCTGCAGCACGAGGCACA | 212 |
| | GAGGGTGCACTGCAGGGAGAAGTGAGGGCAGAGGCCAAGGCGAGGAGGGGGCC | |
| | GGCTCCCGCTCTCTCTCCCTCTGTGTGTGCTGCG | |
| CEACAM21_ATP5SL | GCGTGGGGGAAGGAAGAGGGTATGAGGCTGGCATGAAGTGGGGACTAGAGAAA | 213 |
| | GGGTGAGTAGTTTTCAGAGAAAAGGCCAGTGTCCAGGGCTGTCCAGGAGCGAATC | |
| | TGGTCACTTGTTCTGAAACAGGGGTCCGGGTCTGGCAGTGGCAGCATGGTGGGGT | |
| | GGGTGAGTGGCACTATGGAAGAGCCAAATCTCCACCTCTATCCTCAAAGCCTTTCTT | |
| | CCACACAGCTTTCCGGTTAGCAAGGCTCCATGAGAATG | |
| CCDC8_PPP5D1 | ACGCCCCGGCCTCGGCCTCGGCCGCCCGCGCGGGTTTTGCGGGCCCCGGAAGCGG | 214 |
| | TGGGAGGCGCGCCGGCCGGAGTCAGGCCCCTGGGGGCCGTGCGCGCCCTCTTGGC | |
| | CCGGGGCTTCCTGGATGCCCTGTCCTCCGGCTCCGACGCCTCGCTCTCGGTGTCCTC | |
| | CGACTCCTCCTCGGACTGTTCGTCCGAAGCCTCCTCCGACCCCTCG | |
| MAMSTR_RASIP1 | GCGTGCGGGGCTGGGGCGGCGGTTACCTGGGCGTCCTGGTAGCCCTGGAGCAGCA | 215 |
| | GGAAGTAGGGGCGGTTGCTGGGGGCCTGGATGAGGCACTGAGTCAACTGATCGAA | |
| | GTCCCCGGGGTCTGCAGTTCCGATTTGGGCGTCGGCTGCCCCTGGGGCCATGCTAA | |
| | GTGCCTGCTGTCTCCGCTCCTGCTGCCGCCGCCGCCGCCCCTGAAGGCTAAGCTCCG | |
| | ACACGCTGCGCCGCAAAGACAAGTTTTCTGAGCGCTCCTTGCCTCCAGACCCAGCTG | |
| | GGGCCCCTGATCCGGTCCCCGGGCCAGGACTGGCCAGCGCTGCCCCACCCGACGCC | |
| | GCCCGGGAGCGGTTCTTCTGTGGCCGCCACGAAGGGGCGCCGGTGCCTGCG | |
| ZIM2_USP29 | TCGGGGCCGGAGAAGCATTAAAATGACG | 216 |
| FAM150B_TMEM18 | CCGCGAGGGGCAGGACGAGGCTGCATGGGCCAGCGAGGGGGTCGACACCGAGCC | 217 |
| | AGAGTGAGCGCGGGGCCTGGGGCGCAGAGCCCGCCCAGGGAGCCGGGAGACGCC | |
| | GCGCAAGCTCCCCGGACAAACGCAATGACCGAGGACGCGCGGGCGAGGCCGTCCA | |
| | GGGAGCCCTGGTCCCTCAGCTGCACCGGACTGAGCCGCGACCGCTCAGCACGCGCT | |
| | GCTTATAAATCAGGGGTGCGCTTCCCAAGCCCCG | |
| TPO_SNTG2 | CCG | 218 |
| TPO_SNTG2 | ACGGCTTTTTGGTGGAGGCTAATGTTAAATTCCG | 219 |
| PXDN_MYT1L | CCGTCCTATGACTCTCTTTTGATCAACGCAATGCAGTGCAATTGATGCCATCTGACTT | 220 |
| | GCAGGACTGGGTTAGAAGATGCCTCTCAGATTCCATATAGGTCTCTTGGAAGATCC | |
| | GCCCCCGGGAAAGCCAGGCCATGTAAGACCATTGACCACCTTAGGACCACCAGGCT | |
| | TGGAGGAAGCCAAGACACCCACGTGGAGAGGCTGTGCAGGGAGTGAGGGAGGTG | |
| | CAGCCAACCCTCACCTGGCTCCACTTCAAGGCCCG | |
| SOX11 | GCGGAGAGCTTGGAAGCGGAGAGCAACCTGCCCCGGGAGGCGCTGGACACGGAG | 221 |
| | GAGGGCGAATTCATGGCTTGCAGCCCGGTGGCCCTGGACGAGAGCGACCCAGACT | |
| | GGTGCAAGACGGCGTCGGGCCACATCAAGCGGCCGATGAACGCGTTCATGGTATG | |
| | GTCCAAGATCGAACGCAGGAAGATCATGGAGCAGTCTCCGGACATGCACAACGCC | |
| | G | |
| HPCAL1_ODC1 | CCGTTTCTGAACCCAGGAGACACTCAGGAAACCTTGCTGGTGGAACGGATGCAGCA | 222 |
| | GCGAGGTTTTCCGGGGCAGGAACACCCTCCCAGGAGCTTTTCCACGGCCAAGCGCT | |
| | GGCTGGTGGTGGAGCTGCGCTGAAGTCAGTGTGTGCTTTGGGCCCAGCTGCACTGT | |
| | GCCCGGGGTCCAGGGATGGGTGTGAGGCTGTCTGCCCCCCACTGCACGCCCGGCT | |
| | GTCAGAGGCATCTGTCTCTTCCCCCGCATGCATCTTTCTCCCCGTCTGGCATGGTGTT | |
| | TCTAGTCTTTTGTGGATGGGGACATAAACAAGCCGCCATCAACTGCTTGGTGACATT | |
| | GGCCAATCCTGTGGTGGCCCCAGCTGGGCTTGCTGCCTGTGTGTGGTGAGGGTGCC | |
| | CTTCTTGTCACCCG | |
| NT5C1B- | TCGCGAGGTTGCGGGCAAGACCCCTTGAGGTGCCAAGTCCTGGGCCGCCCCTCCAG | 223 |
| RDH14_OSR1 | GGCTGGCCAGCAGGGGGCAGCGTGGCTCTGAGCGTGGAGGCCAGGGCTGGTCCG | |
| | CGCCGGCAGGGCCAGCCTCCAGTGCCCAGTTGGGTTCCCGGGCCTCGAAGTTCTAG | |
| | CCCGCACAGGACTCAGGAGCGTTCCCGGAGGAGGTGGGGATGGGGTGGTGAAAG | |
| | CCCAGAGCGTTTTAACTTCTGCATCCCCTGCCGCTTTCTCAGCCAGCAGGGCCCGGC | |
| | TTGAGGCTGGGATTTTTGGTGCCTGCAGCAGGGAAGCTTATAGTCCAGTTGTCATC | |
| | CGCGGCCGCCGCGCTCCGGGCGCTGAAGCTGGAGAGGCCATCCTGCGCTTGGGAA | |
| | AGGCCGCGGGCGCCACCGCCTGCGCGGTCCCGCGGTCAGGGCGCTGGAGCTGGG | |
| | GGGAGCCCCGCCTTGCCCCAAGGAGAAGAGCCCCGGCGGCCTGGCTTCTAACTGTG | |
| | GGAAAACTAGACACCCCAGGGAAGGTTCAGCTTATGGAAGGCGGACTCGAATTTTT | |
| | CCTCCTAAGCGTCCCGGGCCTCCCAGGGCGCCCGCCCCCACCATTCCTGACAAGGCT | |
| | TTAAAATTGTAGGGAATCTTCGCGGGTGCAGAGCCTCG | |
| LBH | TCGGAGAAGACGTGGGAGTCAAGGATGGGGGGCGGCGTGCACACCGCCCGCCCA | 224 |
| | CACCTTCTGCCCCCGCTGCAGACCGGGCGTATGTGTGTCTCCAATGGAAAAATCCTA | |
| | CCCAGGACGACACCACATCCTTGCTCCCACAAATAAAACCTTCCACGGAACTCAGGG | |
| | CTGCAGACCAGCCCTTCGCAAGCCAACGCGCCCCGTGGGCACTCGGTCCCCCG | |
| XDH_MEMO1 | GCGGGGCGCGATATGCCACAGGTAACCGCCGCCTGCGCGCAGTTAAGGAACAGTC | 225 |
| | CTGTCCAATAGGTCTCCCCAACCTGAGCTTTCCAGGTCGCCTCCCGCCCGCAGGACC | |
| | TCTTTCTCTCGAGCAGCCAGAGGATTTGGAGCTGCTGAGAGCGGATGAGGTCCTGG | |
| | GGGAGTGAAGGCGGCGTCTGTGCCGCAGCCGCTTGTCAACTCTCTAGCGTCCAAGC | |
| | CCCGGCCCCGGCCCCCGCCAGGTGCG | |
| XDH_MEMO1 | CCGAAGAGGGAGAGGGGCTGCCGGGCGAGGATCCCCGCGGGCACCGCGAAGGAA | 226 |
| | GGCAGCTCCTGCAGGAACCAGGCGGCGCGGGCTGGCAGGCGGGTAGCCGCCGGC | |
| | TTCAGGCTCTCCGTGTGCTTCCCGTAGCCGGAGGGCTTCGCGACGTACAAGGCCAG | |
| | TGCCCCAAGGGCGACCAAAGTGGCGCTGCCTGCCAGCACTGGGCTCTGCTGGCACT | |
| | GAACCTGCATCGCGCCGTGTTCCTCGCCGGTGGCCG | |
| SIX3_CAMKMT | ACGGTGCGGCCGCTTGGGCGTGATCCCTTGGCTGGGGCTGCAGGGGGCCCGTCCT | 227 |
| | CCAGGGGCGCAGAGGGAAGGACCAGCGTTTCCAAGCCGGGCTCTGGCCGCCGGCG | |
| | CGAGAGCGAGGCCAAGGTCTGGGGGCAGTTCAGGGGGACCCCGAAGTCGGGACG | |
| | GCCCAGAAACGCTTTGCCCACAGCCACCGCCCTTTCCTTTGTGAGTTTCCCCAAAGC | |
| | CGTCGGTGCGACCCGGCGCCGACTCTCCTCCTCTTCTCCCTGCGAGGGCCCGCGCCG | |
| | CCCG | |
| SIX2_SIX3 | ACGCTCCCCTGACCTCAGGGCCCAGAGCCTCGCATTACCCCGAGCAGTGCGTTGGTT | 228 |
| | ACTCTCCCTGGAAAGCCGCCCCCGCCGGGGCAAGTGGGAGTTGCTGCACTGCGGTC | |
| | TTTGGAGGCCTAGGTCGCCCAGAGTAGGCGGAGCCCTGTATCCCTCCTGGAGCCGG | |
| | CCTGCGGTGAGGTCGGTACCCAGTACTTAGGGAGGGAGGACGCGCTTGGTGCTCA | |
| | GGGTAGGCTGGGCCGCTGCTAGCTCTTGATTTAGTCTCATGTCCGCCTTTGTGCCG | |
| SIX2_SIX3 | GCGGCCGCCGGCCCGGCCGCCCTGAGTCCGATTTCCCTCCTTCCCTGACCCTTCAGT | 229 |
| | TTCACTGCAAATCCACAGAAGCAGGTTTGCGAGCTCGAATACCTTTGCTCCACTGCC | |
| | ACACGCAGCACCGGGACTGGGCG | |
| TTC7A_CALM2 | TCGGGTTGAGAAAATCCG | 230 |
| TTC7A_CALM2 | TCGGGTCTGCCCTAGACCCATTCCGGCCCTCAAAGATGAAGAAAATGAGAAGGGG | 231 |
| | GCTCTGGCAGAGAGAAGTGTGATGCCTGCAGAGGGCCCG | |
| ETAA1_MEIS1 | GCGGTGGGGGCTATCAGCGAAGGGAGGGGAATGTGCGTGGAGCTGAGGAGGAG | 232 |
| | CCTCCCGGCTCTCCGAGGGCCTTGGGGTTGGGATCCCTAGGTGCAGCCCGTTGACA | |
| | GTCGGCCCCACGGCCATGGACGTCCTTTCCCCAAGTTAGCTGAGCGCCTGCCACCG | |
| | AGATCCCCCGAGCCTGGGCTTCGCGCGGCCGCCTAGGAGGAACCCGCAGGAACCA | |
| | GCCCTCCCCAACTCTCCGCCCGGCGCCTTTCTCCTCCACCGGATCCTGGATGTGCAG | |
| | TGGAGGGGACGAGGGCTTGTCGGGTGGGAAACTTAATTCAAAATGGCTGCTGGAA | |
| | ACGCTTGGGTTTTATTCGTAGCAAATGTTGCCAATTTCTCCGGCCAGATACGCTAAA | |
| | CCGATCCTCAGATACCGTCCATGGCTCAGGGCCTCCGACTTCAGGGCTCCAGGAGG | |
| | AAGGGGAGGTGAGCGGTCACCTGGGTCTGGGGGAGGGGGAGGAAAAGGAAAAA | |
| | AGTAGATGACACAATCG | |
| ARHGAP25_BMP10 | TCGGAGGCGTGAGTCTTCGGCCCTGCCATGCCTCACATCCCCAGGATGCCGCGGTG | 233 |
| | GGAACTGGGCTGTGGCTTTCCTGCCCTGGCACTGCTTGTTTGCTGGGATTTCAGGA | |
| | GGAAAACCCCCAAGCTCCGAAAGAAAGGTATTTCTTTTTTATTTTGTAGTTCACTTCT | |
| | TCCACTAGAAGACTCG | |
| EMX1_SFXN5 | CCGCCGCTTCCTGAGCCATCAGTCCCAGCGGGTACGTTATCGAGTAGCACAAACAG | 234 |
| | TTGGATTTTTCCCTCAAGAACCGAGTCTGGACGCGGAGATGGAGCCAAGTGTGGCT | |
| | GCATTTTCGGACCCGGAAATCCGTTGGGCACTGAAGGACTTTTCGAACCCTGTAGC | |
| | GCTGTTGCTTCGCGGTCCATCGTCGCCGCTGCAGACGGATGCGCTCCCCGGCGGCT | |
| | CTACGCCCTCCAGTCCCGGCCAGGCCTCTGGGCTGGGAGCCGAGCCGTCTCGGGCC | |
| | CTCCGGCGCCGCGTTTTCTAGAGAACCGGGTCTCAGCGATGCTCATTTCAGCCCCGT | |
| | CTTAATGCAACAAACGAAACCCCACACGAACGAAAAGGAACATGTCTGCGCTCTCT | |
| | GCGCAGCGCTTGGGCGGCGCGGTCCCGGCGCGCGGGGAAGCGGCGTCTCCGCTAA | |
| | CCGAGGCGCTGGAAGGGGAAAAGCGAATGCGGAATCGTCCAGGACTCCGAAGGT | |
| | CGGGGCCGCTCGCGAGCACCGAAGGGGAGGAGCCGACGAAGACCAGGAGTGGGC | |
| | CGCATTTCGGTACTGTTTCCCCGAGATCAGGAACTTTCCGGGTCTAGGAGCAACG | |
| MRPL53_LBX2 | ACGGGGAACCAGGAGGAGAGAGGTGAGGAAAAGGCTAAGTCAGAGTCCGCGACC | 235 |
| | TTGCCGGCTCTATACCTTCAGAGGGCTGCAGAGCGCGCGCGTCAAGTCCGCGGAAA | |
| | GTTTTACTAGTCAGCTCCTCCAGCGCGCACAGCGGCGACGTTGGACCCGGACCCGA | |
| | CTCTGGAAGCTGCGGCGCAGAGGGTGCTCGGGGGACCATGCGCGGGGCTAGGAT | |
| | GTCTGCGATGCTTAAGAGTGTCCGGGGTGTTCGGGGCTCGCGTCCCGAGTTCATGG | |
| | TCGGCCGGGCTGGGGCGGTCCGGCTGTCCGTTGCGCTAGGCTCCGCAAACGCCTG | |
| | GGCCCCAGTGCTCGGCTCCCAATCCGGGCCCCCAGCCTCGGACCCGCCCCCGGCTCT | |
| | GGGCCCGAGTCCCGTGTGCCCCTCCTCCTGCG | |
| VAMP5 | TCGCCACTCGCGGAAGGCGCGCCCCCCGCCCTCGCTCGGCGGCCCGCCCCGCCCCG | 236 |
| | CCCCTGCTCTTCCTCCGGGGCCGCTGGCACTGCGGCCGCTCCGCAGGCAGAGAAGC | |
| | CGGGAGCGGGCGAGGCGGCGGCGGCAGCAGCGATGGTGAGGGCCCAGGCGGGG | |
| | CCGGCCAGCCCTGCGACGGGCAGAGGGCGAGTGGCGAGGGTGGGAGAGAGGAG | |
| | TCCAAAGTCCGCGGGCTGGGGCCTCCCCTGGGGCCCACGAGGGCCAGACCTGAGG | |
| | CGGTGACCACTGCTGGAGCAGGACGGGGCGGACCCTCCACTCCCTGCGCGCCGCAT | |
| | GGGAGAGAAATGCGTGAGCCCCGTCCTGGCTGCACCGCGCAGAGCGAGCGGGACT | |
| | CG | |
| ST3GAL5_POLR1A | GCGGGAAGGGGCAGGAGTGGGAGGTCCCTCCTCGGTGCCCGGCTGCGCCAGCTGC | 237 |
| | TGCCGTGTTCTGGTGTACCAGGCCGGACCTTGCGCAATGCCTTTGGGGTAATCTTCA | |
| | AACCTATGTCTGCTGATCACTCTCTTTAGCTGCCTGGCAGTACCGCAAACCCAGTTGT | |
| | GGAAAGTCCCACCACAAGGACCTTGACAGAGGTGGAGGCCCTCCCCATGCAGAAG | |
| | CCAGAGAACTGCGCCCATTCTCCCGGTATCCTTCCG | |
| MGAT4A_TSGA10 | TCGGGGGGAGTCGTGTCCCCCTCAGGGATGGCGGTGGGAAACGGGCTCGCGACGT | 238 |
| | CTTCGGGAGCACAGACCACCTCCTCCGCCTTGTCCGTGGCCGGGGCACACGGGCCT | |
| | GCGGGGGGCGCCTCCCCATCCTGCTTTCCGCCGTCGGGACCG | |
| POU3F3 | GCGAAAGAGGGAGATGCCCGTGTAGAGAACCGAGGAGGGGGGCTGGGGTAGAAT | 239 |
| | AATCAGCTCTAAGGTTGCAGATTTAGATCTCAAGGCTGAAAAGGATAAGCTTCCAC | |
| | CAGAGCATCCTGTAGCGCCTCCTGTCCTGCCCTGCCCTGCCCTGCGCGCGCACCGCA | |
| | CTCACACGTACACCCGGTCCTCGCACGCGCACACACGCACACTGTTCCCCGCCG | |
| POU3F3 | GCGCGGCCTTCGGGGCTCCAGAGCGCGCGGGCCCGGAACGAGGCGCGCGGCCGC | 240 |
| | TGGCACATGCGGGGACTGCCCAGCGCGGACTGGAGAAGGGGAGCGAAGGGGTGG | |
| | GGAGGGGGTGACGCCGGCTGCCCACCCCGCTCCGCG | |
| POU3F3 | ACGTTCACACACCGCTTGCTAAATGCAGTGGCGAGAGGAGGGAGCAGCGTCTACAT | 241 |
| | GAAGCGAACTTTTCAAGCGCAGAGCCCTGACTCCCAGGCGCGGGGGCTCACCGGG | |
| | AGGGGCCCGGGCGAGAGAGCGCGTGGGTGCGTGAGTGCCTGTGTGCGCCCGCCCT | |
| | TTGCTTGCTCGGGGTGTCCGCCTTTGTCCCCCGCCGCGGGCCTCCACGGTGGGATCT | |
| | GCGCGCGGCCGGTGGGCAGCCCTCGACCCGGGGCGCGTCCACAGCGCCCACCCGC | |
| | GGCCCCCAAACACCTCGAGAGCAGATCTTAGGGGTTAACCAGGCACCG | |
| C2orf40 | CCGCTTTCGCTGCGGGCAGCGCTGGCCACGCGGCCCCCGCCGCCGGCGGTTCTCCG | 242 |
| | TGGCCAAGCATCCTTGGCCTTGGAGCCCAGGGGCTGCGTTCCCCTTGGGGCCGGGG | |
| | CGGGAGAGAGGACCTCGGTGGTACTCGCCCGTGCGCTGGGCGCAGCCGCTTGGCC | |
| | CTCAGCCCTCTGGCGCGGCGCCCACCCGCTGGGTCCCGCCCCGGCAGCGACGCAGG | |
| | GATAACCCGCGGCCGCGCCTGCCCGCTCGCACCCCTCTCCCGCGCCCGGTTCTCCCT | |
| | CGCAGCACCTCGAAGTGCGCCCCTCGCCCTCCTGCTCGCGCCCCGCCGCCATGGCTG | |
| | CCTCCCCCGCG | |
| PSD4 | GCGGAAGTCGGAAGCTCCAGCCGTCACAGCCACATTCACTGGGCAAGCCG | 243 |
| PAX8_PSD4 | CCGCCGGAAGGGTCAGGGGAAGGTTAGGAGGAAAGATGGACCTCCAGAGCCGAG | 244 |
| | CAGAAGTGCCATTGCACCAGCTTGGCGCAGAAGTGCCATTGCACCAGCTTGGCATG | |
| | GGCACCGGGCACTGCACATTAGGCCTCAGGGATGGTCCTGGCGATGTCTGGTATCG | |
| | TACCACG | |
| ARHGEF4_FAM168B | GCGGCCGCCGCACCGCCGCCCCCGGCCCAGCCTTCCCCGAGCCTGTGGCTGGAGCT | 245 |
| | CGGGCCCGCCTGCGTGCGGGCGCAGCAATGCCCCAGCGAGTCAAGCGGGCAGACG | |
| | AGTGGCGATCTCGGCACTAGCAGCAGCAGCAGCGCCGGGCTGTCCCCGGGCTCCG | |
| | ACTCGGACAGCAGCGGCGTGGTGTGTGGCGGCCGCGGAGGCAACGGGGGCATGC | |
| | GCGGCGCCGTGTCCCGCTCCTGGAGCCTGGAGAGCCTGCGCTCGGCCACCGCCGGT | |
| | AAGGACGCCGCCATCCCCGCGCCGCACGCGCCCTCCGCGCCCGGGTCTGTGCTCTT | |
| | GGGACCCCCCG | |
| FAM168B_ARHGEF4 | GCGGCCAGTCCTTGTAAGGAATCAGAGTCCCTGGCCCATCCCTCCCCAAAGCGCCG | 246 |
| | GTGCCAGGCGTTTTGGCCTCTGTATCTCTGAAACGAGGAGGTCCCGGGGCATCCCC | |
| | GAGCGCCCCCGTGGCCATCTGTGCCACTGGCCAGCCCAGGGCCAGGACTGCTGTGC | |
| | CGGCGTGGAGATTCCCGACCCTTTCCAAGGAGGTGCCAAGGGCGCAGCG | |
| SLC4A10_TBR1 | CCGAGGGCCTGGCCGCCGAGCGCTCGCCGCTGCCGCCCGGCGCCGCCGAGGACGC | 247 |
| | CAAGCCCAAGGACCTGTCCGATTCCAGCTGGATCGAGACGCCCTCCTCGATCAAGT | |
| | CCATCGACTCCAGCGACTCGGGGATTTACGAGCAGGCCAAGCGGAGGCGGATCTC | |
| | GCCGGCCGACACGCCCGTGTCCGAGAGTTCGTCCCCGCTCAAGAGCGAGGTGCTG | |
| | GCCCAGCGGGACTGCGAGAAGAACTGCG | |
| GALNT3 | ACGCAGCCCAGGGGTACCGCGTCTCCCTCCGCCTGCCGCCGGCTTACCTGGCGGGT | 248 |
| | GGGCAGGGCAGGGTGGCGGGAAGCGGCGGCCGGGCAGGCGCTGGACGTGGGCT | |
| | AGGCGCCAGGTGCAGGTGGCGGCGGCTGCGACTCCGGTTGCTGTCGCCACAGTTG | |
| | CGGCTCAGTAGAGCTCCTCCTCCGCCGCCGCCTCCTGCCTTCCCGCTGGGCCTCCCG | |
| | CGTTGCCTGGAGAGGCAGAACCGAGGCTCG | |
| GORASP2_GAD1 | CCGACTAAAATTCTCTAGCCTTATCGGGCCAGAAAATACGGATGTCCCCGGGCAGA | 249 |
| | GGTTGGAGAGGCGGGGGAAGATTAACGGGCGGCTTATTAAAGAGCCATCCGTCAG | |
| | CTCCTGCGCGCGGGAGATAGCGGCAGAGCAGGCACGGGACACGCCCGCCCGCCCT | |
| | AGCCCCGGAGCGCCGAGAGCCGCCCGCCGCCTGGGTGCTCTCTGCACCTGATCTTC | |
| | CCAGCCTCCCTGGGTCCCGGGGCGAGGGCGGTGGCAGTTTGCAGTCAGAGCAGAG | |
| | TGGCCG | |
| DLX2_DLX1 | CCGGCGCTGAGACTGGCGGCGAAGCACAAGGTGGAGAAGCGCTGGCCCCAGGGT | 250 |
| | GCTGCTCCGAGGGGATCTCACCACTTTTCCACATCTTCTTGAACTTGGACCGGCG | |
| SP9_CIR1 | ACGACTCTTAGAGGCCGGGCGAGAGGCGCGAGCACACAAGCGAGTAGAGACACC | 251 |
| | GAGAACGAACGAGAGGTTCGGAGGGCGAGCGAGCGGGAGGCGGGAGGGCAGGG | |
| | GCTTCAGTGACGCCCCCAGGGCCCGGGCTGGGCGCGAGGTGGAGCCGCTCAGGGC | |
| | TCCCGGGCTGCGGTTCGCCCGCTGTGCGAGGAGCTCCCCTCTGCCTTCCGCGCCCG | |
| | GATAAGAATCGAACGCGTGGTCCGGAAACAAAAGCGAACCATCCTCCGACACAAAC | |
| | ACTTTAAAAACTGTACTCCCAGACG | |
| KIAA1715_HOXD10 | ACGCCGTACGGTAGCGCCGCACTTGATCCGCGCCAGAGCCGGAGCCACCCAGCGCC | 252 |
| | GCGCTCCCGCCGCTGCCTCCGCTGCCTCCATGCAGGCTTCCGAGGCCTGAGCCCGAC | |
| | GCCGACGTCGTGGTGCCGGCAGCCGAGCCGCTCTCTGCGTACCCTGGCAAACAAAC | |
| | GACCAACAGCGCATGAGTGGCTGTAGGACCAACAGCCCGGCGCTGGCGCTGCGCG | |
| | CGGATCGGGGAAGCCCCG | |
| HOXD10_HOXD11_ | GCGAGGCCGGTCGGCTGCTGGAGAGACACAGAAGTTTCACGGTGGGAGGCTGAGT | 253 |
| | GGCTTTCTCCCCCGGCGCCGTTCTCAGGGTCTTTCTGCGGGTCGAAGAAGGACCCG | |
| | CGGGAGCTGAGAGGCCCAGGTCGGAAGCACTCCCGGCTGGCCCAAGAGTAGAGG | |
| | CGAAGAGCG | |
| HOXD10_HOXD11_ | GCGCCCGAAGCGGCCGCTGGGCCAGAGGAGCGCGGTCGTACCCGGCCGTCCTTCG | 254 |
| HOXD12 | CCCCCGAGTCTAGCCTGGCTCCTGCAGTGGCTGCTCTCAAAGCGGCCAAGTATGACT | |
| | ACGCTGGTGTGGGTCGTGCCACGCCGGGCTCCACGACCCTGCTCCAGGGGGCTCCC | |
| | TGCGCCCCTGGCTTCAAGGACGACACCAAGGGCCCG | |
| HOXD10_HOXD11 | TCGGGGTCTTCACGGTAGGTTCTCGAGCGGGACGCGCGGGTCCGGAGGCTGCGGT | 255 |
| | TTTCCCTGGGTTTGGGGAATGGGGGTAGGAACTAGGAGGGAGCTGGGGCCAAAG | |
| | AGCCAAGCGGGCTGGGACTGGAATGAAAGCGCTCTGGGTTGTGGAGTGGGTCGG | |
| | GGGGCAAGGGTCCGCGCTAAGGAGCCGAAAGGGGCCGGCCGCCCCCTTCCCCTAT | |
| | GCACCGGCGCGCCACTGCAGATGGCTCACCCTCCCCCGCCAAATCGCTGCTCCCG | |
| HOXD9 | GCGGGCTCTAATTGCGGCGCTTATGTTGATGATTTTTTTTTTAATCACAGCAGCCCCC | 256 |
| | AGTTTAGCGGACTGATTTACTCCCGGTATTGGTAAATATGATCACGTGGGCCGCGC | |
| | GACCAATGGTGGAGGCTGCAGCCTGCGAACTAGTCGGTGGCTCGGGCGCCGGCGG | |
| | GGAGCTGCTCGGCGGCGGACAGTGTAATGTTGGGTGGGAGTGCGGGACGCCTCAA | |
| | AATGTCTTCCAGTGGCACCCTCAGCAACTACTACGTGGACTCGCTTATAGGCCATGA | |
| | GGGCGACGAGGTGTTCGCGGCGCGCTTCGGGCCGCCGGGGCCAGGCGCGCAGGG | |
| | CCGGCCTGCAGGTGTGGCTGATGGCCCGGCCGCCACCGCCGCCGAGTTCGCCTCGT | |
| | GTAGTTTTGCCCCCAGATCGGCCGTGTTCTCTGCCTCGTGGTCCGCGGTGCCCTCCC | |
| | AGCCCCCGGCAGCGGCGGCGATGAGCGGCCTCTACCACCCGTACGTTCCCCCGCCG | |
| | CCCCTGGCCGCCTCTGCCTCCGAGCCCGGCCGCTACGTGCG | |
| HOXD8_HOXD9 | ACGGACTGAGTGCTCCGTGGCCCGGGAGTCCCAGGGGAGCAGCGGCCCCGAGTTC | 257 |
| | TCGTGCAACTCGTTCCTGCAGGAGAAGGCGGCAGCGGCGACGGGGGGAACCGGG | |
| | CCTGGGGCAGGGATCGGGGCCGCGACTGGGACGGGCGGCTCGTCGGAGCCCTCA | |
| | GCTTGCAGCGACCACCCG | |
| HOXD8 | GCGGGGCAGGTCGCCTGGGGCGTCGGCGATTATATTGCGGCCGAGCCGGGGCGC | 258 |
| | GCCGGGAAAGGCCGGGAGGGCGGCGGCGCGCGGGGGCTGGGCGAGGCCCCGCG | |
| | ACCCGCGAGGGAGGCGGCGCGAAGCCGAGGCGGCGGGCGCAAGAGCCGGGCAT | |
| | GAGCGCCCAGTAGCTGAGCGCCCGCGGCTGCCTGGCCTCAGAAGCGACGCGCGAG | |
| | CGCGGGCGGGCGGCAGCAGCGACGTAGCCCGGCGGTCCCGGCGGCGAGAGCAGC | |
| | CGCCCCACAGGCCCCCGCGGCAGTGCGGCCGAGTCGAGGCTCGCTCTCTGGCTGCT | |
| | TAGCGCCGCCCG | |
| HOXD1_HOXD4 | GCGTGTGCGCCGGGGAGAGGGCGGGAGGGAGGAAGCAAGCGAGCTTGGGAGCG | 259 |
| | CGCGGGGAGGGCCGCGGGCCTCGGGGCGCGCCAGGAAGTGAGCGGCGGAGGCG | |
| | AGGGGCCTAACTAGTGGCCGGGCGCTGACCTGCCTGTCCTGTCTGTTTTGTCTCGCA | |
| | GTGAACCCCAACTACACCGGTGGGGAACCCAAGCGGTCCCGAACGGCCTACACCCG | |
| HOXD1_HOXD4 | CCGTGGTGCGGGATTCCCGAGTGTGGCCCCGGCTGGGGGAGGGTCTTGGGCGCTC | 260 |
| | ATTACAGGCCAGGAGGTCCGCTGCTGGCGCTGGCACGCTTAATTCTTTTTTCCCACA | |
| | TTGCAGAATCATTCCCACCAGCCACTCG | |
| BOLL | GCGGGTGGGGAGAAGCGGACTGCGTCGCCTCGGGTGGCAGGTGGCGGTGCGGGC | 261 |
| | GGGCGCTGCAAGCCGGAGAGGGGCGCGGGAGGGCGAGTTTCGGCTGTGGCCCTG | |
| | GGACTCCGAGCCGGGGCGTCTCAGGGGCAGAGCGCACGGCACAGCGGGGCGGGC | |
| | GTGGGGCG | |
| PTH2R | CCGGGACAGAGTGGAGGGAAGCAGAAACATTGCGAATCGGGGGTGGCGGCAGCA | 262 |
| | GCGACATGAGATCCTTTGCCCTCCGCCCCCTGGGCTGCGGGACCCAGTGACTTCGA | |
| | GGAGGAGCGCGAGCGCAGCCGCGCGGGGCGCACCCGGATCCGCCTGGGGCGGGA | |
| | GCCGCCCCCTTCCCGCCGCAGGCGGCGCGGGGCTGCGAGTCAAGTCCAGGACTCG | |
| | GGCCAGTCTCTCCG | |
| GMPPA_SPEG | TCGGAGCGCGGCGCACCGTGGGGCACCCCCGGGGCCTCGCAGGAAGAACTGCGG | 263 |
| | GCGCCAGGCAGCGTGGCCGAGCGGCGCCGCCTGTTCCAGCAGAAAGCGGCCTCGC | |
| | TGGACGAGCGCACGCGTCAGCGCAGCCCGGCCTCAGACCTCGAGCTGCGCTTCGCC | |
| | CAGGAGCTGGGCCGCATCCGCCGCTCCACGTCGCGGGAGGAGCTGGTGCGCTCGC | |
| | ACGAGTCCCTGCGCGCCACGCTGCAGCGTGCCCCATCCCCTCGAGAGCCCGGCGAG | |
| | CCCCCGCTCTTCTCTCGGCCCTCCACCCCCAAGACATCGCGGGCCGTGAGCCCCGCC | |
| | GCCGCCCAGCCGCCCTCTCCGAGCAGCGCGGAGAAGCCGGGGGACGAGCCTGGGA | |
| | GGCCCAGGAGCCGCG | |
| PAX3 | GCGGGAACCCGCTACGCGGGTAGTTCTGCCCCGGGCCCGGCCGCATCATCCTGGGC | 264 |
| | ACAGCGCCGGCCAGCGTGGTCATCCTGGGGGCAGCTTCGCTCGGAAATTATATCCA | |
| | GGTGAAGGCGAAACGGAAAGGCGAGTGCGGCGCGGATGACCCTCGGGAACTATC | |
| | CGGAGCGTGGAGAGCCCCTCCCCAAAACGGCTGGAGAGAGAGGGAGGGACGCGG | |
| | GGAGGGGGGCTGTCGGTTCCTAGTCCAGAGGCCG | |
| PAX3 | CCGAGTGCGGGGATCCGGGCTCGGGAGCATTTATTAGTTCTTTTACCCAAAGCTTG | 265 |
| | GTCAGGAGCCCTGAGCTGCGATTGGCCGACGGGTAGACCGTCCCGGGTGGCGGAG | |
| | ACACGCGCTGATTGGGCAACAGCGACCACTTTCTCTTCCCATCTCTGGTGGTGCCGA | |
| | GGCCTCTGCTGGCCCCG | |
| INPP5D | CCGCAGCTCAGTTTCCTTTCCCTCACTGAGCGCCTGAAACAGGAAGTCAGTCAGTTA | 266 |
| | AGCTGGTGGCAGCAGCCGAGGCCACCAAGAGGCAACGGGCGGCAGGTTGCAGTG | |
| | GAGGGGCCTCCGCTCCCCTCGGTGGTGTGTGGGTCCTGGGGGTGCCTGCCGGCCC | |
| | GGCCGAGGAGGCCCACGCCCACCATGGTCCCCTGCTGGAACCATGGCAACATCACC | |
| | CGCTCCAAGGCGGAGGAGCTGCTTTCCAGGACAGGCAAGGACGGGAGCTTCCTCG | |
| | TGCGTGCCAGCGAGTCCATCTCCCGGGCATACGCGCTCTGCGTGCTGTGAGTACAA | |
| | CCTGCTCCCTCCCCG | |
| CXXC11 | GCGTGGGTGGCTCCTGGCTGGGGAAGTGAGAAGCCCTCCGTGCGGTGTCTCTGAA | 267 |
| | GCAGCCCCAGGCCAAGGCTGTGGCGTGCTTGGTGGTGCTGTAGGCCCAAGATGTTT | |
| | ATGGGTCGAGGGTCCCCGGGGCCGGGATTCTGATCCCTGGTGAGAGGTGGCTGGG | |
| | AGGAAGTCCAGACGTGTCCTGAGTGGCCATTCCTCACACTGAGGTGACACCGCCTC | |
| | TCCAAACACGTGACGTGGCTGGAAGCAGATGCTGCTGTCCG | |
| EFHD1 | CCTCGAGCCTGCGAGGAGCGCGCCGCCCGCCAGCTCCCTGCGTCCCGTCCCGCGTC | 268 |
| | CCCGCGTTCCCGCGTCCTGCGATCCGCCGCCATG | |
| RASSF2A | GAGGGCCAACGGCCCCCGCGCACCCTGCGCCCCTCTGAAGCGCGCCGCCTCCCCGC | 269 |
| | GCCGGGGACTGGGACCTGCCTCTGGGGAATCCGCCTAGAAGACGGCGGCGGAC | |
| VSX1 | GCGATGGTCTGTGACCCCTGCGCGGCTCAGAGCCTAGGGGACAGGGGCAGGAGCG | 270 |
| | GAAAGCGCGGGCCTGATTACCGGACGTGGAGACGCTGTCGCTGCGCTTCTGGCGG | |
| | CCGAGCGCAGGCGGCGGACGGCTGGGAGCCAGCGGGGCAGCGGGCTCGGGGCCC | |
| | CTGGGCGGCAGGAACGGCACGTCCGCTAGGAGCAGGCAGGGTGCTCGAGCGGCC | |
| | GCCGGCGGCTGCGTGCCG | |
| MAFB_TOP1 | GCGTGGCTGTGTGTCCCGAATTGGTGGGTTCTTGGTCTCACTGACTTCAAGAATGA | 271 |
| | AGCCGCGGACCCTTGCGGTGAGTGTTACAGTTCTTAAAGGCGGCGTGTCCGGAGTT | |
| | TGTTCCTTCTGATGTTCGGATGTGTTTGGAGTTTCTTCCTTCTGGTGGGTTCGTGGTC | |
| | TCGCTGGCTCAGGAAAGAAGCTGCAGACCTTCGCG | |
| SNAI1_UBE2V1 | GCGCGTCGCCAGGCTAACCCTGCGTGGAAAATTCGGAGGTGGAAGGCGAGGCGCC | 272 |
| | TTATTGAGGGGGCCGGCAGCGGCGGCGGCGGCGGCGAGGGGGCGGCGGGGGCT | |
| | GTGCGGCCCGGGCCGGAAACGTGAGCCGGGCTGGGGGCGGCGACCACCCCCG | |
| TFAP2C | CCGTACAGAGGGCGCGGAGGTTGCGCTCCAGTTCGAACGCTTACCCATTGGAAAGA | 273 |
| | GGGCAGCGCCGGGGTCCAGGGAAGCTCCTTGGGAATGAATGGCCTTTGCCAAGCG | |
| | GTTCCGGATCCTCTGGGTCCTTTGGGCCCACGGCACGGTGCTGCGCGAGCCCTCAG | |
| | TGCCCATCGGCTCCCTTCGCCTCCTGCGTAGACGCTCCCAGGCGGGGAGGCATATC | |
| | GGTTCCTCCG | |
| RBM38 | GCGGGAGCTGGGGGAGGGAGAGGTCAGAGGTCAAGGCTGCCGCGTGGAGCGTG | 274 |
| | GGCCGTGGAGTGGGGGAGGGGGCGGGCAGACTCCTCCCCGCCGGCAGCCAGGGC | |
| | AGAGGGCTGGAGGAAACGCGGAGAACTCCTCGGTGCTGGAGGAAACGAGGGGAA | |
| | CTCCTCGCCGGCCTTGCGGTCCCCCACAGCCCACGGAGTGCCACTCCCAGTCCCCAC | |
| | AGACCCCACCTGCGTCG | |
| GATA5_SLCO4A1 | CCGCCTGCAGTAACTGACAGGAAGGGGCGGGAGGCGGATGGGCCGTGACAGCTT | 275 |
| | AATGGCTTCGGTTAAAGCATCCTCTGATCGTGCTGGCGCTGGGAGAGGCTCTGAGC | |
| | TCGGGTGGCACTGCGGGCACTCTGGACACTGTCTCCGGCTGCCGCTGAGCTGGGA | |
| | GGCTCCTTTCCAGCAGGCCACGCGGTCAGGGGCACCTCCTGCCG | |
| SIM2 | CCGCGCCAGAAGGGAAAGACATAGGAGGTGTCCCAATCTGCGGTCACCGCCGATG | 276 |
| | CTCCTGACCACTCTAGTGAGCACCTGCCCGGTACTTTTCCATTCCAACAGAGCTTCCA | |
| | GCTTCATACTAACTATCCCACATACGGCCTGTGGGTATTAGCTCTAAGTGTCCTTTTC | |
| | CGAGGGCCCG | |
| SIM2 | ACGCATTAAATCCTCCCGAAGCCCAGGAGGTGCCAGAGCGGGCTCAGGGGGCCGC | 277 |
| | CTGCGGAAGCTGCGGCAGGGGCTGGGTCCGTAGCCTCTAACCCCTTGGAGCTCCTT | |
| | CTCCCAGAGGCCCGGAGCCGGCAGCTGTCAGCGCAGCCAGGAGCGGGATCCTGGG | |
| | CGCGGAGGTGGGTCCGACTCGCCAGGCTTGGGCATTGGAGACCCGCGCCGCTAGC | |
| | CCATGGCCCTCTGCTCAAGCCGCTGCAACAGGAAAGCGCTCCTGGATCCGAAACCC | |
| | CAAAGGAAAGCGCTGTTACTCTGTGCGTCCGGCTCGCGTGGCGTCGCGGTTTCGGA | |
| | GCACCAAGCCTGCGAGCCCTGGCCACGATGTGGACTCCG | |
| SIM2_HLCS | CCGCAGGCGCAGAGGGGACAATCCGGGAAGTGGTAAAGGGGACACCCGGGCACA | 278 |
| | GGGCCTGTGCTTTCGTTGCAGGCGAGGAAGTGGAGCGCGCGCTGCAGATTCAGCG | |
| | CGGGGCTAGAGGAGGGGACCTGGATCCCTGAACCCCGGGGCGGAAAGGGAGCCT | |
| | CCGGGCGGCTGTGGGTGCCGCGCTCCTCG | |
| C21orf33_ICOSLG | CCGCTGTGGTTGAACTCCTACTTACTCTTTCGGCAGATGGTGTTTGCCAAGTTAGTTT | 279 |
| | TGCAGCTGCCTGGGGGTACTGGGGTGGAAGCAGCCCCGGGAAACCCCATGGGGG | |
| | ACTTTGTGTCTTTTACTCCATCACAGCGAAGCCACGGGGCTGGGCCAGGCCCTGCCC | |
| | TTTGGGAACGGGCTCCTCCG | |
| TBX1_C22orf29 | CCGCCCCCCTGCAGGAGGGAGCACCAGCTCCGTAGAGGAGGGGCAGACGTGGACT | 280 |
| | GGTTCTTGTCAGGGCAGCAGAAAGGCCCTTGGTGCGCTTCTCCTAACACTCCCCTAT | |
| | CCTCCGCCGAGGTCGGGTGGCCCAGGCTGCAGGGCTCCAGCGGCTTGCTCACACCC | |
| | ACCTCCCTGCAGATCACGCAGCTCAAGATTGCCAGCAATCCCTTCGCGAAAGGCTTC | |
| | CGGGACTGTGACCCTGAGGACTGGTGAGTGTCCTCCCCCGAGAGAGTGAGCGCCG | |
| | GGCGCCTGGCGCAGGCGCCGCCCTGATCCGCCTCCCGCCCGCAGGCCCCGGAACCA | |
| | CCGGCCCGGCGCACTGCCGCTCATGAGCGCCTTCGCGCGCTCG | |
| TBX1_C22orf29 | CCGAGACCGCGTCGCCCGCGGCCCGGCCGGCAGTTGCAGTGTAGACAGCCCGAGA | 281 |
| | GCCCCGCCTGCAGGCGGTGTAGATACATGTAGATACTGTAGATACTGTAGATACCG | |
| | CCCCGGCGCCGACTTGATAAACGGTTTCGCCTCTTTTGGAAGCCGCCTGCGTGTCCA | |
| | TTTATTTGTGCCCAGTTAGATCGCGTTGGGAATCTTCGGGACAGCGAGCCCGGGGT | |
| | AGCTCAGGGCCCTCAGGGCCTCCCCAGCCCCAATCCCTGCCG | |
| RTN4R_DGCR6L | ACGGAGAGAGGAGGCAGCACCCACTGGGGCTCGGGCAACCATCCCGGCTACCCCC | 282 |
| | GCCCCGGCCCGCCAGGAGAGGAGGGAAGCCTTGAAGTGCCAGGCCTTTGAATCGC | |
| | CCATCTCCATGGCAACGCGTGGGCACAAAGGGCCGGGCCGGCGAGCAGGCGGCG | |
| | GCTGCG | |
| SCARF2 | CCGGACCAGAGGCCTGGGGGAAGGGGTCTCCGTAGGGACGGATGGGAGAGATAC | 283 |
| | AGAGGAAGTAGAATGGCCAGGCTGTGGACTGCGGTAGGAAGTAGAGGTAAAGAC | |
| | AGAAGGAGACCCCCGGGATGGAAACCCTGCAGTCCTAGTTGAGGAGTGAAGGGG | |
| | GCTGGGGGAGCCTGGGCGGTGGATTCTGCTGGCTGTCG | |
| PPIL2_SDF2L1 | CCGGGCCCTGGGCGGAAGGGATGTCTGCGTGAGTCAGCTGTGTCTGAGGAGGGGA | 284 |
| | TCCTGGGCTGGGCTGGGCGGCCCTACTCGGCGGGTCAGGCGGAGGGGCGCGGCC | |
| | GGGATCCCG | |
| SEZ6L_MYO18B | CCGGAAGTATGTCGTGCAGGGTTCAGTGTTCAGTCAAAGCCCTGTCATCATGGGGA | 285 |
| | CAAGGTAGTTTCCTTGGGAATTTCGATATACAGACTGTAGACCAGAAGTGTTCTCAG | |
| | AGTTCAGCCATGCCTCTGTACCG | |
| MN1 | GCGAGTCGACGGCTCCTTGGTTCGTCACCCTCCGTGGCTCCAGACTGTGGGAATCG | 286 |
| | GAGCCGCTGGAGGACGGCAGGCCGTGGAAGGAGGCGGCTCGGTTAGGGCTCTGG | |
| | TCCAGCGGCAGGCATGGGGCCGGCACGGCGTGGCTGGAGGCACCTGAACTGTGGA | |
| | AGTCCGGGAGGTTCCCCGGTCGCTGCGGGCCGAAGCTCTCAGGCCCCTGGCTCTCC | |
| | GCCATGTGCTCATAGCCCTCGGCGAAGGGCGGCTGGCTGCCCAGGCCTCCGGCTGC | |
| | GCCGCCGTAGCCGAGCAGGCG | |
| PPARA_WNT7B | CCGGCTCCTGTTCTCAGCCTGCCAGGCCCTTGTGCGGTGGCGTCGGGCAGGCAGGG | 287 |
| | CAGGGAGGCCACGGCAGCCATCTTCCCGGGGAGCTGGGGCCTGGCCAGCAGCGTT | |
| | TCCCAGTGGCCTCCTCCTGTGCTCCGAGCTGCATTACCTCATCGGGAAGCCATTCCA | |
| | GAAAGGAGCTGCGGAGCCCCTGGGAGTGGGAGTGGGGAGAGCTGCGTCAGCGCC | |
| | CTCCTGGCAGCCTCGGTGCCAGACGAGGGCAGGCGTCACGCCTCCGGGTGTCTGCC | |
| | TGCCGAGCGACTGCTGGGAGAGCAGCTGGCTTTTGTCAGCGTTTCGGGGTGACCG | |
| | GGCTGGGCTGCAGCAGGCAGGTGGCGTGGCACGGCCCATGGCCGGCCAGCTACCA | |
| | GGTGGGCAGAGGATCTATTTCAAGAGCCG | |
| CELSR1_TRMU | CCGCTGCCAGGGGCCCCAGACCCCATCTACCCACCTATCCCCTTCCTCAACAGGTTCT | 288 |
| | GCTATCGGGTTTCAAAATGTGCAGGCACAGGCACCAGCCCAAACCCAAGGGGACCC | |
| | TTCAGCAGCGACACTGGGGCCAGCGTGGGGTTCTGGCACGCCCAGCAACATGGGC | |
| | CCGCTCCAGGCGTGGCCAGCACCG | |
| TYMP_SYCE3 | ACGTGCTGGCCTTTGCCCAGCAGCACGGAGAGCCCGGCCTGGCGCAGGAGACCTA | 289 |
| | CGCGCTGATGAGCGACAACCTGCTGCGAGTGCTGGGAGACCCGTGCCTCTACCGCC | |
| | GGCTGAGCGCGGCCGACCGCGAGCGCATCCTCAGCCTGCGGACCGGCCGGGGCCG | |
| | GGCGGTGCTGGGCGTCCTCGTACTGCCCAGCCTCTACCAGGGGGGCCGCTCAGGG | |
| | CTCCCCAGGGGCCCTCG | |
| CPT1B | TCGGCACCTAGGACGGGGGCAGATGGGTGCGCGGGCGCGCTTAGGCCGGCCCCGC | 290 |
| | CGCCAGCCGCGCCGAGACGCCCCCAGCCAGTCCGCGACCCCTCGCGCCCCCCACCC | |
| | CGCGACTAGCGGCTGCCCCCGGCCCGCGCCCCCCGCCAGGCCAACCGCCGCCAAAT | |
| | CCTCGCGCCAGCCTTCCGGGTGGGCACAGCCACTGTGGTGCAGGGGATTTGGGCCT | |
| | TGAAAGCTCCAGGAGCCCCAAGGACGGCG | |
| RAD18_SRGAP3 | CCGGGGTGGCTGAAAGCGGGCTCCTAAGCCATCTCTTCGGATTCCTTCTTCGCAGAC | 291 |
| | GCGAGCAAGCTCCTGGCACCCTGTAGTCTCTCCCTCTCCCCTTCCTGTATTCGGCCAA | |
| | CGACCGACATCAGGCCATTCTTTATTAACCTTTATCAAGCCAGGCCGGTCAGCG | |
| ITIH3 | ACGCCAGGGAGTCCCAGGGTCCATTTGTTGGCCCACAGCTTCTGCTTTCCTGCGGGC | 292 |
| | CTCTTCCGAGTCCCCCGGTCTCTCAGGAATGAAGCCCCAACGCTGTTGGCAAGACCC | |
| | ACCAGGCCTTCTTCAGACAGACACATCGAGGGACCCGTCATTCCACCCATGCCCAGC | |
| | TTCCCG | |
| FEZF2_PTPRG | GCGACGCTTGGCTAGGCGGGCGCGACCTCTTCGAGTGAAGAAGTTGTCAAACTTCG | 293 |
| | TAAGCGTCAAGCCGGGTGCTCTCCCGACAAGACCGAGACTGAGTCCCGCGGAGCC | |
| | GCTCTGCGCTCCTGCTCTGCCCGCCACAGAGGCTGGTGCAGCTTCCCTCCCGCCGCG | |
| | CTCCGCGGGCCGGGAAACTTTTGCGTAGCCCAGAGACGCACCGAGTCCTTCTCCTG | |
| | GCTGATGCCTCGCTAGAAGAAATTCGCACG | |
| HEG1_SLC12A8 | ACGCCGCTGGGGGCTGCTGAAATTAGAAGAGGGAGTCGGGAAGTCATACCCCTCC | 294 |
| | CTGTGGGCGTCGGGTTGCACTGTTGACTAACTTAGAAAGCGAGATTTCTAAAAATG | |
| | ATGCTGGGGCTGCAGGCTGCGGGCTGCGGGCTGCGGGCTGCGGGCTGCTGCCGCG | |
| | GCGGGGGCTTCCGGCGGCGCTCTCTTCTGGGTCCCCCACCCCTGGACCAGCGACCG | |
| | ACGACCAGCCAGACAGCCCTTTCCTGCGAATGGACAATGGGAGAGGCTGGCGCAA | |
| | CCGAGAATAGCCAGCGCGGAGGAAGGGCTCCGGACGGAGCTAGGAGGGTGGGGC | |
| | TCGGAGGGCGCAGGAAGAGCGGCTCTGCGAGGAAAGGGAAAGGAGAGGCCGCTT | |
| | CTGGGAAGGGACCCGCACGACGACGCCCGAAGGGCGTCGGGGGAAGTGGTAGGC | |
| | CCCGGAGACTGCGCGAGGCTCCTCAGCAAAGGAAGTGGGCGCGGCGCGCACGCAA | |
| | GACCTCGCACCCGGCCTCGCGCGCCGCCTCTGGACAGCCCAGCGCCTCTCAGCACCT | |
| | GTACCTCGCCAGACGCG | |
| TRH | GCGGGGCCGGCTGCCGTCAGCGCCCCTTCCCGGCGGCCGCGACCCCTCCCCGCTGA | 295 |
| | CCTCACTCGAGCCGCCGCCTGGCGCAGATATAAGCGGCGGCCCATCTGAAGAGGG | |
| | CTCGGCAGGCG | |
| SOX14 | GCGCAAGCCCAAGAACCTGCTCAAGAAGGACAGGTATGTCTTCCCCTTGCCCTACCT | 296 |
| | GGGCGACACGGACCCGCTCAAGGCGGCTGGCCTGCCCGTGGGGGCCTCCGACGGC | |
| | CTCCTGAGCGCGCCCGAGAAAGCCCGGGCCTTCTTGCCGCCGGCCTCGGCGCCCTA | |
| | CTCCCTGCTGGACCCCGCGCAGTTTAGCTCGAGCGCCATCCAGAAGATGGGCGAAG | |
| | TGCCCCACACCTTGGCTACCGGCGCTCTGCCCTACGCGTCCACCCTGGGCTACCAGA | |
| | ACGGCGCCTTCG | |
| PIK3CB_FOXL2 | TCGCGCCCCAAGACCTGGGCTTGCAGCGCCGCCAACAGGCCCGGGGACACGAGGC | 297 |
| | GCTCCAGGCCGGGGTCTTCCCGGCTGCTGGCCCCTCTCGCTCCCCACCCGCTGGCG | |
| | GCGCCTCGGTCGCCCGCAATTGACCCAACCCGCTTCCTGCGTTTGCCCCTCAGGTTT | |
| | CCCGTTTCTCCACAAAGGCCTAGGGGAGCCTCG | |
| PIK3CB_FOXL2 | CCGCTTTGGGGGAAGCGAGAGGGAGGTTGGAGGAGCCCCGGGCGGGGTCTCAGC | 298 |
| | GCCCACCAGCTGTGCCTTCAGGGCTTGGGTGTTCGCTGCAACGGCAACCGCGTGAG | |
| | CCTCACTCCCACGGCCAAGGGGCTAGGGCAGGGTGGATGCAATCGCGTGCGCCTG | |
| | GCCCCGGAAGGTGCTCG | |
| PLSCR1_ZIC4 | CCGCACTGACTTGCGATGTCGACCGGTCTGCCCAGACCACCCCCACCTGGCTGTCGG | 299 |
| | GCCTCTCGGTCCTAAGACGAGGGGTTGGCGCGGTAGGGTCCGCACAGGCCAAATG | |
| | GGATCCGAGGTGTCTACCGCAACCACGCCCTTGAGCGCTGCGGCTTCGGGAAGAAA | |
| | ACAGCTGCTGCTGTCAGGCCAGGCCTGGCTCCGCAGCCCGGAGGGCCACCAGGCG | |
| | GCTGGCATAGGCCGGGGAGGGGCTGGGATCGGTGGCTGCGATGCCCTGTAGAGC | |
| | CGAGGGAAGGCGCGAGTGCACGTTAGAGTGACAATATTGGCCGGACCGAGCCCCA | |
| | ATCGGGGAGCTCACGGCCAGCTGAATTCGCTGACGTGTAGGAGAGGAAAGGACCC | |
| | CGAGAACCCGGAAGCCTAGATTCCTGCCGGAGCTGCAAGTGCTGCGGAAATGGGG | |
| | GAAGAAGGTTTCTGGGCGCTTTAAACAAATGGCTGCCTCCCAGCGCTCTGAGTTAA | |
| | GGGACCG | |
| VEPH1_SHOX2 | GCGGCCTCTGTCCTCCGTTAGTCTTGGGGGAGCAGACGCAAGAGGAGGCAAGGGC | 300 |
| | GCCGCGAGCTCCCCGGATGCACTGGTCCCACAGGCCGTGCCCGAGTGGAGCACTGC | |
| | GAATGGGGCCAAGAAATTTTGGCCTTTCTCGCCGGACCTGGCTGCCTCCGCGGGCC | |
| | TCTCCGCCTACCGCGCTCCCGCCGCGGCCCGACTCCCGCGGGTCTCCGCGCCGAACC | |
| | CACCTGGCTCCTATCGCACGGGACATTCCCGACCCACCCACGCCGCGTCACTGAGCC | |
| | TCTGTACCGATACCCGGCGCCTCCGCCAGCAGGGCCTGGACGCACCGCCTCCTTTGA | |
| | CCTCGGGCTTCCCCCGCGCTCCG | |
| SLC2A2_TNIK | CCGACCTCCGACCGATTCGCAGCACCCCACCCCCAGTCGGGGCCATCCATCCACCTG | 301 |
| | ATTAACTCGCCGGCAGCAACTCCCAGCGTAGAAAGTAGGGCAAATGAACACACACA | |
| | GTCGGTAAAGCAGGAAGCCACAGACCTGGCCAATGCACCCACCCTGTTACCAACCC | |
| | CACCCCGCTGCGCAGGGGGCAGCCG | |
| SLC2A2_TNIK | ACG | 302 |
| TPRG1_LPP | ACGTGTGTAGAGGCTGAAGGAGAGCTGTGTTGCTAGCTTTGTATTTGAACGGTTCG | 303 |
| | TACACAAACAGTTCTCTTTGATTAAGTATCCG | |
| FGF12 | CCGGGCTTCTACTGACCTGGTCTCCGCCTCACCGGCCTCTTGCGGCCGCTGCAGAAG | 304 |
| | CGCACTTTGCTGAACACCCCGAGGACGTGCCTCTCGCACAGGGAGCGCCCGTCTTT | |
| | GCTGGGGCTGGAGCGGCGCTTGGAGGCCGACACTCGGTCGCTGTTGGACTCCCTC | |
| | GCCTGCCGCTTCTGCCGGATCAAGGAGCTGGCTATCGCCGCAGCCATAGCTGCTCA | |
| | GCGAGGGCCTCAGGCCCCAGCCTCTACTGCGCCCTCCGGCTTGCGCTCCGCCGGGG | |
| | CGAGGGCAGGACCTGGGCGGCCAGGGAAAGGGCAGTCGCGGGGAGGCAGTGCTA | |
| | AAATTTGAGGAGGCTGCAGTATCGAAAACCCGGCGCTCACAAGGTTAGTCAAAGTC | |
| | TGGGCAGTGGCGACAAAATGTGTGAAAATCCAGATGTAAACTTCCCCAACCTCTGG | |
| | CGGCCGGGGGGCGGGGCGGGGCGGTCCCAGGCCCTCTTGCGAAGTAGACG | |
| NRROS_CEP19 | ACGTGCCAGTTGGTGGCTGCGACTGGAGGAGGCCGGATCGGGGGTCCTAGGAATG | 305 |
| | GAGCCTCTCCGGACAGGGCTGGTCGGGGCTGCTGTGCTTCCCTAGGGGCTGAGGG | |
| | GACCCCACCGGAGGCTTCTTCATGATGGGCACAGCCCGTTAGGAGTCTGGGTGCTA | |
| | GAAACATTCAGCGTCTGTGGCCCTCCATGCTTTCCTGTGTGCTCCTCACCTGCCG | |
| NRROS_CEP19 | ACGCTTCACATTCGGGAGCACGAGCCCCCCGGAGCGCTCACCGAGCTGGACCTGAG | 306 |
| | CCACAACCAGCTGTCGGAGCTGCACCTGGCTCCGGGGCTGGCCAGCTGCCTGGGCA | |
| | GCCTGCGCTTGTTCAACCTGAGCTCCAACCAGCTCCTGGGCGTCCCCCCTGGCCTCT | |
| | TCGCCAATGCTAGGAACATCACTACACTTGACATGAGCCACAATCAGATCTCACTTT | |
| | GTCCCCTGCCAGCTGCCTCGGACCG | |
| RASSF1A | GCACCACGTGTGCGTGGCGGGCCCCGCGGGCTGGAAGCGGTGGCCACGGCCAGG | 307 |
| | GACCAGCTGCCGTGTGGGGTTGCACGCGGTGCCCCGCGCGATGCGCAGCGCGTTG | |
| | GCACGCTCCAGCCGGGTGCGGCCCTTCCCAGCGCGCCCAGCGGGTGCCAGC | |
| RGS12 | CCGTGTCGGGGAGGAGCTGGGACCCGGGAAATGGCAGGTGTCCTCTGAGGGGAA | 308 |
| | CCGGGCGGGAGAGGAGCTGGGGCCTGGAAGGCCAAGGCAAGGGCTGTCTCCAGT | |
| | CCACG | |
| GPR78 | TCGCTCCAGTTTGGTGCCAGCGCCTGGAGGGAGAGGCGTGGCGAGGGCTGTGCTG | 309 |
| | CCTAGGATCCACTGAGTGGCTCTTGCTGGCGTGTCAGCTGCGCGCGAACCAGGGCT | |
| | GGGAGGCTCGGCTGGAGGTGTGACCAGGGCAGGGACTGACCTGGCCCGGAACAG | |
| | AAGCGCGCAGAGTCCCATCCTGCCACGCCACGAGGAGAGAAGAAGGAAAGATACA | |
| | GTGTTAGGAAAGAGACCTCCCTCGCCCCTACGCCCCGCGCCCCTGCGCCTCGCTTCA | |
| | GCCTCAGGACAGTCCTGCCGGGACGGTGAGCGCATTCAGCACCCTGGACAGCACC | |
| | GCGGTTGCGCTGCCTCCAGGGCGGCCCCG | |
| HMX1_CPZ | CCGACCGCCCCCAAGCCGGTCGAGGCCCCCGTCCATTTGGGGGAAATGGATTTTCG | 310 |
| | CGATTTAAGAAACAAACCCAAATCAAATGAGCGAGGCCCGGATGTGCTGACGCTGC | |
| | GGTTACGCGCGCGGAGCTGGAGCCCCGAGAGCGCTCTAGGAAAGGCGCAGCGGC | |
| | GACCGCGGGAGGGGGTGAGAAGCCG | |
| HMX1_CPZ | TCGGGAAAGGGGGGTAGGGAACGACGGGGGAGCCTCGGTGACCAGGGCAGATGC | 311 |
| | ACGCGCGCGCGGGATCCTCGTGCGCCGCGAAGAGGGACGAGCAGAGGAGCATCG | |
| | GAAGAAGACAGGCGAAGGGGACCGCGGAGCAGCGTAGGCGGAGCCCCGGGGGC | |
| | ACGGCCGAGGCTGCGCTTCAGGAGTGTCCGCCAGGCGCCTTCCCGGGCGGTTGGC | |
| | GAAACCCGAGGAGGCCCACAGCTCTGGCCTGGGGCGCCGTCGTTCCAGGGGCCTC | |
| | TGCG | |
| RAB28_NKX3-2 | GCGGGGCGCCCCGTGCAGGCTACAGCCTACAGCTGTCAGCGCCGGTCCGGAGCCG | 312 |
| | GAGCGCGGGAATCACTCGCTGCCTCAGCCCAAGCGGGTTCACTGGGTGCCTGCGGC | |
| | AGCTGCGCAGGTGGAGAGCGCCCAGCCTGGGAGGCAGTAGTACGGGTAATAGTA | |
| | GGAGGGCTGCAGTGGCAGAAGCGAGGGTGGCCGCAGCACTTCGCCGGGCAGGTA | |
| | TTGTCTCTGGTCGTCGCGCACCAGCACCTTTACGGCCACCTTCTTGGCGGCGGGCGC | |
| | CGAGGCCAGCAGGTCGGCTGCCATCTGCCGGCGCTTTGTCTTGTAGCGACGGTTCT | |
| | GGAACCAGATTTTCACCTGCGTCTCG | |
| SOD3_LGI2 | TCGTGGGCCGGGCCGTGGTCGTCCACGCTGGCGAGGACGACCTGGGCCGCGGCGG | 313 |
| | CAACCAGGCCAGCGTGGAGAACGGGAACGCGGGCCGGCGGCTGGCCTGCTGCGT | |
| | GGTGGGCGTGTGCGGGCCCGGGCTCTGGGAGCGCCAGGCGCGGGAGCACTCAGA | |
| | GCGCAAGAAGCGGCGGCGCGAGAGCGAGTGCAAGGCCGCCTGAGCGCGGCCCCC | |
| | ACCCGGCGGCGGCCAGGGACCCCCGAGGCCCCCCTCTGCCTTTGAGCTTCTCCTCTG | |
| | CTCCAACAGACACCCTCCACTCTGAGGTCTCACCTTCGCCTTTGCTGAAGTCTCCCCG | |
| | CAGCCCTCTCCACCCAGAGGTCTCCCTATACCGAGACCCACCATCCTTCCATCCTGAG | |
| | GACCGCCCCAACCCTCG | |
| KLF3_TLR10 | GCGTACTGAGACAGGGTGGGCAGCAGGGGCCAGTTGGAAGGAGTGGAAACTGTC | 314 |
| | ACTAATGTAAACAGACTGTCCCCACGTTCTGTCTTCTCCG | |
| KLF3_TLR10 | ACG | 315 |
| KCTD8 | GCGGCGGCTCAGCAGGGGGCGAGGGGTGCTGGGAAACGCCGGGGCTGCGAACTT | 316 |
| | ACGGAAGAAAATGTACTCGGTGTAGCTGCTCCAGATCTTGTCGTCGCGGTACTGGT | |
| | TGACGAAGGCGGCGGTGCCCGAGGAGTTACACGCCACCATGTGGAAGCCGGCCTC | |
| | GGACAGGCGATCAAAGGCCTGCTCCAAGTAGGTGAACTTGAGGTAGAAGCGGGAC | |
| | GTGTACTTCTCCGGCTGCCGGTCGGGGTCGCGGCTCTCGTTGAGCGTGTCCCCG | |
| HOPX_ARL9 | TCGGCTGCCGCTGCCGTCAGCTGAAATGTTAGCTATCTACCGTCTTATAAAACGCCA | 317 |
| | GGAAAAACCTCTAAACCTTAGAGCCGGGGAATTTTTTAAAAAATCGGAACCAAATC | |
| | TCCGTGGCTTCGTGCAGCGTGAGTTCTGCAGCTCGGGGGACGCTGCAGTGTGATGT | |
| | GGTGGAGAGAGCATGCTTCACCGCTCCTGCCATCCTGACAGCGCCCTCCCTCCCGGC | |
| | CTCAGCCTCCTGGTTCGCCAAACCGGAGGACTGAATTTATGGCTAGCTGGTCTCTGG | |
| | GGCGCCTTCCAGCTCTGACATTCCCGCCTAGAATAGATCTTCCCGAAGGTTTCGCAG | |
| | ACAGACCAGAGGGGACCGAGCCGGGAAGGCGAGACAGGGACAGGCGAGAGACG | |
| | CTGCTCCCAACTCGCAGAGGGAGAAAGCGTGTATCCCGGGCTGCCGGGGAGAGTG | |
| | GAAAAGAAAGGACTGGTGACCGAGGGGTTTCTGCGCAGCTCCCGGGGAACCACGG | |
| | CTGGATGGGGGTGGCGGGGAGACCGGGCGCCCATGGGAGCGGGGAAGCGGGGA | |
| | GGCGGCGGCGGGAGCCATGCAGGGTCTGGGCCCCTGGGATGCGGGCAGAAGCGA | |
| | TGGGAGATCATGGGGAGGGCAGCCCGGCGGGAGGCGCGGACGAACAGGACCGCC | |
| | CAGCCGCGAGAAGGCTCAGCCCAGGCAGGGGTCGGGGCGCGCTGGGCGCGTGTG | |
| | GGGACG | |
| CXCL5 | TCGAAGGACCGGGGACACGGGCCGCGCGGCTGGACAGGAGGCTCATAGTGGTCA | 318 |
| | AGAGAGCGCTGCGAGCGGTCGCGGGTTCCTGAACTGGGTGGAGGAGCGGAGATT | |
| | GGAGGAGCGAAGATTGGAGGATCCGGAGCACTGTGGCTTCCTCG | |
| SMARCAD1_ATOH1 | GCGGAGCGTCTGGAGCGGAGCACGCGCTGTCAGCTGGTGAGCGCACTCTCCTTTCA | 319 |
| | GGCAGCTCCCCGGGGAGCTGTGCGGCCACATTTAACACCATCATCACCCCTCCCCG | |
| | GCCTCCTCAACCTCGGCCTCCTCCTCGTCGACAGCCTTCCTTGGCCCCCCACCAGCAG | |
| | AGCTCACAGTAGCGAGCGTCTCTCGCCGTCTCCCGCACTCGGCCG | |
| PITX2_ENPEP | ACGCGCCCAAGAATTGGGCTGCCACTGGTATGGGTCCCAAGTCACATTCAATAAGC | 320 |
| | TGCCCACCGCTTTCTGGGGGACAGCAGTGGTGGTTCTAGGTCTCATCTTTCCAGAGC | |
| | GACGAGGATAAAAGTTCCTGCCCAGGACTGTGTGCGAGGGGGTCCCGCACTGCTG | |
| | CAAACTCTCAGCGGAGGCAGAGAGGCTTTGCTGTTTCTGGAGAGAGGAAGCATTG | |
| | GCAGAGGCAGTCTCCGGGCTGTGAGGAATCCACCCTCATGCCTTAGTGTGGGTACG | |
| | TCAGGTCCCAGCATCAGCG | |
| MGST2_MAML3 | CCGTGCGTCCCCGGCAGGACCTAGACTGCCTCTCGGCGCAGGCGGCCCTAACAAAG | 321 |
| | AAGCCCACGAGGCGGTCCCGGGCGCGGGCAGGGGCGGTGCGGCGGCGCTCGGGA | |
| | GACCCGCGAGGGGCCCTGGAGGTCCTCGGCCCGCGCGCG | |
| POU4F2 | GCGCGGGGGTAGGCGCGGGGAGAGGGGAGTATAACTCGCCGGCCGCGAGGAGC | 322 |
| | GGGGGCAGTTTCGGGTGCCGAGGTCTGCAGCTAGCGGCAAGCGGAGTCAGGCATC | |
| | CGTTCAGACTGACAGCAGAGGCGGCGAAGGAGCGCGTAGCCGAGATCAGGCGTAC | |
| | AGAGTCCGGAGGCGGCGGCGGGTGAGCTCAACTTCGCACAGCCCTTCCCAGCTCCA | |
| | GCCCCGGCTGGCCCGGCACTTCTCGGAGGGTCCCGGCAGCCGGGACCAGTGAGTG | |
| | CCTCTACGGACCAGCGCCCCGGCGGGCGGGAAGATGATGATGATGTCCCTGAACA | |
| | GCAAGCAGGCGTTTAGCATGCCGCACGGCGGCAGCCTGCACGTGGAGCCCAAGTA | |
| | CTCGGCACTGCACAGCACCTCGCCGGGCTCCTCGGCTCCCATCGCGCCCTCGGCCAG | |
| | CTCCCCCAGCAGCTCGAGCAACGCTGGTGGTGGCGGCGGCGGCGGCGGCGGCGGC | |
| | GGCGGCG | |
| SFRP2 | TCGGTGGCTGGCAGGAGGTGGTCGCTGCTAGCGAGGGGGATGCAAAGGTCGTTGT | 323 |
| | CCTGGGGGAAACGGTCGCACTCAAGCATGTCGGGCCAGGGGAAGCCGAAGGCGG | |
| | ACATGACCGGGGCGCAGCGGTCCTTCACCTGCACGCAGAGCGAGTGGCATGGCTG | |
| | GATGGTCTCGTCTAGGTCATCGAGGCAGACGGGGGCGAAGAGCGAGCACAGGAA | |
| | CTTCTTGGTGTCCGGGTGGCACTGCTTCATGACCAGCGGGATCCAAGCGCCGGCCT | |
| | GCTCCAGCACCTCCTTCATGGTCTCG | |
| LRAT | GCGGACAAAGTTTCGGTGGGTGAACTGAAGCTGGGTCCATGTGACCCTGAAGCCG | 324 |
| | GAGAAATAAACTTAACATGAATCTTGCTTTCCTGGCGGGCGTTGGGACCCCGCCGTT | |
| | TTTCATGCCAACCGTTGGAAGCTTCGTACTCAACGGCCACAGGTGCCTAGGAGCGC | |
| | AGAGAGGCCTCGGGTTCAAATCACCGGCGCGCAGGGACTGGACTCGCGGGTAGCG | |
| GRIA2 | ACGTAAGACAGCAGGGCCTGGTGAGAGGACGCTTCGCCGCCAACAATTAGCAATTC | 325 |
| | GGCTTCTACACAGCAGCCGGAGATCAGCTTTGCTGCATTTGGTCCAGGTTGGAGCA | |
| | TCTCCGCAGCAGCTGCAACAGCCGCACGAAGGTAGCTCCGGGCGGGGAGCGAGGC | |
| | GCTGTCCTCGGTGCTGAAAGGCCGAGGCGCGCGGTGGGCGCGACAGCCCCGGAGA | |
| | CCCGAGGTCTCGCGGAGGGACAGCGGCTACGGGCCCCGAGCTGTGCTTTCTCAGC | |
| | GCCGCGCACGCGACGCGTCCACGGTGGTGCGGGGTGCCGGGCG | |
| FRG2_FRG1 | TCGGGCGCCTCAGCGGTCCTGCGCGTGGTCTGGCCGCCGGCGATAGCGGGACGCT | 326 |
| | CTGCGAGGCCGGCGGAAAACGCAGCGCGGCGACTGGTGCTTGGGCGTATAGAGG | |
| | GGGAGAGCAGCCCGGCCGCGGGCGAGCGGCTCCGGGGGTGCCTGATCCCAGCCTC | |
| | GCGGCCCCGGGTTGGTGGTGACGCCTGGAATCAGACGCGCG | |
| BMP3 | GTTCAACCCTCGGCTCCGCCGCCGGCTCCTTGCGCCTTCGGAGTGTCCCGCAGCGAC | 327 |
| | GCCGGGAG | |
| SFRP2 | GCCGCCGCTCGCCCGCCCTAGGATTTCTTTAAACAACAAACAGAGAAGCCTGGCCG | 328 |
| | CTGCGCCCCCACAGTGAGCGAGCAGGGCGCGGGCTGCGGGAGTGGGGGGCACGC | |
| | AGGGCACCCCGCG | |
| PLAC8 | GGAGAGAATCTCACCACAAATGAAAACTACGTGAAAGGGGAGAGGTAACTGTGTT | 329 |
| | TCTATCGCAGGGCATAGTACATAGAACAGTTTCAGACGCTCTTATTGGCCAGAGTAA | |
| | TCCAGCAGAA | |
| FGF5 | GCGTTATAAATATCCCGGTGCCAGCGCGGAGATCCGCTCGGGTGGCCTCTCTCTTCC | 330 |
| | CCTCTCCCCTTCTCTTCCCCGAGGCTATGTCCACCCGGTGCGGCGAGGCGGGCAGA | |
| | GCCAGAGGCACGCAGCC | |
| IRX4_NDUFS6 | TCGCTCGCCAGGCCGGGGGCTCCCGCCGCAGCCTTTTGACAGGCACATGAGCCGCG | 331 |
| | AGCTTCCGAACCTCGATAATATCATCTCGAGCGCGAAAGTCAATACGGTGACAGCG | |
| | CGCGGCCGGATACAATCCAATTACGCTCGGCTGCCCGGGCGCTCCTGGGGCTCGGG | |
| | GTCCGGCGGCCGAGGGTCCCCCTCAGGGCCCG | |
| IRX4_NDUFS6 | CCGGTCAGGCTCAGGCCCAGGCGGTGGAGGCCCCGGCGTGGCAGCGCCGGGCTTG | 332 |
| | TCCATGTTCCCAGGAGTCCAAGTTCAGAAGCCCCCTCTCCGGTGGGTTGGCGGCTTC | |
| | GCGGTGGCCGCGCTAGTCTTCCTCTGGAAACTCAGTGAAAAGAGTCGGCGCCGTCC | |
| | GCCTGAGCGCGGGTTCCCTCCTGGGCTCGGGACCCGCCCGCCTCAGGCGCAGAAG | |
| | GGTTTGCCGCCGGCCTTGGGCAGGGCGAGCAGCTCCCTGGCGGCGCCTGCAGCTG | |
| | GGGCGTCCTGGGGCACGGCAGGCGGAAAGGCGCGGGCCAGGGGTGCAGTCAGCA | |
| | CGTTCGCGCCCGCCCCCAGCGAGCGTCCCAGAGGCCCGGGGTCCAGGAGGGCGCC | |
| | CTTGGCGGTGGCCCAGGCCTGGTTCAAAGTGCTGTGCCTGAGGATGGGGTCGTGG | |
| | AAGACCCCGTCCACCCAGTTTCTGAGACTGGTTACCGGGGAGTCCTGGTGCCTGTCC | |
| | AGGGCG | |
| IRX4_IRX2 | CCGAGTGAGCAGCTGGAAGCCCGGGGTTAAGTGTTATTGACTTCAGAGCAGCAGC | 333 |
| | AGCGTGATCGGGTTTCAAGCTAGTTCCCATTGAATTAATTTTTGTGGATCGGTGTTT | |
| | GAAGTTTGGGTGGGAATAATTGGCCTGGGAGAGACTCCTTGCATCCTTGCCGGGTA | |
| | ATGAAGCTGGAGGCAGGCGTGCG | |
| ADAMTS16 | ACGCTGCCGGCCGGGGACCCTCCGGTGGCCCCTAGCCCCTCGGAGCGCTCCTGGAT | 334 |
| | GAAGCCCCGCGCGCGCGGATGGCGGGGCTTGGCGGCGCTGTGGATGCTGTTGGCG | |
| | CAGGTGGCCGAGCAGGTGAGTCCCGGGCGCTCCCACCAGCGCGGAAACCGCGGGT | |
| | CCGGACAGCTGGAGGCG | |
| 11-Mar | CCGGAGAAGCGAGGGGGCGGGAGGGAGGAGCGGCGCGGCGGGGGTGACGGGG | 335 |
| | CGCGGGCGCGGGGTGGGCTGGGGGCGCGGATCAGTGGGACGGAGTTCGGGGTTC | |
| | GGCTCCGAGCGGGCGGGCTGGAAGTGGGGGATCCCTCAGCCGCCTCCACGGGCCG | |
| | GCCCCGCGCTCACGTCGGTTCCGGGGCGGATGACCCCTCTCCAAACGGCGCAGCGC | |
| | TGCGGCTCTCGTGAGCTGGGAAGTAGGGGGCAGGGGAGAGGCCGCGGGTCCAGA | |
| | AACCGTTACTGGATGGGCCGGTGGGATGTGGCGCGGGCCGGGTGGGGCGCGACA | |
| | GTCTGAGCCGAGACCCGCGTGGGCTTAAGGGTGCGCGAGGCGGGTGCCCTGGGC | |
| | GCGCCCGAACTGGCTGAGCAGTGGAGCGGGAAAGGGCGCGGGACCCGGGACTGT | |
| | AACCGCCACTTCCAGGCCCTCGCTCCCCGCGCTTGGAGCCCTCAAGGGCACTCTCAG | |
| | GGATCCTCG | |
| PTGER4_PRKAA1 | GCGGTGATGTTCATCTTCGGGGTGGTGGGCAACCTGGTGGCCATCGTGGTGCTGTG | 336 |
| | CAAGTCGCGCAAGGAGCAGAAGGAGACGACCTTCTACACGCTGGTATGTGGGCTG | |
| | GCTGTCACCGACCTGTTGGGCACTTTGTTGGTGAGCCCGGTGACCATCGCCACGTA | |
| | CATGAAGGGCCAATGGCCCG | |
| TMEM174_FOXD1 | CCGGGCACGGAGGTTTAATGTGAAGCATGTGAGCGGGGCTCAGTTTACAGGTACG | 337 |
| | CGGGCCGATGGCGAAGAGCGCTGTCAAGCGGCCTCGAGGATTTCGGGGGGTTTGC | |
| | GCCGCCGAGGAAACCCTACCCGGACGAGGCGAGCAGCCTGGTGGCCCTGGCGGCC | |
| | GCGAGCTCCCGGCTGCCACCGCTAGGCG | |
| FOXD1_TMEM174 | CCGGGCCGGCGCGGGAGCGGCCGGGCGCAGCTGACCACGGGTACAGATAGGTTA | 338 |
| | ATTTCCACATGGAGCTGCAGAAACCCTATCCGCGGGTTGCGAAGCGTGGGTCAGCC | |
| | AAGGCATGTTAATCTGTTTAGCATGTGCGCCGCGCGGAGGAGCCAGACCACCGGG | |
| | GCGCAGGAGGCGCGGCCGCAGCCGGCG | |
| AGGF1_CRHBP | CCGGACTGACCTATGTTTCTTGCCAGCTGAGGGAAGCGGCGGACTACGATCCTTTCC | 339 |
| | TGCTCTTCAGCGCCAACCTGAAGCGGGAGCTGGCTGGGGAGCAGCCGTACCGCCG | |
| | CGCTCTGCGTGAGTCGAGGCTGCCCGGCTCGCGGGCGCCCGGGACGCGGGGAAG | |
| | GTGGGACTCTGTGCGGGGGGCAGAGGGCTCGCGGACATCTCGGGGAAGGGGCTG | |
| | GCCGGAACCGCCAGGGGCGCGGTCCCCTTAGCTAAGGATCGGTCCGCGGAGGCGC | |
| | GCCAGGAGCGGGAGAGGGTGGCGCGCCCGGGGCGCAGGAACCCAGCGCAGCCTA | |
| | GGCTGGAAGTCGGGGCGCTGGGCACTACAGAGCCCGGGAATGGGGCGCGCGGAG | |
| | AGCGGCCGCCCGAGGACGGCGCTGCGGCG | |
| PITX1 | TCGGGGTCCGGGGCGAAGAGAGCCAGGGCGCGGACCGACGTCTGCTGCTTTTCTG | 340 |
| | CGGCATTGCTGCCCGAACGAACGAACGAACGAACGAACGAAGCGGTTTCGTTTAG | |
| | GAAAAATACCCTCTTGACGCGAAGCCACGGCTGAAGTCCCGGGCCACGCAGAGGG | |
| | GCCAGCAATTCCATGGGTGGTGGGGCCCTCCATCCCTGGACG | |
| PCDHGA11 | ACGCTGCGGGGGTTCCGGGCCAGGCAGATCCGATATTCGGTGCCAGAAGAGACCG | 341 |
| | AAAAGGGCTCCTTCGTGGGCAATATCTCCAAGGACCTGGGGCTGGAGCCCCGGGA | |
| | GCTGGCGAAGCGCGGAGTCCGCATCGTCTCCAGAGGGAAGACACAGCTTTTCGCTG | |
| | TGAATCCGCGAAGCGGCAGCTTGATCACGGCAGGCAGGATAGACCGGGAGGAGCT | |
| | CTGTGAGACG | |
| PCDHGC5_DIAPH1 | GCGGCGTGTCAGTGTGCAGTGGAGTGTGCAGTCTAAGCTTGCGGCTGTCTCCAGGC | 342 |
| | AGAAGAGGAGACCCCGGCGCGGGCGGGGGCGGGTTGGCGCCGGGCAAACGCCTT | |
| | GGGTAGAGGGGAGAGGACGTTTCGTTAGTTCCCGCCCCTTCCTGACTAAAATTGCC | |
| | TACCCGAAGCGCCCCGGAGGGCTTCACGGGAGGAGGGTAGACTCTCCTTTGCCCCC | |
| | G | |
| HAND1 | CCGGGCAATGCGAAGGTCCCTCAAGCCTGGACGTTCTGCAGTGGTGGGGTCTCGCT | 343 |
| | CTTGCCCTAGCCCCTCTCCTACCCTCACCCCTATCCGCGCCCCCCGGACTGGCAGGCC | |
| | TCTGGAAGCCCAGGCCGCGGCGCCTACCGCAAAACCTTCTCCCGCCGCAGTCCCGT | |
| | GACCTTGACGCCACGGGCAATCCCCGCACCGGACCCCTTATCTAAATAGGGCAGTA | |
| | AATCAAGGACCTGTCAGGGCCCGGGTAATTACAGGAACTCCATAAAAAGGACCCG | |
| | GCCGGCCGCCTGTTTATATTAGCGCGGTGTAAAATATTCTCGCTGTCTTGGGGAATC | |
| | GCGTCGCG | |
| PANK3_SLIT3 | GCGTGAGAGAGAGATACGAGCCTAAACCCTCACATTGGACTACAGCCTCATCTCCT | 344 |
| | GCCCCGACCTTTCCTTCTGCCACCTCCTCCTGTCCCCGGTTCCCCTTCCAGAACAAAT | |
| | GTTTTCACCGTGATCTGTCCCAGGGCAAAAGCCATCCACATTCTCAGTGCCTACATCT | |
| | AAAGCCCATGCTCCTCGCAGTCAAGGCTCTCCAGCAACCG | |
| NKX2-5_STC2 | GCGGGGGGCCTAGAACCCGAGGCTGGTAGGAGAGCAAACTCTCAAACGCGCTGAA | 345 |
| | ACCGGCCCATCTGGGAGAAATATTAGGGCGCATGTCTCTCCCGGAGGGCTTCCTTTT | |
| | TTTTTTTTTTCCTAACCACG | |
| PROP1_B4GALT7 | GCGCCGGCCGGGTTGAGCCGGGTTGGTTCCGACCCAAGAGAGCTCGTCCCACGAC | 346 |
| | GGAGCAGGTCCCTTTGCATCCCGCGGGGCCGCCAGGTGCAATTTTCGCTGGGCCGA | |
| | CGGCGCGGAGATGGGCCAGAGTCCGGCCATCCAGAAGTGCCTGGAGCGCACAGCA | |
| | AGGCCCTGCCCTCGGCTCCGTGAAGGTGAGGGGGTAAAGTCGGCCCGGAGTCCCC | |
| | GGGGGTGCAGGAGGGGCCCCGCGGGTTCCAGCAGACCCTCGACGGAACGTTCCAG | |
| | GCAGGCGAGATCTCGCACAGAATCTGCCCTTTTAAAGGCTCGGCTTTGTCCTCGTTA | |
| | AACTTGCGTCTGGCAACGCGACCGCTGCGGCTCCCGAGCAAGATTAGAGGGTTTCC | |
| | GCTCGCAGGGGCGCGCCCGGGGACCGCGCCTCCCCGCCTGGTCTCGGCG | |
| PHYKPL_COL23A1 | CCGCGCGCCAGGCCCTGCGAAAAGCCCCAACGGGTCCCCCGGCGACCGCCGCGCC | 347 |
| | GGCCTCTCGGTCCTGTCCTCCGAGGCGCCAGGCCTCCGCCTCCAGCGCGGGCCTCTC | |
| | GGGCAGCGCCGCCCCTCCCCCTGCGCGCACGGGAGGCCGCCTGGGTTCGGCTTTG | |
| | GACCAGGCGAGCAGCGCGGCGCTGGCCGCTCTGCCGGGTCAGCCCCGCGGAGACG | |
| | TCTTCCCCGCTGCGCCCCGGCCCCAGCGCAGCGCCCGGGGAGCGGCCCCTCCTCGG | |
| | GCAGCGGCCGGCGCCTGTGTCCCTAGCGCGGTACTGCTTCTGCCTGAGGACTCCCC | |
| | GCCG | |
| GFPT2_CNOT6 | CCGGGGCGGAGTGGGTTGTCCAAGAGCTTGTCTTGTCCTCTTGCCCTGGCCACAGC | 348 |
| | CGGGAAGCCCTGGGCAGGCGCCCGTGGATAGCTGGCACGCTCAGCCTTTGGTGGA | |
| | GAACTGAGGTGAGCTGGAAGGACTAATGGGAGGGAGGAGAGGTGTACTGGGGCC | |
| | CCG | |
| BTNL9_OR2V1 | GCGCAGTGGATGTGACGCTGGACCCGGCCTCGGCGCACCCCAGCCTGGAGGTGTC | 349 |
| | GGAGGATGGCAAGAGCGTGTCTTCCCGCGGGGCGCCGCCAGGCCCGGCGCCTGGC | |
| | CACCCGCAGCGGTTCTCGGAGCAGACGTGCGCGCTGAGCCTGGAGCGGTTCTCCGC | |
| | CGGCCGCCACTACTGGGAGGTGCACGTGGGCCGCCGCAGCCGCTGGTTCCTGGGC | |
| | GCCTGCCTGGCCGCGGTGCCGCGCGCGGGGCCTGCGCGCCTGAGCCCTGCGGCCG | |
| | GCTACTGGGTGCTGGGGCTGTGGAACGGCTGCGAGTACTTCGTCCTGGCCCCGCAC | |
| | CGCGTCGCGCTCACCCTGCGCGTGCCCCCGCGGCGCCTGGGCGTCTTCCTGGACTA | |
| | CGAGGCCGGAGAGCTGTCCTTCTTCAACGTGTCCGACGGCTCCCACATCTTCACCTT | |
| | CCACGACACCTTCTCGGGCGCGCTCTGTGCGTACTTCAGGCCCAGGGCCCACGACG | |
| | GCGGCGAACATCCGGATCCCCTGACCATCTGCCCG | |
| APC | CACTGCGGAGTGCGGGTCGGGAAGCGGAGAGAGAAGCAGCTGTGTAATCCGCTG | 350 |
| | GATGCGGACCAGGGCGCTCCCCATTCCCGTCGGGAGCCCGCCGA | |
| CDO1 | CGGAGGCGGGGAGACCCTGCGGGCACGGCTCACGCGCACATCCCCGGCTTCCCCG | 351 |
| | GGCTCCGCGCCTTCCCAAGAGCCCCGTTGTCTCCGGCGTCCCAGGGATCGCGTGGG | |
| | CTCCG | |
| FOXF2_FOXQ1 | CCGGCCTCGAAGCAAAAGACGACCGCCGAAACGCGACCGTTTACCGCCTGCTTTTT | 352 |
| | CCAAGCAAAATTTGGAGACAAGTCCCACCCGGGGAAGAACCTGGCTAAGGGTCGG | |
| | ACATGGAAGAGAAGACGCTAAAACAGAAATTGCCTCCCTGCTTTCCACCTGCAGCTT | |
| | CTAGACGCCGCCCTCGGTGCCACCCCTCGCGGAAGGCG | |
| NRN1_FARS2 | CCGAGGCGCGGGACTGGAAGGACAGGTACCAGGCTGCGGGCGCGCGGCTGTGGC | 353 |
| | CATCTCTTTCCGCCCTGAGGCCGACGAACCCGGCTGGAAGCTGAGTGCCTAGCGGC | |
| | CCAAAGCAGCCCGGGCGCCGGGAGGGCGCCAGAGAAGCACAGCGTTAGGGCGGG | |
| | GAAGAAAGGGTGAATCTCAGAATCGAAATCCGCACTGGCGCCCACGACCCTGGGC | |
| | GCCGGCCTGGTCCTCGGCAGCTTTCTGGCGGCTGCGCTTGTGTGTGAATGTGTCCC | |
| | GGGAGGACCGGACACCTCAATCCCCCGGCCCCCAACGCGGGCGCCTGTCCGCGAG | |
| | CGCCGGGCCAGACGCCGAAGAGGAAGGTGACCGAACCCGTAGCAGCTTCCGAGAG | |
| | CGTACCCG | |
| TFAP2A | CCGCCGAGGGCGCCATTGAGGTGCAGATTGGGACCTGCCGGCTCTGGACTGCCGC | 354 |
| | CCCCGGTGTAGGCGCTGATGAAAGGCCCGGGCGAGCGCCAGGGTCGCCTCTGGAG | |
| | CCAGCCGAGCTGCATTTATGCCAGCGTCATTACCACGCTAAGTCGCTTCATTGCATG | |
| | TCAATGCTCCGGCGGGGCCAGAACCCCGGGACAGCAGCG | |
| GCNT2_TFAP2A | ACGGTGGAAATAGGGCGGTGACTAACTTTTCAGAGTGGAAGACACGCACGAAGGG | 355 |
| | CGCACCTGCAGCTCTCCGGGATTCAGGCGGGGGTCGCTGTGCTCTCTTAAAAGTGA | |
| | GCGGCGGTTTCAGCCTGCCACCGCTTCGCCTCGCCAGCTCGGAGGAAACTCTGGCT | |
| | GGAGGCGACCTCGGGCCCAGCCGGACGGGCCGGGCCGAGCCTAGGAGGGGCTGG | |
| | CAGACGTGTCCCAGGGCCAGGGTGGGGCGTAGGGAGCGCCGTCTCCACCCTCAGT | |
| | ACTTTTGGGGTGGGGGACCTGAGCGTGCGGAGAGCGGGAGGCAGAGCTGAGAGC | |
| | GGGGTTAAGCGCGAAGCTAAGGCGCCGCATAGGGTTGGGTGGGAATGGACAGGG | |
| | TGAGCTGGAAGCGAAGCACCCCAGCCAGGCCTTAGGAGAGAGGACCGTCG | |
| ID4 | CCGGGGCCTTGGAGCTTTCGGATCCTGCCCGCCTTTCATCATGTAAACAAACGCATC | 356 |
| | AGATTTAAAGCTTTCCCATAATTGTTATGCTAACCTTGGAGCGCAACCTCTCCATTTG | |
| | CATTTGAAGGAGCTAAATATTAGGCAGGAAAGAAAGTGCTCTTTTTGAAAGCCTGA | |
| | GAAAATGTCCCCGCTCGGGGCTGCTCCGCCATCTGGGCCGCGGGCTGGGCGCGCG | |
| | GCTCCCGCCCCCAGCTCCTTGGCAGAGGCGCCGGAGGAAGGGGCGCCGCGAAGGG | |
| | CCGTCATCTTGTTGGAAAAGAATGCAGAAATGCCCCCCTAAGGCTGAATGAGCACC | |
| | ACTTCCACACTCAGGGCGGGGGAGGCCGGGGGACGTGGGAGCGGCGCGCCAGGA | |
| | GCGAGGCGTCCCTGGTGACAGCGCGTCCCGAGGGCTCTCCCTTTTCCCAGAGCG | |
| TRIM10_TRIM15 | CCGTTTCCCTCTGCGATTCATGTAAGTGTGACTCGATTTCAGGGAAAGGGAACTCGC | 357 |
| | GTGGGCTGAGGAGACCGGAGTGGACGGGCTGGGGAAGGCACCGTGATGCCCGCA | |
| | ACCCCGTCCCTGAAGGTGGTCCATGAGCTGCCTGCCTGTACCCTCTGTGCGGGGCC | |
| | GCTGGAGGATGCGGTGACCATTCCCTGTGGACACACCTTCTGCCGGCTCTGCCTCCC | |
| | CGCGCTCTCCCAGATGGGGGCCCAATCCTCG | |
| PBX2 | ACGGGGTTTGCTGGGTCTGTGTGGGGTCCCGGAGTGGGGGCACTCACTTGGCCTG | 358 |
| | GGCCTCGTCCAGGCTCTGGTCGGTGATGGTCATTATCTGCTGCAGAATGTCCCCGAT | |
| | GTCTTGCTTCCCTCGGCCTCCCGGGACCCCCCCGCTACCCCCACCGGGGTCTCCGCC | |
| | ACCGGGAGGCTCGCCAGGGCCCCCAGGCTCCCCACTCACCAATCCCAGGCCCCCCC | |
| | GGCCCCCGCCTGGAGGGGGCGGCCCCAGTAGCCGTTCG | |
| PNPLA1_ETV7 | GCGCCCCCTGCTTCCCGCGCGCCCACCACGCACGCTGCTCTGGGAGCAGGGCCGGC | 359 |
| | GGCGCCGCCGCCTCGCAGCGATTGGTTGAACCGGAGGTTGTTGCTAGGCTACCAGT | |
| | GCGCCCTGAGCCTGGGGCCCCGCAGTCCCATCCTCTGTGGCAGATCCATCCCTCACT | |
| | GCAGACCTAATTCCGGTACCCTGTGAACGGCATCCTCAGCAGCTTAAATTATCAGCC | |
| | CCAACTGCCCG | |
| GLO1_DNAH8 | CCGTCAGCCTCGTTCCGGGCCGCGGAGGCCGGAGCAGCTCCCCCGGGGCAGCGCA | 360 |
| | ACCGCTGGGGCCGGCCTCAGTGGGCTGAGTGGTCGGGGCATCGGGGCCCAGAGA | |
| | GCGGCTGGTGAGTACTTGGTCGGAGCGCGCTGTGAGCGCCCGGCCCCTGTCCGGG | |
| | AGGCCCTGATGCAGCCGGGTTCCCCGCCCACTTTCCTTCTTTTTAGGGGACTGGAAT | |
| | CCACG | |
| FOXP4_NCR2 | GCGCCACTGCGGAAGGCCTGACCTGATCCGGCACGGTGTGGCCACCGTGGGCCCA | 361 |
| | CAGAGGGTGAAGGGGTAGCTTATGCTGAGTGGGGGTGTCCACCTGGACAGACCAG | |
| | GCGAGCCTCGCTCCTGGTGCGGGAGCTAGTTTTCCCTGGATCTTCCGCGGCAGAGA | |
| | AGCCTGCGTCCGGGACCAGCAGAGTGAGCCGACCGGCGGATGCAGTTGACCCCAT | |
| | TCGCGTCCAAACTTCACTTCGAGAAAACGCAGCCCTGCGCGCAGTCCACGCAGGAC | |
| | GCGACAGCGCCACCCTCGTTTGTACGGCTGCGCGAATGACTCGAGAGAGTCGCGGT | |
| | GGCTGCACGTGCG | |
| MDFI_FOXP4 | ACGTCAATAAAAATTAATTGATGAGTTGGCAGGGCGGGCGGTGCGGGTTCGCGGC | 362 |
| | GAGGCGCAGGGTGTCATGGCAAATGTTACGGCTCAGATTAAGCGATTGTTAATTAA | |
| | AAAGCGACGGTAATTAATACTCGCTACGCCATATGGGCCCGTGAAAAGGCACAAAA | |
| | GGTTTCTCCGCATGTGGGGTTCCCCTTCTCTTTTCTCCTTCCACAAAAGCACCCCAGC | |
| | CCGTGGGTCCCCCCTTTGGCCCCAAGGTAGGTGGAACTCGTCACTTCCGGCCAGGG | |
| | AGGGGATGGGGCGGTCTCCGGCGAGTTCCAAGGGCGTCCCTCGTTGCGCACTCGC | |
| | CCGCCCAGGTTCTTTGAAGAGCCAGGAGCCTCCGGGGAAGTGGGAGCCCCCAGCG | |
| | GCCCGCAGACTGCCTCAGAGCGGAAGAGGCAGCCGCGGCTTTGACCCAGCTTCCTT | |
| | CCGACGGCATCTGCAGGAGCCTCTAGGCCTGACATAGGCTCCGAGGTGCCCTGGCT | |
| | CCCCCACG | |
| GUCA1A_TAF8 | GCGCCAACAGCGCCCTCTCCCGGTAAGTGGGCCTCCCTCCCGCGTTCTACCTGCAAG | 363 |
| | GCCGAAGGGAGAAAACCAAATGTTTTCTCTTGACGGATGGCCGGGACTCCTTGGCC | |
| | CTCGCCTGGCTTTCCACCCCTCCTGGCTTCCCGCACCAGCCGGGCCCGCAGCTCACC | |
| | TGCCGGCAGCTGGGGCGAAGCCGTAGTCGGCGCTGCCGGGCGCTTTGTGCTTGGC | |
| | CTCCGCGGCGCCCCGGGCGGCGCCCTCCAGGGACAGCCTCGGCGCGTGCAGGCCT | |
| | CCGGGGGGCGCGCGACCCGCCGAGTTCACGCGCCGCATCTCGGGGCCTCCGGGCT | |
| | GCGGCCCGAAGCAGTTGGGAGAGCTCAGGCTGCGGCCGGTGCCACCGTGGGGTA | |
| | GCCCTGGGCCTCGGTGCGGCTCCCCGACGTACAGGCGCTTCTTTATGAGCGAGCGG | |
| | CCCCCTCCCGAGAAGCGCTCCAGGCCCCCAGCCCCGGCGTAGCGCGCGCCCGCGGG | |
| | AAAGCGCGAGAAGCCGAGAGCCGGGGGCGCCCCGGGGCCAGCGTTCGGGAGCTG | |
| | CCTCAAGTCTGAGTAGTTGTTCCGGGGAGGGGAGCTCTGGCGGCCCAGATACTGG | |
| | AGGGCCG | |
| TFAP2B | CCGACACCAGTTGGGAGACTGGGTAATAACACACGCTCCGGGCACAGGGACCGCG | 364 |
| | GGCCAACGAACCGCGCGTGCGCCGCGCCAGCCTGCGTCGAGCCGTCGCACACGGC | |
| | TCCGGGAGCCCGCGTCTAGGCACGCTCTCCAGGTTGCCAAGCAGGGTGTCAACAAG | |
| | TGCGCACGCGCGGACGCCCACGCAGGCGCACGCGCCGTGGCGCCCCCGGGCG | |
| DST_KIAA1586 | TCGATCTCTCATGTTTAGGCAAATTCCAGGGTAAGGTGTCTCCCGGAGCTGGGGAT | 365 |
| | GCGGAGCCAGATTTCTGGCTGAAATCATCCTCATCGGAAAAATCCGCAGAGGAAGA | |
| | CATAGAGCAGCGATAGGACGCGTTCCCGGAACTCTACAGAGAATGACACAGAAAA | |
| | AGCATTAACAGCAAAATACTCACATATGCTCAATGATTTAAACATCTCCCCCACCAAC | |
| | CACCGCCGCCCTCCCTGCCCCCAAACTGGGTCTGGCATATCCTGCACCATCCTCG | |
| TBX18 | GCGACCGGTTTAGAGCTGTGTGGTCCCTAGTGGGTCTCCAAGCTCCGGGGTACCCT | 366 |
| | AGGCCGGTATTACATCATTAAAAAGAAGCGCAAATCCCATTTCTGAAGCTTAGCCG | |
| | AAGGCAGGCGCCGGCAGGGAGAGCTAAGAGGCCGCCTAGAGAGTTTGGGCCGGG | |
| | AGTGGGAGTGGGACAAGGCGGGAGCTAACTTAGCTGGAGTAGACGCCAGAAGAA | |
| | GTTCCGTTCAGCTGAGGTGCCCCG | |
| TBX18 | TCGGCTCCTGGAGAAGGGGCGTCGAATCTCTCTTGGGCATGGGAGGGAAAGACAT | 367 |
| | TCCGAGTTGGCTGGGCGGAGTGGCAGCCTTGAGAGTGACGAGTGACAGCAAAGCC | |
| | TCGTCCTAGCAAGGCCTTTTACCAACAGCGCGGCATGCCCTTTCGAGGAGAGCGCC | |
| | AGGCCCTCGCACTTTGCAAGTCAAGAGAGCAAAGAAAGCGGGGACAGGGCGCGTA | |
| | ATCGCAATGTCCGGTCGCGCGTGTGCACGTGTCTGTGTTTGCATGTGTGCG | |
| PREP_PRDM1 | CCGGCCAGGAGTGAACGCTGTCAATTCATCTTGCCCTTAAGGGAGGGAAACCCTCC | 368 |
| | TACCGAATATAGTGCGAGCCTCAATGGTGGGTCTGTCCTGGGGCCTGGGCAGGGC | |
| | GCCGGGTCTCCGGACTCAGGCAAGCACCTTCTCCTAACCGCAAGCGAAGCGAGGA | |
| | GGAGCGACCAGAGCGCTTCCTCTCCCGCCGGAGCTGAGTCCTCTGGGCCGCAGTCC | |
| | TTCCTGGACGAGCTCTGAGGCCGAAGATGCGTTGCGTGACTATGCTGCTGCCTGGA | |
| | CGCGGGGTCTCTAGTCCGGAGGCACGGAAGGACCTGCCTGCCTGACTCTAGTCTGC | |
| | AAGTCTCGGGCACACGCGCGGCTTCTGCCCACCCGCGTAAATGCCCTGGGGAAAGG | |
| | CGCCCTTTCTTTTATGATGTTTTTTAAGAGACG | |
| OLIG3 | CCGGGCCCGCCCGCTGCTCACTTGAGCAAGTCCTTGGACTCGGCCGACAGCCGGGC | 369 |
| | CATGTTGGCTGTGGAGAGAGCGGACAGGTGCGGCGGCGGCGGCATCTGGCAGAT | |
| | GGTGCAGGGGCAGGGCAGACCAGCCCAGTGCTGGAAGCCGCTGCCCAGCTGCAGC | |
| | GCGGGCGGCGTGGAGGGCGCCTTGAGTAGCGAGTGGGGAGGCCGGATGGTGCCG | |
| | ATGGCGGGAAGTGAGGCGGCGGACAGCGGTGACGAGGCGTTGCCAGATGAGAGC | |
| | GCGCCGCCCAAGATGGGGTGCACCGGGTGCACGGAGTTGGCCGCGTGCGCGGGG | |
| | TGGCCGGCCGAGTGGCCCACGGTCCCGCAGTGAAAGGCCGAGTGGTGGCCCCCAT | |
| | AGATCTCG | |
| HIVEP2_GPR126 | ACGGGAAATGAAACCAAGTAACGTGGTGAGAGCACAACTGATGACAATCACAGAG | 370 |
| | AGCACAGTCG | |
| HIVEP2_GPR126 | ACGCCATCTCGTGGCTCACCATTGTGGCATTTCTTCATCGTCAACATTCCAGATTGAT | 371 |
| | AAAAAGTAGTAAATTAAAGACTGGCCCAGCAAAGTCCCTGATCAGCCGGATCACCA | |
| | GCAGCAAGTTGCACGTTTGCACG | |
| MTHFD1L_PLEKHG1 | CCGGAGGGAAATGACTTCATGGGCTCACTGTTGAGCTGCTTCCCTTTGCATCTCGGG | 372 |
| | GGAAGGTGTGGTTCACCCGCAGCAGGTCCGGTGAAGGAAGCACGTGTGTGTGTGT | |
| | GGAAGGGTGGCGCTGACCTCCCAGACAGGACATTACCCTTCTTCCTCTTCCTGACCA | |
| | CTGCTGTTCCCACAGCAGTCACG | |
| PARK2_QKI | CCGGCGTGAAAAGAGTATTTAGAGGGGAGTTGGTCTGGGCTAATCTGCATGTGAAT | 373 |
| | CAGGGGGGTGGACAAAAGGATGAAAAGGTGGTGGAAACTCGAACACAAACCCTG | |
| | CGGTCTCCAGGGGGTCATTCATCTTGCCCCGGTCGACATCCTCGCGGCCTGGCTTCC | |
| | TTCTGCGCATGAGCGAACAGAGCCTTTTCCCAAAGACAGTTGGCAAAGGGTGCGTG | |
| | TGCTTTGTTCTGTCGGGCACTTTTTTAAGAAACAAAATTTCTTTACCCG | |
| DLL1_C6orf70 | GCGGGGTGGGGCAAAGGTGACCCCAGCACGCAGCACGGTGCCAGGCATGGAACT | 374 |
| | GACACGTGATGCCCGTCTGTTTAACGAGTGAACAAAGGCACCAGAGGCTTTCTTCC | |
| | CTTGAACACCAATCTTCCAACCTAGATTAGCAGCCGAGCGAGAGGTGGCGTCTGAA | |
| | CAGCCTAGATTAGAGGCCGAGCGAGAGGCGGCGTCTGAACAGCACCCTGGGATCA | |
| | GGCAGCGCACG | |
| chr6:3 | GTGTCGTATTTATGTGTGTGTCTGCCTCCCGGTTCCAGCGGAGGGCGAGGCGGGGG | 375 |
| | TCATCGTTCTGAAGGGCATCTTTGTGTCTTCCCAGCACTCAGGACAGTGCCTGGCAC | |
| | ACAGATGCT | |
| PDGFA_FAM20C | TCGGGCTGGTGGGGGCTGCAGAGGAAGCCGGCGGGGCCAAAGCGTTCTGTGATTG | 376 |
| | AAGGCGCTGACATCGGCTTCCTGGTTGTGACACGGCGCTCAGCTTTGCGAGATGGA | |
| | ACCATAGGGGACATTGCAAAAAGGGCACACAGAATCTCTCCG | |
| PDGFA_PRKAR1B | CCGGGCTACCCAACATGCCACTTTTTCATTCCAGATTCCTTACTGAGCATCCTTTGAT | 377 |
| | TCCCTTAAATGTGGCCTTCACCCACACGGGCCCTGCGGATTTACCCTGCATGCGAAG | |
| | GGCCTCCCACATCACAGGAGGGCCCCTGCAGGCAGCTCCTGCGCCCGGCCCCGCCC | |
| | GGCCCCGCCGGGCACTCCCTGACGCCCACCCCTGCCCTGGCTGGAAAATCTGAAGT | |
| | TGATGGAGGTGCTTGGTGTTCGTGCACAGCCGCCTGGGACTCACGGGACAGCCCCA | |
| | TAAGTCACAGCCGGTTCCCGCAGGGGGCCCG | |
| ZFAND2A_UNCX | CCGGGATTGTGGGTTTCCTGCCCCAAGGGTTTCGCGGCGTGGGCATGAGCGCTGGC | 378 |
| | ATCTGCGCGCCCTGAGGTTCGGCCGCTGCGTGGCCTTCTCCGGGAGGTGGGGGGA | |
| | ATCCGAAGAGGTCCCACCCCAGGTTCGGTTCCCGGCTTCCTGGTCTTTGTTTACCAG | |
| | GCTCCGAGGAGGACCTGCCTCTCTCCTCCCGCAGCCCTGGGCCCCCCACTCGACAGT | |
| | TTCACATCCAGGGAGGGACAAAGGGGGACGCGGCCG | |
| PAPOLB_AP5Z1 | ACGGGGACCACATGGGACCCAGCTGCCTGCGGCCACCAAACCCAGGCAGCCACGA | 379 |
| | AGCCACGTGGAAAGTCAGCCGGGGACTCTCCAGGAACACAGAGCCGAAAAATCAC | |
| | AGGTCCCTGAGCTGACTCTTCCTGTGGGGGCCGGAACAAAAGGGGCTCCTAAGCTG | |
| | GCCCCGTCCCCCTGTCACACG | |
| HOXA7 | CCGCCCGCGCCCGGCGGGCCTGGCGCGTCCCGCGGAAAAAGACCTGGAGGCTCCG | 380 |
| | CGGGAGCGCCCAGCTGGCGGCCAACCTCCGCACTGGGGTCTGCGGACGCCAGGCG | |
| | GCCCGGCCCCACGCAGCACCCCCCACCCCGCCCCCCCGCCGACTCCTGCTAGTGAGC | |
| | CCTGGACCAAGCTTGGGATCCTCCCCATCCCTCTCCTGTCCG | |
| HOXA9 | GCGGCCAGCCGCCACCAGGGCGAAGGTTTTGAGGGCCTGGTTGGTTGTGCGGCGC | 381 |
| | GCTCGGTCCCCGGCCCTCGACCCCACGCACACGCGCGCCCAGCCCGCCTTTCTCATC | |
| | AGCTGGCAATCAGGATTCCCAGGCGCAGGCGGCTGGCGACCCAGCCCTGTGCTCCA | |
| | GCCTCAGAGGCTCTAACCATGAGCGCTGCAAGCCTGGTTGCGCTCCG | |
| EVX1_HOXA13 | CCGCCGCCAGACTGACCTGGTGTGGCGGTCGGGCGGGGCCGGGCCAGGCCGCGAC | 382 |
| | CGCGAGAAACCACAGCCCCACGGAGGAGGCCGGGCCGCGGGGCTGGCGGGGACC | |
| | CTGCAGGCCGGGCCGAGGTGCGGTGAGGCCTCCTCCCGACCTGGCCGCGTCCTCA | |
| | GAGTTCGCTCGGGGCTTCGTGTTTGCAGAGCAGCCTCCCGCCTGCCCGGCTTGCCC | |
| | GGGGATGTGGGTGGACCCGCCCCGCGCGGCCGCGGCCCAGTGCAAACCGTGATCC | |
| | ACCCTCTTCCGCTCGGTGGGAGGAACCCGGGGCTTTGCGCCCCTAACCAGCAGCGT | |
| | GACCCTCG | |
| EVX1_HIBADH | GCGCGGAAGCCAGGAGTCCATAAAGGACCGTAAAATTGCGGCCCACTTGGGCAGC | 383 |
| | CCGGGTGCTGCAGCCCTCCGACCAGTTTGCACGTCGGTCAGAGGTCCAAATTACCTT | |
| | GTCACTTCCCGGGCTTCGCGGCGCCAGGTCGGAAATGGTCCCAATGGTCTAATTGC | |
| | CTTTGGTCTCCGGTTGCATTTGAAAAGGCAGAGATCG | |
| PRR15 | TCGCGATGGGGCCAAGGGACAGCTGCTGCGGCAACTTTTACCCAGCGGAGCCCACC | 384 |
| | TACAGCCTCAGCCTCCGGGTCTCAGGTCTCCGCCGTTTCTTCTCAAGGAGTCGGTCG | |
| | GGGGAGCGGCACTGCACAGCTTTTCTCCAATCAGACACCTCAAGGCTGGCGCCTGA | |
| | TCCAATCTCCTCCCCTGGAGGGTGGGAACGCG | |
| WIPF3_PRR15 | GCGCAGTGGCGTCTAATGCTAATGTGGGCTACGTAGCTACGGGATTGGGTCGCTCC | 385 |
| | GACCCTGGCCGATCCGGTGCCAGACAGCATAAGGGAGGAAAGGGGACTGGGGGG | |
| | GGCACGTGACTTCAACCAACCCAGTAACCAAGTTTTGTTTTCTTCCCCAGCACAGGC | |
| | CGCTGCCTCAGCATCCACCCCGCAGCCCACGTGTGGCAAGCCGGGGAAGGGGTGG | |
| | AGTGAACGGCCGGAGACCACGTGGAGAAAGGGGCCGCTTTGGCCCTTCCATCTGG | |
| | GTGCCGGGAGCCCCTAGGCCCTCCGGCCATGGCCGACAGCGGCGATGCTGGCAGC | |
| | TCCGGCCCCTGGTGGAAATCGCTCACCAACAGCAGAAAGAAAAGCAAGGAAGCCG | |
| | CAGTGGGGGTGCCGCCTCCCGCCCAGCCCGCTCCCGGGGAGCCCACGCCACCTGCG | |
| | CCGCCCAGCCCGGACTGGACCAGCAGCTCCCGGGAGAACCAGCACCCCAATCTCCT | |
| | CGGGGGCGCCGGCGAGCCCCCCAAACCAGACAAGTTATACGGGGACAAATCCGGC | |
| | AGCAGCCGCCGCAATTTGAAGATCTCGCGCTCCGGCCGCTTTAAGGAGAAGAGGA | |
| | AAGTGCGCGCCACGCTGCTCCCGGAGGCGGGCAGGTCCCCG | |
| TBX20 | CCGGGATGTCCCAGGCTGAGGTGGCCACCAGCCGAGCGCGGCTGCTAGGACGCTG | 386 |
| | GCGTGGGGAGCGCGGCGCGGAACTACGGACAGTGAGCCCTGGCGCTCGCTGCCCT | |
| | GCGCCTTAATTTGCTGGCGGCGGCGATCCCGGAGGCCCGCAGCCAGTCAGCGCCGT | |
| | CTCACGTCACCGCTTCCTGATTCCGCCGCCGGGGGCGGGGCCGCGGGCCGGGCGC | |
| | GGAGGGCGCGCCCAGGGTGCGGCGCCCGCGTGGCCTGTCGCCCCGGCTGTTCGGT | |
| | ACCCCAGCACAGGTTCAGGGAAAAGGGTGCCACCACTAGGCTGACGCAGCAGCCA | |
| | TGGACATCCCCACCTGGTCTCACAGCCCCGGGCG | |
| TBX20 | CCGTGGGGAGCGCGCGGCGCGGCCTTGGATTTCACCGCGAGTCGGGAGGGCGGG | 387 |
| | TCTGAGCCTTGCCTCCCAGGATCCTTCCGACGAACACCCCGCGGGTTTTAGTTTATC | |
| | GAGCCAAAGTGGTCCCGGAGAAGCGCTCCCTCGCAGCCAAGCTGCAAGAAGTGGC | |
| | CGGGAACCTACAGGCCTCGGGCCGACCCAGGAAGCCTCCG | |
| LANCL2_EGFR | ACGTATTTTGAAACTCAAGATCGCATTCATGCGTCTTCACCTGGAAGGGGTCCATGT | 388 |
| | GCCCCTCCTTCTGGCCACCATGCGAAGCCACACTGACGTGCCTCTCCCTCCCTCCAG | |
| | GAAGCCTACGTGATGGCCAGCGTGGACAACCCCCACGTGTGCCGCCTGCTGGGCAT | |
| | CTGCCTCACCTCCACCGTGCAGCTCATCACGCAGCTCATGCCCTTCG | |
| TYW1 | ACGGCTGGCTTTGTTACAGCCGCAGCCGTGGCTTCCCGTGGCTGCACTTGGAAAAA | 389 |
| | GCACTCGACGCTGCCCGGGCAGCTTTCCATCTCAAGTGGGAACGCGGCTGCCGGCT | |
| | GTCTCCG | |
| WBSCR17 | CCGCTGGAGGGGAGCCCACCGCCTCTGGCCCCCCAAGGGGATTCTCTTTTTCTTTAT | 390 |
| | GCCCAAGAACACTGCCCTGGAAGCATCCCCGGAATGACTGAATCATTGCCATTTGT | |
| | GCGGCATCGAACAGACTGTGCCGCTGACAGCTGTAGGCAAGATTGACTCCGATGCA | |
| | GTGCCAGGAGATCTAGGCCATGCAAGGCGGCTGCTCAAGGCCCG | |
| CALN1 | CCGCGCGCTCCTCTACCCCTCCCGCTCCCGCTGGCCGCGCGGGTTCAGCCCATGTGC | 391 |
| | GCGGCTGCCTCGCTGCGCCCCGGAGCCCAGTGGCCGAGGCCCCGCTGGAGTTGCG | |
| | CGCCCTAGAAACTCCATGCAGCTCCGGCCTCCTCCCCAGCTCCTCCCCAGCGGATCC | |
| | CCCAGGGCCTTGCCGCCGACAGCACCACACTCCTCGCTCTGCCGGCGCCCGCGTTCA | |
| | GGAGCCGGGCTTCTGGGCTCGCCTTGGCCGCCTGCG | |
| TAC1 | GCGGAGCGACCAGCGTGCGCTCGGAGGAACCAGAGAAACTCAGCACCCCGCGGG | 392 |
| | ACTGTCCGTCGCAGTAAGTGCCCGCGCGGTGCTGGCCGCGGCTGCCCGGGTCACCC | |
| | CGCCCCGCATCTGTCCGAGGTGGCCGCGCTGGGGGCGCCGCTGCGGCGAGGGACA | |
| | GTGGGGAGACTGGCTTCCCAAACGCCAACG | |
| TAC1 | ACGCGATTCTCTCGCCTAACCGGTACAGGTGAGACTTCAGTCCTTATGTTTTTGATCT | 393 |
| | TGGTTCATCCG | |
| FEZF1_RNF133 | TCGATAATAGAAATTAAAACAACACAGAGCAAAGAACGAGCTTAGTGAAATGGAG | 394 |
| | AAGCAGTAGAGGTAAATAAAAATCCTCGAGCTAGAAAGCTCTAAGAACCGCTTATA | |
| | AATTCAGTTACCTCCTGAACTCCGGCCGATGGCCACTCCGGCCCGGGAGTGCCCCG | |
| | CGCCGACCCGCTGGCCTTGGCCGTCTCAGCCTTCATTATCGCCACGGCCTTGGCGCC | |
| | CCCTGCCCCCG | |
| FEZF1_RNF133 | GCGGCTGGGAGTTGGGGCGCAACTTCAGTGACCGGGCGCCGCTGCCGGGCTGGG | 395 |
| | GCTCCCAAGCGTCCGGCTCCCGGGGTGGTCGACGCGGCGCTGCCTTCGATCAGGTC | |
| | CCGCCGACCTCGGGCCTCTGGACCACCACCGCCCCAGCTGGTCTGGCAACCCATCCC | |
| | GGGCGCAATCGCG | |
| RBM28_PRRT4 | CCGGATTGGCCGCCGTAGCCCAGGGCGTGCAGCACCTCATAGCCCTGCAGGGCTCC | 396 |
| | GCTCAGCAGCCCGAAGGTGCCCGCCACCGGGGCCGTGCGCGCCGCGCGCCGCCAG | |
| | GACTCCCGAGGGGCGAAGGGGCTGCGCCCCTGCGGCAGGGGTGTGGCGCCCTTGA | |
| | AGCCCG | |
| RBM28_PRRT4 | GCGCAGCCAGGCCGGTGGGGCACCGCGGCGGGCGCGGCCGGGCCAGCAGCAGGC | 397 |
| | AGGCCAGCCCCAGGCCGGCAGCCAAGCAGGGCAGCGGAAGGTCCTGCAGCAGCA | |
| | GCCAGGCGAGCGCGGGCAGTCGATCCCTGTGCCCATAGGCGTCGTAGAAGAGCGG | |
| | GAAGGCCCGCGTGGTCCCGGCCGACAGCAGCAGCAGGTCCAGCAGCGCCAGGCAG | |
| | GGGGCGCCGGGCGGGCACCG | |
| KCNH2_AOC1 | CCGAGGCGTCGGGGTTGAGGCTGTGCGCCCGGGGCGATGGGAGCTGGCCGGGCG | 398 |
| | CGCTGCGGGGCGGAGAGCCGGGACCCACCAGCGCACGCCGCTCCTCCGCGGGCCC | |
| | GAGCCCTGCCACGTGGTTGTCCATGGCTGTCACTTCGTCCAGGGCCAGCGACTCGC | |
| | TGCTGGGTGCCGCGGGCGTCAGGTCCACGTCCACCACCACGGCCCCCGGGGCGCCC | |
| | GCGCCGCCCGCGCCGCCCGACCGCACCGACG | |
| PAXIP1_DPP6 | GCGTCGTGCTTTTTTTCATGGGAAAGAAAACTTGACCCAGAGTGGCTTCATTAAAGA | 399 |
| | AGGGAGAGGGACTTCATAAGTGACCAGTCGAGAGCTAGGCCATAGGGGCTGCAGA | |
| | CCCGGGACTCAAACG | |
| SHH_C7orf13 | CCGAGGGGTAGAAAGCGGATGCCTCCTAAACCTGCGTGCGATCTTCTGAGGATAG | 400 |
| | GAGGACACCAGGCCCAGCCCCTGCAGCCCGGTGGGCTCCGCGGCGCCCCCACCCG | |
| | CTTCCCCTCCAGGCCGTTCCTCCCACTGCGGCCGCAGCGTCCAGCCAGGCTCCTTCC | |
| | TGGCCCTGAACACACGGTGACATTCCTGCCCACACGTCCACCCGAGGAGACTCTTTC | |
| | TCAAGCCCCTGCCTGGGACCCATCCG | |
| MNX1_NOM1 | CCGGGCGCTGGCGGCCCCAGCAGCTCCTCGGCTCCCGGCTCCTCCGCGCCGCCCTT | 401 |
| | CCCCGCGCCCCCGCCGCCGCCCTTCTGTTTCTCCGCTTCCTGCGCCGCCTGCTCTTTG | |
| | GCCTTTTTGCTGCGTTTCCATTTCATCCGCCGGTTCTGGAACCAAATCTTCACCTGCG | |
| | GGCACAAGCGGGCGTGAGAAACCGGCCACCGCCACCCCAGGGCTTCCTGTCCCCG | |
| | GAGTCCCCCGGCCGCGTGCGCCTGGGCCCCATTGGGTCGGCCCTGGAATGGCCTCA | |
| | GGGTGAGACGACTTAGAAGCAGAATGGGGAGGGGGCTCG | |
| UBE3C_MNX1 | CCGTCGCCTTCAGGCACAGGTAAGCGCAGCCCGCGCACCGCTTGGGACGCACCTGG | 402 |
| | CCACCTGCGCTGCCACCCAAGCTTGGGGTATGCGGGTGCCCGAGCAGAACCCCGAA | |
| | CTCGCACCGGGCTCCGAGGTTGGAGCAACTCCTAACACTGGGCTCGGAGCTAGGG | |
| | GCTTGCTGGAGGGGCGCTTGCCGCGCCGGCCCTCGGGGCTCACAGCCGGGCACG | |
| UBE3C_MNX1 | GCGGCCAGCCCAGGCGCGGGGCCAAGCCTATTGCCAAAAACATATTACCCTGCGAC | 403 |
| | ATTCTGTAAATGAGATAATGATCCATAAACCCGGATGATAGATGTGGCGTGCCTGC | |
| | GATGTCTTCTCTAAATGAGCTGCTCGCATCGACTGCTAATAATGGTGAGTTTATGGA | |
| | AGCGATTTCAGCGCAAACTGCG | |
| DNAJB6_PTPRN2 | GCGGCAGGAGGGACCCGGGGCCAGCCGAGGCTGTTCCCAGGGAGGCAGACACCT | 404 |
| | GCTGTCGCCGGGACCCTCGACACGCTCCGCACGCGCGGGAGCGGAACCGGGCCTG | |
| | CTTTGGAGGCCTCCCTTGGCGCGCTTGGATTTACTCAAAGGTCAAAGAAAAATGTCA | |
| | AGGAGAGCGATTGCCTGGAGAGCTCCTGGCTCTCCTCCCGGGTCCCCG | |
| TAC1 | CGGCTAATTAAATATTGAGCAGAAAGTCGCGTGGGGAGAATGTCACGTGGGTCTG | 405 |
| | GAGGCTCAAGGAGGCTGGGATAAATACCGCAAGGCACTGAGCAGGCGAAAGAGC | |
| | GCGCTCGGACCTCCTTCCCGGCGGCAGCTACCGAGAGTGCGGAG | |
| HOXA1 | GCTGCTGCGGCGACTGCAAAGGCCGATTTGGAGTGCTGGAGCGAAGAAGAGCAAA | 406 |
| | AGCTGCGTTCTGCGCG | |
| IKZF1 | GACGACGCACCCTCTCCGTGTCCCGCTCTGCGCCCTTCTGCGCGCCCCGCTCCCTGT | 407 |
| | ACCGGAGCAGCGATCCGGGAGGCGGCCGAGAGGTGCGC | |
| DLGAP2_TDRP | CCGGATCGATTTTCCCTTTTCCTCGGCTCTGTCGTCCATACGCCACTCACAGCAAACC | 408 |
| | CAGGCGGCGGGCCCCCTCCGAGGGCGCTCCTTGCGTCCGGACCCAGGTTCTCGGG | |
| | GCGCCCCCCGGTGGGTCCCCGCGAAGCCGCCGCCGCACACCTTCCTCAGCGTAGCC | |
| | CG | |
| DLGAP2_TDRP | CCGGGGGCGACGGGTGTGACCGGGTCCCCCGCTAACTTTCGGGCGCGGTGAGCGT | 409 |
| | CGCCTGCGCGCGCCGCGGTGGAGGCCGCTGCTTTCCCGCCGGGAGCCCGGCACAG | |
| | TCCCCGGGTGACCCGCGCGCCCCGCGCAACAGTTGGAGCCGGGCTGCCCGCGCGCT | |
| | CCCCAAGCCGGGCCCTTCCCCAGATGCAGCCGCGCGCCGGCCGCCCCCCAGTGCGC | |
| | CG | |
| NONE | ACGGTCTTTGTCCAGCTCATGAGACAGGATGCTGGGCATCTGGTCTCATCATCAGCA | 410 |
| | GAGCCGTCACTCAGCGATCTGCCTGCTCCGGGTGAGATCTCAGTCAACTTCGCAATC | |
| | ATCCTCTGACTCATCTGGAGAGGCCTGGGGAAGCCACTGCATCCGGGTCTCCTATCC | |
| | CAGCCGCTAATGACCATGGCCCTACAACATTGTTTCTCCTGACTTTACGTTGTTATGC | |
| | CCCATACACCTCAGTGTCCTGGGGGCAAAATCCTTCACAGCCCCCTTAGTCGCTATC | |
| | CTGCG | |
| SOX7 | GCGCTGCGACCTGCGAACTCCCCCAGTTTCCCTCATCTGCACACCCTGGTGTAGACC | 411 |
| | GACCGTGCGCGCCGGGCCCACGTGCAGCCTGGGGACTGCAGGCTGGGAGCTCACG | |
| | GCCATCTCTCGGCCGCGCTCACCGCAGCTCCCCTGTCACCCGGCCCCCTGTGAGGAG | |
| | CTCTGTTCCCGCGCTCTCATATAAGCGCCGGCACACAGTAGGCGCTCAAGGCCTGCA | |
| | GAATGAGTGAGCAAATATAGCTCAGACACCTACTGAATGAAAGTCGGCAGGTTTGA | |
| | CTAGATCCTGGAATTTAAAATTTACTGAGCGCCACCCATGTGCG | |
| LZTS1 | GCGGCACTTGCGGAGAGCTCGGAACACTCCGCCGAGAATGACTTTTGGAGCCATTT | 412 |
| | GGCAGAGATTAGGGAAAAGAATAAGTGGACACGCTCCAGTTATGAAGAAAAGACA | |
| | TATGGGGATTTAGATTATGAACAGACGGAAGAGGAAGAATGAGGAATCATTCTTTG | |
| | GAGATAAAGACTCTCCGGAACAGAAGCGATGCTGAAATGCGTAAGTCGACAGTAA | |
| | TGACG | |
| RHOBTB2_TNFRSF10B | TCGACTCCAATGCCTTTCAGGAAAGGACTCGGCACTTCTCTGACTGCGGAGGCCCTG | 413 |
| | ACCCTGCCAGCTGGCTCCGAGGGCAACACAGGGGCCTGGCCTCTAGAGGGCTGGT | |
| | GATTGAGGGGCCCGGGCTGGCGGCAAAGAGGGGTTTGGTCTCGGGGCTTAAATGG | |
| | CACCAGACTCTTGCTTTTGCCCATCTGGAGACTGCAGGCTCCCTTCCTTACCCTCAGA | |
| | GAGTGCTTATGGTGGGTGTTTTTGCG | |
| NKX2-6 | CCGGGCTCTTCCGCACCCGCGGATGTGGCGAAGCCGCGGGGCAGCTCCGCTCGCG | 414 |
| | CTCCAGTCGCAGGATGTCCTTGACCGAGAAGGGGGTGGAGGTGACGGGGCTCAGC | |
| | AGCATCCCGAAGGCGGATGGGGCGGGGCCGAGGAGGTCCGGGTGAGGAGCGGC | |
| | ACCCTGAACTTCCCGTCTTGTCGCTGCAGGCCCCGCAGACAGACCCAAGCTCTGGG | |
| | ACAGACGCCCAGCGTCCCAGACAGCGCCTTCCTCTGGGCCATGCTGGTAGGCCCGG | |
| | GTCCAGGGCCGGGTGACGAGACCGTAGCCCCCCATTGGTTCTCGCAGAAACCACG | |
| PLEKHA2 | TCGGATGTTGTCCACCTGACTTGATGCATATTCAAATGTCTCTCTCCCGACGTGGGA | 415 |
| | GGCCGGAGTCAGAACCTGACAGACCTGCCGTTTACTAACTGGGTACCCAGGGCAAA | |
| | TTACTTCACAAGTCTGAGTCTCGGTTTCCTCACCGTGAACCGGACTGGTACCCATAG | |
| | GTTGCGGCGTGGATCAAATGAGATAGCGCAGGGGCGGGACCCGCGCACAGCAGCT | |
| | CTCTTAGTTCCTCTTGGCGAGGTTTACGTAGTAACACATGCTTGTCTGTTTCCCATTT | |
| | TTTCCCAGAGCACCCTCATGCTCTGGGGGCAGGAAGGGAGTCTTCGCATCACACCG | |
| | AAAAAGTCCCAACGGGCACGGTGTAGGCGCCTGTGGTCCCAGCTACTCG | |
| SOX17 | CCGGATGCGGGATACGCCAGTGACGACCAGAGCCAGACCCAGAGCGCGCTGCCCG | 416 |
| | CGGTGATGGCCGGGCTGGGCCCCTGCCCCTGGGCCGAGTCGCTGAGCCCCATCGG | |
| | GGACATGAAGGTGAAGGGCGAGGCGCCGGCGAACAGCGGAGCACCG | |
| RP1_SOX17 | GCGGGAGCTTAGATTCTCTGTGGGCCACATGGTCTCAGAAGAGGCCCCGCGGCCCG | 417 |
| | GGGGCGCCCGCAGTGTCGCTGGACCGGCGGCAGCGCTGGCCACGCCGTGGGCTG | |
| | GGACTGGCCCGGAACGCGGGTGGCGGTTCGGCCTCGGAGACCCGCGCAGCCGTCG | |
| | GAGCATCTCCGTGCCTCGCTCACCACCTTCTTTTCCTCCGCGTCCGGCGGAGGGTTT | |
| | CGGCGCGCGGGGCAGGCCTGGAGCGCCGTGAGCAGGCCGGATGCGGGATACGCC | |
| | AGTGACTACCAGAGCCAGACCCGGAGCGCGCTGCCGGCGGTGACGGCTAGGCTGG | |
| | GCCCCTGTCCTTGGGCCGAGTTGCCGAGCTCCCTCGGGGACTTGAAGGTGAAGGGC | |
| | GAGGCGCCGGCCGGGGCCGCGGGCCGAGCCAAGGGCGAGTCTCGCATCCGGCG | |
| RP1_SOX17 | GCGAGGTGGGCGCAGGAGGAGGAGCTGCCTTCCTCCGGGAGGCGGCGCAGCGCG | 418 |
| | GGGATCTTGCGGGACCAGGCCAGAGACCAGGACCGTCCCCCAACCGTTCGCGGCC | |
| | GCGTAGCCCTGGGCGGCCTGGGCCTGCCCTTCCCCGCGCAGGGCTTTCCCTCCTGCC | |
| | GGTCGCTGCCCCGCACATGGCTCTGGTCGTACTCCCGCTCCACTGCCACCACTGCCC | |
| | ACGCCCTGCGTCCCCG | |
| RPS20_LYN | CCGGGTATGTGTGCTGAGCAAACAGTCCACAGGGCACATGCCCAGCAAGGCTGGT | 419 |
| | GATGGCTCAGAGCCTGCGCCTCGGGTGGGAGAGAGCTTGCTGGAAGCCGGTTTCA | |
| | CCGTGTGGGATGCTGGGGTTGACAGACTTCTCACTGGGCCTTTGAGAAAAGCG | |
| SLCO5A1_PRDM14 | GCGGCCCGGAGTTGCAGGAAGGGCGCCGGCGTCACTGGCCCCAAGAGCTCGGAAC | 420 |
| | GCGCGCGCCGCAGGAGTGCCGGCTGCGGGGTCGGGTTGAGACTGGCGGGACCCT | |
| | CGGCCTCTGCCGGGGTGCGGAAGGTGGATGCTACGGGCAAAGGGGCGGGGCTTG | |
| | CGGTTCCCAGATCCAGAGGCGGGTTGGGGACGTGAGCCGGCGTCCATGTGTTCTG | |
| | CACCCCTTCTCGCCCG | |
| PRDM14 | CCGGCCATTGAGGGAGAGAAAGGAACGCTTAGTTCCATTCACATTCACAGAAAGAA | 421 |
| | GCGCCGAGGGTGGGGGAAACGCAGTCTTGCCGGGTGAGCCGGGACAGGTTCCTCG | |
| | CCTGCCCCCCGGCCGCTGCTTCCTCTTAGCTGAATGGGGAGCGACCCGCCCCGGGC | |
| | GCGGCCTTCGGGGCTGAAGACTGAGGTGCAGCCTCACCCCCGGCCTGGCAGCGGC | |
| | TTGGAAGAGAGAGGGAAAGGAGGAACATCTACCCGGCTAAGAGACGCCGCCAGA | |
| | GTCCCTAAAGCTGGCG | |
| SLC26A7_RUNX1T1 | GCGGCTGGATGTGAGGGCGATCTGGCTGCAACATGTGTCACCCCATTGATTGCCAG | 422 |
| | GGTTGATTCATCTGATCCGGCTGACTAGGCGAGTGTCCCCTTCCTACCTCACTGCTC | |
| | CATGTGTCTCCCTCCTGAAGCTGCACACTTGGTCGAAGAGGACGACCATCCTGATAG | |
| | AGGAGGACCGGTGTTCTGTCAAGGGTATACG | |
| GDF6 | CCGGCTGACCATCCCACCCAGCGCAGGGACCAACGGAAAACCCGCGCGGCGCCAG | 423 |
| | GACCAGGGGGCTGCCCGACGCCGCTCGCGGACTAGTTCCTCAGACTGTGGGACTCC | |
| | CTAGTGCCGGCTTTGCCCAGGGCTTTCCAAGGCTGTCTCATGCCCTAGATCTGCCCC | |
| | AGCAGCTCAGGCCTTGGACTGCGAACCCAGTATCCCGAGACACCGATTCCATCAGT | |
| | CCCCATCCCGACCCCTCTCCAGCCGGGTTCATCCG | |
| VPS13B_OSR2 | TCGGTGAGGCGTTCGGTATGGATTGGGTAGGAGCGGCCCTGGGCGATGGGCCTGA | 424 |
| | CGTCGGTGGGCGCAGTTGAGGCCACTGCAAGGCCGCTGGATCCCGGATCCGCACC | |
| | CGAGACGGAGCGGGGGCCACACGGGATAACCGAGGGGGCGAACGGGAGTTTCGG | |
| | GCCTCCGCTCCCTCTCCGGGTGGGGGACAGGTCGCCGAGTCCGAGGTCGGGCGCG | |
| | AAGGCCACTCGCATTTTCCCGCCTTCCGCGAGCAACCCAGGGGCCCTGCGGGAGGA | |
| | GGAGAGGGTCCCGGGAGTCCGCCCTTCCCTGCGCCTTCGGGACCGGCAGGAGGCG | |
| | CTGCGCGGGCGAATTAAAAGAAAAGGAAAAGCTCGTAGTGGAGGTGTTACCGCAT | |
| | CCTGCCTTTGGACGCTACTCTTAGTTGAGTGACCCGATTCGGACCTTAGGGGCGTTA | |
| | GGGTCTCCTCCACCG | |
| TRPS1 | CCGCTGTCAGGCATTTAATCACCGGCCAGTGTCCCCTGACCCGCGCGACACATGGC | 425 |
| | GCATCAACCGCATCGCAGAGGAAGTCTGCCCCTTCCTCAGCCCCTACGGAAGCGCC | |
| | CGGGCTGCAAGGCCCTGCCACATGGTACGGACAGGGCACAGACCGCTCGGCCAAG | |
| | CTGTCCTGAGCCGCTCTGAGGCGGGTGCACCAAGGGATGCGACACCCG | |
| ARC_BAI1 | CCGGGTGCAGGTTGCGGGGCAGGCATGAGGGGAGGCAATTCAGGCAGCAAAAGC | 426 |
| | AGCAGGGTCAAAGGTCAGAGGACGTGGGCCCGTAGCCTCGGAGGAACCGGAGGA | |
| | GCAGAGCAGAGGCCAGAGGGCCAGAGTGGGTGGCAGGGAGGCTGGCAAGGGAG | |
| | GTTGTGGCCATTGTCCCAGGACCAGGGGAGCCATCGTGAGCTCTGAACAGGGGAG | |
| | TGGCACAGCCCG | |
| OPLAH_SPATC1 | GCGCCAAAAGCAGCCCTGGGCCCTGGGTATCGCGCTTGGGGGGAGGGTACCCCCG | 427 |
| | CCGGCTGGGCACGCGCCAAGAGCAGCCCTGGGCCCTGGGTATCGTGCTTAGGGGG | |
| | AGGGTATCGGAGCGGGAAGTGGACCTGGGGAGCGCCGTCGGCTGAGGCTCTGGC | |
| | TGATGCCGCCCTCCCCCGGATCCCCCAGGGACCGCGCTGAGCACCTCCGTGCTCCAC | |
| | CAGTCCATGGCCTCCTCCCCCAAGATGCCGAGGCGGTGAGTTGCGACCTGGATGTA | |
| | GGCACTGCCCGCCCGAAGCGCGCGGAGGGGCCCTGGCCTTGATGACACCGCCCCC | |
| | CTACCAGGGCCCTGGAGCAGGAGAAAGGGCGCCACCTCTACCTGGCCGGCCTTCCC | |
| | GGCAGAAGCCGCCGAGCTAAGCCCTGGAGAGGTCGGCGCCTGGACTACATCACGT | |
| | ACCGCGGAGTTCCCGGGTGGCTGGGCCTGCGGCACTGGGACGACCCTCAACCTGA | |
| | CTCCCGCCCCCAGGAGGTGGAGCAGGTGACGTTCAGTACCGCCCTGGAGGGGCTC | |
| | ACGGACCACCGGGCAGTGCGCCTGCAGCTCCGAGTCTCAGTGTCCTCCTAAGGCAA | |
| | GCACAGATGAGGGGCGCGCGGCTGGCGCGCACAGACACGACTCGGAGCACGAAC | |
| | TAGGCGCCGTAGCTGCGTCCCCAGAACCGGGAGACTTAAGGCATCTTTATTGCGGG | |
| | ATCCTCACACGGCCTCCTGGGCCCGGCGATACTCATAGACGCTGCCGTGCTCGGGA | |
| | AAGGCCAGTGCTTGCGGGGGCGACCCCGGCGGTGGGGCGGGGTCCTCCGGGTCCC | |
| | CATAGCCACCGCCGCCGGGCGTGTGGAGACAGAACACATCCTGTTGGCGCGGGGG | |
| | GGGGCGGGGAGGCGGGCTCAGTGCAGGCG | |
| SDC2 | TCGGGAGTGCAGAAACCAACAAGTGAGAGGGCGCCGCGTTCCCGGGGCGCAGCTG | 428 |
| | CGGGCGGCGGGAGCAGGCGCAGGAGGAGGAAGCGAGCGCCCCCGAGCCCCGAG | |
| | CCCGAGTCCCCGAGCCTGAGCCGCAATCGCTGCGGTACTCT | |
| SFRP1 | GAAGCCGAAGAACTGCATGACCGGCTCGCACGAGTCGCGCACGGCCTCGCAGAGC | 429 |
| | CAGCGACACG | |
| SOX17 | TTGGACTGGGACGTGGGACTCGGACCACGGCCTGGGCGTGGGCCTAACGACGCGG | 430 |
| | GACCGGCCCGCCCTC | |
| ATAD2 | ATGACTGTGATACTCAAGTACAGAATTGTGGTGCAGCCAGAAGTGGTTCAAGAGCC | 431 |
| | CTCCCGCAAATCATGACTTGCACTCTGGCTTTTAAGTGAAGACGAGGGAATCTCAAG | |
| | GCAGATGGG | |
| ch8:20 | AAAGTATCAGCGTAGAAGGAATTGTGTCTGCCTAGGAAAAGGGTGTGGCAAGAGG | 432 |
| | AGGAGCGGCACTTGCGGAGAGCTCGGAACACTCCGCCGAGAATGACTTTTGGAGC | |
| | CATTTGGCAGAG | |
| DMRT2_DMRT3 | ACGGAATCTGACCAAGGCTGGACCCTCAATAATTGTGATTTCTTTTCCCCCTTTTCCT | 433 |
| | TCTTGGTAAAATCATCCCACGAATCTACGCAAGTAGGGCCCTTCGTCATTCTTCGGA | |
| | GTAGCCGCTTGAGGGCTGGAAGGAGCAGTGATAGAAACCCCAGAGACGCAGAGA | |
| | CCCTCCGAACTTCGAACTCGATCACTGTCCTCCCCCGACCGCCGAACCCGCTGGAGA | |
| | AGCGGGCGCGACAGGGCGATGAGTTAACGCGGAGGGAGCGCGGAGGCCGCGGA | |
| | AGCCGGGGGCGCTGGGTCTCAGGCCCGGATGCTGAGCGCGGACCGGCGTGTCCTC | |
| | CCCACAGCGCCCCCGCGCGGCCTCCTCCCGCTGCGCCCCGCACGGCGACCCGCCGC | |
| | GGGTAGCCCTGGCGTTTGGCCACGCCGTCGGCTGAGGACCGCTAGAGCTGGGGGG | |
| | AGATCAAAGCATTCCTATGGGGCCCAAAGAGCCTGGGATTGCAGTGTTGTTAGCCT | |
| | GGCCTCGCCGCGTCAATAAATTTTCGGCG | |
| MPDZ_NFIB | TCGTGATCATTGGATGCATCCTCTCGATTCTCATCGTTGCACTGTCGCGGAGAACAC | 434 |
| | TTTGTTATCCGGCGTTTCTCCCTGCGTGATTATCATTCTTCCCCGCATTGTGGCGGGC | |
| | TCTGCAGCTAGCAGGGAACCTGATCTCTGGCTGCTGCCCAAGGAGCTCGGCGAGAC | |
| | CGCCCATCTGTCCGGTCCTGCTCTCCACCAGCTCCTTCGTCG | |
| NFIB_ZDHHC21 | ACGTCAGAACAGGGTCTCCTATCAACTGCTACCTATTGCTGTCTCGCAAACATCCCC | 435 |
| | CTAAACCCGCTGCATCGACAGCTTCGGGTGAGGGTGGGGTAAGAGGCACTTACTGT | |
| | GAGGCCGAGCTCCCGCACGAATTAGCCTCACAACAGGACCTAGGTCTCCTAGGGAG | |
| | ACGAAACTAGGCCAGCGAAATCGCGGCCAGGGAGCCCCTGGCCCCCACTCGGGAG | |
| | ACAACCCGCCCGGCGCGAAGGGTGCGTCTCCTGAGCTCCACGCCGGGAGCTGGAA | |
| | GGCAGGCAGACGCGCG | |
| SLC24A2 | GCGCCCCTCTGCGCGTCTCCCCCGACGGCAGGCCCTGCCCCACGCCCCCCATCCCAA | 436 |
| | GCCAAAAGCAAGGGTAGGAGAGGCGGGGGCTCCAAATCCACGCCCCGGAGCACA | |
| | GAGAGTTGGCTAACTCCTAGCGGGGCCTGGGGCGCCCACATCCACG | |
| SLC24A2 | TCGCCAGCCGGGCTGGGTTCGGGAGGAGACTGAGCCGCTGTGAGCCCGGCGCTCC | 437 |
| | GAGTCTGGCGCTGCCCGGCCCCCGCCGGCCCCTCCCTCTGGGCTGTGCGCTGTGCG | |
| | CTGGGAGCGGGGCCGCAGCGCGCTCAGCTCCCGAGTCCTTTGCTCCACGCCTCCTG | |
| | GGCGCAGAGGCGACGCTGGCAGCCG | |
| C9orf72_LINGO2 | CCGGGGAGGAGCCAAGATGGCCAAATAGGAACAGCTCCGGTCTACAGCTCCCAGC | 438 |
| | GTGAGCGACGCAGAAGACGGTGATTTCTGCATTTCCATCTGAGGTACCGGGTTCAT | |
| | CTCACTAGGGAGTGCCAGACAGTGGGCGCAGGTCAGTGGGTGCGTGCACCGTGCG | |
| | TGAGCTGAAGCAGGGCG | |
| PAX5_MELK | CCGGCGCCCTCGCCCCGGCGCGCATCATCTGCTCCGCTGCCCAGCTCCCGGCTGCCG | 439 |
| | CCGCGCCCGCGCCCCCCGGGGCCCCGGAAAGCTGGCATCCGTTGTTAGCATAACAA | |
| | ACTCAATTGTTCTCAGCGGGGCCCCGGCAAATAAAGTCATTCATTACGGGCCTCTCC | |
| | TGGCCGCCGCGGGCCGCGCGGCAATCAGCGGGCCGAGCCACGCGCCAGCGCTGG | |
| | GACCTGCAGGGCGCGCCGCCGCCTCCACGCTGCGCCCCGGGCCCCGCCGCGGCCG | |
| | CGCCGGCGGGGGCAGCGCCGGCCGCCGATTAGTTTTATCTCGGAACGTCAATTGAC | |
| | TTAGACTGATTGGCTTCCTGCCGCCAATGTCAATTAAATTGCAAATGCTTGGCGGAG | |
| | GCCGGCGCGAGCGGGCGGCCTCCTTCCCGGGGGCGCCGCGCTCAGCCTTCTCTTTG | |
| | CGCCACGTTCGGCCGCAGCTGAATTCATTTCTCCTTCCACGTCGCGCAGGAAATCCA | |
| | GGTGACCTCCTGGAAGTCGTCTGCCCTCCGCCCCCGGCCCTGGGGACTCCTCCGTCG | |
| | GAGCCCGAGCCCCGAGGACTCCCGGCCGGTGGGCGGGAGCTAGGCCCACGGGGC | |
| | GCCCGGACCGCGGGGCCGAGGAGGAAGGGACCGGCCTCCCCGCAGGGACCTCG | |
| PAX5_MELK | TCGAAGGAGATGGTGGCCGGGGTCCCGTCCAGCCCATGCCCAGTGCCTGGGTGTCC | 440 |
| | AGAGGGAGGAAGGCCTGGCAGCATCACCAGCGTTCACCTGGTGCTGACGCTGTGC | |
| | CGAGCCACGGATGGGCACAGTCTAATCTTCCCCCACAGCCCTCCGAAGCAGATACT | |
| | GTTACTGTCCGACTTCTACAGAGGAGCGAAGTGGGGTGCAGGCCAGAGAGTGGCC | |
| | AGTTGGGTTTCAAACGCCTGCG | |
| FOXE1 | GCGCGGCGAGACGGCAGCAGGGGCCGGGGTCCCAGGGGAGGCCACGGGCCGCG | 441 |
| | GGGCGGGCGGGCGGCGCCGCAAGCGCCCCCTGCAGCGCGGGAAGCCGCCCTACA | |
| | GCTACATCGCGCTCATCGCCATGGCCATCGCGCACGCGCCCGAGCGCCGCCTCACG | |
| | CTGGGCGGCATCTACAAGTTCATCACCGAGCGCTTCCCCTTCTACCGCGACAACCCC | |
| | AAAAAGTGGCAGAACAGCATCCGCCACAACCTCACACTCAACGACTGCTTCCTCAA | |
| | GATCCCGCGCGAGGCCGGCCGCCCGGGTAAGGGCAACTACTGGGCGCTTGACCCC | |
| | AACGCGGAGGACATGTTCGAGAGCGGCAGCTTCCTGCGCCGCCGCAAGCGCTTCA | |
| | AGCGCTCGGACCTCTCCACCTACCCG | |
| TLR4 | CCGATGCCCCGAAGTCCTGTGGGCAGCCTAGCCACAGTAACTTGGTGGAACTCATT | 442 |
| | AGCGCAGGCCGTTCTCATCAGCGCCACGGAGGACGGAGACGCCGGGGTTCCCGGC | |
| | TTTGAGCCTCTGGAGCGCCCGCGCCTTCGCGGGCTGCGCGGGGCTCAGGGAGCCG | |
| | CGGCCACGGCTCCCGCGCGCTCGCTCGCCCGCAGGATCTGGGCAGCCCCGCGGGG | |
| | ACCCGGCTCTGCGCGCAGCCCATTGTACAGCTGGCGCAGCCGCGCAAATGACATCT | |
| | GAGCCTCCTTTCAAGCCGCCG | |
| NEK6_LHX2 | GCGGTTCCTTTTGCTCGGCCCGATCCTCCTTTAAAGACAGGTCTCAGTTTTCCCGGAC | 443 |
| | TTTTTCCTCCGAGTTTCCTGGCGCCTGCTGGGGTGAGGGCCGTGACCCTCGGAAGC | |
| | GAGCCCCCCGGGCGGGGACGAGACCGGAGCAGGCCTGGCCTCGCGCCGGGGTGG | |
| | GGTGGGGTGGGGTGAGGTGGGGGGCTTGGTTCGGATTTCCGGCATCTTTGAACCC | |
| | CAGGCCATTCCCGGAGAAGCTCTGCCCCCTCCCGCG | |
| NR5A1_GPR144 | GCGGAGGGACAGCGGGTCAGGGAGGGCCGGCGGAGACCGGCAGCCTGGGGTCCC | 444 |
| | CGCGGCCGCCGCCCCAGCCGCTGTCGCCGGCCCGTCGCGTAATCCCCTCTCTGTGCC | |
| | CAGGCGCTGCCGCCGGCACCCACCGAGCGCCCCGCGCAGCGTCCCGGGGTGGGTC | |
| | CGGTGCAGTCCCCGCGCCCGGCCTTCCCCTGCCAGGCCCCACG | |
| USP20_FNBP1 | TCGTCCCCGTTGGCGGGGGAGCCCATTGTGGAGCTGTGGGGACTGCCACACTCACC | 445 |
| | ATGCACCTGTTGGTTTGCAGGGACAGAGGTGCGGCCCTGACTCTTCTCACCCTGTGT | |
| | CATCCGGGCTTGTCTTTCGTCTGTCAAGTCAGTCCTCCTGCGTGACTGATGGGTGCA | |
| | CCACGCTTAGGTCACCCGTTGCAGGGACCGGAAGTCCATGGCTCTGCCGCAACCCT | |
| | GAGCG | |
| USP20_FNBP1 | CCGGAAGGGTGGTGTGTGGTCAACCTTGGTTGGCTGAGAGGAGCAATTTCCTGGTT | 446 |
| | TCCACAAGTAAAGACAGCCCCATCCCTTGGGACCTGTCCTTTCCG | |
| QRFP | CCGGAGAGGACATGGGGTGGGTGGACATCTACCCGACACACCTACTGCCCAGCTTG | 447 |
| | CAGGATGGCTTTCATGGGCAGGAAAGCCACAGACACCCATGAGGCCCGTGTTTCAC | |
| | AGGCACCGGGCTGCGCGGCTAAGCCAGGTGCACCTCCCCGGCAGGTGGAGCCCTC | |
| | AGCGGCCTGTTACCCAGGAACCAACCAAGGGGGCACGGCAGATGCCCAGGACAGC | |
| | AGTGGAGCATTTGCCTGTGGCCCCCAGCCCCTCCCACCG | |
| GTF3C4_BARHL1 | GCGCGGGCAGAGCGCCGAGCGCGGCGCAGGGACTGGAGTTCTCGCCAGCTTCGG | 448 |
| | GTTCTTTCTCCCCGGAGCTGCCCGGGGGGTCTCGGCCTCGGGCGCTCCCGCCGCCG | |
| | TCCTGTTCCCCTCAGGGTTCATGTCCTGTTCCCGGGGCCCCAGAGGTCCCGTCTGAG | |
| | AGCGGCCCCCGCG | |
| SEC16A_NOTCH1 | GCGGGAGACGGGGGAGTCCACTTCTCAAACCCGGTGCATCCTGCAGGGCCGCTGC | 449 |
| | ACTCACAAAAAGGCTGACTCCACACAGGACCTGCCTCCCTGGGCCTTGGCTCAGGC | |
| | TGGGGCG | |
| CDKN2A | CTGGATCGGCCTCCGACCGTAACTATTCGGTGCGTTGGGCAGCGCCCCCGCCTCCA | 450 |
| | GCAGCGCCCGCACCTCCTCTACCCGACCCCGGGCCGCGGCCGTGGCC | |

1. A method for detecting the presence of a cancer and for identifying the cancer origin in a test subject, the method comprising:
a) bisulfite treating cell free DNA (cfDNA) from a liquid biopsy sample of the test subject;
b) using the bisulfite treated cfDNA to prepare (i) a first sequencing library for a plurality of specific target genomic regions and (ii) a second sequencing library for a genome of the species of the test subject from a flow through of the first sequencing library;
c) sequencing the prepared first and second sequencing libraries, thereby producing a corresponding first and second plurality of sequencing results;
d) analyzing the corresponding first and second plurality of sequencing results by measuring:
i. a plurality of site specific methylation densities, using the first plurality of sequencing results, for the plurality of specific target genomic regions of the test subject relative to a plurality of site specific methylation densities determined using a plurality of sequencing results for the plurality of specific target genomic regions in a plurality of liquid biopsies obtained from a cohort of healthy subjects;
ii. a methylation density for the genome, using the second plurality of sequencing results, of the test subject relative a methylation density for the genome determined from a plurality of genome wide sequencing results for the plurality of liquid biopsies obtained from the cohort of healthy subjects;
iii. a respective copy number of cfDNA in a plurality of first bins across the genome, using the second plurality of sequencing results, of the test subject relative to a respective copy number of cfDNA in the plurality of first bins across the genome determined using a plurality of genome wide sequencing results of the plurality of liquid biopsies obtained from the cohort of healthy subjects, and
iv. a fragment size pattern distribution of cfDNA across the genome, using the second plurality of sequence results, of the test subject relative to a fragment size distribution of cfDNA determined using a plurality of genome sequencing results for a plurality of liquid biopsies obtained from a cohort of a healthy subject; and
e) responsive to inputting into a combination model of each of the analyzed sequencing results from (d)(i)-(d)(iv), receiving as output from the model:
i. a categorical indication of a presence or absence of the cancer in the test subject, and
in the case where the model determines presence of the cancer in the test subject, an origin of the cancer.

2. The method of claim 1, wherein the plurality of specific target genomic regions comprises at least 200, at least 250, at least 300, at least 350, at least 400, at least 450, or at least 500 or more cancer specific regions.

3. The method of claim 1, wherein the plurality of specific target genomic regions comprises between 400 and 500 cancer specific gene regions and wherein the plurality of specific target genomic regions consists of between 17,500 and 18,500 CpG sites.

4. The method of claim 3, wherein the plurality of specific target genomic regions comprises at least five nucleic acid sequences selected from SEQ ID NOs: 1-450; at least 50 nucleic acid sequences selected from SEQ ID NOs: 1-450; at least 200 nucleic acid sequences selected from SEQ ID NOs: 1-450; or at least 300 nucleic acid sequences selected from SEQ ID NOs: 1-450.

5. The method of claim 3, wherein each respective target genomic region in the plurality of specific target genomic regions encompasses a sequence selected from SEQ ID NOs: 1-450.

6. The method of claim 2, wherein at least 20 respective cancer specific genomic regions in the plurality of cancer specific genomic regions encompass an oncogene and/or a tumor suppressor gene listed in Table 23.

7. The method of claim 1, wherein the plurality of specific target genomics regions is captured by a set of DNA probes comprising DNA fragments with a size ranging between 40 base-pair (bp) and 50 bp, between 51 bp and 60 bp, between 61 bp and 70 bp, between 71 bp and 80 bp, between 81 bp and 90 bp, between 91 bp and 100 bp, between 101 bp and 110 bp, between 111 bp and 120 bp, between 121 bp and 130 bp, between 131 bp and 140 bp, between 141 bp and 150 bp, between 151 bp and 160 bp, between 161 bp and 170 bp, between 171 bp and 180 bp, between 181 bp and 190 bp, or between 191 bp and 200 bp.

8. The method of claim 7, wherein the set of DNA probes consists of between 400 DNA probes and 500 DNA probes, between 501 DNA probes and 1000 DNA probes, between 1001 DNA probes and 1500 DNA probes, between 1501 DNA probes and 2000 DNA probes, between 2001 DNA probes and 2100 DNA probes, between 2101 DNA probes and 2150 DNA probes, between 2151 DNA probes and 2200 DNA probes, between 2201 DNA probes and 2250 DNA probes, between 2251 DNA probes and 2300 DNA probes, between 2301 DNA probes and 2350 DNA probes, between 2351 DNA probes and 2400 DNA probes, between 2401 DNA probes and 2450 DNA probes, between 2451 DNA probes and 2500 DNA probes, between 2501 DNA probes and 3000 DNA probes, between 3001 DNA probes and 3500 DNA probes, or between 3501 DNA probes and 4000 DNA probes.

9. The method of claim 8, wherein the set of DNA probes comprises at least 10 nucleic acid sequences selected from SEQ ID NOs: 451-2700; at least 100 nucleic acid sequences selected from SEQ ID NOs: 451-2700; or at least 200 nucleic acid sequences selected from SEQ ID NOs: 451-2700.

10. The method of claim 1, wherein the first sequencing library is prepared for paired-end sequencing, and wherein the second sequencing library comprises universal adapter sequences.

11. The method of claim 1, wherein the plurality of specific target genomic regions have a methylation percentage higher in the test subject as compared to the cohort of healthy subjects.

12. The method of claim 1, the method further comprising converting the second sequencing library into cfDNA sequencing library spheres for genomic sequencing by rolling circle sequencing or MGI-DNBseq sequencing.

13. The method of claim 1, wherein the analysis of the sequencing results from (d)(ii)-(d)(iv) is performed by measuring non-duplicating fragments in the genome.

14. The method of claim 13, wherein the methylation density for the genome in (d)(ii) is determined for each respective second bin, in a plurality of second bins, wherein the plurality of second bins consists of between 2500 second bins and 3000 second bins, and wherein each respective second bin in the plurality of second bins represents a different between 800,000 nucleotides and 1,200,000 nucleotides of the genome.

15. The method of claim 14, wherein the measuring of the methylation density identifies respective second bin regions in the plurality of second bin regions that are differentially methylated between the test subject and the cohort of healthy subjects, and wherein the methylation density in each respective second bin region is evaluated based on a Z score value.

16. The method of claim 1, wherein the plurality of first bins is between 2500 first bins and 3000 first bins, and wherein each first bin in the plurality of first bins represents a different between 800,000 nucleotides and 1,200,000 nucleotides of the genome.

17. The method of claim 1, wherein the measuring of respective copy number of cfDNA identifies a subset of first bins in the plurality of first bins with variation in the number of copies of DNA per bin between the test subject and the cohort of healthy subjects, wherein the variation in the number of copies of DNA between the test subject and the cohort of healthy subjects in each first bin is evaluated based on a Z score value, and wherein the Z score identifies regions of instability in the genome.

18. The method of claim 1, wherein the measuring of the fragment size pattern distribution of cfDNA across the genome comprises determining a fragment size pattern distribution in each third bin in a plurality of third bins, wherein the plurality of third bins consists of between 500 third bins and 600 third bins.

19. The method of claim 18, wherein each respective third bin in the plurality of third bins represents a different between 4.5 million nucleotides (4.5 megabases) and 5.5 million nucleotides (5.5 megabases) of the genome.

20. The method of claim 19, wherein the measuring of the fragment size pattern distribution of cfDNA identifies a subset of third bins in the plurality of third binds with a variation in the fragment size pattern distribution of cfDNA per bin between the test subject and the cohort of healthy subjects.

21. The method of claim 20, wherein the variation in the fragment size pattern distribution of the cfDNA in each third bin in the plurality of third bins is evaluated based on cfDNA fragment length ratio (RF) value, and wherein the RF value identifies presence of cancer, wherein cfDNA fragment length released from tumor cells from the test subject is shorter than cfDNA fragment length released by cells of the cohort of healthy subjects.

22. The method of claim 1, wherein the cohort of healthy subjects consists of between 5 and 50 healthy subjects, between 5 and 100 healthy subjects, between 5 and 1000 healthy subjects, between 5 and 5000 healthy subjects, between 50 and 500 healthy subjects, between 50 and 1000 healthy subjects, between 50 and 5000 healthy subjects, between 100 and 500 healthy subjects, between 100 and 1000 healthy subjects, between 100 and 5000 healthy subjects, between 500 and 1000 healthy subjects, or between 500 and 5000 healthy subjects, or more.

23. The method of claim 1, wherein the liquid biopsy sample comprises a body fluid, blood, or plasma.

24. The method of claim 1, wherein the origin of the cancer comprises colorectal cancer (CRC), liver cancer, lung cancer, breast cancer, or gastric cancer.

25. The method of claim 1, wherein the model is a composite model comprising four attribute models and a combination model, wherein each respective attribute model in the four attribute models produces an initial categorical classification upon input of a different one of the analyzed sequencing results from (d)(i)-(d)(iv), and wherein the combination model combines the respective categorical indication of the presence or absence of cancer in the test subject of each attribute model in the four attribute models by a weighted combination of the four attribute models.

26. The method of claim 26, wherein the combination model is a logistic regression combined linear model of the four attribute models, in which each of the four attribute models is independently assigned a different probability weight.

27. The method of claim 1, wherein the model comprises at least 100 parameters, and wherein the model comprises a logistic regression, a deep neural network, a fully connected neural network, a convolutional neural network, a graph based neural network, or a support vector machine.

28. The method of claim 27, wherein the deep neural network specifies a tissue for cancer origin.

29. A method for monitoring likelihood of cancer recurrence in a subject previously treated for cancer, the method comprising:
a) bisulfite treating cell free DNA (cfDNA) from a liquid biopsy sample of the test subject;
b) using the bisulfite treated cfDNA to prepare (i) a first sequencing library for a plurality of specific target genomic regions and (ii) a second sequencing library for a genome of the species of the test subject from a flow through of the first sequencing library;
c) sequencing the prepared first and second sequencing libraries, thereby producing a corresponding first and second plurality of sequencing results;
d) analyzing the corresponding first and second plurality of sequencing results by measuring:
i. a plurality of site specific methylation densities, using the first plurality of sequencing results, for the plurality of specific target genomic regions of the test subject relative to a plurality of site specific methylation densities determined using a plurality of sequencing results for the plurality of specific target genomic regions in a plurality of liquid biopsies obtained from a cohort of healthy subjects;
ii. a methylation density for the genome, using the second plurality of sequencing results, of the test subject relative a methylation density for the genome determined from a plurality of genome wide sequencing results for a plurality of liquid biopsies obtained from the cohort of healthy subjects;
iii. a respective copy number of cfDNA in a plurality of first bins across the genome, using the second plurality of sequencing results, of the test subject relative to a respective copy number of cfDNA in the plurality of first bins across the genome determined using a plurality of genome wide sequencing results of a plurality of liquid biopsies obtained from the cohort of healthy subjects, and
iv. a fragment size pattern distribution of cfDNA across the genome, using the second plurality of sequence results, of the test subject relative to a fragment size distribution of cfDNA determined using a plurality of genome sequencing results for a plurality of liquid biopsies obtained from the cohort of a healthy subject; and
e) responsive to inputting into a model each of the analyzed sequencing results from (d)(i)-(d)(iv), receiving as output from the model:
i. a categorical indication of a presence or absence of the cancer in the test subject, and
in the case where the model determines presence of the cancer in the test subject, an origin of the cancer,
wherein the detection of a cancer is indicative of cancer recurrence and need of resuming treatment to the subject.

30. A method for assessing the efficacy of a cancer treatment in a subject suffering from cancer, the method comprising:
a) bisulfite treating cell free DNA (cfDNA) from a liquid biopsy sample of the test subject;
b) using the bisulfite treated cfDNA to prepare (i) a first sequencing library for a plurality of specific target genomic regions and (ii) a second sequencing library for a genome of the species of the test subject from a flow through of the first sequencing library;
c) sequencing the prepared first and second sequencing libraries, thereby producing a corresponding first and second plurality of sequencing results;
d) analyzing the corresponding first and second plurality of sequencing results by measuring:
i. a plurality of site specific methylation densities, using the first plurality of sequencing results, for the plurality of specific target genomic regions of the test subject relative to a plurality of site specific methylation densities determined using a plurality of sequencing results for the plurality of specific target genomic regions in a plurality of liquid biopsies obtained from a cohort of healthy subjects;
ii. a methylation density for the genome, using the second plurality of sequencing results, of the test subject relative a methylation density for the genome determined from a plurality of genome wide sequencing results for a plurality of liquid biopsies obtained from the cohort of healthy subjects;
iii. a respective copy number of cfDNA in a plurality of first bins across the genome, using the second plurality of sequencing results, of the test subject relative to a respective copy number of cfDNA in the plurality of first bins across the genome determined using a plurality of genome wide sequencing results of a plurality of liquid biopsies obtained from the cohort of healthy subjects, and
iv. a fragment size pattern distribution of cfDNA across the genome, using the second plurality of sequence results, of the test subject relative to a fragment size distribution of cfDNA determined using a plurality of genome sequencing results for a plurality of liquid biopsies obtained from a cohort of a healthy subject; and
e) responsive to inputting into a model each of the analyzed sequencing results from (d)(i)-(d)(iv), receiving as output from the model:
i. a categorical indication of a presence or absence of the cancer in the test subject, and
in the case where the model determines presence of the cancer in the test subject, an origin of the cancer,
wherein the detection of a cancer is indicative of efficacy of treatment and need of continuing, modifying or discontinuing treatment of the subject.
